(19) United States
(12) Patent Application Publication
(10) Pub. No.: US 2012/0148576 A1
(43) Pub. Date: Jun. 14, 2012

(54) HUMANIZED ANTI-CD 19 ANTIBODY FORMULATIONS

(75) Inventors: Monika S. Sharma, Gaithersburg, MD (US); Ambarish Shah, Gaithersburg, MD (US)

(73) Assignee: Medlmmune, LLC, Gaithersburg, MD (US)

(21) Appl. No.: 13/254,656

(22) PCT Filed: Mar. 8, 2010
(86) PCT No.: PCT/US10/26492

§ 371 Date: Feb. 22, 2012

Related U.S. Application Data
(60) Provisional application No. 61/158,153, filed on Mar. 6, 2009.

(51) Int. Cl. A61K 39/395; A61P 35/04; A61P 37/00

(52) U.S. Cl. 424/133.1; 424/142.1

(57) ABSTRACT

The present invention provides stable liquid formulations comprising chimeric and humanized versions of anti-CD 19 mouse monoclonal antibodies that may mediate ADCC, CDC, and/or apoptosis for the treatment of B cell diseases and disorders.

### 1. PRIORITY DATA

This application claims priority to U.S. Application Ser. No. 61/158,153, filed Mar. 6, 2009, which is hereby incorporated by reference in its entirety.

### SEQUENCE LISTING

The instant application contains a Sequence Listing which has been submitted via EFS-Web and is hereby incorporated by reference in its entirety. Said ASCII copy, created on Mar. 5, 2010, is named 00058000.txt, and is 79,932 bytes in size.

### 2. INTRODUCTION

The present invention relates to liquid formulations of human, humanized, or chimeric antibodies that specifically bind to the human CD19 antigen and may mediate one or more of the following: complement-dependent cell-mediated cytotoxicity (CDC), antigen-dependent cell-mediated-cytotoxicity (ADCC), and programmed cell death (apoptosis). Said formulations exhibit stability, low to undetectable levels of antibody fragmentation, low to undetectable levels of aggregation, and very little to no loss of the biological activities of the antibodies, even during long periods of storage.

The present invention is further directed to methods for the treatment of B cell disorders or diseases in human subjects, including B cell malignancies, utilizing liquid formulations comprising therapeutic human, humanized, or chimeric anti-CD19 antibodies that bind to the human CD19 antigen. The present invention is directed to methods for the treatment and prevention of autoimmune disease as well as the treatment and prevention of graft-versus-host disease (GVHD), humoral rejection, and post-transplantation lymphoproliferative disorder in human transplant recipients utilizing liquid formulations comprising therapeutic human, humanized, or chimeric anti-CD19 antibodies that bind to the human CD19 antigen.

### 3. BACKGROUND

B cells express a wide array of cell surface molecules during their differentiation and proliferation. Examples include the CD10, CD19, CD20, CD21, CD22, CD23, CD24, CD37, CD53, CD72, CD74, CD75, CD77, CD79a, CD79b, CD80, CD81, CD82, CD83, CD84, CD85, and CD86 leukocyte surface markers. These markers have been generally suggested as therapeutic targets for the treatment of B cell disorders or diseases such as B cell malignancies, autoimmune diseases, and transplant rejection. Antibodies that specifically bind them have been developed, and some have been tested as therapeutic agents for the treatment of diseases and disorders.

For example, chimeric or radiolabeled monoclonal antibody (mAb)-based therapies directed against the CD20 cell surface molecule specific for mature B cells and their malignant counterparts have been shown to be an effective in vivo treatment for non-Hodgkin's lymphoma (Tedder et al., Immunol. Today 15:450-454 (1994); Press et al., Hematology:221-240 (2001); Kaminski et al., N. Engl. J. Med. 329:459-465 (1993); Weiner, Semin. Oncol. 26:43-51 (1999); Onrust et al., Drugs 58:79-88 (1999); McLaughlin et al., Oncology 12:1763-1769 (1998); Reff et al., Blood 83:435-445 (1994); Maloney et al., Blood 90:2188-2195 (1997); Malone et al., J. Clin. Oncol. 15:3266-3274 (1997); Anderson et al., Biochem. Soc. Transac. 25:705-708 (1997)). Anti-CD20 monoclonal antibody therapy has also been found to be partially effective in attenuating the manifestations of rheumatoid arthritis, systemic lupus erythematosus, idiopathic thrombocytopenic purpura and hemolytic anemia, as well as other immune-mediated diseases (Silverman et al., Arthritis Rheum. 48:1484-1492 (2002); Edwards et al., Rheumatology 40:1-7 (2001); De Vita et al., Arthritis Rheumatism 46:2029-2033 (2002); Leandro et al., Ann. Rheum. Dis. 61:883-888 (2002); Leandro et al., Arthritis Rheum. 46:2673-2677 (2001)). The anti-CD20 (IgG1) antibody, RITUXAN, has successfully been used in the treatment of certain diseases such as adult immune thrombocytopenic purpura, rheumatoid arthritis, and autoimmune hemolytic anemia (Cured et al., WO 00/67796). Despite the effectiveness of these therapies, B cell depletion is less effective where B cells do not express CD20 or express CD20 at low levels, (e.g., on pre-B cells or immature B cells) or have lost CD20 expression following CD20 immunotherapy (Smith et al., Oncogene 22:7359-7368 (2003)).

Murine monoclonal anti-CD19 antibodies have been described in the art, for example, HD37 (IgG1, kappa) (DAKO North America, Inc, Carpinteria, Calif.), BU12 (Callard et al., J. Immunology, 148(10):2983-7 (1992)), 4G7 (IgG1) (Meeker et al., Hybridoma, 3(4):305-20 (1984 Winter)), J4.119 (Beckman Coulter, Krefeld, Germany), B43 (PharMingen, San Diego, Calif.), SJ25C1 (BD PharMingen, San Diego, Calif.), FMC63 (IgG2a) (Zola et al., Immunol Cell.Biol. 69(PT6): 411-22 (1991); Nicholson et al., Mol. Immunol., 34:1157-1165 (1997); Pietersz et al., Cancer Immunol. Immunotherapy, 41:53-60 (1995)), 89B(B4) (IgG1) (Beckman Coulter, Miami, Fla.; Nadler et al., J. Immunol., 131:244-250 (1983)), and/or HD237 (IgG2b) (Fourth International Workshop on Human Leukocyte Differentiation Antigens, Vienna, Austria, 1989; and Pezzutto et al., J. Immunol., 138(9):2793-2799 (1987)). Anti-CD19 antibodies or conjugates thereof have also shown therapeutic potential in various animal models of B cell disorders and diseases (Falvell et al., Br. J. Hematol. 134(2):157-70 (2006); Vallera et al., Clin. Cancer Res. 11(21):7920-8 (2005); Yazawa et al., Proc. Natl. Acad. Sci. USA 102(42):15178-83 (2005)).

In particular, the use of humanized CD19 antibodies has been described for the treatment of B-cell disease such as lymphoma, leukemia, or autoimmune disease (see, Hansen U.S. Patent Application Publication No. US2005/0070693).

Despite recent advances in cancer therapy, B cell malignancies, such as the B cell subtypes of non-Hodgkin's lymphomas, and chronic lymphocytic leukemia, are major contributors of cancer-related deaths. Accordingly, there is a great need for further, improved therapeutic regimens for the treatment of B cell malignancies.

Both cellular (T cell-mediated) and humoral (antibody, B cell-mediated) immunity are now known to play significant roles in graft rejection. While the importance of T cell-mediated immunity in graft rejection is well established, the critical role of humoral immunity in acute and chronic rejection has only recently become evident. Consequently, most of the advances in the treatment and prevention of graft rejection have developed from therapeutic agents that target T cell activation. The first therapeutic monoclonal antibody that was FDA approved for the treatment of graft rejection was the murine monoclonal antibody ORTHOCLONE-OKT3™ (muromonab-CD3), directed against the CD3 receptor of T cells. OKT3 has been joined by a number of other anti-lymphocyte directed antibodies, including the monoclonal anti-CD52 CAMPATH™ antibodies, CAMPATH-1G, CAMPATH-1H (alemtuzumab), and CAMPATH-1M), and polyclonal anti-thymocyte antibody preparations (referred to as anti-thymocyte globulin, or “ATG,” also called “thymoglobin” or “thymoglobulin”). Other T cell antibodies approved for the prevention of transplant rejection include the chimeric monoclonal antibody SIMULECT™ (basiliximab) and the humanized monoclonal antibody ZENAPAX™ (daclizumab), both of which target the high-affinity IL-2 receptor of activated T cells.

The importance of humoral immunity in graft rejection was initially thought to be limited to hyperacute rejection, in which the graft recipient possesses anti-donor HLA antibodies prior to transplantation, resulting in rapid destruction of the graft in the absence of an effective therapeutic regimen of antibody suppression. Recently, it has become evident that humoral immunity is also an important factor mediating both acute and chronic rejection. For example, clinical observations demonstrated that graft survival in patients capable of developing class I or class II anti-HLA alloantibodies (also referred to as “anti-MHC alloantibodies”) was reduced compared to graft survival in patients that could not develop such antibodies. Clinical and experimental data also indicate that other donor-specific alloantibodies and autoantibodies are critical mediators of rejection. For a current review of the evidence supporting a role for donor-specific antibodies in allograft rejection, see Rifle et al., Transplantation, 79:S14-S18 (2005). Thus, due to the relatively recent appreciation of the role of humoral immunity in acute and chronic graft rejection, current therapeutic agents and strategies for targeting humoral immunity are less well developed than those for targeting cellular immunity. Accordingly, there is a need in the art for improved reagents and methods for treating and preventing graft rejection, i.e. graft-versus-host disease (GVHD), humoral rejection, and post-transplantation lymphoproliferative disorder in human transplant recipients.

Autoimmune diseases as a whole cause significant morbidity and disability. Based on incidence data collected from 1965 to 1995, it has been estimated that approximately 1.2 million persons will develop a new autoimmune disease over the next five years. Jacobsen et al. (Clin Immunol. Immunopathol. 84:223 (1997)) evaluated over 130 published studies and estimated that in 1996, 8.5 million people in the United States (3.2% of the population) had at least one of the 24 autoimmune diseases examined in these studies. Considering the major impact of autoimmune diseases on public health, effective and safe treatments are needed to address the burden of these disorders. Thus, there is a need in the art for improved reagents and methods for treating autoimmune disease.

Prior liquid antibody preparations have short shelf lives and may lose biological activity of the antibodies resulting from chemical and physical instabilities during the storage. Chemical instability may be caused by deamidation, racemization, hydrolysis, oxidation, beta elimination or disulfide exchange, and physical instability may be caused by antibody denaturation, aggregation, precipitation or adsorption. Among those, aggregation, deamidation and oxidation are known to be the most common causes of the antibody degradation (Wang et al., 1988, J. of Parenteral Science & Technology 42(Suppl):S4-S26; Cleland et al., 1993, Critical Reviews in Therapeutic Drug Carrier Systems 10(4):307-377). Thus, there is a need for a stable liquid formulation of antibodies, in particular, stable liquid formulation of anti-human CD19 antibodies.

### 4. SUMMARY

The present invention provides sterile, stable aqueous formulations comprising a chimeric, human, or humanized antibody that specifically binds the human CD19 antigen. In one embodiment, the present invention provides formulations comprising chimeric and humanized versions of anti-CD19 mouse monoclonal antibodies, HB12A and HB12B. In another embodiment, the present invention provides formulations comprising an anti-CD19 antibodies described in U.S. patent application Ser. No. 11/852,106 filed on Sep. 7, 2007, the disclosure of which is incorporated herein in its entirety for all purposes. In a further embodiment, the present invention provides a formulation comprising an anti-CD19 antibody of the invention that may mediate one or more of the following: complement-dependent cell-mediated cytotoxicity (CDC), antigen-dependent cell-mediated-cytotoxicity (ADCC), and programmed cell death (apoptosis) and has enhanced effector functions. In another embodiment, a formulation of the invention comprises an anti-CD19 antibody of the invention comprising an Fc region having complex N-glycoside-linked sugar chains in which fucose is not bound to N-acetylglucosamine in the reducing end in the sugar chain. In a specific embodiment, a formulation of the invention comprises an anti-CD19 antibody comprising a heavy chain variable region of SEQ ID NO:104, a light chain variable sequence of SEQ ID NO:111 and an Fc region having complex N-glycoside-linked sugar chains in which fucose is not bound to N-acetylglucosamine in the reducing end in the sugar chain.

The present invention provides methods of stabilizing a chimeric, human, or humanized anti-CD19 antibodies of the invention.

The present invention further relates to processes of making a sterile, stable aqueous formulation comprising chimeric, human, or humanized antibodies of the invention that specifically bind the human CD19 antigen.

The present invention is further directed to methods for the treatment of B cell disorders or diseases in human subjects, including B cell malignancies, utilizing liquid formulations comprising therapeutic human, humanized, or chimeric anti-CD19 antibodies of the invention that bind to the human CD19 antigen. The present invention is directed to methods for the treatment and prevention of autoimmune disease as well as the treatment and prevention of graft-versus-host disease (GVHD), humoral rejection, and post-transplantation lymphoproliferative disorder in human transplant recipients utilizing liquid formulations comprising therapeutic human, humanized, or chimeric anti-CD19 antibodies of the invention that bind to the human CD19 antigen.

The present invention relates to human, humanized, or chimeric anti-CD19 antibodies that bind to the human CD19 antigen, as well as to compositions comprising those antibodies. In one embodiment, the present invention provides chimeric and humanized versions of anti-CD19 mouse monoclonal antibodies, HB12A and HB12B.

In another embodiment, anti-CD19 antibodies of the invention comprise one, two, three, four, five, or all six of the CDRs of HB12A (clone B410F12-2-A6-C2 was deposited with the American Type Culture Collection (“ATCC”) on Feb. 11, 2005, ATCC Patent Deposit Designation: PTA-6580) or HB12B (clone B43H12-3-B2-B6 was deposited with the American Type Culture Collection (“ATCC”) on Feb. 11, 2005, ATCC Patent Deposit Designation: PTA-6581).

The amino acid sequences for CDR1, CDR2, and CDR3 of the heavy chain variable region of HB12A defined according to Kabat (see, Kabat et al., Sequences of Proteins of Immunological Interest, 5th Ed. Public Health Service, National Institutes of Health, Bethesda, Md. (1991) are identified as SEQ ID NO:6, SEQ ID NO:8, and SEQ ID NO:10, respectively. The amino acid sequences for CDR1, CDR2 and CDR3 of the light chain variable region of HB12A defined according to Kabat are identified as SEQ ID NO:12, SEQ ID NO:14, and SEQ ID NO:16, respectively.

The amino acid sequences for CDR1, CDR2, and CDR3 of the heavy chain variable region of HB12B defined according to Kabat are identified as SEQ ID NO:22, SEQ ID NO:24, and SEQ ID NO:26, respectively. The amino acid sequences for CDR1, CDR2 and CDR3 of the light chain variable region of HB12B defined according to Kabat are identified as SEQ ID NO:28, SEQ ID NO:30, and SEQ ID NO:32, respectively.

In one embodiment, an anti-CD19 antibody of the invention comprises one, two, three, four, five, or six CDRs having the amino acid sequence of a CDR listed in Table 1, infra.

TABLE 1
Residues that are different from the amino acid sequence of the corresponding HB12B
parental CDR appear in bold, underlined. Amino acid residues corresponding to a given
variable position within the consensus CDR sequences (SEQ ID NO.: 230-235) are listed
in parenthesis. In specific embodiments, a CDR of the invention may comprise any
permutation of the individual amino acid residues corresponding to variable positions
within the CDR.

| Antibody | VH | VH | VH | VH | VK | VK | VK | VK |
|---|---|---|---|---|---|---|---|---|
| Name | Domain | CDR1 | CDR2 | CDR3 | Domain | CDR1 | CDR2 | CDR3 |
| HB12A | SEQ. ID | SYVMH | YFNPYNDG | GTYYYGSS | SEQ. ID | KSSQSLLYS | LVSKLDS | VQGTHFPY |
| | NO.: 2 | (SEQ. ID | TDYYEKFK | YPFDY | NO.: 4 | NGKTYLN | (SEQ. ID | T |
| | | NO.: 6) | G | (SEQ. ID | | (SEQ. ID | NO.: 14) | (SEQ. ID |
| | | | (SEQ. ID | NO.: 10) | | NO.: 12) | | NO.: 16) |
| | | | NO.: 8) | | | | | |
| HB12B | SEQ. ID | SSWMN | RIYPGDGDT | SGFITTVLD | SEQ. ID | RASESVDTF | AASNQGS | QQSKEVPFT |
| | NO.: 18 | (SEQ. ID | NYNGKFKG | FDY | NO.: 20 | GISFMN | (SEQ. ID | (SEQ. ID |
| | | NO.: 22) | (SEQ. ID | (SEQ. ID | | (SEQ. ID | NO.: 30) | NO.: 32) |
| | | | NO.: 24) | NO.: 26) | | NO.: 28) | | |
| 3649 | SEQ. ID | SSWMN | RIYPGDGDT | SGFITTVLD | SEQ. ID | RASESVDTF | AASNQGS | QQSKEVPFT |
| | NO.: 34 | (SEQ. ID | NYNGKFKG | FDY | NO.: 68 | GISFMN | (SEQ. ID | (SEQ. ID |
| | | NO.: 22) | (SEQ. ID | (SEQ. ID | | (SEQ. ID | NO.: 30) | NO.: 32) |
| | | | NO.: 24) | NO.: 26) | | NO.: 28) | | |
| 7E12 | SEQ. ID | STWMN | RIYPGDGDT | SGFITTVYD | SEQ. ID | RASESVDTF | EASNQGS | QQTKEVPFT |
| | NO.: 102 | (SEQ. ID | NYNGKFKG | FDY | NO.: 110 | GISFIN | (SEQ. ID | (SEQ. ID |
| | | NO.: 114) | (SEQ. ID | (SEQ. ID | | (SEQ. ID | NO.: 125) | NO.: 126) |
| | | | NO.: 24) | NO.: 120) | | NO.: 123) | | |
| 14H5 | SEQ. ID | SSWMN | RIYPGDGDT | SGFITTVRD | SEQ. ID | RASESVDTF | EASNQGS | QQSKEVPFT |
| | NO.: 103 | (SEQ. ID | NYNGKFKG | FDY | NO.: 111 | GISFMN | (SEQ. ID | (SEQ. ID |
| | | NO.: 22) | (SEQ. ID | (SEQ. ID | | (SEQ. ID | NO.: 125) | NO.: 32) |
| | | | NO.: 24) | NO.: 121) | | NO.: 28) | | |
| 16C9 | SEQ. ID | SSWMN | RIYPGDGDT | SGFITTVRD | SEQ. ID | RASESVDTF | AASNQGS | QQSKEVPIT |
| | NO.: 103 | (SEQ. ID | NYNGKFKG | FDY | NO.: 113 | GISFMN | (SEQ. ID | (SEQ. ID |
| | | NO.: 22) | (SEQ. ID | (SEQ. ID | | (SEQ. ID | NO.: 30) | NO.: 127) |
| | | | NO.: 24) | NO.: 121) | | NO.: 28) | | |
| 15D1 | SEQ. ID | SSWMN | RIYPGDGDT | SGFITTVRD | SEQ. ID | RASESVDHF | EASNQGS | QQSKEVPIT |
| | NO.: 104 | (SEQ. ID | NYNAKFKG | FDY | NO.: 112 | GISFMN | (SEQ. ID | (SEQ. ID |
| | | NO.: 22) | (SEQ. ID | (SEQ. ID | | (SEQ. ID | NO.: 125) | NO.: 127) |
| | | | NO.: 115) | NO.: 121) | | NO.: 124) | | |
| 15D7 | SEQ. ID | SSWMN | RIYPGDGDT | SGFITTVHD | SEQ. ID | RASESVDTF | EASNQGS | QQSKEVPFT |
| | NO.: 105 | (SEQ. ID | NYNGKFKG | FDY | NO.: 111 | GISFMN | (SEQ. ID | (SEQ. ID |
| | | NO.: 22) | (SEQ. ID | (SEQ. ID | | (SEQ. ID | NO.: 125) | NO.: 32) |
| | | | NO.: 24) | NO.: 122) | | NO.: 28) | | |
| 16C4 | SEQ. ID | SSWMN | RIYPGDGDT | SGFITTVRD | SEQ. ID | RASESVDTF | EASNQGS | QQSKEVPFT |
| | NO.: 106 | (SEQ. ID | NYNVKFKG | FDY | NO.: 111 | GISFMN | (SEQ. ID | (SEQ. ID |
| | | NO.: 22) | (SEQ. ID | (SEQ. ID | | (SEQ. ID | NO.: 125) | NO.: 32) |
| | | | NO.:116) | NO.: 121) | | NO.: 28) | | |
| 14H5-YG | SEQ. ID | SSWMN | RIYPGDGDT | SGFITTVRD | SEQ. ID | RASESVDTF | EASNQGS | QQSKEVPFT |
| | NO.: 107 | (SEQ. ID | NYYGKFKG | FDY | NO.: 111 | GISFMN | (SEQ. ID | (SEQ. ID |
| | | NO.: 22) | (SEQ. ID | (SEQ. ID | | (SEQ. ID | NO.: 125) | NO.: 32) |
| | | | NO.: 117) | NO.: 121) | | NO.: 28) | | |
| 14H5-DG | SEQ. ID | SSWMN | RIYPGDGDT | SGFITTVRD | SEQ. ID | RASESVDTF | EASNQGS | QQSKEVPFT |
| | NO.: 108 | (SEQ. ID | NYDGKFKG | FDY | NO.: 111 | GISFMN | (SEQ. ID | (SEQ. ID |
| | | NO.: 22) | (SEQ. ID | (SEQ. ID | | (SEQ. ID | NO.: 125) | NO.: 32) |
| | | | NO.: 118) | NO.: 121) | | NO.: 28) | | |
| 14H5-LG | SEQ. ID | SSWMN | RIYPGDGDT | SGFITTVRD | SEQ. ID | RASESVDTF | EASNQGS | QQSKEVPFT |
| | NO.: 109 | (SEQ. ID | NYLGKFKG | FDY | NO.: 111 | GISFMN | (SEQ. ID | (SEQ. ID |
| | | NO.: 22) | (SEQ. ID | (SEQ. ID | | (SEQ. ID | NO.: 125) | NO.: 32) |
| | | | NO.: 119) | NO.: 121) | | NO.: 28) | | |
| 1A7 | SEQ. ID | SVWMN | RIYPGDGDT | SGFITTVRD | SEQ. ID | RASESVDTF | EASNQGS | QQSKEVPFT |
| | NO.: 191 | (SEQ. ID | NYNVKFKG | FDY | NO.: 111 | GISFMN | (SEQ. ID | (SEQ. ID |
| | | NO.: 208) | (SEQ. ID | (SEQ. ID | | (SEQ. ID | NO.: 125) | NO.: 32) |
| | | | NO.: 116) | NO.: 121) | | NO.: 28) | | |
| 3C3 | SEQ. ID | SVWMN | RIYPGDGDT | SGFITTVRD | SEQ. ID | RASESVDHF | EASNPYS | AQSKEVPIT |
| | NO.: 191 | (SEQ. ID | NYNVKFKG | FDY | NO.: 193 | GISFIN | (SEQ. ID | (SEQ. ID |
| | | NO.: 208) | (SEQ. ID | (SEQ. ID | | (SEQ. ID | NO.: 218) | NO.: 222) |
| | | | NO.: 116) | NO.: 121) | | NO.: 211) | | |
| 3E5 | SEQ. ID | SVWMN | RIYPGDGDT | SGFITTVRD | SEQ. ID | RASESVDTF | EASNQGS | AQSKEVPFT |
| | NO.: 191 | (SEQ. ID | NYNVKFKG | FDY | NO.: 194 | GISFMN | (SEQ. ID | (SEQ. ID |
| | | NO.: 208) | (SEQ. ID | (SEQ. ID | | (SEQ. ID | NO.: 125) | NO.: 223) |
| | | | NO.: 116) | NO.: 121) | | NO.: 28) | | |
| 3D4 | SEQ. ID | SVWMN | RIYPGDGDT | SGFITTVRD | SEQ. ID | RASESVDTF | EASNQGS | AQSKRVPFT |
| | NO.: 191 | (SEQ. ID | NYNVKFKG | FDY | NO.: 195 | GISFMN | (SEQ. ID | (SEQ. ID |
| | | NO.: 208) | (SEQ. ID | (SEQ. ID | | (SEQ. ID | NO.: 125) | NO.: 224) |
| | | | NO.: 116) | NO.: 121) | | NO.: 28) | | |
| 3F11 | SEQ. ID | SVWMN | RIYLGDGDT | SGFITTVRD | SEQ. ID | RASESVITF | EASNQGS | QQSKEVPFT |
| | NO.: 192 | (SEQ. ID | NYNVKFKG | FDY | NO.: 196 | GISFMN | (SEQ. ID | (SEQ. ID |
| | | NO.: 208) | (SEQ. ID | (SEQ. ID | | (SEQ. ID | NO.: 125) | NO.: 32) |
| | | | NO.: 210) | NO.: 121) | | NO.: 212) | | |
| 5B5 | SEQ. ID | SVWMN | RIYPGDGDT | SGFITTVRD | SEQ. ID | RASESVDTF | EASNQGS | AQTKRVPF |
| | NO.: 191 | (SEQ. ID | NYNVKFKG | FDY | NO.: 197 | GISFMN | (SEQ. ID | T |
| | | NO.: 208) | (SEQ. ID | (SEQ. ID | | (SEQ. ID | NO.: 125) | (SEQ. ID |
| | | | NO.: 116) | NO.: 121) | | NO.: 28) | | NO.: 225) |
| 6F7 | SEQ. ID | SVWMN | RIYPGDGDT | SGFITTVRD | SEQ. ID | RASESVDTF | EASNQGS | QQSKEVPIT |
| | NO.: 191 | (SEQ. ID | NYNVKFKG | FDY | NO.: 198 | GISFMN | (SEQ. ID | (SEQ. ID |
| | | NO.: 208) | (SEQ. ID | (SEQ. ID | | (SEQ. ID | NO.: 125) | NO.: 226) |
| | | | NO.: 116) | NO.: 121) | | NO.: 28) | | |
| 1C11 | SEQ. ID | SSWMN | RIYPGDGDT | SGFITTVRD | SEQ. ID | RASESVDTF | EASNPYS | QQSKEVPFT |
| | NO.: 106 | (SEQ. ID | NYNVKFKG | FDY | NO.: 199 | GISFIN | (SEQ. ID | (SEQ. ID |
| | | NO.: 22) | (SEQ. ID | (SEQ. ID | | (SEQ. ID | NO.: 218) | NO.: 32) |
| | | | NO.: 116) | NO.: 121) | | NO.: 213) | | |
| 2B11 | SEQ. ID | SSWMN | RIYPGDGDT | SGFITTVRD | SEQ. ID | RASESVDTF | EASNQGS | AQTKEVPFT |
| | NO.: 106 | (SEQ. ID | NYNVKFKG | FDY | NO.: 200 | GISFMN | (SEQ. ID | (SEQ. ID |
| | | NO.: 22) | (SEQ. ID | (SEQ. ID | | (SEQ. ID | NO.: 125) | NO.: 227) |
| | | | NO.: 116) | NO.: 121) | | NO.: 28) | | |
| 2D10 | SEQ. ID | SVWMN | RIYPGDGDT | SGFITTVRD | SEQ. ID | RASESVDTF | EASNQGS | AQTKEVPN |
| | NO.: 191 | (SEQ. ID | NYNVKFKG | FDY | NO.: 201 | GISFMN | (SEQ. ID | T |
| | | NO.: 208) | (SEQ. ID | (SEQ. ID | | (SEQ. ID | NO.: 125) | (SEQ. ID |
| | | | NO.: 116) | NO.: 121) | | NO.: 28) | | NO.: 228) |
| 3B4 | SEQ. ID | STWMN | RIYPGDGDT | SGFITTVRD | SEQ. ID | RASESVDTF | EASNQGS | QQSKEVPFT |
| | NO.: 236 | (SEQ. ID | NYNVKFKG | FDY | NO.: 111 | GISFMN | (SEQ. ID | (SEQ. ID |
| | | NO.: 209) | (SEQ. ID | (SEQ. ID | | (SEQ. ID | NO.: 125) | NO.: 32) |
| | | | NO.: 116) | NO.: 121) | | NO.: 28) | | |
| 5C11 | SEQ. ID | SVWMN | RIYPGDGDT | SGFITTVRD | SEQ. ID | RASESVITF | EASNTYS | AQSKRVPFT |
| | NO.: 191 | (SEQ. ID | NYNVKFKG | FDY | NO.: 202 | GISFMN | (SEQ. ID | (SEQ. ID |
| | | NO.: 208) | (SEQ. ID | (SEQ. ID | | (SEQ. ID | NO.: 219) | NO.: 224) |
| | | | NO.: 116) | NO.: 121) | | NO.: 212) | | |
| 5D4 | SEQ. ID | SSWMN | RIYPGDGDT | SGFITTVRD | SEQ. ID | RASESVDTF | EASNQGS | QQSKEVPFT |
| | NO.: 106 | (SEQ. ID | NYNVKFKG | FDY | NO.: 203 | GISFRN | (SEQ. ID | (SEQ. ID |
| | | NO.: 22) | (SEQ. ID | (SEQ. ID | | (SEQ. ID | NO.: 125) | NO.: 32) |
| | | | NO.: 116) | NO.: 121) | | NO.: 214) | | |
| 6C2 | SEQ. ID | SSWMN | RIYPGDGDT | SGFITTVRD | SEQ. ID | RASESVDTF | EASNQGS | QQSKEVPIT |
| | NO.: 106 | (SEQ. ID | NYNVKFKG | FDY | NO.: 198 | GISFMN | (SEQ. ID | (SEQ. ID |
| | | NO.: 22) | (SEQ. ID | (SEQ. ID | | (SEQ. ID | NO.: 125) | NO.: 226) |
| | | | NO.: 116) | NO.: 121) | | NO.: 28) | | |
| 6C11 | SEQ. ID | SVWMN | RIYLGDGDT | SGFITTVRD | SEQ. ID | RASESVDTF | EASNPGS | QQTKRVPF |
| | NO.: 192 | (SEQ. ID | NYNVKFKG | FDY | NO.: 204 | GISFMN | (SEQ. ID | T |
| | | NO.: 208) | (SEQ. ID | (SEQ. ID | | (SEQ. ID | NO.: 220) | (SEQ. ID |
| | | | NO.: 210) | NO.: 121) | | NO.: 28) | | NO.: 229) |
| 9G7 | SEQ. ID | SVWMN | RIYPGDGDT | SGFITTVRD | SEQ. ID | RASESVIHF | EASNRGS | AQSKEVPIT |
| | NO.: 191 | (SEQ. ID | NYNVKFKG | FDY | NO.: 205 | GISFMN | (SEQ. ID | (SEQ. ID |
| | | NO.: 208) | (SEQ. ID | (SEQ. ID | | (SEQ. ID | NO.: 221) | NO.: 222) |
| | | | NO.: 116) | NO.: 121) | | NO.: 215) | | |
| 1H4 | SEQ. ID | SVWMN | RIYPGDGDT | SGFITTVRD | SEQ. ID | RASESVDTF | EASNPYS | QQSKEVPFT |
| | NO.: 191 | (SEQ. ID | NYNVKFKG | FDY | NO.: 206 | GLSFMN | (SEQ. ID | (SEQ. ID |
| | | NO.: 208) | (SEQ. ID | (SEQ. ID | | (SEQ. ID | NO.: 218) | NO.: 32) |
| | | | NO.: 116) | NO.: 121) | | NO.: 216) | | |
| 3C6 | SEQ. ID | SVWMN | RIYPGDGDT | SGFITTVRD | SEQ. ID | RASESVDTF | EASNQGS | AQTKRVPF |
| | NO.: 191 | (SEQ. ID | NYNVKFKG | FDY | NO.: 197 | GISFMN | (SEQ. ID | T |
| | | NO.: 208) | (SEQ. ID | (SEQ. ID | | (SEQ. ID | NO.: 125) | (SEQ. ID |
| | | | NO.: 116) | NO.: 121) | | NO.: 28) | | NO.: 225) |
| 5C4 | SEQ. ID | SVWMN | RIYPGDGDT | SGFITTVRD | SEQ. ID | RASESVITF | EASNPYS | AQTKRVPF |
| | NO.: 191 | (SEQ. ID | NYNVKFKG | FDY | NO.: 207 | GISFIN | (SEQ. ID | T |
| | | NO.: 208) | (SEQ. ID | (SEQ. ID | | (SEQ. ID | NO.: 218) | (SEQ. ID |
| | | | NO.:116) | NO.: 121) | | NO.: 217) | | NO.: 225) |
| Consensus | SEQ ID | S(S/T/V)WM | RIY(P/L)GD | SGFITTV(R/ | SEQ ID | RASESV(I/D) | (E/A)ASN(P/ | (Q/A)Q(S/T) |
| | NO.: 237 | N | GDTNY(N/Y/ | L/Y/H)DFDY | NO.: 238 | (T/H)FG(I/L) | Q/T)(Y/G)S | K(E/R)VP(F/I/ |
| | | (SEQ. ID | D/L)(G/A/V) | (SEQ. ID | | SF(I/M/R)N | (SEQ. ID | N)T |
| | | NO.: 230) | KFKG | NO.: 232) | | (SEQ. ID | NO.: 234) | (SEQ. ID |
| | | | (SEQ. ID | | | NO.: 233) | | NO.: 235) |
| | | | NO.: 231) | | | | | |

In one embodiment, an anti-CD19 antibody of the invention may comprise one or more framework regions of HB12A or HB12B. In one embodiment, an antibody of the invention may further comprise heavy and/or light chain framework (FW) regions from a human antibody (e.g., from a human germline antibody sequence such as VH3-72, JH4, Vk A10, or Jk4), wherein said human framework regions may comprise one or more mutations in which a human FW residue is exchanged for the corresponding residue present in the parental mouse (e.g., HB12A or HB12B) heavy or light chain.

In one embodiment, an anti-CD19 antibody of the invention may comprise one or more CDRs having the amino acid sequence of a CDR listed in Table 1. supra and may further comprise one or more heavy chain framework (FW) regions of the VH region designated HB12B-(3-72/JH4) (SEQ ID NO:34). In another embodiment, an anti-CD19 antibody of the invention comprises one or more CDRs having the amino acid sequence of a CDR listed in Table 1. supra and further comprises one or more heavy chain framework (FW) regions of the VH region designated HB12B-(3-72/JH4) (SEQ ID NO:34). In one embodiment, an anti-CD19 antibody of the invention may comprise one or more CDRs having the amino acid sequence of a CDR listed in Table 1. supra and may further comprise one or more light chain framework (FW) regions of the VK region designated HB12B-(A10-Jk4) (SEQ ID NO:52). In one embodiment, an anti-CD19 antibody of the invention comprises one or more CDRs having the amino acid sequence of a CDR listed in Table 1. supra and further comprises one or more light chain framework (FW) regions of the VK region designated HB12B-(A10-Jk4) (SEQ ID NO:52). In another embodiment, an anti-CD19 antibody described herein may comprise one or more CDRs having the amino acid sequence of a CDR listed in Table 1. supra, one or more light chain framework regions of the VK region designated HB12B-(A10-Jk4), and one or more heavy chain framework regions of the VH region designated HB12B-(3-72/JH4). In a further embodiment, an anti-CD19 antibody described herein comprises one or more CDRs having the amino acid sequence of a CDR listed in Table 1. supra, one or more light chain framework regions of the VK region designated HB12B-(A10-Jk4), and one or more heavy chain framework regions of the VH region designated HB12B-(3-72/JH4).

For instance, in one embodiment a humanized anti-CD19 antibody of the invention may comprise a heavy chain variable region which comprises four framework regions, FW1, FW2, FW3, and FW4, wherein FW1 comprises the amino acid sequence of SEQ ID NO:36, FW2 comprises the amino acid sequence of SEQ ID NO:38, FW3 comprises the amino acid sequence of SEQ ID NO:40, and FW4 comprises the amino acid sequence of SEQ ID NO:42. In one embodiment, a humanized anti-CD19 antibody of the invention comprises a heavy chain variable region which comprises four framework regions, FW1, FW2, FW3, and FW4, wherein FW1 comprises the amino acid sequence of SEQ ID NO:36, FW2 comprises the amino acid sequence of SEQ ID NO:38, FW3 comprises the amino acid sequence of SEQ ID NO:40, and FW4 comprises the amino acid sequence of SEQ ID NO:42.

In addition, a humanized anti-CD19 monoclonal antibody of the invention may comprise a light chain variable region comprising four framework regions, FW1, FW2, FW3, and FW4, wherein FW1 comprises the amino acid sequence of SEQ ID NO:54; those in which FW2 comprises an amino acid sequence selected from the group consisting of SEQ ID NO:56, SEQ ID NO:64, and SEQ ID NO:72; those in which FW3 comprises an amino acid sequence selected from the group consisting of SEQ ID NO:58, and SEQ ID NO:66; and those in which FW4 comprises the amino acid sequence of SEQ ID NO:60. In one embodiment, a humanized anti-CD19 monoclonal antibody of the invention comprises a light chain variable region comprising four framework regions, FW1, FW2, FW3, and FW4, wherein FW1 comprises the amino acid sequence of SEQ ID NO:54; those in which FW2 comprises an amino acid sequence selected from the group consisting of SEQ ID NO:56, SEQ ID NO:64, and SEQ ID NO:72; those in which FW3 comprises an amino acid sequence selected from the group consisting of SEQ ID NO:58, and SEQ ID NO:66; and those in which FW4 comprises the amino acid sequence of SEQ ID NO:60.

In one embodiment, an anti-CD19 antibody of the invention may comprise a VH comprising the amino acid sequence of SEQ ID NO.:237 or a VL comprising the amino acid sequence of SEQ ID NO.:238, wherein said antibody binds a human CD19 antigen. In another embodiment, an anti-CD19 antibody of the invention comprises a VH comprising the amino acid sequence of SEQ ID NO.:237 and a VL comprising the amino acid sequence of SEQ ID NO.:238.

In particular embodiments, an anti-CD19 antibody of the invention may comprise a light chain variable region selected from the group consisting of HB12B VK (SEQ ID NO:20), HB12B-(A10-Jk4) (SEQ ID NO:52), HB12B-364987 (SEQ ID NO:62), HB12B-3649 (SEQ ID NO:68), HB12B-36 (SEQ ID NO:70), HB12A VK (SEQ ID NO:4), 7E12 VK (SEQ ID NO:110), 14H5 VK (SEQ ID NO:111), 15D1 VK (SEQ ID NO:112), 16C9 VK (SEQ ID NO:113), 3C3 VK (SEQ ID NO:193), 3E5 VK (SEQ ID NO:194), 3D4 VK (SEQ ID NO:195), 3F1 VK (SEQ ID NO:196), 5B5 VK (SEQ ID NO:197), 6F7 VK (SEQ ID NO:198), 1C11 VK (SEQ ID NO:199), 2B11 VK (SEQ ID NO:200), 2D10 VK (SEQ ID NO:201), 5C11 VK (SEQ ID NO:202), 5D4 VK (SEQ ID NO:203), 6C11 VK (SEQ ID NO:204), 9G7 VK (SEQ ID NO:205), 1H4 VK (SEQ ID NO:206), and 5C4 VK (SEQ ID NO:207.

In specific embodiments, the present invention further relates to an anti-CD19 antibody comprising a heavy chain variable region selected from the group consisting of HB12B VH (SEQ ID NO:18), HB12B-(3-72/JH4) (SEQ ID NO:34), HB12A VH (SEQ ID NO:2), 7E12 VH (SEQ ID NO:102), 14H5 VH (SEQ ID NO:103), 15D1 VH (SEQ ID NO:104), 15D7 VH (SEQ ID NO:105), 16C4 VH (SEQ ID NO:106), 14H5-YG (SEQ ID NO:107), 14H5-DG (SEQ ID NO:108), 14H5-LG (SEQ ID NO:109), 1A7 VH (SEQ ID NO:191), 3C3 VH (SEQ ID NO:191), 6C11 VH (SEQ ID NO:191), 9G7 (SEQ ID NO:191), 3B4 VH (SEQ ID NO:236), and 3F11 VH (SEQ ID NO:192).

In a particular embodiment, an anti-CD19 antibody of the invention comprises the HB12B-3649 (SEQ ID NO:68) light chain variable region and the HB12B-(3-72/JH4) (SEQ ID NO:34) heavy chain variable region. A DNA clone comprising the humanized anti-hCD19 VH HB12B-(3-72/JH4) was deposited with the American Type Culture Collection (“ATCC”) on Oct. 26, 2006; “3649 Heavy in TOPO” ATCC Patent Deposit Designation PTA-7952. A DNA clone comprising the humanized anti-hCD19 VK HB12B-3649 was deposited with the American Type Culture Collection (“ATCC”) on Oct. 26, 2006; “3649 Light in TOPO” ATCC Patent Deposit Designation PTA-7953.

In one embodiment, a humanized anti-CD19 antibody of the invention may bind to human CD19 with an affinity comparable to that of the mouse monoclonal antibodies HB12A and/or HB12B, or with an affinity comparable to that of the chHB12B antibody comprising HB12B VH (SEQ ID NO:18) and HB12B VK (SEQ ID NO:20).

The invention further provides polynucleotides comprising a nucleotide sequence encoding a human, humanized, or chimeric anti-CD19 antibody of the invention or fragments thereof. The invention also encompasses polynucleotides that hybridize under stringent or lower stringency hybridization conditions, as defined herein, to polynucleotides that encode a human, humanized, or chimeric antibody that specifically binds to human CD19.

Another embodiment of the invention is a vector comprising one or more nucleotide sequences encoding a human, humanized, or chimeric anti-CD19 antibody described herein or fragments thereof.

The present invention further relates to an isolated cell comprising a vector wherein said vector comprises one or more nucleotide sequences encoding a human, humanized, or chimeric anti-CD19 antibody of the invention or fragments thereof.

Chimeric, human, and humanized anti-CD19 monoclonal antibodies described herein include those of the IgG1, IgG2, IgG3, or IgG4 human isotype.

In one embodiment, a humanized anti-CD19 antibody described herein mediates antibody-dependent cellular cytotoxicity (ADCC), complement-dependent cell-mediated cytotoxicity (CDC), and/or apoptosis.

In a further embodiment, a humanized anti-CD19 antibody described herein inhibits anti-IgM/CpG stimulated B cell proliferation.

The present invention further relates to pharmaceutical compositions comprising a chimeric, human, and humanized anti-CD19 antibody.

In still another other aspect, the present invention is directed toward a method of treating a B cell malignancy in a human, comprising administering to a human in need thereof a therapeutically-effective amount of a chimeric, human, or humanized anti-CD19 monoclonal antibody.

In a further aspect, the present invention relates to a method of treating an autoimmune disease or disorder in a human, comprising administering to a human in need thereof a therapeutically-effective amount of a chimeric, human, or humanized anti-CD19 monoclonal antibody.

The present invention further relates to a method of treating or preventing humoral rejection in a human transplant patient, comprising administering to a human in need thereof a therapeutically-effective amount of a chimeric, human, or humanized anti-CD19 monoclonal antibody.

The present invention relates to a method of enhancing the storage stability of an aqueous formulation comprising a chimeric, humanized or human anti-CD19 antibody, wherein said antibody comprises a heavy chain variable region comprising the sequence of SEQ ID NO:104, a light chain variable region comprising the sequence of SEQ ID NO:111 and an Fc region having complex N-glycoside-linked sugar chains in which fucose is not bound to N-acetylglucosamine in the reducing end in the sugar chain, comprising adding a surfactant to said formulation in an amount effective to enhance the storage stability of the formulation. In accordance with this aspect of the invention, the surfactant may be polysorbate 20 or polysorbate 80. In one embodiment, the surfactant is polysorbate 80 and is present in an amount of about 0.01%, about 0.02%, about 0.04%, about 0.08% or about 0.1%. Moreover, the invention further relates to additional sterilization of the formulation for storage stability, including filtering the composition prior to and/or after the addition of the surfactant.

### 4.1. Definitions

As used herein, the terms “antibody” and “antibodies” (immunoglobulins) encompass monoclonal antibodies (including full-length monoclonal antibodies), polyclonal antibodies, multispecific antibodies (e.g., bispecific antibodies) formed from at least two intact antibodies, human antibodies, humanized antibodies, camelised antibodies, chimeric antibodies, single-chain Fvs (scFv), single-chain antibodies, single domain antibodies, domain antibodies, Fab fragments, F(ab′)2 fragments, antibody fragments that exhibit the desired biological activity, disulfide-linked Fvs (sdFv), and anti-idiotypic (anti-Id) antibodies (including, e.g., anti-Id antibodies to antibodies of the invention), intrabodies, and epitope-binding fragments of any of the above including but not limited to Fab, Fab′, F(ab′)₂, and Fv fragments; diabodies; linear antibodies; single-chain antibody molecules; and multispecific antibodies formed from antibody fragments. In particular, antibodies include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

Native antibodies are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Light chains are classified as either lambda chains or kappa chains based on the amino acid sequence of the light chain constant region. The variable domain of a kappa light chain may also be denoted herein as VK. The term “variable region” may also be used to describe the variable domain of a heavy chain or light chain. Particular amino acid residues are believed to form an interface between the light and heavy chain variable domains. Such antibodies may be derived from any mammal, including, but not limited to, humans, monkeys, pigs, horses, rabbits, dogs, cats, mice, etc.

The term “variable” refers to the fact that certain portions of the variable domains differ extensively in sequence among antibodies and are responsible for the binding specificity of each particular antibody for its particular antigen. However, the variability is not evenly distributed through the variable domains of antibodies. It is concentrated in segments called Complementarity Determining Regions (CDRs) both in the light chain and the heavy chain variable domains. The more highly conserved portions of the variable domains are called the framework regions (FW). The variable domains of native heavy and light chains each comprise four FW regions, largely adopting a β-sheet configuration, connected by three CDRs, which form loops connecting, and in some cases forming part of, the β-sheet structure. The CDRs in each chain are held together in close proximity by the FW regions and, with the CDRs from the other chain, contribute to the formation of the antigen-binding site of antibodies (see, Kabat et al., Sequences of Proteins of Immunological Interest, 5th Ed. Public Health Service, National Institutes of Health, Bethesda, Md. (1991)). The constant domains are generally not involved directly in antigen binding, but may influence antigen binding affinity and may exhibit various effector functions, such as participation of the antibody in ADCC, CDC, and/or apoptosis.

The term “hypervariable region” when used herein refers to the amino acid residues of an antibody which are associated with its binding to an antigen. The hypervariable regions encompass the amino acid residues of the “complementarity determining regions” or “CDRs” (e.g., residues 24-34 (L1), 50-56 (L2) and 89-97 (L3) of the light chain variable domain and residues 31-35 (H1), 50-65 (H2) and 95-102 (H3) of the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, J. Mol. Biol., 196:901-917 (1987)). “Framework” or “FW” residues are those variable domain residues flanking the CDRs. FW residues are present in chimeric, humanized, human, domain antibodies, diabodies, vaccibodies, linear antibodies, and bispecific antibodies.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, monoclonal antibodies are advantageous in that they can be synthesized by hybridoma cells that are uncontaminated by other immunoglobulin producing cells. Alternative production methods are known to those trained in the art, for example, a monoclonal antibody may be produced by cells stably or transiently transfected with the heavy and light chain genes encoding the monoclonal antibody.

The modifier “monoclonal” indicates the character of the antibody as being obtained from a substantially homogeneous population of antibodies, and is not to be construed as requiring engineering of the antibody by any particular method. The term “monoclonal” is used herein to refer to an antibody that is derived from a clonal population of cells, including any eukaryotic, prokaryotic, or phage clone, and not the method by which the antibody was engineered. For example, the monoclonal antibodies to be used in accordance with the present invention may be made by the hybridoma method first described by Kohler et al., Nature, 256:495 (1975), or may be made by any recombinant DNA method (see, e.g., U.S. Pat. No. 4,816,567), including isolation from phage antibody libraries using the techniques described in Clackson et al., Nature, 352:624-628 (1991) and Marks et al., J. Mol. Biol., 222:581-597 (1991), for example. These methods can be used to produce monoclonal mammalian, chimeric, humanized, human, domain antibodies, diabodies, vaccibodies, linear antibodies, and bispecific antibodies.

The term “chimeric” antibodies includes antibodies in which at least one portion of the heavy and/or light chain is identical with or homologous to corresponding sequences in antibodies derived from a particular species or belonging to a particular antibody class or subclass, and at least one other portion of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include “primatized” antibodies comprising variable domain antigen-binding sequences derived from a nonhuman primate (e.g., Old World Monkey, such as baboon, rhesus or cynomolgus monkey) and human constant region sequences (U.S. Pat. No. 5,693,780).

“Humanized” forms of nonhuman (e.g., murine) antibodies are chimeric antibodies that contain minimal sequence derived from nonhuman immunoglobulin. For the most part, humanized antibodies are human immunoglobulins (recipient antibody) in which the native CDR residues are replaced by residues from the corresponding CDR of a nonhuman species (donor antibody) such as mouse, rat, rabbit or nonhuman primate having the desired specificity, affinity, and capacity. In some instances, FW region residues of the human immunoglobulin are replaced by corresponding nonhuman residues. Furthermore, humanized antibodies may comprise residues that are not found in the recipient antibody or in the donor antibody. These modifications are made to further refine antibody performance In general, a humanized antibody heavy or light chain will comprise substantially all of at least one or more variable domains, in which all or substantially all of the CDRs correspond to those of a nonhuman immunoglobulin and all or substantially all of the FWs are those of a human immunoglobulin sequence. In certain embodiments, the humanized antibody will comprise at least a portion of an immunoglobulin constant region (Fc), typically that of a human immunoglobulin. For further details, see, Jones et al., Nature, 321:522-525 (1986); Riechmann et al., Nature, 332:323-329 (1988); and Presta, Curr. Op. Struct. Biol., 2:593-596 (1992).

A “human antibody” can be an antibody derived from a human or an antibody obtained from a transgenic organism that has been “engineered” to produce specific human antibodies in response to antigenic challenge and can be produced by any method known in the art. In certain techniques, elements of the human heavy and light chain loci are introduced into strains of the organism derived from embryonic stem cell lines that contain targeted disruptions of the endogenous heavy chain and light chain loci. The transgenic organism can synthesize human antibodies specific for human antigens, and the organism can be used to produce human antibody-secreting hybridomas. A human antibody can also be an antibody wherein the heavy and light chains are encoded by a nucleotide sequence derived from one or more sources of human DNA. A fully human antibody also can be constructed by genetic or chromosomal transfection methods, as well as phage display technology, or in vitro activated B cells, all of which are known in the art.

“Antibody-dependent cell-mediated cytotoxicity” and “ADCC” refer to a cell-mediated reaction in which non-specific cytotoxic cells (e.g., Natural Killer (NK) cells, neutrophils, and macrophages) recognize bound antibody on a target cell and subsequently cause lysis of the target cell. In one embodiment, such cells are human cells. While not wishing to be limited to any particular mechanism of action, these cytotoxic cells that mediate ADCC generally express Fc receptors (FcRs). The primary cells for mediating ADCC, NK cells, express FcγRIII, whereas monocytes express FcγRI, FcγRII, FcγRIII and/or FcγRIV. FcR expression on hematopoietic cells is summarized in Ravetch and Kinet, Annu. Rev. Immunol., 9:457-92 (1991). To assess ADCC activity of a molecule, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecules of interest may be assessed in vivo, e.g., in an animal model such as that disclosed in Clynes et al., Proc. Natl. Acad. Sci. (USA), 95:652-656 (1998).

“Complement dependent cytotoxicity” or “CDC” refers to the ability of a molecule to initiate complement activation and lyse a target in the presence of complement. The complement activation pathway is initiated by the binding of the first component of the complement system (C1q) to a molecule (e.g., an antibody) complexed with a cognate antigen. To assess complement activation, a CDC assay, e.g., as described in Gazzano-Santaro et al., J. Immunol. Methods, 202:163 (1996), may be performed.

“Effector cells” are leukocytes which express one or more FcRs and perform effector functions. The cells express at least FcγRI, FCγRII, FcγRIII and/or FcγRIV and carry out ADCC effector function. Examples of human leukocytes which mediate ADCC include peripheral blood mononuclear cells (PBMC), natural killer (NK) cells, monocytes, cytotoxic T cells and neutrophils.

The terms “Fc receptor” or “FcR” are used to describe a receptor that binds to the Fc region of an antibody. In one embodiment, the FcR is a native sequence human FcR. Moreover, in certain embodiments, the FcR is one which binds an IgG antibody (a gamma receptor) and includes receptors of the FcγRI, FcγRII, FcγRIII, and FcγRIV subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (See, Daëron, Annu. Rev. Immunol., 15:203-234 (1997)). FcRs are reviewed in Ravetch and Kinet, Annu. Rev. Immunol., 9:457-92 (1991); Capel et al., Immunomethods, 4:25-34 (1994); and de Haas et al., J. Lab. Clin. Med., 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., Immunol., 117:587 (1976) and Kim et al., J. Immunol., 24:249 (1994)).

“Fv” is the minimum antibody fragment which contains a complete antigen-recognition and binding site. This region consists of a dimer of one heavy and one light chain variable domain in tight, non-covalent or covalent association. It is in this configuration that the three CDRs of each variable domain interact to define an antigen-binding site on the surface of the VH-VL dimer Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

“Affinity” of an antibody for an epitope to be used in the treatment(s) described herein is a term well understood in the art and means the extent, or strength, of binding of antibody to epitope. Affinity may be measured and/or expressed in a number of ways known in the art, including, but not limited to, equilibrium dissociation constant (KD or Kd), apparent equilibrium dissociation constant (KD′ or Kd′), and IC50 (amount needed to effect 50% inhibition in a competition assay). It is understood that, for purposes of this invention, an affinity is an average affinity for a given population of antibodies which bind to an epitope. Values of KD′ reported herein in terms of mg IgG per mL or mg/mL indicate mg Ig per mL of serum, although plasma can be used. When antibody affinity is used as a basis for administration of the treatment methods described herein, or selection for the treatment methods described herein, antibody affinity can be measured before and/or during treatment, and the values obtained can be used by a clinician in assessing whether a human patient is an appropriate candidate for treatment.

As used herein, the term “avidity” is a measure of the overall binding strength (i.e., both antibody arms) with which an antibody binds an antigen. Antibody avidity can be determined by measuring the dissociation of the antigen-antibody bond in antigen excess using any means known in the art, such as, but not limited to, by the modification of indirect fluorescent antibody as described by Gray et al., J. Virol. Meth., 44:11-24. (1993)

An “epitope” is a term well understood in the art and means any chemical moiety that exhibits specific binding to an antibody. An “antigen” is a moiety or molecule that contains an epitope, and, as such, also specifically binds to an antibody.

A “B cell surface marker” as used herein is an antigen expressed on the surface of a B cell which can be targeted with an agent which binds thereto. B cell surface markers include the CD10, CD19, CD20, CD21, CD22, CD23, CD24, CD25, CD37, CD53, CD72, CD73, CD74, CD75, CD77, CD79a, CD79b, CD80, CD81, CD82, CD83, CD84, CD85, and CD86 leukocyte surface markers. A B cell surface marker of particular interest is preferentially expressed on B cells compared to other non-B cell tissues of a mammal and may be expressed on both precursor B cells and mature B-lineage cells. In one embodiment, the marker is CD19, which is found on B cells at various stages of differentiation.

The term “antibody half-life” as used herein means a pharmacokinetic property of an antibody that is a measure of the mean survival time of antibody molecules following their administration. Antibody half-life can be expressed as the time required to eliminate 50 percent of a known quantity of immunoglobulin from the patient's body or a specific compartment thereof, for example, as measured in serum or plasma, i.e., circulating half-life, or in other tissues. Half-life may vary from one immunoglobulin or class of immunoglobulin to another. In general, an increase in antibody half-life results in an increase in mean residence time (MRT) in circulation for the antibody administered.

The term “isotype” refers to the classification of an antibody's heavy or light chain constant region. The constant domains of antibodies are not involved in binding to antigens or an antigen, but exhibit various effector functions. Depending on the amino acid sequence of the heavy chain constant region, a given human antibody or immunoglobulin can be assigned to one of five major classes of immunoglobulins: IgA, IgD, IgE, IgG, and IgM. Several of these classes may be further divided into subclasses (isotypes), e.g., IgG1 (gamma 1), IgG2 (gamma 2), IgG3 (gamma 3), and IgG4 (gamma 4), and IgA1 and IgA2. The heavy chain constant regions that correspond to the different classes of immunoglobulins are called α, δ, ε, γ, and μ, respectively. The structures and three-dimensional configurations of different classes of immunoglobulins are well-known. Of the various human immunoglobulin classes, only human IgG1, IgG2, IgG3, IgG4, and IgM are known to activate complement. Human IgG1 and IgG3 are known to mediate ADCC in humans. Human light chain constant regions may be classified into two major classes, kappa and lambda

As used herein, the term “immunogenicity” means that a compound is capable of provoking an immune response (stimulating production of specific antibodies and/or proliferation of specific T cells).

As used herein, the term “antigenicity” means that a compound is recognized by an antibody or may bind to an antibody and induce an immune response.

By the terms “treat,” “treating” or “treatment of” (or grammatically equivalent terms) it is meant that the severity of the subject's condition is reduced or at least partially improved or ameliorated and/or that some alleviation, mitigation or decrease in at least one clinical symptom is achieved and/or there is an inhibition or delay in the progression of the condition and/or prevention or delay of the onset of a disease or illness. Thus, the terms “treat,” “treating” or “treatment of” (or grammatically equivalent terms) refer to both prophylactic and therapeutic treatment regimes.

As used herein, a “sufficient amount” or “an amount sufficient to” achieve a particular result refers to an amount of an antibody or composition of the invention that is effective to produce a desired effect, which is optionally a therapeutic effect (i.e., by administration of a therapeutically effective amount). For example, a “sufficient amount” or “an amount sufficient to” can be an amount that is effective to deplete B cells.

A “therapeutically effective” amount as used herein is an amount that provides some improvement or benefit to the subject. Stated in another way, a “therapeutically effective” amount is an amount that provides some alleviation, mitigation, and/or decrease in at least one clinical symptom. Clinical symptoms associated with the disorders that can be treated by the methods of the invention are well-known to those skilled in the art. Further, those skilled in the art will appreciate that the therapeutic effects need not be complete or curative, as long as some benefit is provided to the subject.

The term “excipient” as used herein refers to an inert substance which is commonly used as a diluent, vehicle, preservative, binder or stabilizing agent for drugs which imparts a beneficial physical property to a formulation, such as increased protein stability, increased protein solubility, and decreased viscosity. Examples of excipients include, but are not limited to, proteins (for example, but not limited to, serum albumin), amino acids (for example, but not limited to, aspartic acid, glutamic acid, lysine, arginine, glycine), surfactants (for example, but not limited to, SDS, Tween 20, Tween 80, polysorbate and nonionic surfactants), saccharides (for example, but not limited to, glucose, sucrose, maltose and trehalose), polyols (for example, but not limited to, mannitol and sorbitol), fatty acids and phospholipids (for example, but not limited to, alkyl sulfonates and caprylate). For additional information regarding excipients, see Remington's Pharmaceutical Sciences (by Joseph P. Remington, 18^{th} ed., Mack Publishing Co., Easton, Pa.), which is incorporated herein in its entirety.

The phrase “pharmaceutically acceptable” as used herein means approved by a regulatory agency of the Federal or a state government, or listed in the U.S. Pharmacopeia, European Pharmacopia or other generally recognized pharmacopeia for use in animals, and more particularly in humans.

The terms “stability” and “stable” as used herein in the context of a liquid formulation comprising an antibody (including antibody fragment thereof) that specifically binds to an antigen of interest (e.g., CD19) refer to the resistance of the antibody (including antibody fragment thereof) in the formulation to aggregation, degradation or fragmentation under given manufacture, preparation, transportation and storage conditions. The “stable” formulations of the invention retain biological activity under given manufacture, preparation, transportation and storage conditions. The stability of said antibody (including antibody fragment thereof) can be assessed by degrees of aggregation, degradation or fragmentation, as measured by HPSEC, reverse phase chromatography, static light scattering (SLS), Fourier Transform Infrared Spectroscopy (FTIR), circular dichroism (CD), urea unfolding techniques, intrinsic tryptophan fluorescence, differential scanning calorimetry, and/or ANS binding techniques, compared to a reference formulation. For example, a reference formulation may be a reference standard frozen at −70° C. consisting of 10 mg/ml of an antibody (including antibody fragment thereof) (for example, but not limited to, an antibody comprising the 16C4 variable region and an Fc region having complex N-glycoside-linked sugar chains in which fucose is not bound to N-acetylglucosamine in the reducing end in the sugar chain) in 10 mM histidine (pH 6.0) that contains 75 mMNaCl and 4% trehalose, which reference formulation regularly gives a single monomer peak (e.g., ≧95% area) by HPSEC. The overall stability of a formulation comprising an antibody (including antibody fragment thereof) can be assessed by various immunological assays including, for example, ELISA and radioimmunoassay using isolated antigen molecules.

The phrase “low to undetectable levels of aggregation” as used herein refers to samples containing no more than about 5%, no more than about 4%, no more than about 3%, no more than about 2%, no more than about 1% and no more than about 0.5% aggregation by weight of protein as measured by high performance size exclusion chromatography (HPSEC) or static light scattering (SLS) techniques.

The term “low to undetectable levels of fragmentation” as used herein refers to samples containing equal to or more than about 80%, about 85%, about 90%, about 95%, about 98% or about 99% of the total protein, for example, in a single peak as determined by HPSEC or reverse phase echromatography, or in two peaks (e.g., heavy- and light-chains) (or as many peaks as there are subunits) by reduced Capillary Gel Electrophoresis (rCGE), representing the non-degraded antibody or a non-degraded fragment thereof, and containing no other single peaks having more than about 5%, more than about 4%, more than about 3%, more than about 2%, more than about 1%, or more than about 0.5% of the total protein in each. The term “reduced Capillary Gel Electrophoresis” as used herein refers to capillary gel electrophoresis under reducing conditions sufficient to reduce disulfide bonds in an antibody.

As used herein, the terms “manage,” “managing,” and “management” refer to the beneficial effects that a subject derives from a therapy (e.g., a prophylactic or therapeutic agent), which does not result in a cure of the disease. In certain embodiments, a subject is administered one or more therapies (e.g., one or more prophylactic or therapeutic agents) to “manage” a disease so as to prevent the progression or worsening of the disease.

As used herein, the terms “prevent,” “preventing,” and “prevention” refer to the inhibition of the development or onset of disease or disorder, or the prevention of the recurrence, onset, or development of one or more symptoms of a disease or disorder in a subject resulting from the administration of a therapy (e.g., a prophylactic or therapeutic agent), or the administration of a combination of therapies (e.g., a combination of prophylactic or therapeutic agents).

As used herein, a “sufficient amount” or “an amount sufficient to” achieve a particular result refers to an amount of an antibody or composition of the invention that is effective to produce a desired effect, which is optionally a therapeutic effect (i.e., by administration of a therapeutically effective amount). For example, a “sufficient amount” or “an amount sufficient to” can be an amount that is effective to deplete CD19 expressing B cells.

A “therapeutically effective” amount as used herein is an amount that provides some improvement or benefit to the subject. Stated in another way, a “therapeutically effective” amount is an amount that provides some alleviation, mitigation, and/or decrease in at least one clinical symptom. Clinical symptoms associated with the disorders that can be treated by the methods of the invention are well-known to those skilled in the art. Further, those skilled in the art will appreciate that the therapeutic effects need not be complete or curative, as long as some benefit is provided to the subject.

As used herein, the term “subject” includes any human or nonhuman animal. The term “nonhuman animal” includes all vertebrates, for example, but not limited to, mammals and non-mammals, such as nonhuman primates, sheep, dogs, cats, horses, cows, chickens, amphibians, reptiles, etc.

Concentrations, amounts, cell counts, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

### 5. BRIEF DESCRIPTION OF THE DRAWINGS

FIG. 1A-B: (A) Amino acid sequence alignment of the HB12B VK (SEQ ID NO:20), HB12B-(A10-Jk4) (SEQ ID NO:52), HB12B-364987 (SEQ ID NO:62), HB12B-3649 (SEQ ID NO:68), and HB12B-36 (SEQ ID NO:70) light chains variable regions. Sequence residues are numbered according to Kabat. CDR residues, defined according to Kabat, are boxed. Vernier, Interchain, and Canonical residues of HB12B VK (SEQ ID NO:20) are highlighted in light gray. Amino acid substitutions of HB12B-364987 (SEQ ID NO:62) (Y40F, K53H, Y91F), HB12B-3649 (SEQ ID NO:68) (Y40F, K53H), and HB12B-36 (SEQ ID NO:70) (Y40F) relative to the grafted antibody HB12B-(A10-Jk4) (SEQ ID NO:52) variable domain are highlighted in dark gray. (B) Amino acid sequence alignment of the HB12B VH (SEQ ID NO:18), HB12B-(3-72/JH4) (SEQ ID NO:34), and HB12B-9m (SEQ ID NO:44) heavy chain variable regions. Sequence residues are numbered according to Kabat. CDR residues, defined according to Kabat, are boxed. Vernier, Interchain, and Canonical residues of HB12B VH are highlighted in light gray. Amino acid substitutions of HB12B-9m (SEQ ID NO:44) (L20I, F27Y, T28A, R38I, V49I, F67A, R71A, L80M, I91Y) relative to the grafted antibody HB12B-(3-72/JH4) (SEQ ID NO:34) variable domain are highlighted in dark gray.

FIG. 2. DSC profile of the 551 afucosylated humanized anti-CD19 antibody.

FIG. 3. Colloidal stability of the 551 afucosylated humanized anti-CD19 antibody at various pHs. The graph shows the absorbance at 350 nm as a function of temperature.

FIG. 4. Thermal stability of the 551 afucosylated humanized anti-CD19 antibody at various pHs. The graph shows tryptophan fluorescence measured at 326 nm as a function of temperature.

FIG. 5. Thermal destabilization rate of the 551 afucosylated humanized anti-CD19 antibody at various temperatures. The graph shows absorbance at 350 nm as a function of time.

FIG. 6. Thermal destabilization rate of the 551 afucosylated humanized anti-CD19 antibody in formulations comprising various excipients. The graph shows absorbance at 350 nm as a function of time.

FIG. 7. Thermal destabilization rate of the 551 afucosylated humanized anti-CD19 antibody in formulations comprising various concentrations of NaCl. The graph shows absorbance at 350 nm as a function of time.

FIG. 8. Thermal destabilization rate of the 551 afucosylated humanized anti-CD19 antibody in formulations comprising various concentrations of trehalose. The graph shows absorbance at 350 nm as a function of time.

FIG. 9. Thermal destabilization rate of the 551 afucosylated humanized anti-CD19 antibody in formulations comprising various combinations of NaCl and trehalose. The graph shows absorbance at 350 nm as a function of time.

FIG. 10. Aggregation rate of various 551 afucosylated humanized anti-CD19 antibody formulations at 40° C. Monomer concentration (% of total protein) as a function of time is shown.

FIG. 11. Aggregation rate of various 551 afucosylated humanized anti-CD19 antibody formulations at 40° C. Aggregate concentration (% of total protein) as a function of time is shown.

FIG. 12. Fragmentation rate of various 551 afucosylated humanized anti-CD19 antibody formulations at 40° C. Fragment concentration (% of total protein) as a function of time is shown.

FIG. 13. Aggregation rate of a 551 afucosylated humanized anti-CD19 antibody formulation comprising various antibody concentrations measured at 40° C. Monomer concentration (% of total protein) as a function of time is shown.

FIG. 14. Dimer formation rate of a 551 afucosylated humanized anti-CD19 antibody formulation comprising various antibody concentrations measured at 40° C. Dimer concentration (% of total protein) as a function of time is shown.

FIG. 15. Aggregation rate of a 551 afucosylated humanized anti-CD19 antibody formulation comprising various antibody concentrations measured at 40° C. Multimer concentration (% of total protein) as a function of time is shown.

FIG. 16. Fragmentation rate of a 551 afucosylated humanized anti-CD19 antibody formulation comprising various antibody concentrations measured at 40° C. Fragment concentration (% of total protein) as a function of time is shown.

FIG. 17. DSC profile of the 551 antibody at various pHs.

FIG. 18. DSC profile of the 551 antibody at various pHs.

FIG. 19. Aggregation rate of various 551 afucosylated humanized anti-CD19 antibody formulations comprising 10 mg/ml antibody at 5° C. Monomer concentration (% of total protein) as a function of time is shown.

FIG. 20. Aggregation rate of various 551 afucosylated humanized anti-CD19 antibody formulations comprising 10 mg/ml antibody at 5° C. Multimer concentration (% of total protein) as a function of time is shown.

FIG. 21. Aggregation rate of various 551 afucosylated humanized anti-CD19 antibody formulations comprising 10 mg/ml antibody at 5° C. Total aggregate concentration (% of total protein) as a function of time is shown.

FIG. 22. Aggregation rate of a 551 afucosylated humanized anti-CD19 antibody formulations comprising 10 mg/ml antibody at various temperatures. Monomer concentration (% of total protein) as a function of time is shown.

FIG. 23. Aggregation rate of a 551 afucosylated humanized anti-CD19 antibody formulations comprising 10 mg/ml antibody at various temperatures. Total aggregate and multimer concentration (% of total protein) as a function of time is shown.

FIG. 24. Fragmentation rate of a 551 afucosylated humanized anti-CD19 antibody formulations comprising 10 mg/ml antibody at various temperatures. Fragment concentration (% of total protein) as a function of time is shown.

FIG. 25. Stability profile of the 551 afucosylated humanized anti-CD19 antibody formulation comprising 10 mM Histidine, 75 mM NaCl, and 4% Trehalose at pH 6.0.

FIG. 26. Aggregate formation in 551 afucosylated humanized anti-CD19 antibody formulations comprising various concentrations of polysorbate 80 (PS80) subjected to 4 hrs of shaking. Final monomer concentration (% of total protein) is shown.

FIG. 27. Aggregate formation in 551 afucosylated humanized anti-CD19 antibody formulations comprising various concentrations of polysorbate 80 (PS80) subjected to 4 hrs of shaking. Final total aggregate concentration (% of total protein) is shown.

FIG. 28. Aggregate formation in 551 afucosylated humanized anti-CD19 antibody formulations comprising various concentrations of polysorbate 80 (PS80) subjected to 24 hrs of shaking in a syringe. Final total aggregate concentration (% of total protein) is shown.

FIG. 29. Aggregation rate of 551 afucosylated humanized anti-CD19 antibody formulations in the presence and absence of polysorbate 80 (PS80) at 25° C. and 40° C. Aggregate concentration (% of total protein) as a function of time is shown.

FIG. 30. Aggregation rate of 551 afucosylated humanized anti-CD19 antibody formulations in the presence and absence of polysorbate 80 (PS80) at 25° C. and 40° C. Total multimer concentration (% of total protein) as a function of time is shown.

FIG. 31. CD138/CD19 expression profile of the AN BL6, H929 and RPMI8226 multiple myeloma cell lines.

FIG. 32. The fraction of cells expressing CD138 or CD19 in an ANBL6 culture changes over time. The ratio of CD19 and CD138 expressing cells as a function of time is shown.

FIG. 33. The CD19+CD138− cells, but not the CD19−CD138+ cells isolated from an

ANBL6 culture are able to form colonies in soft agar.

FIG. 34. ADCC activity of the anti-CD19 mAb on multiple myeloma cells. An anti-CD19-aFuc specifically depletes CD19+ cells from unsorted multiple myeloma cell cultures.

FIG. 35. The 551 afucosylated anti-CD19 antibody suppresses myeloma xenograft growth in SCID mice. Tumor volume as a function of time is shown.

FIG. 36. Double logarithmic plots of particle count vs. size for anti-CD19 sample vials subjected to PVDF filtration only.

FIG. 37. Double logarithmic plots of particle count vs. size for anti-CD19 sample vials subjected to PVDF filtration followed by PS80 addition.

FIG. 38. Double logarithmic plots of particle count vs. size for anti-CD19 sample vials subjected to PVDF filtration, then PS80 addition, and another round of PVDF filtration.

### 6. DETAILED DESCRIPTION

The present invention provides sterile, stable aqueous formulations comprising a chimeric, human, or humanized antibody that specifically binds the human CD19 antigen. In one embodiment, the present invention provides formulations comprising chimeric and humanized versions of anti-CD19 mouse monoclonal antibodies, HB12A and HB12B. In another embodiment, the present invention provides formulations comprising an anti-CD19 antibodies described in U.S. patent application Ser. No. 11/852,106 filed on Sep. 7, 2007, the disclosure of which is incorporated herein in its entirety for all purposes. In a further embodiment, the present invention provides a formulation comprising an anti-CD19 antibody of the invention that may mediate one or more of the following: complement-dependent cell-mediated cytotoxicity (CDC), antigen-dependent cell-mediated-cytotoxicity (ADCC), and programmed cell death (apoptosis) and has enhanced effector functions. In another embodiment, a formulation of the invention comprises an anti-CD19 antibody of the invention comprising an Fc region having complex N-glycoside-linked sugar chains in which fucose is not bound to N-acetylglucosamine in the reducing end in the sugar chain. In a specific embodiment, a formulation of the invention comprises an anti-CD19 antibody comprising a heavy chain variable region of SEQ ID NO:104, a light chain variable sequence of SEQ ID NO:111 and an Fc region having complex N-glycoside-linked sugar chains in which fucose is not bound to N-acetylglucosamine in the reducing end in the sugar chain. In certain embodiments, a stable liquid formulation comprising an anti-CD19 antibody of the invention is suitable for parenteral administration to a human subject. In a specific embodiment, a stable liquid formulation of the invention is suitable for subcutaneous administration to a human subject.

### 6.1. Antibody Formulations

In specific embodiments, the present invention encompasses stable liquid formulations of antibodies that specifically bind to human CD19 and have an enhanced effector function (e.g., antibody-dependent cellular cytotoxicity (ADCC), complement-dependent cell-mediated cytotoxicity (CDC), and/or antibody-dependent phagocytosis), wherein the formulations exhibit low to undetectable levels of antibody aggregation and/or fragmentation with very little to no loss of the biological activities during manufacture, preparation, transportation, and long periods of storage. The present invention also encompasses stable liquid formulations of antibodies that specifically bind to human CD19, have an enhanced effector function and have increased in vivo half-lives, said formulations exhibiting low to undetectable levels of antibody aggregation and/or fragmentation, and very little to no loss of the biological activities (e.g., antibody-dependent cellular cytotoxicity (ADCC), complement-dependent cell-mediated cytotoxicity (CDC), and/or antibody-dependent phagocytosis) of the antibodies during manufacture, preparation, transportation, and long periods of storage. In specific embodiments, a formulation of the invention comprises an anti-human CD19 antibody having increased in vivo ADCC activity, said formulation exhibiting low to undetectable levels of antibody aggregation and/or fragmentation, and very little to no loss of the biological activities of the antibodies during manufacture, preparation, transportation, and long periods of storage.

In one embodiment, a liquid formulation of the invention is an aqueous formulation. In a specific embodiment, a liquid formulation of the invention is an aqueous formulation wherein the aqueous carrier is distilled water.

In one embodiment, a formulation of the invention is sterile.

In one embodiment, a formulation of the invention is homogeneous.

In one embodiment, a formulation of the invention is isotonic.

In specific embodiments, the present invention provides stable liquid formulations comprising an anti-CD19 antibody having an enhanced effector function. In one embodiment, a formulation of the invention comprises an anti-CD19 antibody described in U.S. patent application Ser. No. 11/852,106 filed on Sep. 7, 2007, the disclosure of which is incorporated herein in its entirety for all purposes.

In one embodiment, a formulation of the invention comprises an anti-CD19 antibody of the invention, wherein said antibody comprises a VH domain having the amino acid sequence of SEQ ID NO:104 and a VL domain having the amino acid sequence of SEQ ID NO:111. In a specific embodiment, a formulation of the invention comprises an anti-human CD19 antibody comprising an Fc region having complex N-glycoside-linked sugar chains in which fucose is not bound to N-acetylglucosamine in the reducing end in the sugar chain.

The invention encompasses stable liquid formulations comprising a single antibody of interest (including antibody fragment thereof), for example, an antibody of the invention that specifically binds to a CD19 polypeptide. The invention also encompasses stable liquid formulations comprising two or more antibodies of interest (including antibody fragments thereof), for example, antibodies of the invention that specifically bind to a CD19 polypeptide(s).

In one embodiment, a formulation of the invention comprises at least about 1 mg/ml, at least about 2 mg/ml, at least about 3 mg/ml, at least about 4 mg/ml, at least about 5 mg/ml, at least about 6 mg/ml, at least about 7 mg/ml, at least about 8 mg/ml, at least about 9 mg/ml, at least about 10 mg/ml, at least about 11 mg/ml, at least about 12 mg/ml, at least about 13 mg/ml, at least about 14 mg/ml, at least about 15 mg/ml, at least about 16 mg/ml, at least about 17 mg/ml, at least about 18 mg/ml, at least about 19 mg/ml, at least about 20 mg/ml, at least about 30 mg/ml, at least about 40 mg/ml, at least about 50 mg/ml, at least about 60 mg/ml, at least about 70 mg/ml, at least about 80 mg/ml, at least about 90 mg/ml, at least about 100 mg/ml, at least about 110 mg/ml, at least about 120 mg/ml, at least about 130 mg/ml, at least about 140 mg/ml, at least about 150 mg/ml, at least about 160 mg/ml, at least about 170 mg/ml, at least about 180 mg/ml, at least about 190 mg/ml, at least about 200 mg/ml, at least about 250 mg/ml, or at least about 300 mg/ml of an anti-CD19 antibody of the invention. In a specific embodiment, a formulation of the invention comprises at least about 5 mg/ml of an anti-CD19 antibody of the invention. In a specific embodiment, a formulation of the invention comprises at least about 10 mg/ml of an anti-CD19 antibody of the invention. In a specific embodiment, a formulation of the invention comprises at least about 15 mg/ml of an anti-CD19 antibody of the invention. In a specific embodiment, a formulation of the invention comprises at least about 20 mg/ml of an anti-CD19 antibody of the invention. In a specific embodiment, a formulation of the invention comprises at least about 30 mg/ml of an anti-CD19 antibody of the invention. In another embodiment, a formulation of the invention comprises between about 1 mg/ml and about 25 mg/ml, between about 1 mg/ml and about 30 mg/ml, between about 5 mg/ml and about 25 mg/ml, between about 5 mg/ml and about 30 mg/ml, between about 7.5 mg/ml and about 25 mg/ml, or between about 7.5 mg/ml and about 30 mg/ml of an anti-CD19 antibody of the invention. In a specific embodiment, a formulation of the invention comprises between about 1 mg/ml and about 25 mg/ml of an anti-CD19 antibody of the invention. In a specific embodiment, a formulation of the invention comprises between about 5 mg/ml and about 25 mg/ml of an anti-CD19 antibody of the invention. In a further embodiment, a formulation described herein comprises about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 30 mg/ml, about 40 mg/ml, about 50 mg/ml, about 60 mg/ml, about 70 mg/ml, about 80 mg/ml, about 90 mg/ml, about 100 mg/ml, about 110 mg/ml, about 120 mg/ml, about 130 mg/ml, about 140 mg/ml, about 150 mg/ml, about 160 mg/ml, about 170 mg/ml, about 180 mg/ml, about 190 mg/ml, about 200 mg/ml, about 250 mg/ml, or about 300 mg/ml of an anti-CD19 antibody of the invention. In a specific embodiment, a formulation of the invention comprises about 5 mg/ml of an anti-CD19 antibody of the invention. In a specific embodiment, a formulation of the invention comprises about 10 mg/ml of an anti-CD19 antibody of the invention. In a specific embodiment, a formulation of the invention comprises about 15 mg/ml of an anti-CD19 antibody of the invention. In a specific embodiment, a formulation of the invention comprises about 25 mg/ml of an anti-CD19 antibody of the invention. In a specific embodiment, a formulation of the invention comprises about 50 mg/ml of an anti-CD19 antibody of the invention. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention comprises at least 1 mg/ml, at least 2 mg/ml, at least 3 mg/ml, at least 4 mg/ml, at least 5 mg/ml, at least 6 mg/ml, at least 7 mg/ml, at least 8 mg/ml, at least 9 mg/ml, at least 10 mg/ml, at least 11 mg/ml, at least 12 mg/ml, at least 13 mg/ml, at least 14 mg/ml, at least 15 mg/ml, at least 16 mg/ml, at least 17 mg/ml, at least 18 mg/ml, at least 19 mg/ml, at least 20 mg/ml, at least 30 mg/ml, at least 40 mg/ml, at least 50 mg/ml, at least 60 mg/ml, at least 70 mg/ml, at least 80 mg/ml, at least 90 mg/ml, at least 100 mg/ml, at least 110 mg/ml, at least 120 mg/ml, at least 130 mg/ml, at least 140 mg/ml, at least 150 mg/ml, at least 160 mg/ml, at least 170 mg/ml, at least 180 mg/ml, at least 190 mg/ml, at least 200 mg/ml, at least 250 mg/ml, or at least 300 mg/ml of an anti-CD19 antibody of the invention. In a specific embodiment, a formulation of the invention comprises at least 5 mg/ml of an anti-CD19 antibody of the invention. In a specific embodiment, a formulation of the invention comprises at least 10 mg/ml of an anti-CD19 antibody of the invention. In a specific embodiment, a formulation of the invention comprises at least 15 mg/ml of an anti-CD19 antibody of the invention. In a specific embodiment, a formulation of the invention comprises at least 20 mg/ml of an anti-CD19 antibody of the invention. In a specific embodiment, a formulation of the invention comprises at least 30 mg/ml of an anti-CD19 antibody of the invention. In another embodiment, a formulation of the invention comprises between 1 mg/ml and 25 mg/ml, between 1 mg/ml and 30 mg/ml, between 5 mg/ml and 25 mg/ml, between 5 mg/ml and 30 mg/ml, between 7.5 mg/ml and 25 mg/ml, or between 7.5 mg/ml and 30 mg/ml of an anti-CD19 antibody of the invention. In a specific embodiment, a formulation of the invention comprises between 1 mg/ml and 25 mg/ml of an anti-CD19 antibody of the invention. In a specific embodiment, a formulation of the invention comprises between 5 mg/ml and 25 mg/ml of an anti-CD19 antibody of the invention. In a further embodiment, a formulation described herein comprises 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 30 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, 150 mg/ml, 160 mg/ml, 170 mg/ml, 180 mg/ml, 190 mg/ml, 200 mg/ml, 250 mg/ml, or 300 mg/ml of an anti-CD19 antibody of the invention. In a specific embodiment, a formulation of the invention comprises 5 mg/ml of an anti-CD19 antibody of the invention. In a specific embodiment, a formulation of the invention comprises 10 mg/ml of an anti-CD19 antibody of the invention. In a specific embodiment, a formulation of the invention comprises 15 mg/ml of an anti-CD19 antibody of the invention. In a specific embodiment, a formulation of the invention comprises 25 mg/ml of an anti-CD19 antibody of the invention. In a specific embodiment, a formulation of the invention comprises 50 mg/ml of an anti-CD19 antibody of the invention. In a specific embodiment, a formulation of the invention comprises 100 mg/ml of an anti-CD19 antibody of the invention. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

Optionally, the formulations of the invention may further comprise common excipients and/or additives such as buffering agents, saccharides, salts and surfactants. Additionally or alternatively, the formulations of the invention may further comprise common excipients and/or additives, such as, but not limited to, solubilizers, diluents, binders, stabilizers, salts, lipophilic solvents, amino acids, chelators, preservatives, or the like.

In certain embodiments, the buffering agent is selected from the group consisting of histidine, citrate, phosphate, glycine, and acetate. In other embodiments the saccharide excipient is selected from the group consisting of trehalose, sucrose, mannitol, maltose and raffinose. In still other embodiments the surfactant is selected from the group consisting of polysorbate 20, polysorbate 40, polysorbate 80, and Pluronic F68. In yet other embodiments the salt is selected from the group consisting of NaCl, KCl, MgCl₂, and CaCl₂

Optionally, the formulations of the invention may further comprise other common auxiliary components, such as, but not limited to, suitable excipients, polyols, solubilizers, diluents, binders, stabilizers, lipophilic solvents, chelators, preservatives, or the like.

The formulations of the invention include a buffering or pH adjusting agent to provide improved pH control. In one embodiment, a formulation of the invention has a pH of between about 3.0 and about 9.0, between about 4.0 and about 8.0, between about 5.0 and about 8.0, between about 5.0 and about 7.0, between about 5.0 and about 6.5, between about 5.5 and about 8.0, between about 5.5 and about 7.0, or between about 5.5 and about 6.5. In a further embodiment, a formulation of the invention has a pH of about 3.0, about 3.5, about 4.0, about 4.5, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.5, about 8.0, about 8.5, or about 9.0. In a specific embodiment, a formulation of the invention has a pH of about 6.0.

The formulations of the invention include a buffering or pH adjusting agent to provide improved pH control. In one embodiment, a formulation of the invention has a pH of between 3.0 and 9.0, between 4.0 and 8.0, between 5.0 and 8.0, between 5.0 and 7.0, between 5.0 and 6.5, between 5.5 and 8.0, between 5.5 and 7.0, or between 5.5 and 6.5. In a further embodiment, a formulation of the invention has a pH of 3.0, 3.5, 4.0, 4.5, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.5, 8.0, 8.5, or 9.0. In a specific embodiment, a formulation of the invention has a pH of 6.0.

The pH of the formulation generally should not be equal to the isoelectric point of the particular antibody (including antibody fragment thereof) to be used in the formulation (for example, but not limited to, the isoelectric point of the anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain) and may range from about 4.0 to about 8.0, or may range from about 5.5 to about 6.5.

Typically, the buffering agent is a salt prepared from an organic or inorganic acid or base. Representative buffering agents include, but are not limited to, organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. In addition, amino acid components can also function in a buffering capacity. Representative amino acid components which may be utilized in the formulations of the invention as buffering agents include, but are not limited to, glycine and histidine. In certain embodiments, the buffering agent is selected from the group consisting of histidine, citrate, phosphate, glycine, and acetate. In a specific embodiment, the buffering agent is histidine. In another specific embodiment, the buffering agent is citrate. The purity of the buffering agent should be at least 98%, or at least 99%, or at least 99.5%. As used herein, the term “purity” in the context of histidine refers to chemical purity of histidine as understood in the art, e.g., as described in The Merck Index, 13th ed., O'Neil et al. ed. (Merck & Co., 2001).

Buffering agents are typically used at concentrations between about 1 mM and about 200 mM or any range or value therein, depending on the desired ionic strength and the buffering capacity required. The usual concentrations of conventional buffering agents employed in parenteral formulations can be found in: Pharmaceutical Dosage Form: Parenteral Medications, Volume 1, 2^{nd} Edition, Chapter 5, p. 194, De Luca and Boylan, “Formulation of Small Volume Parenterals”, Table 5: Commonly used additives in Parenteral Products. In one embodiment, the buffering agent is at a concentration of about 1 mM, or of about 5 mM, or of about 10 mM, or of about 15 mM, or of about 20 mM, or of about 25 mM, or of about 30 mM, or of about 35 mM, or of about 40 mM, or of about 45 mM, or of about 50 mM, or of about 60 mM, or of about 70 mM, or of about 80 mM, or of about 90 mM, or of about 100 mM. In one embodiment, the buffering agent is at a concentration of 1 mM, or of 5 mM, or of 10 mM, or of 15 mM, or of 20 mM, or of 25 mM, or of 30 mM, or of 35 mM, or of 40 mM, or of 45 mM, or of 50 mM, or of 60 mM, or of 70 mM, or of 80 mM, or of 90 mM, or of 100 mM. In a specific embodiment, the buffering agent is at a concentration of between about 5 mM and about 50 mM. In another specific embodiment, the buffering agent is at a concentration of between 5 mM and 20 mM.

Buffering agents are typically used at concentrations between 1 mM and 200 mM or any range or value therein, depending on the desired ionic strength and the buffering capacity required. The usual concentrations of conventional buffering agents employed in parenteral formulations can be found in: Pharmaceutical Dosage Form: Parenteral Medications, Volume 1, 2^{nd} Edition, Chapter 5, p. 194, De Luca and Boylan, “Formulation of Small Volume Parenterals”, Table 5: Commonly used additives in Parenteral Products. In one embodiment, the buffering agent is at a concentration of 1 mM, or of 5 mM, or of 10 mM, or of 15 mM, or of 20 mM, or of 25 mM, or of 30 mM, or of 35 mM, or of 40 mM, or of 45 mM, or of 50 mM, or of 60 mM, or of 70 mM, or of 80 mM, or of 90 mM, or of 100 mM. In one embodiment, the buffering agent is at a concentration of 1 mM, or of 5 mM, or of 10 mM, or of 15 mM, or of 20 mM, or of 25 mM, or of 30 mM, or of 35 mM, or of 40 mM, or of 45 mM, or of 50 mM, or of 60 mM, or of 70 mM, or of 80 mM, or of 90 mM, or of 100 mM. In a specific embodiment, the buffering agent is at a concentration of between 5 mM and 50 mM. In another specific embodiment, the buffering agent is at a concentration of between 5 mM and 20 mM.

In certain embodiments, a formulation of the invention comprises a buffering agent. In one embodiment, said buffering agent is selected from the group consisting of histidine, citrate, phosphate, glycine, and acetate. In a specific embodiment, a formulation of the invention comprises histidine as a buffering agent.

In one embodiment, a formulation of the invention comprises at least about 1 mM, at least about 5 mM, at least about 10 mM, at least about 20 mM, at least about 30 mM, at least about 40 mM, at least about 50 mM, at least about 75 mM, at least about 100 mM, at least about 150 mM, or at least about 200 mM histidine. In another embodiment, a formulation of the invention comprises between about 1 mM and about 200 mM, between about 1 mM and about 150 mM, between about 1 mM and about 100 mM, between about 1 mM and about 75 mM, between about 10 mM and about 200 mM, between about 10 mM and about 150 mM, between about 10 mM and about 100 mM, between about 10 mM and about 75 mM, between about 10 mM and about 50 mM, between about 10 mM and about 40 mM, between about 10 mM and about 30 mM, between about 20 mM and about 75 mM, between about 20 mM and about 50 mM, between about 20 mM and about 40 mM, or between about 20 mM and about 30 mM histidine. In a further embodiment of the invention comprises about 1 mM, about 5 mM, about 10 mM, about 20 mM, about 25 mM, about 30 mM, about 35 mM, about 40 mM, about 45 mM, about 50 mM, about 60 mM, about 70 mM, about 80 mM, about 90 mM, about 100 mM, about 150 mM, or about 200 mM histidine. In a specific embodiment, a formulation of the invention comprises about 10 mM histidine.

In one embodiment, a formulation of the invention comprises at least 1 mM, at least 5 mM, at least 10 mM, at least 20 mM, at least 30 mM, at least 40 mM, at least 50 mM, at least 75 mM, at least 100 mM, at least 150 mM, or at least 200 mM histidine. In another embodiment, a formulation of the invention comprises between 1 mM and 200 mM, between 1 mM and 150 mM, between 1 mM and 100 mM, between 1 mM and 75 mM, between 10 mM and 200 mM, between 10 mM and 150 mM, between 10 mM and 100 mM, between 10 mM and 75 mM, between 10 mM and 50 mM, between 10 mM and 40 mM, between 10 mM and 30 mM, between 20 mM and 75 mM, between 20 mM and 50 mM, between 20 mM and 40 mM, or between 20 mM and 30 mM histidine. In a further embodiment of the invention comprises 1 mM, 5 mM, 10 mM, 20 mM, 25 mM, 30 mM, 35 mM, 40 mM, 45 mM, 50 mM, 60 mM, 70 mM, 80 mM, 90 mM, 100 mM, 150 mM, or 200 mM histidine. In a specific embodiment, a formulation of the invention comprises 10 mM histidine.

In certain embodiments, the formulations of the invention comprise a carbohydrate excipient. Carbohydrate excipients can act, e.g., as viscosity enhancing agents, stabilizers, bulking agents, solubilizing agents, and/or the like. Carbohydrate excipients are generally present at between about 1% to about 99% by weight or volume. In one embodiment, the carbohydrate excipient is present at between about 0.1% to about 20%. In another embodiment, the carbohydrate excipient is present at between about 0.1% to about 15%. In a specific embodiment, the carbohydrate excipient is present at between about 0.1% to about 5%, or between about 1% to about 15%, or between about 2% to about 10%, or between about 2% to about 8%, or between about 2% and about 6%. In a specific embodiment, the carbohydrate excipient is present at between 0.1% to 5%, or between 1% to 15%, or between 2% to 10%, or between 2% to 8%, or between 2% and 6%. In still another specific embodiment, the carbohydrate excipient is present at between about 0.1% to about 10%. In still another specific embodiment, the carbohydrate excipient is present at between about 1% to about 8%. In yet another specific embodiment, the carbohydrate excipient is present at between about 2% to about 6%. In still other specific embodiments, the carbohydrate excipient is present at about 1%, or at about 1.5%, or at about 2%, or at about 2.5%, or at about 3%, or at about 4%, or at about 5%, or at about 10%, or at about 15%, or at about 20%.

In certain embodiments, the formulations of the invention comprise a carbohydrate excipient. Carbohydrate excipients can act, e.g., as viscosity enhancing agents, stabilizers, bulking agents, solubilizing agents, and/or the like. Carbohydrate excipients are generally present at between 1% to 99% by weight or volume. In one embodiment, the carbohydrate excipient is present at between 0.1% to 20%. In another embodiment, the carbohydrate excipient is present at between 0.1% to 15%. In a specific embodiment, the carbohydrate excipient is present at between 0.1% to 5%, or between 1% to 15%, or between 2% to 10%, or between 2% to 8%, or between 2% and 6%. In a specific embodiment, the carbohydrate excipient is present at between 0.1% to 5%, or between 1% to 15%, or between 2% to 10%, or between 2% to 8%, or between 2% and 6%. In still another specific embodiment, the carbohydrate excipient is present at between 0.1% to 10%. In still another specific embodiment, the carbohydrate excipient is present at between 1% to 8%. In yet another specific embodiment, the carbohydrate excipient is present at between 2% to 6%. In still other specific embodiments, the carbohydrate excipient is present at 1%, or at 1.5%, or at 2%, or at 2.5%, or at 3%, or at 4%, or at 5%, or at 10%, or at 15%, or at 20%.

Carbohydrate excipients suitable for use in the formulations of the invention include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and the like. In one embodiment, the carbohydrate excipients for use in the present invention are selected from the group consisting of, sucrose, trehalose, lactose, mannitol, and raffinose. In a specific embodiment, the carbohydrate excipient is trehalose. In another specific embodiment, the carbohydrate excipient is mannitol. In yet another specific embodiment, the carbohydrate excipient is sucrose. In still another specific embodiment, the carbohydrate excipient is raffinose. The purity of the carbohydrate excipient should be at least 98%, or at least 99%, or at least 99.5%.

In one embodiment, a formulation of the invention comprises at least about 1%, at least about 2%, at least about 3%, at least about 4%, at least about 5%, at least about 6%, at least about 8%, at least about 20%, at least about 30%, or at least about 40% trehalose. In another embodiment, a formulation of the invention comprises between about 1% and about 30%, between about 1% and about 20%, between about 1% and about 10%, between about 2% and about 30%, between about 2% and about 20%, between about 2% and about 10%, between about 2% and about 8%, between about 2% and about 6%, or between about 3% and about 6% trehalose. In a further embodiment, a formulation of the invention comprises about 1%, about 2%, about 3%, about 4%, about 5%, about 6%, about 8%, about 20%, about 30%, or about 40% trehalose. In a specific embodiment, a formulation of the invention comprises about 4% trehalose.

In one embodiment, a formulation of the invention comprises at least 1%, at least 2%, at least 3%, at least 4%, at least 5%, at least 6%, at least 8%, at least 20%, at least 30%, or at least 40% trehalose. In another embodiment, a formulation of the invention comprises between 1% and 30%, between 1% and 20%, between 1% and 10%, between 2% and 30%, between 2% and 20%, between 2% and 10%, between 2% and 8%, between 2% and 6%, or between 3% and 6% trehalose. In a further embodiment, a formulation of the invention comprises 1%, 2%, 3%, 4%, 5%, 6%, 8%, 20%, 30%, or 40% trehalose. In a specific embodiment, a formulation of the invention comprises 4% trehalose.

In one embodiment, a formulation of the invention comprises an excipient. In a specific embodiment, a formulation of the invention comprises at least one excipient selected from the group consisting of: sugar, salt, surfactant, amino acid, polyol, chelating agent, emulsifier and preservative. In one embodiment, a formulation of the invention comprises a salt. In one embodiment, a formulation of the invention comprises a salt selected from the group consisting of: NaCl, KCl, CaCl₂, and MgCl₂. In a specific embodiment, a formulation of the invention comprises NaCl.

In one embodiment, a formulation of the invention comprises at least about 10 mM, at least about 25 mM, at least about 50 mM, at least about 75 mM, at least about 80 mM, at least about 100 mM, at least about 125 mM, at least about 150 mM, at least about 175 mM. at least about 200 mM, or at least about 300 mM sodium chloride. In a further embodiment, a formulation described herein comprises between about 10 mM and about 300 mM, between about 10 mM and about 200 mM, between about 10 mM and about 175 mM, between about 10 mM and about 150 mM, between about 25 mM and about 300 mM, between about 25 mM and about 200 mM, between about 25 mM and about 175 mM, between about 25 mM and about 150 mM, between about 50 mM and about 300 mM, between about 50 mM and about 200 mM, between about 50 mM and about 175 mM, between about 50 mM and about 150 mM, between about 60 mM and about 300 mM, between about 60 mM and about 200 mM, between about 60 mM and about 175 mM, between about 60 mM and about 150 mM, between about 100 mM and about 300 mM, between about 100 mM and about 200 mM, between about 100 mM and about 175 mM, or between about 100 mM and about 150 mM sodium chloride. In a further embodiment, a formulation of the invention comprises about 10 mM. about 25 mM, about 50 mM, about 75 mM, about 80 mM, about 100 mM, about 125 mM, about 150 mM, about 175 mM, about 200 mM, or about 300 mM sodium chloride. In a specific embodiment, a formulation of the invention comprises 75 mM sodium chloride.

In one embodiment, a formulation of the invention comprises at least 10 mM, at least 25 mM, at least 50 mM, at least 75 mM, at least 80 mM, at least 100 mM, at least 125 mM, at least 150 mM, at least 175 mM. at least 200 mM, or at least 300 mM sodium chloride. In a further embodiment, a formulation described herein comprises between 10 mM and 300 mM, between 10 mM and 200 mM, between 10 mM and 175 mM, between 10 mM and 150 mM, between 25 mM and 300 mM, between 25 mM and 200 mM, between 25 mM and 175 mM, between 25 mM and 150 mM, between 50 mM and 300 mM, between 50 mM and 200 mM, between 50 mM and 175 mM, between 50 mM and 150 mM, between 60 mM and 300 mM, between 60 mM and 200 mM, between 60 mM and 175 mM, between 60 mM and 150 mM, between 100 mM and 300 mM, between 100 mM and 200 mM, between 100 mM and 175 mM, or between 100 mM and 150 mM sodium chloride. In a further embodiment, a formulation of the invention comprises 10 mM. 25 mM, 50 mM, 75 mM, 80 mM, 100 mM, 125 mM, 150 mM, 175 mM, 200 mM, or 300 mM sodium chloride. In a specific embodiment, a formulation of the invention comprises 75 mM sodium chloride.

The formulations of the invention may further comprise a surfactant. The term “surfactant” as used herein refers to organic substances having amphipathic structures; namely, they are composed of groups of opposing solubility tendencies, typically an oil-soluble hydrocarbon chain and a water-soluble ionic group. Surfactants can be classified, depending on the charge of the surface-active moiety, into anionic, cationic, and nonionic surfactants. Surfactants are often used as wetting, emulsifying, solubilizing, and dispersing agents for various pharmaceutical compositions and preparations of biological materials. Pharmaceutically acceptable surfactants like polysorbates (e.g. polysorbates 20 or 80); polyoxamers (e.g. poloxamer 188); Triton; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; and the MONAQUA™ series (Mona Industries, Inc., Paterson, N.J.), polyethyl glycol, polypropyl glycol, and copolymers of ethylene and propylene glycol (e.g. Pluronics, PF68 etc), can optionally be added to the formulations of the invention to reduce aggregation. Surfactants are particularly useful if a pump or plastic container is used to administer the formulation. The presence of a pharmaceutically acceptable surfactant mitigates the propensity for the protein to aggregate. In a specific embodiment, the formulations of the invention comprise a polysorbate which is at a concentration ranging from between about 0.001% to about 1%, or about 0.001% to about 0.1%, or about 0.01% to about 0.1%. In other specific embodiments, the formulations of the invention comprise a polysorbate which is at a concentration of 0.001%, or 0.002%, or 0.003%, or 0.004%, or 0.005%, or 0.006%, or 0.007%, or 0.008%, or 0.009%, or 0.01%, or 0.015%, or 0.02%. In another specific embodiment, the polysorbate is polysorbate-80. In a specific embodiment, the formulations of the invention comprise a polysorbate which is at a concentration ranging from between 0.001% to 1%, or 0.001% to 0.1%, or 0.01% to 0.1%. In other specific embodiments, the formulations of the invention comprise a polysorbate which is at a concentration of 0.001%, or 0.002%, or 0.003%, or 0.004%, or 0.005%, or 0.006%, or 0.007%, or 0.008%, or 0.009%, or 0.01%, or 0.015%, or 0.02%. In another specific embodiment, the polysorbate is polysorbate-80.

In one embodiment, a formulation of the invention comprises a surfactant. In one embodiment, a formulation of the invention comprises Polysorbate 20, Polysorbate 40, Polysorbate 60, or Polysorbate 80. In a specific embodiment, a formulation of the invention comprises Polysorbate 80.

In one embodiment, a formulation of the invention comprises at least about 0.001%, at least about 0.002%, at least about 0.005%, at least about 0.01%, at least about 0.02%, at least about 0.05%, at least about 0.1%, at least about 0.2%, or at least about 0.5% Polysorbate 80. In another embodiment, a formulation of the invention comprises between about 0.001% and about 0.5%, between about 0.001% and about 0.2%, between about 0.001% and about 0.1%, between about 0.001% and about 0.05%, between about 0.002% and about 0.5%, between about 0.002% and about 0.2%, between about 0.002% and about 0.1%, between about 0.002% and about 0.05%, between about 0.005% and about 0.5%, between about 0.005% and about 0.2%, between about 0.005% and about 0.1%, between about 0.005% and about 0.05%, between about 0.01% and about 0.5%, between about 0.01% and about 0.2%, between about 0.01% and about 0.1%, or between about 0.01% and about 0.05% Polysorbate 80. In a further embodiment, a formulation of the invention comprises about 0.001%, about 0.002%, about 0.005%, about 0.01%, about 0.02%, about 0.05%, about 0.1%, about 0.2%, and about 0.5% Polysorbate 80. In a specific embodiment, a formulation of the invention comprises about 0.02% Polysorbate 80. In a specific embodiment, a formulation of the invention comprises about 0.04% Polysorbate 80. In a specific embodiment, a formulation of the invention comprises about 0.05% Polysorbate 80.

In one embodiment, a formulation of the invention comprises at least 0.001%, at least 0.002%, at least 0.005%, at least 0.01%, at least 0.02%, at least 0.05%, at least 0.1%, at least 0.2%, or at least 0.5% Polysorbate 80. In another embodiment, a formulation of the invention comprises between 0.001% and 0.5%, between 0.001% and 0.2%, between 0.001% and 0.1%, between 0.001% and 0.05%, between 0.002% and 0.5%, between 0.002% and 0.2%, between 0.002% and 0.1%, between 0.002% and 0.05%, between 0.005% and 0.5%, between 0.005% and 0.2%, between 0.005% and 0.1%, between 0.005% and 0.05%, between 0.01% and 0.5%, between 0.01% and 0.2%, between 0.01% and 0.1%, or between 0.01% and 0.05% Polysorbate 80. In a further embodiment, a formulation of the invention comprises 0.001%, 0.002%, 0.005%, 0.01%, 0.02%, 0.05%, 0.1%, 0.2%, and 0.5% Polysorbate 80. In a specific embodiment, a formulation of the invention comprises 0.02% Polysorbate 80. In a specific embodiment, a formulation of the invention comprises 0.04% Polysorbate 80. In a specific embodiment, a formulation of the invention comprises 0.05% Polysorbate 80.

Optionally, the formulations of the invention may further comprise other common excipients and/or additives including, but not limited to, diluents, binders, stabilizers, lipophilic solvents, preservatives, adjuvants, or the like. Pharmaceutically acceptable excipients and/or additives may be used in the formulations of the invention. Commonly used excipients/additives, such as pharmaceutically acceptable chelators (for example, but not limited to, EDTA, DTPA or EGTA) can optionally be added to the formulations of the invention to reduce aggregation. These additives are particularly useful if a pump or plastic container is used to administer the formulation.

Preservatives, such as phenol, m-cresol, p-cresol, o-cresol, chlorocresol, benzyl alcohol, phenylmercuric nitrite, phenoxyethanol, formaldehyde, chlorobutanol, magnesium chloride (for example, but not limited to, hexahydrate), alkylparaben (methyl, ethyl, propyl, butyl and the like), benzalkonium chloride, benzethonium chloride, sodium dehydroacetate and thimerosal, or mixtures thereof can optionally be added to the formulations of the invention at any suitable concentration such as between about 0.001% to about 5%, or any range or value therein. The concentration of preservative used in the formulations of the invention is a concentration sufficient to yield an anti-microbial effect. Such concentrations are dependent on the preservative selected and are readily determined by the skilled artisan.

Other contemplated excipients/additives, which may be utilized in the formulations of the invention include, for example, flavoring agents, antimicrobial agents, sweeteners, antioxidants, antistatic agents, lipids such as phospholipids or fatty acids, steroids such as cholesterol, protein excipients such as serum albumin (human serum albumin (HSA), recombinant human albumin (rHA)), gelatin, casein, salt-forming counterions such as sodium and the like. These and additional known pharmaceutical excipients and/or additives suitable for use in the formulations of the invention are known in the art, e.g., as listed in “Remington: The Science & Practice of Pharmacy”, 21^{st} ed., Lippincott Williams & Wilkins, (2005), and in the “Physician's Desk Reference”, 60^{th} ed., Medical Economics, Montvale, N.J. (2005). Pharmaceutically acceptable carriers can be routinely selected that are suitable for the mode of administration, solubility and/or stability of Fc variant protein as well known in the art or as described herein.

It will be understood by one skilled in the art that the formulations of the invention may be isotonic with human blood, that is the formulations of the invention have essentially the same osmotic pressure as human blood. Such isotonic formulations will generally have an osmotic pressure from about 250 mOSm to about 350 mOSm. Isotonicity can be measured by, for example, using a vapor pressure or ice-freezing type osmometer. Tonicity of a formulation is adjusted by the use of tonicity modifiers. “Tonicity modifiers” are those pharmaceutically acceptable inert substances that can be added to the formulation to provide an isotonity of the formulation. Tonicity modifiers suitable for this invention include, but are not limited to, saccharides, salts and amino acids.

In certain embodiments, the formulations of the present invention have an osmotic pressure from about 100 mOSm to about 1200 mOSm, or from about 200 mOSm to about 1000 mOSm, or from about 200 mOSm to about 800 mOSm, or from about 200 mOSm to about 600 mOSm, or from about 250 mOSm to about 500 mOSm, or from about 250 mOSm to about 400 mOSm, or from about 250 mOSm to about 350 mOSm.

In certain embodiments, the formulations of the present invention have an osmotic pressure from 100 mOSm to 1200 mOSm, or from 200 mOSm to 1000 mOSm, or from 200 mOSm to 800 mOSm, or from 200 mOSm to 600 mOSm, or from 250 mOSm to 500 mOSm, or from 250 mOSm to 400 mOSm, or from 250 mOSm to 350 mOSm.

Concentration of any one or any combination of various components of the formulations of the invention are adjusted to achieve the desired tonicity of the final formulation. For example, the ratio of the carbohydrate excipient to antibody may be adjusted according to methods known in the art (e.g., U.S. Pat. No. 6,685,940). In certain embodiments, the molar ratio of the carbohydrate excipient to antibody may be from about 100 moles to about 1000 moles of carbohydrate excipient to about 1 mole of antibody, or from about 200 moles to about 6000 moles of carbohydrate excipient to about 1 mole of antibody, or from about 100 moles to about 510 moles of carbohydrate excipient to about 1 mole of antibody, or from about 100 moles to about 600 moles of carbohydrate excipient to about 1 mole of antibody.

Concentration of any one or any combination of various components of the formulations of the invention are adjusted to achieve the desired tonicity of the final formulation. For example, the ratio of the carbohydrate excipient to antibody may be adjusted according to methods known in the art (e.g., U.S. Pat. No. 6,685,940). In certain embodiments, the molar ratio of the carbohydrate excipient to antibody may be from 100 moles to 1000 moles of carbohydrate excipient to 1 mole of antibody, or from 200 moles to 6000 moles of carbohydrate excipient to 1 mole of antibody, or from 100 moles to 510 moles of carbohydrate excipient to 1 mole of antibody, or from 100 moles to 600 moles of carbohydrate excipient to 1 mole of antibody.

The desired isotonicity of the final formulation may also be achieved by adjusting the salt concentration of the formulations. Salts that are pharmaceutically acceptable and suitable for this invention as tonicity modifiers include, but are not limited to, sodium chloride, sodium succinate, sodium sulfate, potassium chloride, magnesium chloride, magnesium sulfate, and calcium chloride. In specific embodiments, formulations of the inventions comprise NaCl, MgCl₂, and/or CaCl₂. In one embodiment, concentration of NaCl is between about 75 mM and about 150 mM. In another embodiment, concentration of MgCl₂ is between about 1 mM and about 100 mM Amino acids that are pharmaceutically acceptable and suitable for this invention as tonicity modifiers include, but are not limited to, proline, alanine, L-arginine, asparagine, L-aspartic acid, glycine, serine, lysine, and histidine.

In one embodiment, a formulation of the invention comprises histidine, sodium chloride, trehalose, and Polysorbate 80. In one embodiment, a formulation of the invention comprises histidine, sodium chloride, and trehalose.

In one embodiment, a formulation of the invention comprises histidine, sodium chloride, and trehalose. In one embodiment, a formulation of the invention comprises between about 5 mM and about 100 mM histidine, between about 10 mM and about 300 mM sodium chloride, and between about 0.3% and about 10% trehalose, wherein said formulation has a pH of between about 5.0 and about 7.0. In another embodiment, a formulation of the invention comprises between about 5 mM and about 50 mM histidine, between about 50 mM and about 200 mM sodium chloride, and between about 1% and about 8% trehalose, wherein said formulation has a pH of between about 5.5 and about 6.5. In a further embodiment, a formulation of the invention comprises about 10 mM histidine, about 75 mM sodium chloride, and about 4% trehalose, wherein said formulation has a pH of about 6.0.

In one embodiment, a formulation of the invention comprises histidine, sodium chloride, trehalose and Polysorbate 80. In one embodiment, a formulation of the invention comprises between about 5 mM and about 100 mM histidine, between about 10 mM and about 300 mM sodium chloride, between about 0.3% and about 10% trehalose, and between about 0.005% and about 0.1% Polysorbate 80, wherein said formulation has a pH of between about 5.0 and about 7.0. In another embodiment, a formulation of the invention comprises between about 5 mM and about 50 mM histidine, between about 50 mM and about 200 mM sodium chloride, between about 1% and about 8% trehalose, and between about 0.01% and about 0.05% Polysorbate 80, wherein said formulation has a pH of between about 5.5 and about 6.5. In a further embodiment, a formulation of the invention comprises about 10 mM histidine, about 75 mM sodium chloride, about 4% trehalose and about 0.02% Polysorbate 80, wherein said formulation has a pH of about 6.0.

In one embodiment, a formulation of the invention comprises histidine, sodium chloride, and trehalose. In one embodiment, a formulation of the invention comprises between 5 mM and 100 mM histidine, between 10 mM and 300 mM sodium chloride, and between 0.3% and 10% trehalose, wherein said formulation has a pH of between 5.0 and 7.0. In another embodiment, a formulation of the invention comprises between 5 mM and 50 mM histidine, between 50 mM and 200 mM sodium chloride, and between 1% and 8% trehalose, wherein said formulation has a pH of between 5.5 and 6.5. In a further embodiment, a formulation of the invention comprises 10 mM histidine, 75 mM sodium chloride, and 4% trehalose, wherein said formulation has a pH of 6.0.

In one embodiment, a formulation of the invention comprises histidine, sodium chloride, trehalose and Polysorbate 80. In one embodiment, a formulation of the invention comprises between 5 mM and 100 mM histidine, between 10 mM and 300 mM sodium chloride, between 0.3% and 10% trehalose, and between 0.005% and 0.1% Polysorbate 80, wherein said formulation has a pH of between 5.0 and 7.0. In another embodiment, a formulation of the invention comprises between 5 mM and 50 mM histidine, between 50 mM and 200 mM sodium chloride, between 1% and 8% trehalose, and between 0.01% and 0.05% Polysorbate 80, wherein said formulation has a pH of between 5.5 and 6.5. In a further embodiment, a formulation of the invention comprises 10 mM histidine, 75 mM sodium chloride, 4% trehalose and 0.02% Polysorbate 80, wherein said formulation has a pH of 6.0.

In one embodiment, a formulation of the invention comprises between about 1 mg/ml and about 100 mg/ml anti-CD19 antibody of the invention, about 20 mM histidine, about 75 mM sodium chloride, about 4% trehalose and about 0.02% Polysorbate 80, wherein said formulation has a pH of about 6.0. In another embodiment, a formulation of the invention comprises about 100 mg/ml anti-CD19 antibody of the invention, about 20 mM histidine, about 75 mM sodium chloride, about 4% trehalose and about 0.02% Polysorbate 80, wherein said formulation has a pH of about 6.0. In another embodiment, a formulation of the invention comprises about 100 mg/ml anti-CD19 antibody of the invention, about 20 mM histidine; about 75 mM sodium chloride; about 4% trehalose; and about 0.01%, about 0.02% about 0.04% about 0.08% or about 0.1% Polysorbate 80; and a pH of about 6.0.

In one embodiment, a formulation of the invention comprises between about 1 mg/ml and about 60 mg/ml anti-CD19 antibody of the invention, about 10 mM histidine, about 75 mM sodium chloride, and about 4% trehalose, wherein said formulation has a pH of about 6.0. In another embodiment, a formulation of the invention comprises about 10 mg/ml anti-CD19 antibody of the invention, about 10 mM histidine, about 75 mM sodium chloride, and about 4% trehalose, wherein said formulation has a pH of about 6.0. In another embodiment, a formulation of the invention comprises about 50 mg/ml anti-CD19 antibody of the invention, about 10 mM histidine, about 75 mM sodium chloride, and about 4% trehalose, wherein said formulation has a pH of about 6.0. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention comprises between about 1 mg/ml and about 60 mg/ml anti-CD19 antibody of the invention, about 10 mM histidine, about 75 mM sodium chloride, about 4% trehalose and about 0.02% Polysorbate 80, wherein said formulation has a pH of about 6.0. In another embodiment, a formulation of the invention comprises about 10 mg/ml anti-CD19 antibody of the invention, about 10 mM histidine, about 75 mM sodium chloride, about 4% trehalose and about 0.02% Polysorbate 80, wherein said formulation has a pH of about 6.0. In another embodiment, a formulation of the invention comprises about 50 mg/ml anti-CD19 antibody of the invention, about 10 mM histidine, about 75 mM sodium chloride, about 4% trehalose and about 0.02% Polysorbate 80, wherein said formulation has a pH of about 6.0. In another embodiment, a formulation of the invention comprises up to 100 mg/ml anti-CD19 antibodies of the invention, about 10 mM histidine, about 75 mM sodium chloride, about 4% trehalose and about 0.02% Polysorbate 80, wherein said formulation has a pH of about 6.0. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention comprises between 1 mg/ml and 60 mg/ml anti-CD19 antibody of the invention, 10 mM histidine, 75 mM sodium chloride, and 4% trehalose, wherein said formulation has a pH of 6.0. In another embodiment, a formulation of the invention comprises 10 mg/ml anti-CD19 antibody of the invention, 10 mM histidine, 75 mM sodium chloride, and 4% trehalose, wherein said formulation has a pH of 6.0. In another embodiment, a formulation of the invention comprises 50 mg/ml anti-CD19 antibody of the invention, 10 mM histidine, 75 mM sodium chloride, and 4% trehalose, wherein said formulation has a pH of 6.0. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention comprises between 1 mg/ml and 60 mg/ml anti-CD19 antibody of the invention, 10 mM histidine, 75 mM sodium chloride, 4% trehalose and 0.02% Polysorbate 80, wherein said formulation has a pH of 6.0. In another embodiment, a formulation of the invention comprises 10 mg/ml anti-CD19 antibody of the invention, 10 mM histidine, 75 mM sodium chloride, 4% trehalose and 0.02% Polysorbate 80, wherein said formulation has a pH of 6.0. In another embodiment, a formulation of the invention comprises 50 mg/ml anti-CD19 antibody of the invention, 10 mM histidine, 75 mM sodium chloride, 4% trehalose and 0.02% Polysorbate 80, wherein said formulation has a pH of 6.0. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention consists of between about 1 mg/ml and about 60 mg/ml anti-CD19 antibody of the invention, about 10 mM histidine, about 75 mM sodium chloride, and about 4% trehalose, wherein said formulation has a pH of about 6.0. In another embodiment, a formulation of the invention consists of about 10 mg/ml anti-CD19 antibody of the invention, about 10 mM histidine, about 75 mM sodium chloride, and about 4% trehalose, wherein said formulation has a pH of about 6.0. In another embodiment, a formulation of the invention consists of about 50 mg/ml anti-CD19 antibody of the invention, about 10 mM histidine, about 75 mM sodium chloride, and about 4% trehalose, wherein said formulation has a pH of about 6.0. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention consists of between about 1 mg/ml and about 60 mg/ml anti-CD19 antibody of the invention, about 10 mM histidine, about 75 mM sodium chloride, about 4% trehalose and about 0.02% Polysorbate 80, wherein said formulation has a pH of about 6.0. In another embodiment, a formulation of the invention consists of about 10 mg/ml anti-CD19 antibody of the invention, about 10 mM histidine, about 75 mM sodium chloride, about 4% trehalose and about 0.02% Polysorbate 80, wherein said formulation has a pH of about 6.0. In another embodiment, a formulation of the invention consists of about 50 mg/ml anti-CD19 antibody of the invention, about 10 mM histidine, about 75 mM sodium chloride, about 4% trehalose and about 0.02% Polysorbate 80, wherein said formulation has a pH of about 6.0. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention consists of between 1 mg/ml and 60 mg/ml anti-CD19 antibody of the invention, 10 mM histidine, 75 mM sodium chloride, and 4% trehalose, wherein said formulation has a pH of 6.0. In another embodiment, a formulation of the invention consists of 10 mg/ml anti-CD19 antibody of the invention, 10 mM histidine, 75 mM sodium chloride, and 4% trehalose, wherein said formulation has a pH of 6.0. In another embodiment, a formulation of the invention consists of 50 mg/ml anti-CD19 antibody of the invention, 10 mM histidine, 75 mM sodium chloride, and 4% trehalose, wherein said formulation has a pH of 6.0. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention consists of 10, 25, 50, or 100 mg/mL 60 mg/ml anti-CD19 antibody of the invention, 10 mM histidine, 75 mM sodium chloride, and 4% trehalose, wherein said formulation has a pH of 6.0. In another embodiment, a formulation of the invention consists of 10 mg/ml anti-CD19 antibody of the invention, 10 mM histidine, 75 mM sodium chloride, and 4% trehalose, wherein said formulation has a pH of 6.0. In another embodiment, a formulation of the invention consists of 25 mg/ml anti-CD19 antibody of the invention, 10 mM histidine, 75 mM sodium chloride, and 4% trehalose, wherein said formulation has a pH of 6.0. In another embodiment, a formulation of the invention consists of 50 mg/ml anti-CD19 antibody of the invention, 10 mM histidine, 75 mM sodium chloride, and 4% trehalose, wherein said formulation has a pH of 6.0. In another embodiment, a formulation of the invention consists of 100 mg/ml anti-CD19 antibody of the invention, 10 mM histidine, 75 mM sodium chloride, and 4% trehalose, wherein said formulation has a pH of 6.0. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention consists of between 1 mg/ml and 60 mg/ml anti-CD19 antibody of the invention, 10 mM histidine, 75 mM sodium chloride, 4% trehalose and 0.02% Polysorbate 80, wherein said formulation has a pH of 6.0. In another embodiment, a formulation of the invention consists of 10 mg/ml anti-CD19 antibody of the invention, 10 mM histidine, 75 mM sodium chloride, 4% trehalose and 0.02% Polysorbate 80, wherein said formulation has a pH of 6.0. In another embodiment, a formulation of the invention consists of 50 mg/ml anti-CD19 antibody of the invention, 10 mM histidine, 75 mM sodium chloride, 4% trehalose and 0.02% Polysorbate 80, wherein said formulation has a pH of 6.0. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention consists of 10, 25, 50, or 100 mg/mL anti-CD19 antibody of the invention, 10 mM histidine, 75 mM sodium chloride, 4% trehalose and 0.02% Polysorbate 80, wherein said formulation has a pH of 6.0. In another embodiment, a formulation of the invention consists of 10 mg/ml anti-CD19 antibody of the invention, 10 mM histidine, 75 mM sodium chloride, 4% trehalose and 0.02% Polysorbate 80, wherein said formulation has a pH of 6.0. In another embodiment, a formulation of the invention consists of 25 mg/ml anti-CD19 antibody of the invention, 10 mM histidine, 75 mM sodium chloride, 4% trehalose and 0.02% Polysorbate 80, wherein said formulation has a pH of 6.0. In another embodiment, a formulation of the invention consists of 50 mg/ml anti-CD19 antibody of the invention, 10 mM histidine, 75 mM sodium chloride, 4% trehalose and 0.02% Polysorbate 80, wherein said formulation has a pH of 6.0. In another embodiment, a formulation of the invention consists of 100 mg/ml anti-CD19 antibody of the invention, 10 mM histidine, 75 mM sodium chloride, 4% trehalose and 0.02% Polysorbate 80, wherein said formulation has a pH of 6.0. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment the formulations of the invention are pyrogen-free formulations which are substantially free of endotoxins and/or related pyrogenic substances. Endotoxins include toxins that are confined inside a microorganism and are released only when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). When therapeutic proteins are administered in amounts of several hundred or thousand milligrams per kilogram body weight, as can be the case with antibodies, even trace amounts of harmful and dangerous endotoxin must be removed. In certain specific embodiments, the endotoxin and pyrogen levels in the composition are less then 10 EU/mg, or less then 5 EU/mg, or less then 1 EU/mg, or less then 0.1 EU/mg, or less then 0.01 EU/mg, or less then 0.001 EU/mg.

When used for in vivo administration, the formulations of the invention should be sterile. The formulations of the invention may be sterilized by various sterilization methods, including sterile filtration, radiation, etc. In one embodiment, the antibody formulation is filter-sterilized with a presterilized 0.22-micron filter. Sterile compositions for injection can be formulated according to conventional pharmaceutical practice as described in “Remington: The Science & Practice of Pharmacy”, 21^{st} ed., Lippincott Williams & Wilkins, (2005). Formulations comprising antibodies, such as those disclosed herein, ordinarily will be stored in lyophilized form or in solution. It is contemplated that sterile compositions comprising antibodies are placed into a container having a sterile access port, for example, an intravenous solution bag or vial having an adapter that allows retrieval of the formulation, such as a stopper pierceable by a hypodermic injection needle. In one embodiment, a composition of the invention is provided as a pre-filled syringe. In one embodiment the syringe has high transparency, low water vapor and oxygen permeability, and no tungsten residuals. In another embodiment, a composition of the invention is provided in a pre-filled tungsten-free syringe, such as the “ultra-100” syringe. In another embodiment the syringe is a Clearject™ (Geresheimer, AG, Germany) or InJentle™ (SCHOTT Pharmaceutical Packaging, Germany) syringe.

### 6.2. Stability of Formulations

In one embodiment, a formulation of the invention stabilizes an anti-CD19 antibody. In one embodiment, a formulation of the invention prevents aggregation of an anti-CD19 antibody or fragment thereof. In another embodiment, a formulation of the invention prevents fragmentation of an anti-CD19 antibody or fragment thereof. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention is stable upon storage at about 40° C. for at least about 1 week, at least about 2 weeks, at least about 3 weeks, or at least about 4 weeks. In one embodiment, a formulation of the invention is stable upon storage at about 40° C. for at least about 1 month, at least about 2 months, at least about 3 months, at least about 4 months, at least about 5 months, or at least about 6 months. In a specific embodiment, a formulation of the invention is stable upon storage in a pre-filled syringe.

In one embodiment, a formulation of the invention is stable upon storage at about 25° C. for at least about 1 week, at least about 2 weeks, at least about 3 weeks, or at least about 4 weeks. In one embodiment, a formulation of the invention is stable upon storage at about 25° C. for at least about 1 month, at least about 2 months, at least about 3 months, at least about 4 months, at least about 5 months, or at least about 6 months. In a specific embodiment, a formulation of the invention is stable upon storage in a pre-filled syringe.

In one embodiment, a formulation of the invention is stable upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention is stable upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stable upon storage in a pre-filled syringe.

In one embodiment, a formulation of the invention is stable upon storage at about 40° C. for about 1 week, about 2 weeks, about 3 weeks, or about 4 weeks. In one embodiment, a formulation of the invention is stable upon storage at about 40° C. for about 1 month, about 2 months, about 3 months, about 4 months, about 5 months, or about 6 months. In a specific embodiment, a formulation of the invention is stable upon storage in a pre-filled syringe.

In one embodiment, a formulation of the invention is stable upon storage at about 25° C. for about 1 week, about 2 weeks, about 3 weeks, or about 4 weeks. In one embodiment, a formulation of the invention is stable upon storage at about 25° C. for about 1 month, about 2 months, about 3 months, about 4 months, about 5 months, or about 6 months. In a specific embodiment, a formulation of the invention is stable upon storage in a pre-filled syringe.

In one embodiment, a formulation of the invention is stable upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention is stable upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stable upon storage in a pre-filled syringe.

In one embodiment, a formulation of the invention is stable upon storage at about −10° C., about −15° C., about −25° C., about −30° C., about −35° C., about −35° C., about −45° C., about −50° C., about −55° C., about −60° C., about −65° C., about −70° C. about −75° C., about −80° C. In a specific embodiment, a formulation of the invention is stable upon storage at these temperatures in a prefilled syringe.

The present inventions provide stable liquid formulations comprising anti-CD19 antibodies of the invention. The stability of said antibody can be assessed by degrees of aggregation, degradation or fragmentation, as measured by HPSEC, reverse phase chromatography, static light scattering (SLS), Fourier Transform Infrared Spectroscopy (FTIR), circular dichroism (CD), urea unfolding techniques, intrinsic tryptophan fluorescence, differential scanning calorimetry, and/or ANS binding techniques, compared to a reference formulation comprising a reference antibody. For example, a reference formulation may be a reference standard frozen at −70° C. consisting of 10 mg/ml of a reference antibody antibody (including antibody fragment thereof) (for example, but not limited to, an antibody comprising the 16C4 variable region and an Fc region having complex N-glycoside-linked sugar chains in which fucose is not bound to N-acetylglucosamine in the reducing end in the sugar chain) in 10 mM histidine (pH 6.0) that contains 75 mMNaCl and 4% trehalose, which reference formulation regularly gives a single monomer peak (e.g., ≧95% area) by HPSEC. In certain embodiments, a reference formulation is identical to the formulation whose stability is tested; the reference formulation may be stored frozen at −70° C. during the stability testing to preserve the reference formulation in its original condition. For example, the reference standard for assessing any loss of CD19 antigen binding activity in a formulation stored at 40° C. may be the identical formulation stored at −70° C. for 30 days. The overall stability of a formulation comprising an antibody (including antibody fragment thereof) may also be assessed by various immunological assays including, for example, ELISA and radioimmunoassay using isolated antigen molecules. Furthermore, the stability of a formulation comprising an antibody may also be assessed using various assays designed to measure a functional characteristic of the antibody, for example, assays designed to measure antigen binding affinity, in vitro ADCC activity, in vivo depletion activity, in vitro CDC activity.

In one embodiment, a formulation of the invention comprises an anti-CD19 antibody that has a CD19 binding activity that is at least 50%, at least 60%, at least 70%, at least 80%, at least 90%, at least 95%, or at least 99% of the CD19 binding activity of a reference antibody, wherein said formulation was stored at about 40° C. for at least about 1 week, at least about 2 weeks, at least about 3 weeks, or at least about 4 weeks. In one embodiment, a formulation of the invention comprises an anti-CD19 antibody that has a CD19 binding activity that is at least 50%, at least 60%, at least 70%, at least 80%, at least 90%, at least 95%, or at least 99% of the CD19 binding activity of a reference antibody, wherein said formulation was stored at about 40° C. for at least about 1 month, at least about 2 months, at least about 3 months, at least about 4 months, at least about 5 months, or at least about 6 months. In a specific embodiment, a formulation of the invention is stored in a pre-filled syringe. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention comprises an anti-CD19 antibody that has a CD19 binding activity that is at least 50%, at least 60%, at least 70%, at least 80%, at least 90%, at least 95%, or at least 99% of the CD19 binding activity of a reference antibody, wherein said formulation was stored at about 25° C. for at least about 1 week, at least about 2 weeks, at least about 3 weeks, or at least about 4 weeks. In one embodiment, a formulation of the invention comprises an anti-CD19 antibody that has a CD19 binding activity that is at least 50%, at least 60%, at least 70%, at least 80%, at least 90%, at least 95%, or at least 99% of the CD19 binding activity of a reference antibody, wherein said formulation was stored at about 25° C. for at least about 1 month, at least about 2 months, at least about 3 months, at least about 4 months, at least about 5 months, or at least about 6 months. In a specific embodiment, a formulation of the invention is stored in a pre-filled syringe. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention comprises an anti-CD19 antibody that has a CD19 binding activity that is at least 50%, at least 60%, at least 70%, at least 80%, at least 90%, at least 95%, or at least 99% of the CD19 binding activity of a reference antibody, wherein said formulation was stored at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-CD19 antibody that has a CD19 binding activity that is at least 50%, at least 60%, at least 70%, at least 80%, at least 90%, at least 95%, or at least 99% of the CD19 binding activity of a reference antibody, wherein said formulation was stored at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention comprises an anti-CD19 antibody that has a CD19 binding activity that is at least 50%, at least 60%, at least 70%, at least 80%, at least 90%, at least 95%, or at least 99% of the CD19 binding activity of a reference antibody, wherein said formulation was stored at about 40° C. for about 1 week, about 2 weeks, about 3 weeks, or about 4 weeks. In one embodiment, a formulation of the invention comprises an anti-CD19 antibody that has a CD19 binding activity that is at least 50%, at least 60%, at least 70%, at least 80%, at least 90%, at least 95%, or at least 99% of the CD19 binding activity of a reference antibody, wherein said formulation was stored at about 40° C. for about 1 month, about 2 months, about 3 months, about 4 months, about 5 months, or about 6 months. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention comprises an anti-CD19 antibody that has a CD19 binding activity that is at least 50%, at least 60%, at least 70%, at least 80%, at least 90%, at least 95%, or at least 99% of the CD19 binding activity of a reference antibody, wherein said formulation was stored at about 25° C. for about 1 week, about 2 weeks, about 3 weeks, or about 4 weeks. In one embodiment, a formulation of the invention comprises an anti-CD19 antibody that has a CD19 binding activity that is at least 50%, at least 60%, at least 70%, at least 80%, at least 90%, at least 95%, or at least 99% of the CD19 binding activity of a reference antibody, wherein said formulation was stored at about 25° C. for about 1 month, about 2 months, about 3 months, about 4 months, about 5 months, or about 6 months. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention comprises an anti-CD19 antibody that has a CD19 binding activity that is at least 50%, at least 60%, at least 70%, at least 80%, at least 90%, at least 95%, or at least 99% of the CD19 binding activity of a reference antibody, wherein said formulation was stored at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-CD19 antibody that has a CD19 binding activity that is at least 50%, at least 60%, at least 70%, at least 80%, at least 90%, at least 95%, or at least 99% of the CD19 binding activity of a reference antibody, wherein said formulation was stored at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention comprises an anti-CD19 antibody, wherein the antibody loses at most 50%, at most 40%, at most 30%, at most 20%, at most 10%, at most 5%, or at most 1% of its CD19 binding activity during storage of the formulation at about 40° C. for at least about 1 week, at least about 2 weeks, at least about 3 weeks, or at least about 4 weeks. In one embodiment, a formulation of the invention comprises an anti-CD19 antibody, wherein the antibody loses at most 50%, at most 40%, at most 30%, at most 20%, at most 10%, at most 5%, or at most 1% of its CD19 binding activity during storage of the formulation at about 40° C. for at least about 1 month, at least about 2 months, at least about 3 months, at least about 4 months, at least about 5 months, or at least about 6 months. In a specific embodiment, a formulation of the invention is stored in a pre-filled syringe. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain. As used herein, the terms “at most’ and “no more than” have the same meaning.

In one embodiment, a formulation of the invention comprises an anti-CD19 antibody, wherein the antibody loses at most 50%, at most 40%, at most 30%, at most 20%, at most 10%, at most 5%, or at most 1% of its CD19 binding activity during storage of the formulation at about 40° C. for at least about 1 week, at least about 2 weeks, at least about 3 weeks, or at least about 4 weeks. In one embodiment, a formulation of the invention comprises an anti-CD19 antibody, wherein the antibody loses at most 50%, at most 40%, at most 30%, at most 20%, at most 10%, at most 5%, or at most 1% of its CD19 binding activity during storage of the formulation at about 40° C. for at least about 1 month, at least about 2 months, at least about 3 months, at least about 4 months, at least about 5 months, or at least about 6 months. In a specific embodiment, a formulation of the invention is stored in a pre-filled syringe. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention comprises an anti-CD19 antibody, wherein the antibody loses at most 50%, at most 40%, at most 30%, at most 20%, at most 10%, at most 5%, or at most 1% of its CD19 binding activity during storage of the formulation at about 25° C. for at least about 1 week, at least about 2 weeks, at least about 3 weeks, or at least about 4 weeks. In one embodiment, a formulation of the invention comprises an anti-CD19 antibody, wherein the antibody loses at most 50%, at most 40%, at most 30%, at most 20%, at most 10%, at most 5%, or at most 1% of its CD19 binding activity during storage of the formulation at about 25° C. for at least about 1 month, at least about 2 months, at least about 3 months, at least about 4 months, at least about 5 months, or at least about 6 months. In a specific embodiment, a formulation of the invention is stored in a pre-filled syringe. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention comprises an anti-CD19 antibody, wherein the antibody loses at most 50%, at most 40%, at most 30%, at most 20%, at most 10%, at most 5%, or at most 1% of its CD19 binding activity during storage of the formulation at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-CD19 antibody, wherein the antibody loses at most 50%, at most 40%, at most 30%, at most 20%, at most 10%, at most 5%, or at most 1% of its CD19 binding activity during storage of the formulation at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention comprises an anti-CD19 antibody, wherein the antibody loses at most 50%, at most 40%, at most 30%, at most 20%, at most 10%, at most 5%, or at most 1% of its CD19 binding activity during storage of the formulation at about 40° C. for about 1 week, about 2 weeks, about 3 weeks, or about 4 weeks. In one embodiment, a formulation of the invention comprises an anti-CD19 antibody, wherein the antibody loses at most 50%, at most 40%, at most 30%, at most 20%, at most 10%, at most 5%, or at most 1% of its CD19 binding activity during storage of the formulation at about 40° C. for about 1 month, about 2 months, about 3 months, about 4 months, about 5 months, or about 6 months. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention comprises an anti-CD19 antibody, wherein the antibody loses at most 50%, at most 40%, at most 30%, at most 20%, at most 10%, at most 5%, or at most 1% of its CD19 binding activity during storage of the formulation at about 25° C. for about 1 week, about 2 weeks, about 3 weeks, or about 4 weeks. In one embodiment, a formulation of the invention comprises an anti-CD19 antibody, wherein the antibody loses at most 50%, at most 40%, at most 30%, at most 20%, at most 10%, at most 5%, or at most 1% of its CD19 binding activity during storage of the formulation at about 25° C. for about 1 month, about 2 months, about 3 months, about 4 months, about 5 months, or about 6 months. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention comprises an anti-CD19 antibody, wherein the antibody loses at most 50%, at most 40%, at most 30%, at most 20%, at most 10%, at most 5%, or at most 1% of its CD19 binding activity during storage of the formulation at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-CD19 antibody, wherein the antibody loses at most 50%, at most 40%, at most 30%, at most 20%, at most 10%, at most 5%, or at most 1% of its CD19 binding activity during storage of the formulation at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention comprises an anti-CD19 antibody, wherein less than 1%, less than 2%, less than 3%, less than 4%, less than 5%, less than 7% or less than 10% of said antibody forms an aggregate as determined by HPSEC upon storage at about 40° C. for at least about 1 week, at least about 2 weeks, at least about 3 weeks, or at least about 4 weeks. In one embodiment, a formulation of the invention comprises an anti-CD19 antibody, wherein less than 1%, less than 2%, less than 3%, less than 4%, less than 5%, less than 7% or less than 10% of said antibody forms an aggregate as determined by HPSEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention comprises an anti-CD19 antibody, wherein less than 1%, less than 2%, less than 3%, less than 4%, less than 5%, less than 7% or less than 10% of said antibody forms an aggregate as determined by HPSEC upon storage at about 25° C. for at least about 1 week, at least about 2 weeks, at least about 3 weeks, or at least about 4 weeks. In one embodiment, a formulation of the invention comprises an anti-CD19 antibody, wherein less than 1%, less than 2%, less than 3%, less than 4%, less than 5%, less than 7% or less than 10% of said antibody forms an aggregate as determined by HPSEC upon storage at about 25° C. for at least about 1 month, at least about 2 months, at least about 3 months, at least about 4 months, at least about 5 months, or at least about 6 months. In a specific embodiment, a formulation of the invention is stored in a pre-filled syringe. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention comprises an anti-CD19 antibody, wherein less than 1%, less than 2%, less than 3%, less than 4%, less than 5%, less than 7% or less than 10% of said antibody forms an aggregate as determined by HPSEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-CD19 antibody, wherein less than 1%, less than 2%, less than 3%, less than 4%, less than 5%, less than 7% or less than 10% of said antibody forms an aggregate as determined by HPSEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention comprises an anti-CD19 antibody, wherein less than 1%, less than 2%, less than 3%, less than 4%, less than 5%, less than 7% or less than 10% of said antibody forms an aggregate as determined by HPSEC upon storage at about 40° C. for about 1 week, about 2 weeks, about 3 weeks, or about 4 weeks. In one embodiment, a formulation of the invention comprises an anti-CD19 antibody, wherein less than 1%, less than 2%, less than 3%, less than 4%, less than 5%, less than 7% or less than 10% of said antibody forms an aggregate as determined by HPSEC upon storage at about 40° C. for about 1 month, about 2 months, about 3 months, about 4 months, about 5 months, or about 6 months. In a specific embodiment, a formulation of the invention is stored in a pre-filled syringe. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention comprises an anti-CD19 antibody, wherein less than 1%, less than 2%, less than 3%, less than 4%, less than 5%, less than 7% or less than 10% of said antibody forms an aggregate as determined by HPSEC upon storage at about 25° C. for about 1 week, about 2 weeks, about 3 weeks, or about 4 weeks. In one embodiment, a formulation of the invention comprises an anti-CD19 antibody, wherein less than 1%, less than 2%, less than 3%, less than 4%, less than 5%, less than 7% or less than 10% of said antibody forms an aggregate as determined by HPSEC upon storage at about 25° C. for about 1 month, about 2 months, about 3 months, about 4 months, about 5 months, or about 6 months. In a specific embodiment, a formulation of the invention is stored in a pre-filled syringe. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention comprises an anti-CD19 antibody, wherein less than 1%, less than 2%, less than 3%, less than 4%, less than 5%, less than 7% or less than 10% of said antibody forms an aggregate as determined by HPSEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-CD19 antibody, wherein less than 1%, less than 2%, less than 3%, less than 4%, less than 5%, less than 7% or less than 10% of said antibody forms an aggregate as determined by HPSEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention comprises an anti-CD19 antibody, wherein less than 1%, less than 2%, less than 3%, less than 4%, less than 5%, less than 7% or less than 10% of said antibody is fragmented as determined by RP-HPLC or SEC upon storage at about 40° C. for at least about 1 week, at least about 2 weeks, at least about 3 weeks, or at least about 4 weeks. In one embodiment, a formulation of the invention comprises an anti-CD19 antibody, wherein less than 1%, less than 2%, less than 3%, less than 4%, less than 5%, less than 7% or less than 10% of said antibody is fragmented as determined by RP-HPLC or SEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention comprises an anti-CD19 antibody, wherein less than 1%, less than 2%, less than 3%, less than 4%, less than 5%, less than 7% or less than 10% of said antibody is fragmented as determined by RP-HPLC or SEC upon storage at about 25° C. for at least about 1 week, at least about 2 weeks, at least about 3 weeks, or at least about 4 weeks. In one embodiment, a formulation of the invention comprises an anti-CD19 antibody, wherein less than 1%, less than 2%, less than 3%, less than 4%, less than 5%, less than 7% or less than 10% of said antibody is fragmented as determined by RP-HPLC or SEC upon storage at about 25° C. for at least about 1 month, at least about 2 months, at least about 3 months, at least about 4 months, at least about 5 months, or at least about 6 months. In a specific embodiment, a formulation of the invention is stored in a pre-filled syringe. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention comprises an anti-CD19 antibody, wherein less than 1%, less than 2%, less than 3%, less than 4%, less than 5%, less than 7% or less than 10% of said antibody is fragmented as determined by RP-HPLC or SEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-CD19 antibody, wherein less than 1%, less than 2%, less than 3%, less than 4%, less than 5%, less than 7% or less than 10% of said antibody is fragmented as determined by RP-HPLC or SEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention comprises an anti-CD19 antibody, wherein less than 1%, less than 2%, less than 3%, less than 4%, less than 5%, less than 7% or less than 10% of said antibody is fragmented as determined by RP-HPLC or SEC upon storage at about 40° C. for about 1 week, about 2 weeks, about 3 weeks, or about 4 weeks. In one embodiment, a formulation of the invention comprises an anti-CD19 antibody, wherein less than 1%, less than 2%, less than 3%, less than 4%, less than 5%, less than 7% or less than 10% of said antibody is fragmented as determined by RP-HPLC or SEC upon storage at about 40° C. for about 1 month, about 2 months, about 3 months, about 4 months, about 5 months, or about 6 months. In a specific embodiment, a formulation of the invention is stored in a pre-filled syringe. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention comprises an anti-CD19 antibody, wherein less than 1%, less than 2%, less than 3%, less than 4%, less than 5%, less than 7% or less than 10% of said antibody is fragmented as determined by RP-HPLC or SEC upon storage at about 25° C. for about 1 week, about 2 weeks, about 3 weeks, or about 4 weeks. In one embodiment, a formulation of the invention comprises an anti-CD19 antibody, wherein less than 1%, less than 2%, less than 3%, less than 4%, less than 5%, less than 7% or less than 10% of said antibody is fragmented as determined by RP-HPLC or SEC upon storage at about 25° C. for about 1 month, about 2 months, about 3 months, about 4 months, about 5 months, or about 6 months. In a specific embodiment, a formulation of the invention is stored in a pre-filled syringe. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention comprises an anti-CD19 antibody, wherein less than 1%, less than 2%, less than 3%, less than 4%, less than 5%, less than 7% or less than 10% of said antibody is fragmented as determined by RP-HPLC or SEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-CD19 antibody, wherein less than 1%, less than 2%, less than 3%, less than 4%, less than 5%, less than 7% or less than 10% of said antibody is fragmented as determined by RP-HPLC or SEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention is clear and colorless as determined by visual inspection upon storage at about 40° C. for at least about 1 week, at least about 2 weeks, at least about 3 weeks, or at least about 4 weeks. In one embodiment, a formulation of the invention is clear and colorless as determined by visual inspection upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention is clear and colorless as determined by visual inspection upon storage at about 25° C. for at least about 1 week, at least about 2 weeks, at least about 3 weeks, or at least about 4 weeks. In one embodiment, a formulation of the invention is clear and colorless as determined by visual inspection upon storage at about 25° C. for at least about 1 month, at least about 2 months, at least about 3 months, at least about 4 months, at least about 5 months, or at least about 6 months. In a specific embodiment, a formulation of the invention is stored in a pre-filled syringe. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention is clear and colorless as determined by visual inspection upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention is clear and colorless as determined by visual inspection upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention is clear and colorless as determined by visual inspection upon storage at about 40° C. for about 1 week, about 2 weeks, about 3 weeks, or about 4 weeks. In one embodiment, a formulation of the invention is clear and colorless as determined by visual inspection upon storage at about 40° C. for about 1 month, about 2 months, about 3 months, about 4 months, about 5 months, or about 6 months. In a specific embodiment, a formulation of the invention is stored in a pre-filled syringe. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention is clear and colorless as determined by visual inspection upon storage at about 25° C. for about 1 week, about 2 weeks, about 3 weeks, or about 4 weeks. In one embodiment, a formulation of the invention is clear and colorless as determined by visual inspection upon storage at about 25° C. for about 1 month, about 2 months, about 3 months, about 4 months, about 5 months, or about 6 months. In a specific embodiment, a formulation of the invention is stored in a pre-filled syringe. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention is clear and colorless as determined by visual inspection upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention is clear and colorless as determined by visual inspection upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In certain embodiments, the formulations of the invention maintain improved aggregation profiles upon storage, for example, for extended periods (for example, but not limited to 1 week, 1 month, 6 months, 1 year, 2 years, 3 years or 5 years) at room temperature or 4° C. or for periods (such as, but not limited to 1 week, 2 weeks, 3 weeks, 1 month, 2 months, 3 months, or 6 months) at elevated temperatures such as 38° C.-42° C. In certain embodiments, the formulations maintain improved aggregation profiles upon storage while exposed to light or stored in the dark in a variety of humidity conditions including but not limited to a relative humidity of up to 10%, or up to 20%, or up to 30%, or up to 40%, or up to 50%, or up to 60%, or up to 70%, or up to 80%, or up to 90%, or up to 100%. It will be understood in the art that the term “ambient” conditions generally refers to temperatures of about 20° C. at a relative humidity of between 10% and 60% with exposure to light. Similarly, temperatures between about 2° C. and about 8° C. at a relative humidity of less then about 10% are collectively referred to as “4° C.” or “5° C.”, temperatures between about 23° C. and about 27° C. at a relative humidity of about 60% are collectively referred to as “25° C.” and temperatures between about 38° C. and about 42° C. at a relative humidity of about 75% are collectively referred to as “40° C.” In a specific embodiment, a formulation of the invention is stored in a pre-filled syringe.

In certain embodiments, after storage at 4° C. for at least one month, the formulations of the invention comprise (or consists of as the aggregate fraction) a particle profile of less than about 3.4 E+5 particles/ml of diameter 2-4 μm, less than about 4.0 E+4 particles/ml of diameter 4-10 μm, less than about 4.2 E+3 particles/ml of diameter 10-20 μm, less than about 5.0 E+2 particles/ml of diameter 20-30 μm, less than about 7.5 E+1 particles/ml of diameter 30-40 μm, and less than about 9.4 particles/ml of diameter 40-60 μm as determined by a particle multisizer. In certain embodiments, the formulations of the invention contain no detectable particles greater than 40 μm, or greater than 30 μm. In a specific embodiment, a formulation of the invention is stored in a pre-filled syringe.

In certain embodiments, a surfactant may be added to the formulation to enhance the storage stability of a composition comprising the anti-CD19 antibody. It has been found that formulations of the invention containing such surfactants have enhanced storage stability over even long periods of time while under varying storage conditions. This enhanced stability may include the inhibition of particle formations in the stored formulation, while retaining the purity of the anti-CD19 antibody. This enhanced stability may further include the retention of the activity of the anti-CD19 antibody under various conditions. In certain embodiments, the formulation of the invention comprises the surfactant, Polysorbate 80. In another embodiment, Polysorbate 80 is added to the anti-CD19 antibody formulation in an amount sufficient to storage stabilize the composition, such as about 0.01%, about 0.02% about 0.04% about 0.08% or about 0.1% Polysorbate 80 weight per volume. “Storage stabilize” hereby means inhibiting the production of contaminants or particles in formulations of the invention over time and under storage conditions to within pharmaceutically acceptable levels, while retaining the purity of the composition.

Numerous methods useful for determining the degree of aggregation, and/or types and/or sizes of aggregates present in a protein formulation (e.g., antibody formulation of the invention) are known in the art, including but not limited to, size exclusion chromatography (SEC), high performance size exclusion chromatography (HPSEC), static light scattering (SLS), Fourier Transform Infrared Spectroscopy (FTIR), circular dichroism (CD), urea-induced protein unfolding techniques, intrinsic tryptophan fluorescence, differential scanning calorimetry, and 1-anilino-8-naphthalenesulfonic acid (ANS) protein binding techniques. For example, size exclusion chromatography (SEC) may be performed to separate molecules on the basis of their size, by passing the molecules over a column packed with the appropriate resin, the larger molecules (e.g. aggregates) will elute before smaller molecules (e.g. monomers). The molecules are generally detected by UV absorbance at 280 nm and may be collected for further characterization. High pressure liquid chromatographic columns are often utilized for SEC analysis (HP-SEC). Specific SEC methods are detailed in the section entitled “Examples” infra. Alternatively, analytical ultracentrifugation (AUC) may be utilized. AUC is an orthogonal technique which determines the sedimentation coefficients (reported in Svedberg, S) of macromolecules in a liquid sample. Like SEC, AUC is capable of separating and detecting antibody fragments/aggregates from monomers and is further able to provide information on molecular mass. Protein aggregation in the formulations may also be characterized by particle counter analysis using a coulter counter or by turbidity measurements using a turbidimeter. Turbidity is a measure of the amount by which the particles in a solution scatter light and, thus, may be used as a general indicator of protein aggregation. In addition, non-reducing polyacrylamide gel electrophoresis (PAGE) or capillary gel electrophoresis (CGE) may be used to characterize the aggregation and/or fragmentation state of antibodies or a fragment thereof in a formulation of the invention.

In one embodiment, a formulation of the invention is for parenteral administration. In one embodiment, a formulation of the invention is an injectable formulation. In one embodiment, a formulation of the invention is for intravenous, subcutaneous, or intramuscular administration. In a specific embodiment, a formulation of the invention comprises an anti-CD19 antibody wherein said formulation is for subcutaneous injection. In a specific embodiment, a formulation of the invention is stored in a pre-filled syringe. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention is for intravenous administration wherein said formulation comprises between about 1 mg/ml and about 40 mg/ml of an anti-CD19 antibody of the invention. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention is for subcutaneous administration wherein said formulation comprises between about 1 mg/ml and about 100 mg/ml of an anti-CD19 antibody of the invention. In a specific embodiment, a formulation of the invention is provided in a pre-filled syringe. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention is for aerosol administration.

The present invention also provides a pharmaceutical unit dosage form suitable for parenteral administration to a human which comprises an anti-CD19 antibody formulation in a suitable container. In one embodiment, a pharmaceutical unit dosage of the invention comprises an intravenously, subcutaneously, or intramuscularly delivered anti-CD19 antibody formulation. In another embodiment, a pharmaceutical unit dosage of the invention comprises aerosol delivered anti-CD19 antibody formulation. In a specific embodiment, a pharmaceutical unit dosage of the invention comprises a subcutaneously delivered anti-CD19 antibody formulation. In another embodiment, a pharmaceutical unit dosage of the invention comprises an aerosol delivered anti-CD19 antibody formulation. In a further embodiment, a pharmaceutical unit dosage of the invention comprises an intranasally administered anti-CD19 antibody formulation. In one embodiment, a suitable container is a pre-filled syringe. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

In one embodiment, a formulation of the invention is provided in a sealed container. In a specific embodiment, a formulation of the invention is provided in a pre-filled syringe. In a specific embodiment, a formulation of the invention comprises an anti-CD19 antibody comprising a heavy chain variable region of SEQ ID NO:104, a light chain variable region of SEQ ID NO:111 and an Fc region having complex N-glycoside-linked sugar chains in which fucose is not bound to N-acetylglucosamine in the reducing end in the sugar chain.

The present invention further provided a kit comprising an anti-CD19 antibody formulation of the invention.

The present invention also relates to methods of treating and preventing B cell-mediated diseases and disorders. The present invention also provides methods of preventing, managing, treating or ameliorating B cell-mediated diseases and disorders such as an inflammatory disease or disorder, an autoimmune disease or disorder, or a proliferative disease or disorder. In one embodiment, a method of the invention comprises administering to a subject in need thereof a prophylactically or therapeutically effective amount of an anti-CD19 antibody formulation.

In one embodiment, a method of the invention for the prevention, treatment, management or amelioration of a disease or disorder further comprises administering to said subject a prophylactically or therapeutically effective amount of a prophylactic or therapeutic agent other than an antibody or antibody fragment that specifically binds to CD19.

In one embodiment, a method of the invention for the prevention, treatment, management or amelioration of a disease or disorder further comprises administering to said subject a prophylactically or therapeutically effective amount of a prophylactic or therapeutic agent other than an antibody or antibody fragment that specifically binds CD19, wherein said prophylactic or therapeutic agent is an anti-inflammatory agent, immunomodulatory agent, anti-angiogenic agent, or anti-cancer agent.

### 6.3. Anti-CD-19 Antibodies

The present invention relates to human, humanized, or chimeric anti-CD19 antibodies that bind to the human CD19 antigen, as well as to compositions comprising those antibodies. In certain embodiments a human, humanized, or chimeric anti-CD19 antibody may mediate antigen-dependent-cell-mediated-cytotoxicity (ADCC). In other embodiments, the present invention is directed toward compositions comprising a human, humanized, or chimeric anti-CD19 antibody of the IgG1 and/or IgG3 human isotype, as well as to a human, humanized, or chimeric anti-CD19 antibody of the IgG2 and/or IgG4 human isotype, that may mediate human ADCC, CDC, and/or apoptosis. In further embodiments a human, humanized, or chimeric anti-CD19 antibody may inhibit anti-IgM/CpG stimulated B cell proliferation.

The present invention provides chimeric and humanized versions of the anti-CD19 mouse monoclonal antibodies HB12A and HB12B. In one embodiment, a humanized anti-CD19 antibody of the invention may bind to human CD19 with an affinity comparable to the binding affinity of HB12A or HB12B or comparable to the binding affinity of a chimeric HB12B antibody.

In one embodiment, a humanized anti-CD19 monoclonal antibody of the invention may comprise a VH and a VK, wherein the VH comprises the four framework regions, FW1, FW2, and FW3 of the human germline VH segment of V3-72 (described as DP29 in Tomlinson, I. M. et al., (1992) J. Mol. Biol., 227, 776-798), and FW4 of the human germline JH4 segment (Mattila, P. S. et al., (1995) Eur. J. Immunol., 25, 2578-2582); and the three VH CDR sequences of the HB12B antibody, CDR1 (SEQ ID NO:22), CDR2 (SEQ ID NO:24), and CDR3 (SEQ ID NO:26); and the VK comprises the four framework regions, FW1, FW2, FW3 of the human germline V kappa segment A10 (Straubinger, B. I et al., (1988) Biol. Chem. Hoppe-Seyler, 369, 601-607), and FW4 of the human germline immunoglobulin kappa J4 segment (Hieter, P. A. et al., (1982) J. Biol. Chem., 257, 1516-1522); and the three VK CDR sequences of the HB12B antibody, CDR1 (SEQ ID NO:28), CDR2 (SEQ ID NO:30), and CDR3 (SEQ ID NO:32). In one embodiment, an anti-CD19 antibody of the invention may comprise a VH and a VK, wherein the VH comprises the four framework regions, FW1, FW2, and FW3 of the human germline VH segment of V3-72 (described as DP29 in Tomlinson, I. M. et al., (1992) J. Mol. Biol., 227, 776-798), and FW4 of the human germline JH4 segment (Mattila, P. S. et al., (1995) Eur. J. Immunol., 25, 2578-2582); and at least one CDR having the amino acid sequence of a CDR listed on Table 1 supra; and the VK comprises the four framework regions, FW1, FW2, FW3 of the human germline V kappa segment A10 (Straubinger, B. I et al., (1988) Biol. Chem. Hoppe-Seyler, 369, 601-607), and FW4 of the human germline immunoglobulin kappa J4 segment (Hieter, P. A. et al., (1982) J. Biol. Chem., 257, 1516-1522); and at least one CDR having the amino acid sequence of a CDR listed on Table 1 supra. In one embodiment, this antibody may comprise one or more VK framework mutations selected from the group consisting of Y40F, K53H and Y91F. In one embodiment, the VK framework region may contain each of the point mutations Y40F, K53H and Y91F. In another embodiment, the VK framework region may contain only the Y40F and K53H point mutations. In another embodiment the VK framework may comprise only the Y40F point mutation.

### 6.3.1. CDR Regions of Anti-CD19 Antibodies

In certain embodiments, an anti-CD19 antibody of the invention may comprise a heavy chain variable region, VH, comprising at least one CDR having the amino acid sequence selected from the group consisting of SEQ ID NO:22, SEQ ID NO:24, and SEQ ID NO:26; and may further comprise at least one FW region having the amino acid sequence selected from the group consisting of SEQ ID NO:36, SEQ ID NO:38, SEQ ID NO:40, and SEQ ID NO:42. In another embodiment, an anti-CD19 antibody of the invention may comprise a heavy chain variable region, VH, comprising at least one CDR having the amino acid sequence selected from the group consisting of SEQ ID NO:22, SEQ ID NO:24, and SEQ ID NO:121 and may further comprise at least one FW region having the amino acid sequence selected from the group consisting of SEQ ID NO:36, SEQ ID NO:38, SEQ ID NO:40, and SEQ ID NO:42. In a further embodiment, an anti-CD19 antibody of the invention may comprise a heavy chain variable region, VH, comprising at least one CDR having the amino acid sequence selected from the group consisting of SEQ ID NO:22, SEQ ID NO:116, and SEQ ID NO:121 and may further comprise at least one FW region having the amino acid sequence selected from the group consisting of SEQ ID NO:36, SEQ ID NO:38, SEQ ID NO:40, and SEQ ID NO:42. In a further embodiment, an anti-CD19 antibody of the invention may comprise a heavy chain variable region, VH, comprising at least one CDR having the amino acid sequence selected from the group consisting of SEQ ID NO:208, SEQ ID NO:116, and SEQ ID NO:121 and may further comprise at least one FW region having the amino acid sequence selected from the group consisting of SEQ ID NO:36, SEQ ID NO:38, SEQ ID NO:40, and SEQ ID NO:42. In a further embodiment, an anti-CD19 antibody of the invention may comprise a heavy chain variable region, VH, comprising at least one CDR having the amino acid sequence selected from the group consisting of SEQ ID NO:208, SEQ ID NO:210, and SEQ ID NO:121 and may further comprise at least one FW region having the amino acid sequence selected from the group consisting of SEQ ID NO:36, SEQ ID NO:38, SEQ ID NO:40, and SEQ ID NO:42. In another embodiment, an anti-CD19 antibody of the invention may comprise a heavy chain variable region, VH, comprising at least one CDR having the amino acid sequence of a VH CDR1, VH CDR2, or VH CDR3 listed in Table 1 supra; and may further comprise at least one FW region having the amino acid sequence selected from the group consisting of SEQ ID NO:36, SEQ ID NO:38, SEQ ID NO:40, and SEQ ID NO:42.

In further embodiments, an anti-CD19 antibody of the invention may comprise a heavy chain variable region, VH, comprising at least one CDR sequence selected from the group consisting of SEQ ID NO:22, SEQ ID NO:24, and SEQ ID NO:26.

In additional embodiments, an anti-CD19 antibody may comprise a heavy chain variable region, VH, comprising at least one CDR sequence selected from the group consisting of SEQ ID NO:6, SEQ ID NO:8, and SEQ ID NO:10.

In one embodiment, an anti-CD19 antibody of the invention may comprise a heavy chain variable region, VH, comprising at least one CDR having the amino acid sequence selected from the group consisting of SEQ ID NO:22, SEQ ID NO:24 and SEQ ID NO:121. In another embodiment, an anti-CD19 antibody of the invention may comprise a heavy chain variable region, VH, comprising at least one CDR having the amino acid sequence selected from the group consisting of SEQ ID NO:22, SEQ ID NO:116 and SEQ ID NO:121. In another embodiment, an anti-CD19 antibody of the invention may comprise a heavy chain variable region, VH, comprising at least one CDR having the amino acid sequence selected from the group consisting of SEQ ID NO:208, SEQ ID NO:116 and SEQ ID NO:121. In another embodiment, an anti-CD19 antibody of the invention may comprise a heavy chain variable region, VH, comprising at least one CDR having the amino acid sequence selected from the group consisting of SEQ ID NO:208, SEQ ID NO:210 and SEQ ID NO:121.

In another embodiment, an anti-CD19 antibody of the invention may comprise a heavy chain variable region, VH, comprising at least one CDR having the amino acid sequence of a VH CDR1, VH CDR2, or VH CDR3 listed in Table 1 supra.

In another embodiment, an anti-CD19 antibody of the invention may comprise a heavy chain variable region, VH, comprising the amino acid sequences of a VH CDR1, VH CDR2, and VH CDR3 of any one of the antibodies listed in Table 1 supra. The anti-CD19 antibody of the invention may further comprise a light chain variable region, VL.

In another embodiment, an anti-CD19 antibody of the invention may comprise a light chain variable region, VL, comprising the amino acid sequences of a VL CDR1, VL CDR2, and VL CDR3 of any one of the antibodies listed in Table 1 supra. The anti-CD19 antibody of the invention may further comprise a heavy chain variable region, VH.

In another embodiment, an anti-CD19 antibody of the invention may comprise the amino acid sequences of a VH CDR1, VH CDR2, VH CDR3, VL CDR1, VL CDR2, and VL CDR3 of any one of the antibodies listed in Table 1 supra.

In certain embodiments, an anti-CD19 antibody may comprise the VH domain sequence of the humanized VH designated HB12B-(3-72/JH4) (SEQ ID NO:34).

In one embodiment, an anti-CD19 antibody described herein may comprise a heavy chain variable region, VH, having the amino acid sequence selected from the group consisting of SEQ ID NOs:103, 106, 191, and 192. In another embodiment, an anti-CD19 antibody described herein may comprise a heavy chain variable region, VH, having the amino acid sequence of a VH Domain listed in Table 1. supra.

In certain embodiments, an anti-CD19 antibody of the invention may comprise a light chain variable region, VK, comprising at least one CDR having an amino acid sequence selected from the group consisting of SEQ ID NO: 28, SEQ ID NO: 30, and SEQ ID NO:32 and may further comprise at least one FW region having an amino acid sequence selected from the group consisting of SEQ ID NO:54, SEQ ID NO:56, SEQ ID NO:72, SEQ ID NO:82, SEQ ID NO:64, SEQ ID NO:58, SEQ ID NO:66, and SEQ ID NO:60.

In one embodiment, an anti-CD19 antibody of the invention may comprise a light chain variable region, VK, comprising at least one CDR having an amino acid sequence selected from the group consisting of SEQ ID NO: 28, SEQ ID NO: 125, and SEQ ID NO:32 and may further comprise at least one FW region having an amino acid sequence selected from the group consisting of SEQ ID NO:54, SEQ ID NO:56, SEQ ID NO:72, SEQ ID NO:82, SEQ ID NO:64, SEQ ID NO:58, SEQ ID NO:66, and SEQ ID NO:60. In a further embodiment, an anti-CD19 antibody of the invention may comprise a light chain variable region, VK, comprising at least one CDR having an amino acid sequence selected from the group consisting of SEQ ID NO:211, SEQ ID NO:218, and SEQ ID NO:222 and may further comprise at least one FW region having an amino acid sequence selected from the group consisting of SEQ ID NO:54, SEQ ID NO:56, SEQ ID NO:72, SEQ ID NO:82, SEQ ID NO:64, SEQ ID NO:58, SEQ ID NO:66, and SEQ ID NO:60. In a further embodiment, an anti-CD19 antibody of the invention may comprise a light chain variable region, VK, comprising at least one CDR having an amino acid sequence selected from the group consisting of SEQ ID NO:28, SEQ ID NO:220, and SEQ ID NO:229 and may further comprise at least one FW region having an amino acid sequence selected from the group consisting of SEQ ID NO:54, SEQ ID NO:56, SEQ ID NO:72, SEQ ID NO:82, SEQ ID NO:64, SEQ ID NO:58, SEQ ID NO:66, and SEQ ID NO:60. In a further embodiment, an anti-CD19 antibody of the invention may comprise a light chain variable region, VK, comprising at least one CDR having an amino acid sequence selected from the group consisting of SEQ ID NO:215, SEQ ID NO:221, and SEQ ID NO:222 and may further comprise at least one FW region having an amino acid sequence selected from the group consisting of SEQ ID NO:54, SEQ ID NO:56, SEQ ID NO:72, SEQ ID NO:82, SEQ ID NO:64, SEQ ID NO:58, SEQ ID NO:66, and SEQ ID NO:60. In another embodiment, an anti-CD19 antibody of the invention may comprise a light chain variable region, VK, comprising at least one CDR having an amino acid sequence of a VK CDR1, VK CDR2, or VK CDR3 listed in Table 1 supra; and may further comprise at least one FW region having an amino acid sequence selected from the group consisting of SEQ ID NO:54, SEQ ID NO:56, SEQ ID NO:72, SEQ ID NO:82, SEQ ID NO:64, SEQ ID NO:58, SEQ ID NO:66, and SEQ ID NO:60.

In further embodiments, an anti-CD19 antibody of the invention may comprise a light chain variable region, VK, comprising at least one CDR sequence selected from the group consisting of SEQ ID NO:28, 30, and 32.

In further embodiments, an anti-CD19 antibody of the invention may comprise a light chain variable region, VK, comprising at least one CDR sequence selected from the group consisting of SEQ ID NO:12, 14, and 16.

In one embodiment, an anti-CD19 antibody of the invention may comprise a light chain variable region, VK, comprising at least one CDR having an amino acid sequence selected from the group consisting of SEQ ID NO: 28, SEQ ID NO: 125, and SEQ ID NO:32. In one embodiment, an anti-CD19 antibody of the invention may comprise a light chain variable region, VK, comprising at least one CDR having an amino acid sequence selected from the group consisting of SEQ ID NO: 211, SEQ ID NO: 218, and SEQ ID NO:222. In one embodiment, an anti-CD19 antibody of the invention may comprise a light chain variable region, VK, comprising at least one CDR having an amino acid sequence selected from the group consisting of SEQ ID NO: 28, SEQ ID NO: 220, and SEQ ID NO:229. In one embodiment, an anti-CD19 antibody of the invention may comprise a light chain variable region, VK, comprising at least one CDR having an amino acid sequence selected from the group consisting of SEQ ID NO: 215, SEQ ID NO: 221, and SEQ ID NO:222. In another embodiment, an anti-CD19 antibody of the invention may comprise a light chain variable region, VK, comprising at least one CDR having an amino acid sequence of a VK CDR1, VK CDR2, or VK CDR3 listed in Table 1 supra.

In certain embodiments, an anti-CD19 antibody may comprise the humanized VK domain sequence selected from a group consisting of HB12B-(A10-Jk4) (SEQ ID NO:52), HB12B-364987 (SEQ ID NO:62), HB12B-3649 (SEQ ID NO:68), HB12B-36 (SEQ ID NO:70), 7E12 VK (SEQ ID NO:110), 14H5 VK (SEQ ID NO:111), 16C9 VK (113), 15D1 VK (SEQ ID NO:112), 3C3 VK (SEQ ID NO:193), 6C11 VK (SEQ ID NO:204), and 9G7 VK (SEQ ID NO:205).

The present invention encompasses antibodies that bind to human CD19, comprising derivatives of the VH domains, VH CDR1s, VH CDR2s, VH CDR3s, VK domains, VK CDR1s, VK CDR2s, or VK CDR3s described herein that may bind to human CD19 (see for example the variants listed in Table 1. supra). Standard techniques known to those of skill in the art can be used to introduce mutations (e.g., additions, deletions, and/or substitutions) in the nucleotide sequence encoding an antibody, including, for example, site-directed mutagenesis and PCR-mediated mutagenesis that are routinely used to generate amino acid substitutions. In one embodiment, the VH and/or VK CDRs derivatives may include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, less than 2 amino acid substitutions, or 1 amino acid substitution relative to the original VH and/or VK CDRs of the HB12A or HB12B anti-CD19 antibody. In another embodiment, the VH and/or VK CDRs derivatives may have conservative amino acid substitutions (e.g. supra) made at one or more predicted non-essential amino acid residues (i.e., amino acid residues which are not critical for the antibody to specifically bind to human CD19). Mutations can also be introduced randomly along all or part of the VH and/or VK CDR coding sequences, such as by saturation mutagenesis, and the resultant mutants can be screened for biological activity to identify mutants that retain activity. Following mutagenesis, the encoded antibody can be expressed and the activity of the antibody can be determined. In one embodiment, antibodies of the invention disclosed herein may exclude the VH CDR1 and VH CDR2 of the hA19 antibody described in US20050070693A1.

In one embodiment, a human or humanized anti-CD19 antibody described herein may comprise a variant of any one of the VH CDRs listed in Table 1 supra wherein said variant VH CDR comprises an amino acid substitution. In a specific embodiment, an anti-CD19 antibody of the invention comprises a variant of a VH CDR listed in Table 1 wherein said variant VH CDR comprises one or more of the following natural or substituted amino acid residues: a threonine (T) at position 32 of VH CDR1, a tyrosine (Y) at position 60 of VH CDR2, an aspartic acid (D) at position 60 of VH CDR2, a leucine (L) at position 60 of VH CDR2, an alanine (A) at position 61 of VH CDR2, a valine (V) at position 61 of VH CDR2, a tyrosine (Y) at position 100B of VH CDR3, an arginine (R) at position 100B of VH CDR3, and an asparagine (N) at position 100B of VH CDR3, numbered according to Kabat (see, Kabat et al., Sequences of Proteins of Immunological Interest, 5th Ed. Public Health Service, National Institutes of Health, Bethesda, Md. (1991).

In one embodiment, a human or humanized anti-CD19 antibody described herein may comprise a variant of a VH CDR listed in Table 1. wherein said variant VH CDR comprises one or more of the following natural or substituted amino acid residues: a glutamic acid (E) at position 33 of VH CDR1, a leucine (L) at position 33 of VHCDR1, phenylalanine (F) at position 35 of VH CDR1, a tyrosine (Y) at position 35 of VH CDR1, an aspartic acid (D) at position 35 of VH CDR1, a leucine (L) at position 35 of VH CDR1, a serine (S) at position 57 of VH CDR2, a proline (P) at position 57 of VH CDR2, an asparagine (N) at position 57 of VH CDR2, a histidine (H) at position 100B of VH CDR3, a phenylalanine (F) at position 100B of VH CDR3, and a proline (P) at position 99 of VH CDR3, numbered according to Kabat (see, Kabat et al., Sequences of Proteins of Immunological Interest, 5th Ed. Public Health Service, National Institutes of Health, Bethesda, Md. (1991).

In one embodiment, a human or humanized anti-CD19 antibody described herein may comprise a variant of a VH CDR listed in Table 1. wherein said variant VH CDR comprises one or more of the following natural or substituted amino acid residues: a valine (V) at position 32 of VH CDR1, and a leucine (L) at position 52A of VHCDR2, numbered according to Kabat.

In another embodiment, a human or humanized anti-CD19 antibody of the invention may comprise a variant of a VK CDR listed in Table 1 wherein said VK CDR comprises one or more of the following natural or substituted amino acid residues: a histidine (H) at position 27D of VK CDR1, an isoleucine (I) at position 33 of VK CDR1, a glutamic acid (E) at position 50 of VK CDR2, a threonine (T) at position 91 in VK CDR3, and an isoleucine (I) at position 96 of VK CDR3, numbered according to Kabat.

In another embodiment, a human or humanized anti-CD19 antibody of the invention may comprise a variant of a VK CDR listed in Table 1 wherein said VK CDR comprises one or more of the following natural or substituted amino acid residues: a isoleucine (I) at position 27C of VK CDR1, a leucine (L) at position 30 of VK CDR1, an arginine (R) at position 33 of VK CDR1, a threonine (T) at position 33 of VK CDR1, a tyrosine (Y) at position 50 of VK CDR2, a threonine (T) at position 54 of VK CDR2, a proline (P) at position 54 of VK CDR2, a tyrosine (Y) at position 55 of VK CDR2, and an asparagine (N) at position 96 of VK CDR3, numbered according to Kabat.

In another embodiment, a human or humanized anti-CD19 antibody of the invention may comprise a variant of a VK CDR listed in Table 1 wherein said VK CDR comprises one or more of the following natural or substituted amino acid residues: an arginine (R) at position 54 of VK CDR2, a threonine (T) at position 54 of VK CDR2, an alanine (A) at position 54 of VK CDR2, and an alanine (A) at position 89 of VK CDR3, numbered according to Kabat.

The present invention further encompasses antibodies that bind to human CD19, said antibodies or antibody fragments comprising one or more CDRs wherein said CDRs comprise an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of one or more CDRs of the HB12A or HB12B anti-CD19 antibody. The percent identity of two amino acid sequences can be determined by any method known to one skilled in the art, including, but not limited to, BLAST protein searches.

### 6.3.2. Framework Regions of Anti-CD19 Antibodies

In one embodiment, the VH of a humanized anti-CD19 monoclonal antibody of the invention may comprise a framework region that has an amino acid sequence identity with the corresponding framework regions (i.e., FW1 of antibody X as compared to FW1 of antibody Y) of HB12B-(3-72/JH4) VH (SEQ ID NO:34) within the range of from about 64% to about 100%. In certain aspects of this embodiment, the human or humanized VH framework regions of antibodies described herein may have an amino acid sequence identity with the HB12B-(3-72/JH4) VH (SEQ ID NO:34) that is at least 64%, at least 70%, at least 75%, at least 80%, at least 85%, at least 90%, or at least 95%.

In particular embodiments, the human or humanized VH framework regions of anti-CD19 antibodies described herein may have an amino acid sequence identity with the corresponding framework regions of HB12B-(3-72/JH4) VH (SEQ ID NO:34) of at least 56 out of 87 amino acids (56/87) In particular embodiments, the VH framework amino acid sequence identity may be at least 56/87, 57/87, 58/87, 59/87, 60/87, 61/87, 62/87, 63/87, 64/87, 65/87, 66/87, 67/87, 68/87, 69/87, 70/87, 71, 87, 72/87, 73/87 74/87, 75/87, 76/87, 77.87, 78/87, 79/87, 80/87, 81/87, 82/87, 83/87, 84/87, 85/87, 86/87, or 87/87 amino acids. VH sequences of anti-CD19 antibodies described herein may have high sequence identity to the Vernier amino acid residues of HB12B-(3-72/JH4), for example a Vernier sequence identity of at least 10 out of 16 (10/16), at least 11/16, at least 12/16, at least 13/16, at least 14/16, or at least 15/16 Vernier residues. In another embodiment, the mismatch of a Vernier amino acid residue may be a conservative amino acid substitution. A mismatch that is a conservative amino acid substitution is one in which the mismatched amino acid has physical and chemical properties similar to the Vernier amino acid, e.g., the mismatched residue has similar characteristics of polarity (polar or nonpolar), acidity (acidic or basic), side chain structure (e.g., branched or straight, or comprising a phenyl ring, a hydroxyl moiety, or a sulfur moiety) to the Vernier residue.

In other embodiments, the mismatch of a Vernier amino acid residue may be a non-conservative amino acid substitution. A mismatch that is a non-conservative amino acid substitution is one in which the mismatched amino acid does not have physical and chemical properties similar to the Vernier amino acid, e.g., the mismatched residue has a different polarity, acidity, or side chain structure (e.g., branched or straight, or comprising a phenyl ring, a hydroxyl moiety, or a sulfur moiety) as compared to the Vernier residue to be replaced.

In other embodiments, a human or humanized anti-CD19 antibody of the invention may comprise VH framework regions wherein said VH framework regions may comprise one or more of the following residues: a leucine (L) at position 20 of framework region 1, a phenylalanine (F) at position 27 of framework region 1, a threonine (T) at position 28 of framework region 1, an arginine (R) at position 38 in framework region 2, a valine (V) at position 48 of framework region 2, a phenylalanine (F) at position 67 of framework region 3, an arginine (R) at position 71 of framework region 3, a leucine (L) at position 80 of framework region 3, and a tyrosine (Y) at position 91 of framework region 3, numbered according to Kabat.

Kabat numbering is based on the seminal work of Kabat et al. (1991) Sequences of Proteins of Immunological Interest, Publication No. 91-3242, published as a three volume set by the National Institutes of Health, National Technical Information Service (hereinafter “Kabat”). Kabat provides multiple sequence alignments of immunoglobulin chains from numerous species antibody isotypes. The aligned sequences are numbered according to a single numbering system, the Kabat numbering system. The Kabat sequences have been updated since the 1991 publication and are available as an electronic sequence database (latest downloadable version 1997). Any immunoglobulin sequence can be numbered according to Kabat by performing an alignment with the Kabat reference sequence. Accordingly, the Kabat numbering system provides a uniform system for numbering immunoglobulin chains. Unless indicated otherwise, all immunoglobulin amino acid sequences described herein are numbered according to the Kabat numbering system. Similarly, all single amino acid positions referred to herein are numbered according to the Kabat numbering system.

In further particular embodiments, the human or humanized VH framework regions of anti-CD19 antibodies described herein may have framework regions selected for identity or conservative mismatches at one or more of the following Vernier, Interface or Canonical residue positions: 20, 22, 24, 26, 27, 28, 29, 30, 36, 37, 39, 45, 47, 48, 49, 67, 69, 71, 73, 78, 80, 90, 91, 92, 93, 94, and 103. One or more of the mismatched Vernier, Interface and Canonical residues may be changed, e.g., by mutagenesis, to match the corresponding amino acid residue of the HB12A or HB12B VH framework region.

In one embodiment of the invention, the human or humanized VK framework regions of anti-CD19 antibodies described herein may have an amino acid sequence identity with the framework regions of HB12B-(A10-Jk4) VK (SEQ ID NO:52) within the range of from about 65% to about 100%. In certain aspects of this embodiment, the human or humanized VK framework regions of antibodies described herein may have an amino acid sequence identity with the HB12B-(A10-Jk4) antibody VK that is at least 65%, at least 70%, at least 75%, at least 80%, at least 85%, at least 90%, or at least 95%.

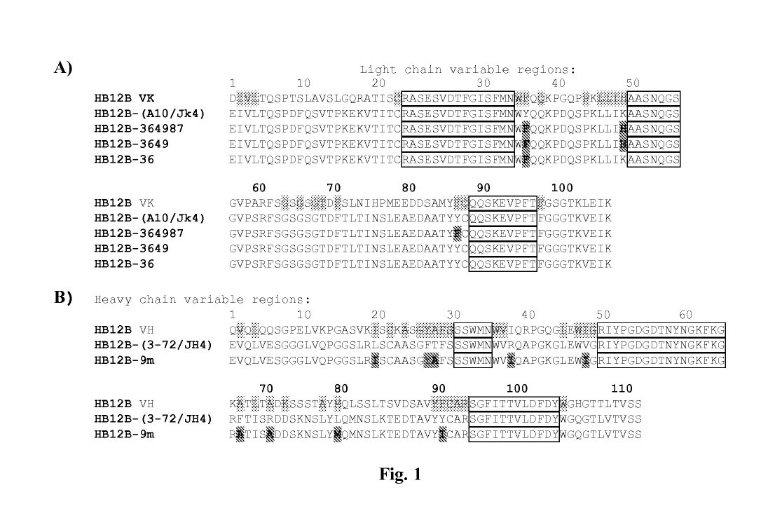

In particular embodiments, the human or humanized VK framework regions of antibodies described herein may have an amino acid sequence identity with the corresponding framework regions (i.e., FW1 of antibody X as compared to FW1 of antibody Y) of HB12B-(A10-Jk4) VH (SEQ ID NO:52) of at least 52 out of 80 amino acids (52/80) In particular embodiments, the VH framework amino acid sequence identity may be at least 52/80, 53/80, 54/80, 55/80, 56/80, 57/80, 58/80, 59/80, 60/80, 61/80, 62/80, 63/80, 64/80, 65/80, 66/80, 67/80, 68/80, 69/80, 70/80, 71, 80, 72/80, 73/80 74/80, 75/80, 76/80, 77/80, 78/80, 79/80, or 80/80, amino acids. VK sequences of anti-CD19 antibodies described herein may have high sequence identity to the Vernier amino acid residues of HB12B (see FIG. 1), for example a Vernier sequence identity of at least 9 out of 14 (9/14), at least 10/14, at least 11/14, at least 12/14, at least 13/14 Vernier residues. In another embodiment, the mismatch of a Vernier amino acid residue may be a conservative amino acid substitution. A mismatch that is a conservative amino acid substitution is one in which the mismatched amino acid has physical and chemical properties similar to the Vernier amino acid, e.g., the mismatched residue has similar characteristics of polarity (polar or nonpolar), acidity (acidic or basic), side chain structure (e.g., branched or straight, or comprising a phenyl ring, a hydroxyl moiety, or a sulfur moiety) to the Vernier residue.

In other embodiments, the mismatch of a Vernier amino acid residue may be a non-conservative amino acid substitution. A mismatch that is a non-conservative amino acid substitution is one in which the mismatched amino acid does not have physical and chemical properties similar to the Vernier amino acid, e.g., the mismatched residue has a different polarity, acidity, or side chain structure (e.g., branched or straight, or comprising a phenyl ring, a hydroxyl moiety, or a sulfur moiety) as compared to the Vernier residue to be replaced.

In other embodiments, the human or humanized VK framework regions described herein may comprise one or more of the following residues: a phenylalanine (F) at position 36 of framework region 2, a histidine (H) at position 49 of framework region 2, and a phenylalanine (F) at position 87 of framework region 3, numbered according to Kabat.

In further particular embodiments, the human or humanized VK framework regions of antibodies described herein may have framework regions selected for identity or conservative mismatches at one or more of the following Vernier, Interface or Canonical residue positions: 2, 3, 4, 23, 35, 36, 38, 44, 56, 47, 48, 49, 64, 66, 68, 69, 71, 87, 88, and 98. One or more of the mismatched Vernier, Interface and Canonical residues may be changed, e.g., by mutagenesis, to match the corresponding amino acid residue of the HB12A or HB12B framework region.

In particular embodiments, a heavy chain comprising a humanized VH of the invention may be expressed with a light chain comprising a humanized VK of the invention to produce a humanized anti-CD19 antibody. In a specific embodiment, a humanized anti-CD19 antibody of the invention may comprise a VH sequence selected from the group consisting of the sequences designated HB12B-(3-72/JH4) (SEQ ID NO:34), 7E12 VH (SEQ ID NO:102), 14H5 VH (SEQ ID NO:103), 15D1 VH (SEQ ID NO:104), 15D7 VH (SEQ ID NO:105), 16C4 VH (SEQ ID NO:106), 14H5-YG (SEQ ID NO:107), 14H5-DG (SEQ ID NO:108), 14H5-LG (SEQ ID NO:109), 1A7 VH (SEQ ID NO:191), 3C3 VH (SEQ ID NO:191), 6C11 VH (SEQ ID NO:191), 9G7 (SEQ ID NO:191), 3B4 VH (SEQ ID NO:236), and 3F11 VH (SEQ ID NO:192); and may further comprise a VK sequence selected from the group consisting of the sequences designated HB12B-(A10/JK4) (SEQ ID NO:52); HB12B-364987 (or 364987) (SEQ ID NO:62); HB12B-3649 (or 3649) (SEQ ID NO:68); HB12B-36 (or 36) (SEQ ID NO:70), 7E12 VK (SEQ ID NO:110), 14H5 (SEQ ID NO:111), 15D1 (SEQ ID NO:112), 16C9 (SEQ ID NO:113), 3C3 VK (SEQ ID NO:193), 3E5 VK (SEQ ID NO:194), 3D4 VK (SEQ ID NO:195), 3F1 VK (SEQ ID NO:196), 5B5 VK (SEQ ID NO:197), 6F7 VK (SEQ ID NO:198), 1C11 VK (SEQ ID NO:199), 2B11 VK (SEQ ID NO:200), 2D10 VK (SEQ ID NO:201), 5C11 VK (SEQ ID NO:202), 5D4 VK (SEQ ID NO:203), 6C11 VK (SEQ ID NO:204), 9G7 VK (SEQ ID NO:205), 1H4 VK (SEQ ID NO:206), and 5C4 VK (SEQ ID NO:207). In a particular embodiment, a humanized anti-CD19 antibody comprises the VH sequence HB12B-(3-72/JH4) (SEQ ID NO:34) and the VK sequence HB12B-364987 (SEQ ID NO:62). In a particular embodiment, a humanized anti-CD19 antibody comprises the VH sequence HB12B-(3-72/JH4) (SEQ ID NO:34) and the VK sequence HB12B-3649 (SEQ ID NO:68). In yet another embodiment, a humanized anti-CD19 antibody comprises the VH sequence HB12B-(3-72/JH4) (SEQ ID NO:34) and the VK sequence HB12B-36 (SEQ ID NO:70).

In a specific embodiment, an anti-CD19 antibody of the invention comprises the VH sequence 7E12 VH (SEQ ID NO:102) and the VK sequence 7E12 VK (SEQ ID NO:110). In a specific embodiment, an anti-CD19 antibody of the invention comprises the VH sequence 14H5 VH (SEQ ID NO:103) and the VK sequence 14H5 VK (SEQ ID NO:111). In a specific embodiment, an anti-CD19 antibody of the invention comprises the VH sequence 14H5-YG VH (SEQ ID NO:107) and the VK sequence 14H5 VK (SEQ ID NO:111). In a specific embodiment, an anti-CD19 antibody of the invention comprises the VH sequence 14H5-DG VH (SEQ ID NO:108) and the VK sequence 14H5 VK (SEQ ID NO:111). In a specific embodiment, an anti-CD19 antibody of the invention comprises the VH sequence 14H5-LG VH (SEQ ID NO:109) and the VK sequence 14H5 VK (SEQ ID NO:111). In a specific embodiment, an anti-CD19 antibody of the invention comprises the VH sequence 14H5 VH (SEQ ID NO:103) and the VK sequence 16C9 VK (SEQ ID NO:113). In a specific embodiment, an anti-CD19 antibody of the invention comprises the VH sequence 15D1 VH (SEQ ID NO:104) and the VK sequence 15D1 VK (SEQ ID NO:112). In a specific embodiment, an anti-CD19 antibody of the invention comprises the VH sequence 15D7 VH (SEQ ID NO:105) and the VK sequence 14H5 VK (SEQ ID NO:111). In a specific embodiment, an anti-CD19 antibody of the invention comprises the VH sequence 16C4 VH (SEQ ID NO:106) and the VK sequence 14H5 VK (SEQ ID NO:111). In a specific embodiment, an anti-CD19 antibody of the invention comprises the VH sequence 1A7 VH (SEQ ID NO:191) and the VK sequence 14H5 VK (SEQ ID NO:111). In a specific embodiment, an anti-CD19 antibody of the invention comprises the VH sequence 1A7 VH (SEQ ID NO:191) and the VK sequence 3C3 VK (SEQ ID NO:193). In a specific embodiment, an anti-CD19 antibody of the invention comprises the VH sequence 1A7 VH (SEQ ID NO:191) and the VK sequence 3E5 VK (SEQ ID NO:194). In a specific embodiment, an anti-CD19 antibody of the invention comprises the VH sequence 1A7 VH (SEQ ID NO:191) and the VK sequence 3D4 VK (SEQ ID NO:195). In a specific embodiment, an anti-CD19 antibody of the invention comprises the VH sequence 1A7 VH (SEQ ID NO:191) and the VK sequence 5B5 VK (SEQ ID NO:197). In a specific embodiment, an anti-CD19 antibody of the invention comprises the VH sequence 1A7 VH (SEQ ID NO:191) and the VK sequence 6F7 VK (SEQ ID NO:198). In a specific embodiment, an anti-CD19 antibody of the invention comprises the VH sequence 1A7 VH (SEQ ID NO:191) and the VK sequence 2D10 VK (SEQ ID NO:201). In a specific embodiment, an anti-CD19 antibody of the invention comprises the VH sequence 1A7 VH (SEQ ID NO:191) and the VK sequence 5C11 VK (SEQ ID NO:202). In a specific embodiment, an anti-CD19 antibody of the invention comprises the VH sequence 1A7 VH (SEQ ID NO:191) and the VK sequence 9G7 VK (SEQ ID NO:205). In a specific embodiment, an anti-CD19 antibody of the invention comprises the VH sequence 1A7 VH (SEQ ID NO:191) and the VK sequence 1H4 VK (SEQ ID NO:206). In a specific embodiment, an anti-CD19 antibody of the invention comprises the VH sequence 1A7 VH (SEQ ID NO:191) and the VK sequence 5C4 VK (SEQ ID NO:207). In a specific embodiment, an anti-CD19 antibody of the invention comprises the VH sequence 3B4 VH (SEQ ID NO:236) and the VK sequence 14H5 VK (SEQ ID NO:111). In a specific embodiment, an anti-CD19 antibody of the invention comprises the VH sequence 3F11 VH (SEQ ID NO:192) and the VK sequence 3F11 VK (SEQ ID NO:196). In a specific embodiment, an anti-CD19 antibody of the invention comprises the VH sequence 16C4 VH (SEQ ID NO:106) and the VK sequence 1C11 VK (SEQ ID NO:199). In a specific embodiment, an anti-CD19 antibody of the invention comprises the VH sequence 16C4 VH (SEQ ID NO:106) and the VK sequence 2B11 VK (SEQ ID NO:200). In a specific embodiment, an anti-CD19 antibody of the invention comprises the VH sequence 16C4 VH (SEQ ID NO:106) and the VK sequence 5D4 VK (SEQ ID NO:203). In a specific embodiment, an anti-CD19 antibody of the invention comprises the VH sequence 16C4 VH (SEQ ID NO:106) and the VK sequence 6F7 VK (SEQ ID NO:198). In a specific embodiment, an anti-CD19 antibody of the invention comprises the VH sequence 3F11 VH (SEQ ID NO:192) and the VK sequence 6C11 VK (SEQ ID NO:204). In a specific embodiment, an anti-CD19 antibody of the invention comprises any combination of a VH and a VL listed in Table 1.

In certain embodiments, a light chain comprising a humanized VK of the invention may be expressed with a heavy chain comprising a humanized VH of the invention to produce a humanized anti-CD19 antibody. In one embodiment, a humanized anti-CD19 antibody described herein comprises a VK sequence selected from the group consisting of the sequences designated HB12B-(A10/JK4) (SEQ ID NO:52); HB12B-364987 (or 364987) (SEQ ID NO:62); HB12B-3649 (or 3649) (SEQ ID NO:68); HB12B-36 (or 36) (SEQ ID NO:70), 7E12 VK (SEQ ID NO:110), 14H5 (SEQ ID NO:111), 15D1 (SEQ ID NO:112), 16C9 (SEQ ID NO:113), 3C3 (SEQ ID NO:193), 3E5 (SEQ ID NO:194), 3D4 (SEQ ID NO:195), 3F11 (SEQ ID NO:196), 5B5 (SEQ ID NO:197), 6F7 (SEQ ID NO:198), 1C11 (SEQ ID NO:199), 2B11 (SEQ ID NO:200), 2D10 (SEQ ID NO:201), 5C11 (SEQ ID NO:202), 5D4 (SEQ ID NO:203), 6C11 (SEQ ID NO:204), 9G7 (SEQ ID NO:205), 1H4 (SEQ ID NO:206), AND 5C4 (SEQ ID NO:207). The aforementioned VK sequence may be paired with a VH sequence comprising an amino acid sequence in its framework region selected from the group consisting of SEQ ID NO:36, 38, 40, and 42.

In certain embodiments, a heavy chain comprising a humanized VH of the invention may be expressed with a light chain comprising a humanized VK of the invention to produce a humanized anti-CD19 antibody. In one embodiment, a humanized anti-CD19 antibody described herein comprises a VH sequence selected from the group consisting of the sequences designated HB12B-(3-72/JH4) (SEQ ID NO:34), 7E12 VH (SEQ ID NO:102), 14H5 VH (SEQ ID NO:103), 15D1 VH (SEQ ID NO:104), 15D7 VH (SEQ ID NO:105), 16C4 VH (SEQ ID NO:106), 14H5-YG (SEQ ID NO:107), 14H5-DG (SEQ ID NO:108), 14H5-LG (SEQ ID NO:109), 1A7 (SEQ ID NO:191), 3C3 VH (SEQ ID NO:191), 6C11 VH (SEQ ID NO:191), 9G7 (SEQ ID NO:191), 3B4 VH (SEQ ID NO:236), and 3F11 VH (SEQ ID NO:192). The aforementioned VH sequence may be paired with a VK sequence comprising an amino acid sequence in its framework region selected from the group consisting of SEQ ID NO:54, SEQ ID NO:56, SEQ ID NO:72, SEQ ID NO:82, SEQ ID NO:64, SEQ ID NO:58, SEQ ID NO:66, and SEQ ID NO:60.

In certain embodiments, a humanized VH or VK derived from the parental HB12A or HB12B hybridoma may be expressed as a chimeric immunoglobulin light chain or a chimeric immunoglobulin heavy chain to produce a chimeric anti-CD19 antibody. In a particular embodiment, a humanized VH may be expressed as a chimeric antibody comprising the HB12A VK (SEQ ID NO:4) or HB12B VK (SEQ ID NO:20). In another particular embodiment, a humanized VK may be expressed as a chimeric antibody comprising the HB12A VH (SEQ ID NO:2) or HB12B VH (SEQ ID NO:18). In another embodiment, a chimeric anti-CD19 antibody may comprise the VK sequence of HB12A VK (SEQ ID NO:4) or HB12B VK (SEQ ID NO:20) and may further comprise the VH sequence of HB12A VH (SEQ ID NO:2) or HB12B VH (SEQ ID NO:18).

In a particular embodiment, a humanized VH of the invention may further comprise a leader sequence of MGDNDIHFAFLSTGVHS (SEQ ID NO:83).

In another embodiment, a humanized VK of the invention may further comprise a leader sequence MDMRVPAQLLGLLLLWLPGAKC (SEQ ID NO:84) selected from the leader peptide of the human VKI-L12 gene.

Anti-CD19 antibodies described herein may have a high binding affinity for the human CD19 (hCD19) antigen. For example, an antibody described herein may have an association rate constant or kₒₙ rate (antibody (Ab)+antigen (Ag)^{k}^{ₒₙ}→Ab−Ag) of at least 2×10⁵M⁻¹s⁻¹, at least 5×10⁵M⁻¹s⁻¹, at least 10⁶M⁻¹s⁻¹, at least 5×10⁶ M⁻¹s⁻¹, at least 10⁷M⁻¹s⁻¹, at least 5×10⁷M⁻¹s⁻¹, or at least 10⁸ M⁻¹s⁻¹.

In another embodiment, an anti-CD19 antibody of the invention may have a k_{off} rate ((Ab−Ag)^{k}^{_{off}}→antibody (Ab)+antigen (Ag)) of less than 5×10⁻¹ s⁻¹, less than 10⁻¹ s⁻¹, less than 5×10⁻² s⁻¹, less than 10⁻² s⁻¹, less than 5×10⁻³ s⁻¹, less than 10⁻³ s⁻¹, less than 5×10⁻⁴ s⁻¹, or less than 10⁻⁴ s^{-1.}In a another embodiment, an antibody of the invention has a k_{off} of less than 5×10⁻⁵ s⁻¹, less than 10⁻⁵ s⁻¹, less than 5×10⁻⁶ s⁻¹, less than 10⁻⁶ s⁻¹, less than 5×10⁻⁷ s⁻¹, less than 10⁻⁷ s⁻¹, less than 5×10⁻⁸ s⁻¹, less than 10⁻⁸ s⁻¹, less than 5×10⁻⁹ s⁻¹, less than 10⁻⁹ s⁻¹, or less than 10⁻¹⁰ s⁻¹.

In another embodiment, an anti-CD19 antibody of the invention may have an affinity constant or Kₐ (kₒₙ/k_{off}) of at least 10² M⁻¹, at least 5×10² M⁻¹, at least 10³ M⁻¹, at least 5×10³ M⁻¹, at least 10⁴ M⁻¹, at least 5×10⁴ M⁻¹, at least 10⁵ M⁻¹, at least 5×10⁵M⁻¹, at least 10⁶M⁻¹, at least 5×10⁶ M⁻¹, at least 10⁷ M⁻¹, at least 5×10⁷ M⁻¹, at least 10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹²M⁻¹, at least 5×10¹² M⁻¹, at least 10¹³ M⁻¹, at least 5×10¹³ M⁻¹, at least 10¹⁴ M⁻¹, at least 5×10¹⁴ M⁻¹, at least 10¹⁵ M⁻¹, or at least 5×10¹⁵ M. In yet another embodiment, an anti-CD19 antibody of the invention may have a dissociation constant or K_{d} (k_{off}/kₒₙ) of less than 5×10⁻² M, less than 10⁻² M, less than 5×10⁻³ M, less than 10⁻³ M, less than 5×10⁻⁴ M, less than 10⁻⁴ M, less than 5×10⁻⁵ M, less than 10⁻⁵ M, less than 5×10⁻⁶ M, less than 10⁻⁶ M, less than 5×10⁻⁷ M, less than 10⁻⁷ M, less than 5×10⁻⁸ M, less than 10⁻⁸ M, less than 5×10⁻⁹ M, less than 10⁻⁹ M, less than 5×10⁻¹⁰ M, less than 10⁻¹⁰ M, less than 5×10⁻¹¹ M, less than 10⁻¹¹ M, less than 5×10⁻¹² M, less than 10⁻¹² M, less than 5×10⁻¹³ M, less than 10⁻¹³ M, less than 5×10⁻¹⁴ M, less than 10⁻¹⁴ M, less than 5×10⁻¹⁵ M, or less than 10⁻¹⁵ M.

In one embodiment, an antibody of the invention used in accordance with a method described herein may immunospecifically bind to human CD19 and may have a dissociation constant (K_{d}) of less than 3000 pM, less than 2500 pM, less than 2000 pM, less than 1500 pM, less than 1000 pM, less than 750 pM, less than 500 pM, less than 250 pM, less than 200 pM, less than 150 pM, less than 100 pM, less than 75 pM as assessed using a method described herein or known to one of skill in the art (e.g., a BIAcore assay, ELISA) (Biacore International AB, Uppsala, Sweden). In a specific embodiment, an antibody of the invention used in accordance with a method described herein may immunospecifically bind to a human CD19 antigen and may have a dissociation constant (K_{d}) of between 25 to 3400 pM, 25 to 3000 pM, 25 to 2500 pM, 25 to 2000 pM, 25 to 1500 pM, 25 to 1000 pM, 25 to 750 pM, 25 to 500 pM, 25 to 250 pM, 25 to 100 pM, 25 to 75 pM, 25 to 50 pM as assessed using a method described herein or known to one of skill in the art (e.g., a BIAcore assay, ELISA). In another embodiment, an anti-CD19 antibody of the invention used in accordance with a method described herein may immunospecifically bind to hCD19 and may have a dissociation constant (K_{d}) of 500 pM, 100 pM, 75 pM or 50 pM as assessed using a method described herein or known to one of skill in the art (e.g., a BIAcore assay, ELISA).

The invention further provides polynucleotides comprising a nucleotide sequence encoding a human, humanized, or chimeric anti-CD19 antibody described herein or fragments thereof. The invention also encompasses polynucleotides that hybridize under stringent or lower stringency hybridization conditions, e.g., as defined herein, to polynucleotides that encode a human, humanized, or chimeric antibody described herein that binds to hCD19.

Stringent hybridization conditions include, but are not limited to, hybridization to filter-bound DNA in 6× sodium chloride/sodium citrate (SSC) at about 45° C. followed by one or more washes in 0.2×SSC/0.1% SDS at about 50-65° C., highly stringent conditions such as hybridization to filter-bound DNA in 6×SSC at about 45° C. followed by one or more washes in 0.1×SSC/0.2% SDS at about 60° C., or any other stringent hybridization conditions known to those skilled in the art (see, for example, Ausubel, F. M. et al., eds. 1989 Current Protocols in Molecular Biology, vol. 1, Green Publishing Associates, Inc. and John Wiley and Sons, Inc., NY at pages 6.3.1 to 6.3.6 and 2.10.3).

The polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

A polynucleotide encoding an antibody may also be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, including polyA+RNA, isolated from, any tissue or cells expressing the antibody, such as hybridoma cells selected to express an antibody) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

The present invention also provides polynucleotide sequences encoding VH and VK framework regions and CDRs of antibodies described herein as well as expression vectors for their efficient expression in mammalian cells.

The present invention further provides for antibodies that may efficiently deplete B cells expressing a recombinant human CD19 molecule in a hCD19 transgenic mouse model system (see, Yazawa et al., Proc Natl Acad Sci USA. 102(42):15178-83 (2005)). In a specific embodiment, an anti-CD-19 antibody of the invention may achieve B cell depletion that is at least 20%, at least 30%, at least 40%, at least 50%, at least 60%, at least 70%, at least 80%, at least 90%, at least 95%, or at least 100% of the depletion achieved by the HB12B monoclonal antibody. In a further embodiment, an anti-CD19 antibody of the invention may achieve B cell depletion that is more complete than the depletion achieved by the HB12B antibody. In one embodiment, an anti-CD19 antibody of the invention may deplete circulating B cells, blood B cells, splenic B cells, marginal zone B cells, follicular B cells, peritoneal B cells, and/or bone marrow B cells. In a one embodiment, an anti-CD19 antibody of the invention may achieve depletion of progenitor B cells, early pro-B cells, late pro-B cells, large-pre-B cells, small pre-B cells, immature B cells, mature B cells, antigen stimulated B cells, and/or plasma cells. In one embodiment, B cell depletion by an anti-CD19 antibody of the invention may persist for at least 1 day, at least 2 days, at least 3 days, at least 4 days, at least 5 days at least 6 days, at least 7 days, at least 8 days, at least 9 days, at least 10 days, at least 15 days, at least 20 days, at least 25 days, or at least 30 days. In another embodiment, B cell depletion by an anti-CD19 antibody of the invention may persist for at least 1 week, at least 2 weeks, at least 3 weeks, at least 4 weeks, at least 5 weeks, at least 6 weeks, at least 7 weeks, at least 8 weeks, at least 9 weeks, or at least 10 weeks. In a further embodiment, B cell depletion by an anti-CD19 antibody of the invention may persist for at least 1 month, at least 2 months, at least 3 months, at least 4 months, at least 5 months, at least 6 months, at least 7 months, at least 8 months, at least 9 months, at least 10 months, at least 11 months or at least 12 months.

The present invention also provides for antibodies that efficiently deplete B cells in a human subject. In a specific embodiment, an anti-CD-19 antibody of the invention may achieve at least about 20%, at least about 30%, at least about 40%, at least about 50%, at least about 60%, at least about 70%, at least about 80%, at least about 90%, at least about 95%, or about 100% B cell depletion. In another embodiment, an anti-CD19 antibody of the invention may deplete B cell subsets in a human subject. In a specific embodiment, an anti-CD19 antibody of the invention may deplete circulating B cells, blood B cells, splenic B cells, marginal zone B cells, follicular B cells, peritoneal B cells, and/or bone marrow B cells. CD19 is present on the surface of B cells at all developmental stages. An anti-CD19 antibody may therefore deplete B cells of all developmental stages. In a specific embodiment, an anti-CD19 antibody of the invention may achieve depletion of progenitor B cells, early pro-B cells, late pro-B cells, large-pre-B cells, small pre-B cells, immature B cells, mature B cells, antigen stimulated B cells, and/or plasma cells. Depletion of B cells may persist for extended periods of time. In one embodiment, B cell depletion by an anti-CD19 antibody of the invention may persist for at least 1 day, at least 2 days, at least 3 days, at least 4 days, at least 5 days at least 6 days, at least 7 days, at least 8 days, at least 9 days, at least 10 days, at least 15 days, at least 20 days, at least 25 days, or at least 30 days. In another embodiment, B cell depletion by an anti-CD19 antibody of the invention may persist for at least 1 week, at least 2 weeks, at least 3 weeks, at least 4 weeks, at least 5 weeks, at least 6 weeks, at least 7 weeks, at least 8 weeks, at least 9 weeks, or at least 10 weeks. In a further embodiment, B cell depletion by an anti-CD19 antibody of the invention may persist for at least 1 month, at least 2 months, at least 3 months, at least 4 months, at least 5 months, at least 6 months, at least 7 months, at least 8 months, at least 9 months, at least 10 months, at least 11 months or at least 12 months.

In one embodiment, an anti-CD19 antibody of the invention depletes at least about 20%, at least about 30%, at least about 40%, at least about 50%, at least about 60%, at least about 70%, at least about 80%, at least about 90%, at least about 95%, or about 100% of circulating B cells. In one embodiment, an anti-CD19 antibody of the invention depletes at least about 20%, at least about 30%, at least about 40%, at least about 50%, at least about 60%, at least about 70%, at least about 80%, at least about 90%, at least about 95%, or about 100% of blood B cells. In one embodiment, an anti-CD19 antibody of the invention depletes at least about 20%, at least about 30%, at least about 40%, at least about 50%, at least about 60%, at least about 70%, at least about 80%, at least about 90%, at least about 95%, or about 100% of splenic B cells. In one embodiment, an anti-CD19 antibody of the invention depletes at least about 20%, at least about 30%, at least about 40%, at least about 50%, at least about 60%, at least about 70%, at least about 80%, at least about 90%, at least about 95%, or about 100% of marginal zone B cells. In one embodiment, an anti-CD19 antibody of the invention depletes at least about 20%, at least about 30%, at least about 40%, at least about 50%, at least about 60%, at least about 70%, at least about 80%, at least about 90%, at least about 95%, or about 100% of follicular B cells. In one embodiment, an anti-CD19 antibody of the invention depletes at least about 20%, at least about 30%, at least about 40%, at least about 50%, at least about 60%, at least about 70%, at least about 80%, at least about 90%, at least about 95%, or about 100% of peritoneal B cells. In one embodiment, an anti-CD19 antibody of the invention depletes at least about 20%, at least about 30%, at least about 40%, at least about 50%, at least about 60%, at least about 70%, at least about 80%, at least about 90%, at least about 95%, or about 100% of bone marrow B cells. In one embodiment, an anti-CD19 antibody of the invention depletes at least about 20%, at least about 30%, at least about 40%, at least about 50%, at least about 60%, at least about 70%, at least about 80%, at least about 90%, at least about 95%, or about 100% of progenitor B cells. In one embodiment, an anti-CD19 antibody of the invention depletes at least about 20%, at least about 30%, at least about 40%, at least about 50%, at least about 60%, at least about 70%, at least about 80%, at least about 90%, at least about 95%, or about 100% of early pro-B cells. In one embodiment, an anti-CD19 antibody of the invention depletes at least about 20%, at least about 30%, at least about 40%, at least about 50%, at least about 60%, at least about 70%, at least about 80%, at least about 90%, at least about 95%, or about 100% of late pro-B cells. In one embodiment, an anti-CD19 antibody of the invention depletes at least about 20%, at least about 30%, at least about 40%, at least about 50%, at least about 60%, at least about 70%, at least about 80%, at least about 90%, at least about 95%, or about 100% of large pre-B cells. In one embodiment, an anti-CD19 antibody of the invention depletes at least about 20%, at least about 30%, at least about 40%, at least about 50%, at least about 60%, at least about 70%, at least about 80%, at least about 90%, at least about 95%, or about 100% of small pre-B cells. In one embodiment, an anti-CD19 antibody of the invention depletes at least about 20%, at least about 30%, at least about 40%, at least about 50%, at least about 60%, at least about 70%, at least about 80%, at least about 90%, at least about 95%, or about 100% of immature B cells. In one embodiment, an anti-CD19 antibody of the invention depletes at least about 20%, at least about 30%, at least about 40%, at least about 50%, at least about 60%, at least about 70%, at least about 80%, at least about 90%, at least about 95%, or about 100% of mature B cells. In one embodiment, an anti-CD19 antibody of the invention depletes at least about 20%, at least about 30%, at least about 40%, at least about 50%, at least about 60%, at least about 70%, at least about 80%, at least about 90%, at least about 95%, or about 100% of antigen stimulated B cells. In one embodiment, an anti-CD19 antibody of the invention depletes at least about 20%, at least about 30%, at least about 40%, at least about 50%, at least about 60%, at least about 70%, at least about 80%, at least about 90%, at least about 95%, or about 100% of plasma cells. Depletion of B cells may persist for extended periods of time. In one embodiment, B cell depletion by an anti-CD19 antibody of the invention may persist for at least 1 day, at least 2 days, at least 3 days, at least 4 days, at least 5 days at least 6 days, at least 7 days, at least 8 days, at least 9 days, at least 10 days, at least 15 days, at least 20 days, at least 25 days, or at least 30 days. In another embodiment, B cell depletion by an anti-CD19 antibody of the invention may persist for at least 1 week, at least 2 weeks, at least 3 weeks, at least 4 weeks, at least 5 weeks, at least 6 weeks, at least 7 weeks, at least 8 weeks, at least 9 weeks, or at least 10 weeks. In a further embodiment, B cell depletion by an anti-CD19 antibody of the invention may persist for at least 1 month, at least 2 months, at least 3 months, at least 4 months, at least 5 months, at least 6 months, at least 7 months, at least 8 months, at least 9 months, at least 10 months, at least 11 months or at least 12 months.

B cell malignancies are characterized by the pathological expansion of specific B cell subsets, for example, precursor B cell acute lymphoblastic leukemia is characterized by an abnormal expansion of B cells corresponding to pro-B cell/Pre-B cell developmental stages. The malignant B cells maintain cell surface expression of normal B cell markers such as CD19. An anti-CD19 antibody may therefore deplete malignant B cells in a human subject. In a specific embodiment, an anti-CD19 antibody of the invention may achieve at least 20%, at least 30%, at least 40%, at least 50%, at least 60%, at least 70%, at least 80%, at least 90%, at least 95%, or at least 100% depletion of malignant B cells in a human subject.

In one embodiment, a humanized anti-CD19 antibody described herein mediates antibody-dependent cellular cytotoxicity (ADCC), complement-dependent cell-mediated cytotoxicity (CDC), and/or apoptosis. In one embodiment, a humanized anti-CD19 antibody of the invention mediates antibody-dependent cellular cytotoxicity (ADCC) and/or apoptosis. In one embodiment, an anti-CD19 antibody of the invention has enhanced antibody-dependent cellular cytotoxicity (ADCC). In one embodiment, an anti-CD19 antibody of the invention comprises a variant Fc region that mediates enhanced antibody-dependent cellular cytotoxicity (ADCC). In one embodiment, an anti-CD19 antibody of the invention comprises an Fc region having complex N-glycoside-linked sugar chains linked to Asn297 in which fucose is not bound to N-acetylglucosamine in the reducing end, wherein said Fc region mediates enhanced antibody-dependent cellular cytotoxicity (ADCC).

The present invention further provides for anti-CD19 antibodies that may efficiently inhibit in vitro stimulated B cell proliferation. Proliferation of isolated peripheral B cells may be induced by various stimuli, for example, but not limited to stimulation by anti-IgM antibody, CD40 or CpG. These stimuli may be delivered in alone or in combination with each other.

In one embodiment, an anti-CD19 antibody of the invention inhibits in vitro stimulated B cell proliferation. In a another embodiment, an anti-CD19 antibody described herein inhibits in vitro B cell proliferation induced by anti-IgM/CpG or anti-IgM/CD40 stimulation. In one embodiment, an anti-CD19 antibody of the invention inhibits in vitro stimulated B cell proliferation by at least about 10%, at least about 20%, at least about 30%, at least about 40%, at least about 50% or at least about 75%.

In one embodiment, an Fc variant anti-CD19 antibody of the invention inhibits in vitro B cell proliferation induced by anti-IgM/CpG or anti-IgM/CD40 stimulation, wherein said Fc variant has altered binding affinity to one or more Fc ligands relative to a comparable non-variant molecule. In a specific embodiment, an Fc variant anti-CD19 antibody of the invention inhibits in vitro B cell proliferation induced by anti-IgM/CpG or anti-IgM/CD40 stimulation, wherein said Fc variant has enhanced binding to Fc gamma receptor IIB relative to a comparable non-variant Fc domain. In a further specific embodiment, an Fc variant anti-CD19 antibody of the invention inhibits in vitro stimulated B cell proliferation by at least about 10%, at least about 20%, at least about 30%, at least about 40%, at least about 50% or at least about 75%. In another embodiment, an Fc variant anti-CD19 antibody of the invention inhibits in vitro stimulated B cell proliferation, wherein said variant Fc domain has an affinity for Fc gamma receptor IIB that is at least 2 fold, or at least 3 fold, or at least 5 fold, or at least 7 fold, or a least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold greater than that of a comparable non-variant Fc domain.

The present invention also relates to a method of treating a B cell malignancy in a human comprising administering to a human in need thereof, a human, humanized or chimeric anti-CD19 antibody that may mediate human antibody-dependent cellular cytotoxicity (ADCC), complement-dependent cell-mediated cytotoxicity (CDC), and/or apoptosis in an amount sufficient to deplete circulating B cells. In a particular aspect, the present invention also concerns methods of treating a B cell malignancy in a human comprising administration of a therapeutically effective regimen of a human, humanized, or chimeric anti-CD19 antibody, which is of the IgG1 or IgG3 human isotype.

The present invention further relates to a method of treating an autoimmune disease or disorder in a human comprising administering to a human in need thereof a human, humanized, or chimeric anti-CD19 antibody that may mediate human ADCC, CDC, and/or apoptosis in an amount sufficient to deplete circulating B cells. The present invention also concerns methods of treating autoimmune disorders comprising administration of a therapeutically effective regimen of a human, humanized, or chimeric anti-CD19 antibody which is of the IgG1 or IgG3 human isotype.

The present invention also provides methods for treating or preventing humoral rejection in a human transplant recipient in need thereof comprising administering to the recipient a human, humanized, or chimeric anti-CD19 antibody of the invention in an amount that may deplete circulating B cells, or circulating immunoglobulin, or both. In other embodiments, the invention provides methods for preventing graft rejection or graft versus host disease in a human transplant recipient in need thereof comprising contacting a graft prior to transplantation with an amount of a human, humanized, or chimeric anti-CD19 antibody that may deplete B cells from the graft.

### 6.4. Production of Humanized Anti-CD19 Antibodies

Humanized antibodies described herein can be produced using a variety of techniques known in the art, including, but not limited to, CDR-grafting (see e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology 28(4/5):489-498; Studnicka et al., 1994, Protein Engineering, 7(6):805-814; and Roguska et al., 1994, Proc. Natl. Acad. Sci., 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Pat. No. 6,407,213, U.S. Pat. No. 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Often, FW residues in the FW regions will be substituted with the corresponding residue from the CDR donor antibody to alter, such as, improve, antigen binding. These FW substitutions are identified by methods well known in the art, e.g., by modeling of the interactions of the CDR and FW residues to identify FW residues important for antigen binding and sequence comparison to identify unusual FW residues at particular positions. (See, e.g., Queen et al., U.S. Pat. No. 5,585,089; and Riechmann et al., 1988, Nature, 332:323, which are incorporated herein by reference in their entireties.)

A humanized anti-CD19 antibody has one or more amino acid residues introduced into it from a source which is nonhuman. These nonhuman amino acid residues are often referred to as “import” residues, which are typically taken from an “import” variable domain. Thus, humanized antibodies comprise one or more CDRs from nonhuman immunoglobulin molecules and framework regions from human. Humanization of antibodies is well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated by reference herein in their entirety). In such humanized chimeric antibodies, substantially less than an intact human variable domain has been substituted by the corresponding sequence from a nonhuman species. In practice, humanized antibodies are typically human antibodies in which some CDR residues and possibly some FW residues are substituted by residues from analogous sites in rodent antibodies. Humanization of anti-CD19 antibodies can also be achieved by veneering or resurfacing (EP 592,106; EP 519,596; Padlan, 1991, Molecular Immunology 28(4/5):489-498; Studnicka et al., Protein Engineering, 7(6):805-814 (1994); and Roguska et al., Proc. Natl. Acad. Sci., 91:969-973 (1994)) or chain shuffling (U.S. Pat. No. 5,565,332), the contents of which are incorporated herein by reference in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequences which are most closely related to that of the rodent are then screened for the presences of specific residues that may be critical for antigen binding, appropriate structural formation and/or stability of the intended humanized mAb (Sims et al., J. Immunol., 151:2296 (1993); Chothia et al., J. Mol. Biol., 196:901 (1987), the contents of which are incorporated herein by reference in their entirety). The resulting FW sequences matching the desired criteria are then be used as the human donor FW regions for the humanized antibody.

Another method uses a particular FW derived from the consensus sequence of all human antibodies of a particular subgroup of light or heavy chains. The same FW may be used for several different humanized anti-CD19 antibodies (Carter et al., Proc. Natl. Acad. Sci. USA, 89:4285 (1992); Presta et al., J. Immunol., 151:2623 (1993), the contents of which are incorporated herein by reference in their entirety).

Anti-CD19 antibodies can be humanized with retention of high affinity for CD19 and other favorable biological properties. According to one aspect of the invention, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind CD19. In this way, FW residues can be selected and combined from the recipient and import sequences so that the desired antibody characteristic, for example affinity for CD19, is achieved. In general, the CDR residues are directly and most substantially involved in influencing antigen binding.

A “humanized” antibody may retain a similar antigenic specificity as the original antibody, i.e., in the present invention, the ability to bind human CD19 antigen. However, using certain methods of humanization, the affinity and/or specificity of binding of the antibody for human CD19 antigen may be altered using methods of “directed evolution,” as described by Wu et al., J. Mol. Biol., 294:151 (1999), the contents of which are incorporated herein by reference herein in their entirety.

Humanized anti-CD19 antibodies described herein can be constructed by the selection of distinct human framework regions for grafting of the HB12A or HB12B complementarity determining regions, or “CDR's” as described in the sections that follow. The invention encompasses a number of humanized versions of the mouse HB12A and HB12B antibody as well as chimeric versions, designated chHB12A and chHB12B.

### 6.5. Monoclonal Anti-CD19 Antibodies

A monoclonal anti-CD19 antibody exhibits binding specificity to human CD19 antigen and may mediate human ADCC, CDC and/or apoptotic mechanisms. Such an antibody can be generated using a wide variety of techniques known in the art including the use of hybridoma, recombinant, and phage display technologies, or a combination thereof. Antibodies are highly specific, being directed against a single antigenic site. An engineered anti-CD19 antibody can be produced by any means known in the art, including, but not limited to, those techniques described below and improvements to those techniques. Large-scale high-yield production typically involves culturing a host cell that produces the engineered anti-CD19 antibody and recovering the anti-CD19 antibody from the host cell culture.

### 6.5.1. Hybridoma Technique

Monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling et al., in Monoclonal Antibodies and T Cell Hybridomas, 563-681 (Elsevier, N.Y., 1981) (said references incorporated herein by reference in their entireties). For example, in the hybridoma method, a mouse or other appropriate host animal, such as a hamster or macaque monkey, is immunized to elicit lymphocytes that produce or are capable of producing antibodies that will specifically bind to the protein used for immunization. Lymphocytes may also be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Specific embodiments employ myeloma cells that fuse efficiently, support stable high-level production of antibody by the selected antibody-producing cells, and are sensitive to a medium such as HAT medium. Among these myeloma cell lines are murine myeloma lines, such as those derived from MOPC-21 and MPC-11 mouse tumors available from the Salk Institute Cell Distribution Center, San Diego, Calif., USA, and SP-2 or X63-Ag8.653 cells available from the American Type Culture Collection, Rockville, Md., USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the human CD19 antigen. The binding specificity of monoclonal antibodies produced by hybridoma cells can be determined by immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI 1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

### 6.5.2. Recombinant DNA Techniques

DNA encoding an anti-CD19 antibody described herein is readily isolated and sequenced using conventional procedures (e.g., by using oligonucleotide probes that are capable of binding specifically to genes encoding the heavy and light chains of anti-CD19 antibodies). The hybridoma cells serve as a source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of anti-CD19 antibodies in the recombinant host cells.

In phage display methods, functional antibody domains are displayed on the surface of phage particles which carry the polynucleotide sequences encoding them. In particular, DNA sequences encoding V_{H} and V_{L} domains are amplified from animal cDNA libraries (e.g., human or murine cDNA libraries of affected tissues). The DNA encoding the V_{H} and V_{L} domains are recombined together with an scFv linker by PCR and cloned into a phagemid vector. The vector is electroporated in E. coli and the E. coli is infected with helper phage. Phage used in these methods is typically filamentous phage including fd and M13 and the V_{H} and V_{L} domains are usually recombinantly fused to either the phage gene III or gene VIII. Phage expressing an antigen-binding domain that binds to a particular antigen can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods, 182:41-50; Ames et al., 1995, J. Immunol. Methods, 184:177-186; Kettleborough et al., 1994, Eur. J. Immunol., 24:952-958; Persic et al., 1997, Gene, 187:9-18; Burton et al., 1994, Advances in Immunology, 57:191-280; International Application No. PCT/GB91/O1 134; International Publication Nos. WO 90/02809, WO 91/10737, WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743, and 5,969,108; each of which is incorporated herein by reference in its entirety.

As described in the above references, after phage selection, the antibody coding regions from the phage can be isolated and used to generate whole antibodies, including human antibodies, or any other desired antigen-binding fragment, and expressed in any desired host, including mammalian cells, insect cells, plant cells, yeast, and bacteria, e.g., as described below. Techniques to recombinantly produce Fab, Fab′ and F(ab′)₂ fragments can also be employed using methods known in the art such as those disclosed in PCT Publication No. WO 92/22324; Mullinax et al., 1992, BioTechniques, 12(6):864-869; Sawai et al., 1995, AJRI, 34:26-34; and Better et al., 1988, Science, 240:1041-1043 (said references incorporated by reference in their entireties).

Antibodies may be isolated from antibody phage libraries generated using the techniques described in McCafferty et al., Nature, 348:552-554 (1990). Clackson et al., Nature, 352:624-628 (1991). Marks et al., J. Mol. Biol., 222:581-597 (1991) describe the isolation of murine and human antibodies, respectively, using phage libraries. Chain shuffling can be used in the production of high affinity (nM range) human antibodies (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of anti-CD19 antibodies.

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a heavy chain constant region, e.g., the human gamma 4 constant region, and the PCR amplified VL domains can be cloned into vectors expressing a light chain constant region, e.g., human kappa or lambda constant regions. The vectors for expressing the VH or VL domains may comprise an EF-1α promoter, a secretion signal, a cloning site for the variable domain, constant domains, and a selection marker such as neomycin. The VH and VL domains may also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

The DNA also may be modified, for example, by substituting the coding sequence for human heavy and light chain constant domains in place of the homologous murine sequences (U.S. Pat. No. 4,816,567; Morrison et al., Proc. Natl. Acad. Sci. USA, 81:6851 (1984)), or by covalently joining to the immunoglobulin coding sequence all or part of the coding sequence for a non-immunoglobulin polypeptide.

### 6.6. Chimeric Antibodies

The anti-CD19 antibodies herein specifically include chimeric antibodies (immunoglobulins) in which a portion of the heavy and/or light chain is identical with or homologous to corresponding sequences in antibodies derived from a particular species or belonging to a particular antibody class or subclass, while another portion of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include “primatized” antibodies comprising variable domain antigen-binding sequences derived from a nonhuman primate (e.g., Old World Monkey, such as baboon, rhesus or cynomolgus monkey) and human constant region sequences (U.S. Pat. No. 5,693,780).

### 6.7. Altered/Mutant Antibodies

Anti-CD19 antibodies of compositions and methods described herein can be mutant antibodies. As used herein, “antibody mutant” or “altered antibody” refers to an amino acid sequence variant of an anti-CD19 antibody wherein one or more of the amino acid residues of an anti-CD19 antibody have been modified. The modifications to the amino acid sequence of an anti-CD19 antibody include modifications to the sequence that may improve affinity or avidity of the antibody for its antigen, and/or modifications to the Fc portion of the antibody that may improve effector function.

The present invention therefore relates to human, humanized, and chimeric anti-CD19 antibodies disclosed herein as well as altered/mutant derivatives thereof including, but not limited to ones exhibiting altered human CD19 binding characteristics; e.g. altered association constants k_{ON}, dissociation constants k_{OFF}, and/or equilibrium constant or binding affinity, K_{D}. In certain embodiments the K_{D} of an anti-CD19 antibody described herein, or an altered/mutant derivative thereof, for human CD19 may be no more than about 10⁻⁶ M, 10⁻⁷ M, 10⁻⁸ M, or 10⁻⁹ M. Methods and reagents suitable for determination of such binding characteristics of an antibody of the present invention, or an altered/mutant derivative thereof, are known in the art and/or are commercially available (se above and, e.g., U.S. Pat. No. 6,849,425, U.S. Pat. No. 6,632,926, U.S. Pat. No. 6,294,391, and U.S. Pat. No. 6,143,574, each of which is hereby incorporated by reference in its entirety). Moreover, equipment and software designed for such kinetic analyses are commercially available (e.g. Biacore® A100, and Biacore® 2000 instruments; Biacore International AB, Uppsala, Sweden).

The modifications may be made to any known anti-CD19 antibodies or anti-CD19 antibodies identified as described herein. Such altered antibodies necessarily have less than 100% sequence identity or similarity with a known anti-CD19 antibody. By way of example, an altered antibody may have an amino acid sequence that is within the range of from about 25% to about 95% identical or similar to the amino acid sequence of either the heavy or light chain variable domain of an anti-CD19 antibody as described herein. An altered antibody may have an amino acid sequence having at least 25%, 35%, 45%, 55%, 65%, 75%, 80%, 85%, 90%, or 95% amino acid sequence identity or similarity with the amino acid sequence of either the heavy or light chain variable domain of an anti-CD19 antibody as described herein. In another embodiment, an altered antibody may have an amino acid sequence having at least 25%, 35%, 45%, 55%, 65%, 75%, 80%, 85%, 90%, or 95% amino acid sequence identity or similarity with the amino acid sequence of the heavy chain CDR1, CDR2, or CDR3 of an anti-CD19 antibody as described herein. In one embodiment, an altered antibody may maintain human CD19 binding capability. In certain embodiments, an anti-CD19 antibody as described herein may comprise a VH that is at least or about 10%, 15%, 20%, 25%, 30%, 35%, 40%, 45%, 50%, 55%, 60%, 65%, 70%, 75%, 80%, 85%, 90%, 95% or more identical to the amino acid sequence of HB12B-(3-72/JH4) (SEQ ID NO:34), HB12A VH (SEQ ID NO:2) HB12B VH (SEQ ID NO:18), 7E12 VH (SEQ ID NO:102), 14H5 VH (SEQ ID NO:103), 15D1 VH (SEQ ID NO:104), 15D7 VH (SEQ ID NO:105), 16C4 VH (SEQ ID NO:106), 14H5-YG (SEQ ID NO:107), 14H5-DG (SEQ ID NO:108), 14H5-LG (SEQ ID NO:109), 1A7 VH, 3C3 VH, 3E5 VH, 3D4 VH, 9G7 VH (SEQ ID NO:191), 3B4 VH (SEQ ID NO: 236), 3F11 VH or 6C11 VH (SEQ ID NO:192). In certain embodiments, an anti-CD19 antibody as described herein may comprise a VH that is at least or about 10%, 15%, 20%, 25%, 30%, 35%, 40%, 45%, 50%, 55%, 60%, 65%, 70%, 75%, 80%, 85%, 90%, 95% or more identical to the amino acid sequence of any of the VH domains, VL domains, or CDRs listed in Table 1.

In another embodiment, an altered antibody may have an amino acid sequence having at least 25%, 35%, 45%, 55%, 65%, 75%, 80%, 85%, 90%, or 95% amino acid sequence identity or similarity with the amino acid sequence of FW1, FW2, FW3, or FW4 regions of HB12B-(3-72/JH4) (SEQ ID NO:34), HB12A VH (SEQ ID NO:2) HB12B VH (SEQ ID NO:18), 7E12 VH (SEQ ID NO:102), 14H5 VH (SEQ ID NO:103), 15D1 VH (SEQ ID NO:104), 15D7 VH (SEQ ID NO:105), 16C4 VH (SEQ ID NO:106), 14H5-YG (SEQ ID NO:107), 14H5-DG (SEQ ID NO:108), 14H5-LG (SEQ ID NO:109), 1A7 VH, 3C3 VH, 3E5 VH, 3D4 VH, 9G7 VH (SEQ ID NO:191), 3B4 VH (SEQ ID NO: 236), 3F11 VH or 6C11 VH (SEQ ID NO:192). In another embodiment, an altered antibody may have an amino acid sequence having at least 25%, 35%, 45%, 55%, 65%, 75%, 80%, 85%, 90%, or 95% amino acid sequence identity or similarity with the amino acid sequence of FW1, FW2, FW3, or FW4 regions of any of the VH or VL domains listed in Table 1.

In another embodiment, an altered antibody may have an amino acid sequence having at least 25%, 35%, 45%, 55%, 65%, 75%, 80%, 85%, 90%, or 95% amino acid sequence identity or similarity with the amino acid sequence of the light chain CDR1, CDR2, or CDR3 of an anti-CD19 antibody as described herein. In certain embodiments, an anti-CD19 antibody of the invention may comprise a VL that is at least or about 10%, 15%, 20%, 25%, 30%, 35%, 40%, 45%, 50%, 55%, 60%, 65%, 70%, 75%, 80%, 85%, 90%, 95% or more identical to an amino acid sequence of HB12A VK (SEQ ID NO:4), HB12B VK (SEQ ID NO:20), HB12B-(A10-Jk4) (SEQ ID NO:52), HB12B-364987 (or 364987) (SEQ ID NO:62), HB12B-3649 (or 3649) (SEQ ID NO:68), HB12B-36 (or 36) (SEQ ID NO:70), 7E12 VK (SEQ ID NO:110), 14H5 (SEQ ID NO:111), 15D1 (SEQ ID NO:112), 16C9 (SEQ ID NO:113), 3C3 VK (SEQ ID NO:193), 3E5 VK (SEQ ID NO:194), 3D4 VK (SEQ ID NO:195), 3F1 VK (SEQ ID NO:196), 5B5 VK (SEQ ID NO:197), 6F7 VK (SEQ ID NO:198), 1C11 VK (SEQ ID NO:199), 2B11 VK (SEQ ID NO:200), 2D10 VK (SEQ ID NO:201), 5C11 VK (SEQ ID NO:202), 5D4 VK (SEQ ID NO:203), 6C11 VK (SEQ ID NO:204), 9G7 VK (SEQ ID NO:205), 1H4 VK (SEQ ID NO:206), or 5C4 VK (SEQ ID NO:207).

In another embodiment, an altered antibody may have an amino acid sequence having at least 25%, 35%, 45%, 55%, 65%, 75%, 80%, 85%, 90%, or 95% amino acid sequence identity or similarity with the amino acid sequence of FW1, FW2, FW3, or FW4 regions of HB12A VK (SEQ ID NO:4), HB12B VK (SEQ ID NO:20), HB12B-(A10-Jk4) (SEQ ID NO:52), HB12B-364987 (or 364987) (SEQ ID NO:62), HB12B-3649 (or 3649) (SEQ ID NO:68), HB12B-36 (or 36) (SEQ ID NO:70), 7E12 VK (SEQ ID NO:110), 14H5 (SEQ ID NO:111), 15D1 (SEQ ID NO:112), 16C9 (SEQ ID NO:113), 3C3 VK (SEQ ID NO:193), 3E5 VK (SEQ ID NO:194), 3D4 VK (SEQ ID NO:195), 3F1 VK (SEQ ID NO:196), 5B5 VK (SEQ ID NO:197), 6F7 VK (SEQ ID NO:198), 1C11 VK (SEQ ID NO:199), 2B11 VK (SEQ ID NO:200), 2D10 VK (SEQ ID NO:201), 5C11 VK (SEQ ID NO:202), 5D4 VK (SEQ ID NO:203), 6C11 VK (SEQ ID NO:204), 9G7 VK (SEQ ID NO:205), 1H4 VK (SEQ ID NO:206), or 5C4 VK (SEQ ID NO:207).

Identity or similarity with respect to a sequence is defined herein as the percentage of amino acid residues in the candidate sequence that are identical (i.e., same residue) or similar (i.e., amino acid residue from the same group based on common side-chain properties, see below) with anti-CD19 antibody residues, after aligning the sequences and introducing gaps, if necessary, to achieve the maximum percent sequence identity. None of N-terminal, C-terminal, or internal extensions, deletions, or insertions into the antibody sequence outside of the variable domain shall be construed as affecting sequence identity or similarity.

“% identity,” as known in the art, is a measure of the relationship between two polynucleotides or two polypeptides, as determined by comparing their sequences. In general, the two sequences to be compared are aligned to give a maximum correlation between the sequences. The alignment of the two sequences is examined and the number of positions giving an exact amino acid or nucleotide correspondence between the two sequences determined, divided by the total length of the alignment and multiplied by 100 to give a % identity figure. This % identity figure may be determined over the whole length of the sequences to be compared, which is particularly suitable for sequences of the same or very similar length and which are highly homologous, or over shorter defined lengths, which is more suitable for sequences of unequal length or which have a lower level of homology.

For example, sequences can be aligned with the software clustalw under Unix which generates a file with an “.aln” extension, this file can then be imported into the Bioedit program (Hall, T. A. 1999, BioEdit: a user-friendly biological sequence alignment editor and analysis program for Windows 95/98/NT. Nucl. Acids. Symp. Ser. 41:95-98) which opens the .aln file. In the Bioedit window, one can choose individual sequences (two at a time) and alignment them. This method allows for comparison of the entire sequence.

Methods for comparing the identity of two or more sequences are well known in the art. Thus for instance, programs are available in the Wisconsin Sequence Analysis Package, version 9.1 (Devereux J. et al., Nucleic Acids Res., 12:387-395, 1984, available from Genetics Computer Group, Madison, Wis., USA). The determination of percent identity between two sequences can be accomplished using a mathematical algorithm. For example, the programs BESTFIT and GAP, may be used to determine the % identity between two polynucleotides and the % identity between two polypeptide sequences. BESTFIT uses the “local homology” algorithm of Smith and Waterman (Advances in Applied Mathematics, 2:482-489, 1981) and finds the best single region of similarity between two sequences. BESTFIT is more suited to comparing two polynucleotide or two polypeptide sequences which are dissimilar in length, the program assuming that the shorter sequence represents a portion of the longer. In comparison, GAP aligns two sequences finding a “maximum similarity” according to the algorithm of Neddleman and Wunsch (J. Mol. Biol., 48:443-354, 1970). GAP is more suited to comparing sequences which are approximately the same length and an alignment is expected over the entire length. For example, the parameters “Gap Weight” and “Length Weight” used in each program are 50 and 3 for polynucleotides and 12 and 4 for polypeptides, respectively. In some aspects of the invention, % identities and similarities are determined when the two sequences being compared are optimally aligned.

Other programs for determining identity and/or similarity between sequences are also known in the art, for instance the BLAST family of programs (Karlin & Altschul, 1990, Proc. Natl. Acad. Sci. USA, 87:2264-2268, modified as in Karlin & Altschul, 1993, Proc. Natl. Acad. Sci. USA, 90:5873-5877, available from the National Center for Biotechnology Information (NCB), Bethesda, Md., USA and accessible through the home page of the NCBI at www.ncbi.nlm.nih.gov). These programs are non-limiting examples of a mathematical algorithm utilized for the comparison of two sequences. Such an algorithm is incorporated into the NBLAST and XBLAST programs of Altschul et al., 1990, J. Mol. Biol., 215:403-410. BLAST nucleotide searches can be performed with the NBLAST program, score=100, wordlength=12 to obtain nucleotide sequences homologous to a nucleic acid molecule encoding all or a portion if an anti-CD19 antibody of the invention. BLAST protein searches can be performed with the XBLAST program, score=50, wordlength=3 to obtain amino acid sequences homologous to a protein molecule of the invention. To obtain gapped alignments for comparison purposes, Gapped BLAST can be utilized as described in Altschul et al., 1997, Nucleic Acids Res., 25:3389-3402. PSI-Blast can also be used to perform an iterated search which detects distant relationships between molecules (Id.). When utilizing BLAST, Gapped BLAST, and PSI-Blast programs, the default parameters of the respective programs (e.g., XBLAST and NBLAST) can be used. See, http://www.ncbi.nlm.nih.gov.

Another non-limiting example of a program for determining identity and/or similarity between sequences known in the art is FASTA (Pearson W. R. and Lipman D. J., Proc. Natl. Acad. Sci. USA, 85:2444-2448, 1988, available as part of the Wisconsin Sequence Analysis Package). The BLOSUM62 amino acid substitution matrix (Henikoff S, and Henikoff J. G., Proc. Natl. Acad. Sci. USA, 89:10915-10919, 1992) may be used in polypeptide sequence comparisons including where nucleotide sequences are first translated into amino acid sequences before comparison.

Yet another non-limiting example of a program known in the art for determining identity and/or similarity between amino acid sequences is SeqWeb Software (a web-based interface to the GCG Wisconsin Package: Gap program) which is utilized with the default algorithm and parameter settings of the program: blosum62, gap weight 8, length weight 2.

The percent identity between two sequences can be determined using techniques similar to those described above, with or without allowing gaps. In calculating percent identity, typically exact matches are counted.

The program BESTFIT may be used to determine the % identity of a query polynucleotide or a polypeptide sequence with respect to a polynucleotide or a polypeptide sequence of the present invention, the query and the reference sequence being optimally aligned and the parameters of the program set at the default value.

To generate an altered antibody, one or more amino acid alterations (e.g., substitutions) are introduced in one or more of the hypervariable regions of the species-dependent antibody. One or more alterations (e.g., substitutions) of framework region residues may also be introduced in an anti-CD19 antibody where these result in an improvement in the binding affinity of the antibody mutant for the antigen from the second mammalian species. Examples of framework region residues to modify include those which non-covalently bind antigen directly (Amit et al., Science, 233:747-753 (1986)); interact with/effect the conformation of a CDR (Chothia et al., J. Mol. Biol., 196:901-917 (1987)); and/or participate in the V_{L}-V_{H} interface (EP 239 400B1). In certain embodiments, modification of one or more of such framework region residues results in an enhancement of the binding affinity of the antibody for the antigen from the second mammalian species. For example, from about one to about five framework residues may be altered in this embodiment of the invention. Sometimes, this may be sufficient to yield an antibody mutant suitable for use in preclinical trials, even where none of the hypervariable region residues have been altered. Normally, however, an altered antibody will comprise additional hypervariable region alteration(s).

The hypervariable region residues which are altered may be changed randomly, especially where the starting binding affinity of an anti-CD19 antibody for the antigen from the second mammalian species is such that such randomly produced altered antibody can be readily screened.

One useful procedure for generating such an altered antibody is called “alanine scanning mutagenesis” (Cunningham and Wells, Science, 244:1081-1085 (1989)). Here, one or more of the hypervariable region residue(s) are replaced by alanine or polyalanine residue(s) to affect the interaction of the amino acids with the antigen from the second mammalian species. Those hypervariable region residue(s) demonstrating functional sensitivity to the substitutions then are refined by introducing additional or other mutations at or for the sites of substitution. Thus, while the site for introducing an amino acid sequence variation is predetermined, the nature of the mutation per se need not be predetermined. The Ala-mutants produced this way are screened for their biological activity as described herein.

Another procedure for generating such an altered antibody involves affinity maturation using phage display (Hawkins et al., J. Mol. Biol., 254:889-896 (1992) and Lowman et al., Biochemistry, 30(45):10832-10837 (1991)). Briefly, several hypervariable region sites (e.g., 6-7 sites) are mutated to generate all possible amino acid substitutions at each site. The antibody mutants thus generated are displayed in a monovalent fashion from filamentous phage particles as fusions to the gene III product of M13 packaged within each particle. The phage-displayed mutants are then screened for their biological activity (e.g., binding affinity) as herein disclosed.

Mutations in antibody sequences may include substitutions, deletions, including internal deletions, additions, including additions yielding fusion proteins, or conservative substitutions of amino acid residues within and/or adjacent to the amino acid sequence, but that result in a “silent” change, in that the change produces a functionally equivalent anti-CD19 antibody. Conservative amino acid substitutions may be made on the basis of similarity in polarity, charge, solubility, hydrophobicity, hydrophilicity, and/or the amphipathic nature of the residues involved. For example, non-polar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. In addition, glycine and proline are residues that can influence chain orientation. Non-conservative substitutions will entail exchanging a member of one of these classes for a member of another class. Furthermore, if desired, non-classical amino acids or chemical amino acid analogs can be introduced as a substitution or addition into the antibody sequence. Non-classical amino acids include, but are not limited to, the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general.

In another embodiment, the sites selected for modification are affinity matured using phage display (see above).

Any technique for mutagenesis known in the art can be used to modify individual nucleotides in a DNA sequence, for purposes of making amino acid substitution(s) in the antibody sequence, or for creating/deleting restriction sites to facilitate further manipulations. Such techniques include, but are not limited to, chemical mutagenesis, in vitro site-directed mutagenesis (Kunkel, Proc. Natl. Acad. Sci. USA, 82:488 (1985); Hutchinson, C. et al., J. Biol. Chem., 253:6551 (1978)), oligonucleotide-directed mutagenesis (Smith, Ann. Rev. Genet., 19:423-463 (1985); Hill et al., Methods Enzymol., 155:558-568 (1987)), PCR-based overlap extension (Ho et al., Gene, 77:51-59 (1989)), PCR-based megaprimer mutagenesis (Sarkar et al., Biotechniques, 8:404-407 (1990)), etc. Modifications can be confirmed by double-stranded dideoxy DNA sequencing.

In certain embodiments of the invention, an anti-CD19 antibody can be modified to produce fusion proteins; i.e., the antibody, or a fragment thereof, fused to a heterologous protein, polypeptide or peptide. In certain embodiments, the protein fused to the portion of an anti-CD19 antibody is an enzyme component of Antibody-Directed Enzyme Prodrug Therapy (ADEPT). Examples of other proteins or polypeptides that can be engineered as a fusion protein with an anti-CD19 antibody include, but are not limited to toxins such as ricin, abrin, ribonuclease, DNase I, Staphylococcal enterotoxin-A, pokeweed anti-viral protein, gelonin, diphtherin toxin, Pseudomonas exotoxin, and Pseudomonas endotoxin. See, for example, Pastan et al., Cell, 47:641 (1986), and Goldenberg et al., Cancer Journal for Clinicians, 44:43 (1994). Enzymatically active toxins and fragments thereof which can be used include diphtheria A chain, non-binding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232 published Oct. 28, 1993.

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of the antiCD19 antibody or fragments thereof (e.g., an antibody or a fragment thereof with higher affinities and lower dissociation rates). See, generally, U.S. Pat. Nos. 5,605,793; 5,811,238; 5,830,721; 5,834,252; and 5,837,458, and Patten et al., 1997, Curr. Opinion Biotechnol., 8:724-33; Harayama, 1998, Trends Biotechnol. 16(2):76-82; Hansson et al., 1999, J. Mol. Biol., 287:265-76; and Lorenzo and Blasco, 1998, Biotechniques 24(2):308-313 (each of these patents and publications are hereby incorporated by reference in its entirety). The antibody can further be a binding-domain immunoglobulin fusion protein as described in U.S. Publication 20030118592, U.S. Publication 200330133939, and PCT Publication WO 02/056910, all to Ledbetter et al., which are incorporated herein by reference in their entireties.

In certain embodiments of the invention, anti-CD19 antibody is a parent antibody. A “parent antibody” is an antibody comprising an amino acid sequence which may lack, or may be deficient in, one or more amino acid residues in or adjacent to one or more hypervariable regions thereof compared to an altered/mutant antibody as herein disclosed. Thus, the parent antibody may have a shorter hypervariable region than the corresponding hypervariable region of an antibody mutant as herein disclosed. The parent polypeptide may comprise a native antibody sequence (i.e., a naturally occurring, including a naturally occurring allelic variant) or an antibody sequence with pre-existing amino acid sequence modifications (such as other insertions, deletions and/or substitutions) of a naturally occurring sequence. The parent antibody may be a humanized antibody or a human antibody.

### 6.8. Bispecific Antibodies

Bispecific antibodies are antibodies that have binding specificities for at least two different epitopes. Exemplary bispecific antibodies may bind to two different epitopes of the B cell surface marker. Other such antibodies may bind a first B cell marker and further bind a second B cell surface marker. An anti-B cell marker binding arm may also be combined with an arm which binds to a triggering molecule on a leukocyte such as a T cell receptor molecule (e.g., CD2 or CD3), or Fc receptors for IgG (FcγR), so as to focus cellular defense mechanisms to the B cell. Bispecific antibodies may also be used to localize cytotoxic agents to the B cell. These antibodies possess a B cell marker-binding arm and an arm which binds the cytotoxic agent (e.g., saporin, anti-interferon-α, vinca alkaloid, ricin A chain, methola-exate or radioactive isotope hapten). Bispecific antibodies can be prepared as full-length antibodies or antibody fragments (e.g., F(ab′): bispecific antibodies).

Methods for making bispecific antibodies are known in the art. (See, for example, Millstein et al., Nature, 305:537-539 (1983); Traunecker et al., EMBO J., 10:3655-3659 (1991); Suresh et al., Methods in Enzymology, 121:210 (1986); Kostelny et al., J. Immunol., 148(5):1547-1553 (1992); Hollinger et al., Proc. Natl. Acad. Sci. USA, 90:6444-6448 (1993); Gruber et al., J. Immunol., 152:5368 (1994); U.S. Pat. Nos. 4,474,893; 4,714,681; 4,925,648; 5,573,920; 5,601,81; 95,731,168; 4,676,980; and 4,676,980, WO 94/04690; WO 91/00360; WO 92/200373; WO 93/17715; WO 92/08802; and EP 03089.)

In one embodiment, where an anti-CD19 antibody of compositions and methods of the invention is bispecific, the anti-CD19 antibody may be human or humanized and may have specificity for human CD19 and an epitope on a T cell or may be capable of binding to a human effector cell such as, for example, a monocyte/macrophage and/or a natural killer cell to effect cell death.

In one embodiment, an anti-CD19 antibody of the invention is a bispecific antibody capable of specifically binding to a first and second antigen, wherein said first antigen is human CD19 and said second antigen is an Fc gamma receptor selected from the group consisting of FcγRI, FcγRIIA, FcγRIIB, FcγRIIIA and/or FcγRIV. In a further embodiment, an anti-CD19 antibody of the invention is a bispecific antibody capable of specifically binding to human CD19 and FcγRIIB. In another embodiment, an anti-CD19 antibody of the invention is a bispecific antibody capable of specifically binding to human CD19 and human FcγRIIB.

### 6.9. Variant Fc Regions

The present invention provides formulation of proteins comprising a variant Fc region. That is, a non naturally occurring Fc region, for example an Fc region comprising one or more non naturally occurring amino acid residues. Also encompassed by the variant Fc regions of present invention are Fc regions which comprise amino acid deletions, additions and/or modifications.

It will be understood that Fc region as used herein includes the polypeptides comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as in Kabat et al. (1991, NIH Publication 91-3242, National Technical Information Service, Springfield, Va.). The “EU index as set forth in Kabat” refers to the residue numbering of the human IgG1 EU antibody as described in Kabat et al. supra. Fc may refer to this region in isolation, or this region in the context of an antibody, antibody fragment, or Fc fusion protein. An Fc variant protein may be an antibody, Fc fusion, or any protein or protein domain that comprises an Fc region including, but not limited to, proteins comprising variant Fc regions, which are non naturally occurring variants of an Fc. Note: Polymorphisms have been observed at a number of Fc positions, including but not limited to Kabat 270, 272, 312, 315, 356, and 358, and thus slight differences between the presented sequence and sequences in the prior art may exist.

The present invention encompasses Fc variant proteins which have altered binding properties for an Fc ligand (e.g., an Fc receptor, C1q) relative to a comparable molecule (e.g., a protein having the same amino acid sequence except having a wild type Fc region). Examples of binding properties include but are not limited to, binding specificity, equilibrium dissociation constant (K_{D}), dissociation and association rates (k_{off} and kₒₙ respectively), binding affinity and/or avidity. It is generally understood that a binding molecule (e.g., a Fc variant protein such as an antibody) with a low K_{D} may be preferable to a binding molecule with a high K_{D}. However, in some instances the value of the kₒₙ or k_{off} may be more relevant than the value of the K_{D}. One skilled in the art can determine which kinetic parameter is most important for a given antibody application.

The affinities and binding properties of an Fc domain for its ligand may be determined by a variety of in vitro assay methods (biochemical or immunological based assays) known in the art for determining Fc-FcγR interactions, i.e., specific binding of an Fc region to an FcγR including but not limited to, equilibrium methods (e.g., enzyme-linked immunoabsorbent assay (ELISA), or radioimmunoassay (RIA)), or kinetics (e.g., BIACORE® analysis), and other methods such as indirect binding assays, competitive inhibition assays, fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions.

In one embodiment, the Fc variant protein has enhanced binding to one or more Fc ligands relative to a comparable molecule. In another embodiment, the Fc variant protein has an affinity for an Fc ligand that is at least 2 fold, or at least 3 fold, or at least 5 fold, or at least 7 fold, or a least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold greater than that of a comparable molecule. In a specific embodiment, the Fc variant protein has enhanced binding to an Fc receptor. In another specific embodiment, the Fc variant protein has enhanced binding to the Fc receptor FcγRIIIA. In a further specific embodiment, the Fc variant protein has enhanced binding to the Fc receptor FcγRIIB. In still another specific embodiment, the Fc variant protein has enhanced binding to the Fc receptor FcRn. In yet another specific embodiment, the Fc variant protein has enhanced binding to C1q relative to a comparable molecule.

In one embodiment, an anti-CD19 antibody of the invention comprises a variant Fc domain wherein said variant Fc domain has enhanced binding affinity to Fc gamma receptor IIB relative to a comparable non-variant Fc domain. In a further embodiment, an anti-CD19 antibody of the invention comprises a variant Fc domain wherein said variant Fc domain has an affinity for Fc gamma receptor IIB that is at least 2 fold, or at least 3 fold, or at least 5 fold, or at least 7 fold, or a least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold greater than that of a comparable non-variant Fc domain.

The serum half-life of proteins comprising Fc regions may be increased by increasing the binding affinity of the Fc region for FcRn. In one embodiment, the Fc variant protein has enhanced serum half life relative to comparable molecule.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enables these cytotoxic effector cells to bind specifically to an antigen-bearing target cell and subsequently kill the target cell with cytotoxins. Specific high-affinity IgG antibodies directed to the surface of target cells “arm” the cytotoxic cells and are absolutely required for such killing. Lysis of the target cell is extracellular, requires direct cell-to-cell contact, and does not involve complement. It is contemplated that, in addition to antibodies, other proteins comprising Fc regions, specifically Fc fusion proteins, having the capacity to bind specifically to an antigen-bearing target cell will be able to effect cell-mediated cytotoxicity. For simplicity, the cell-mediated cytotoxicity resulting from the activity of an Fc fusion protein is also referred to herein as ADCC activity.

The ability of any particular Fc variant protein to mediate lysis of the target cell by ADCC can be assayed. To assess ADCC activity an Fc variant protein of interest is added to target cells in combination with immune effector cells, which may be activated by the antigen antibody complexes resulting in cytolysis of the target cell. Cytolysis is generally detected by the release of label (e.g. radioactive substrates, fluorescent dyes or natural intracellular proteins) from the lysed cells. Useful effector cells for such assays include peripheral blood mononuclear cells (PBMC) and Natural Killer (NK) cells. Specific examples of in vitro ADCC assays are described in Wisecarver et al., 1985 J Immunol Methods 79:277-282; Bruggemann et al., 1987, J Exp Med 166:1351-1361; Wilkinson et al., 2001, J Immunol Methods 258:183-191; Patel et al., 1995 J Immunol Methods 184:29-38. ADCC activity of the Fc variant protein of interest may also be assessed in vivo, e.g., in an animal model such as that disclosed in Clynes et al., 1998, Proc. Natl. Acad. Sci. USA 95:652-656.

In one embodiment, an Fc variant protein has enhanced ADCC activity relative to a comparable molecule. In a specific embodiment, an Fc variant protein has ADCC activity that is at least 2 fold, or at least 3 fold, or at least 5 fold, or at least 10 fold, or at least 50 fold, or at least 100 fold greater than that of a comparable molecule. In another specific embodiment, an Fc variant protein has enhanced binding to the Fc receptor FcγRIIIA and has enhanced ADCC activity relative to a comparable molecule. In other embodiments, the Fc variant protein has both enhanced ADCC activity and enhanced serum half life relative to a comparable molecule.

In one embodiment, an Fc variant protein has reduced ADCC activity relative to a comparable molecule. In a specific embodiment, an Fc variant protein has ADCC activity that is at least 2 fold, or at least 3 fold, or at least 5 fold, or at least 10 fold, or at least 50 fold, or at least 100 fold lower than that of a comparable molecule. In another specific embodiment, an Fc variant protein has reduced binding to the Fc receptor FcγRIIIA and has reduced ADCC activity relative to a comparable molecule. In other embodiments, the Fc variant protein has both reduced ADCC activity and enhanced serum half life relative to a comparable molecule.

“Complement dependent cytotoxicity” and “CDC” refer to the lysing of a target cell in the presence of complement. The complement activation pathway is initiated by the binding of the first component of the complement system (Clq) to a molecule, an antibody for example, complexed with a cognate antigen. To assess complement activation, a CDC assay, e.g. as described in Gazzano-Santoro et al., 1996, J. Immunol. Methods, 202:163, may be performed. In one embodiment, an Fc variant protein has enhanced CDC activity relative to a comparable molecule. In a specific embodiment, an Fc variant protein has CDC activity that is at least 2 fold, or at least 3 fold, or at least 5 fold, or at least 10 fold, or at least 50 fold, or at least 100 fold greater than that of a comparable molecule. In other embodiments, the Fc variant protein has both enhanced CDC activity and enhanced serum half life relative to a comparable molecule.

In one embodiment, the Fc variant protein has reduced binding to one or more Fc ligand relative to a comparable molecule. In another embodiment, the Fc variant protein has an affinity for an Fc ligand that is at least 2 fold, or at least 3 fold, or at least 5 fold, or at least 7 fold, or a least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold lower than that of a comparable molecule. In a specific embodiment, the Fc variant protein has reduced binding to an Fc receptor. In another specific embodiment, the Fc variant protein has reduced binding to the Fc receptor FcγRIIIA. In a further specific embodiment, an Fc variant described herein has an affinity for the Fc receptor FcγRIIIA that is at least about 5 fold lower than that of a comparable molecule, wherein said Fc variant has an affinity for the Fc receptor FcγRIIB that is within about 2 fold of that of a comparable molecule. In still another specific embodiment, the Fc variant protein has reduced binding to the Fc receptor FcRn. In yet another specific embodiment, the Fc variant protein has reduced binding to C1q relative to a comparable molecule.

In one embodiment, the present invention provides Fc variants, wherein the Fc region comprises a non naturally occurring amino acid residue at one or more positions selected from the group consisting of 234, 235, 236, 237, 238, 239, 240, 241, 243, 244, 245, 247, 251, 252, 254, 255, 256, 262, 263, 264, 265, 266, 267, 268, 269, 279, 280, 284, 292, 296, 297, 298, 299, 305, 313, 316, 325, 326, 327, 328, 329, 330, 331, 332, 333, 334, 339, 341, 343, 370, 373, 378, 392, 416, 419, 421, 440 and 443 as numbered by the EU index as set forth in Kabat. Optionally, the Fc region may comprise a non naturally occurring amino acid residue at additional and/or alternative positions known to one skilled in the art (see, e.g., U.S. Pat. Nos. 5,624,821; 6,277,375; 6,737,056; PCT Patent Publications WO 01/58957; WO 02/06919; WO 04/016750; WO 04/029207; WO 04/035752; WO 04/074455; WO 04/099249; WO 04/063351; WO 05/070963; WO 05/040217, WO 05/092925 and WO 06/020114).

In one embodiment, the present invention provides formulations, wherein the Fc region comprises a non naturally occurring amino acid residue at one or more positions selected from the group consisting of 234, 235, 236, 237, 238, 239, 240, 241, 243, 244, 245, 247, 251, 252, 254, 255, 256, 262, 263, 264, 265, 266, 267, 268, 269, 279, 280, 284, 292, 296, 297, 298, 299, 305, 313, 316, 325, 326, 327, 328, 329, 330, 331, 332, 333, 334, 339, 341, 343, 370, 373, 378, 392, 416, 419, 421, 440 and 443 as numbered by the EU index as set forth in Kabat. Optionally, the Fc region may comprise a non naturally occurring amino acid residue at additional and/or alternative positions known to one skilled in the art (see, e.g., U.S. Pat. Nos. 5,624,821; 6,277,375; 6,737,056; PCT Patent Publications WO 01/58957; WO 02/06919; WO 04/016750; WO 04/029207; WO 04/035752; WO 04/074455; WO 04/099249; WO 04/063351; WO 05/070963; WO 05/040217, WO 05/092925 and WO 06/020114).

In a specific embodiment, the present invention provides an Fc variant, wherein the Fc region comprises at least one non naturally occurring amino acid residue selected from the group consisting of 234D, 234E, 234N, 234Q, 234T, 234H, 234Y, 234I, 234V, 234F, 235A, 235D, 235R, 235W, 235P, 235S, 235N, 235Q, 235T, 235H, 235Y, 235I, 235V, 235F, 236E, 239D, 239E, 239N, 239Q, 239F, 239T, 239H, 239Y, 240I, 240A, 240T, 240M, 241W, 241 L, 241Y, 241E, 241R. 243W, 243L 243Y, 243R, 243Q, 244H, 245A, 247L, 247V, 247G, 251F, 252Y, 254T, 255L, 256E, 256M, 262I, 262A, 262T, 262E, 263I, 263A, 263T, 263M, 264L, 264I, 264W, 264T, 264R, 264F, 264M, 264Y, 264E, 265G, 265N, 265Q, 265Y, 265F, 265V, 265I, 265L, 265H, 265T, 266I, 266A, 266T, 266M, 267Q, 267L, 268E, 269H, 269Y, 269F, 269R, 270E, 280A, 284M, 292P, 292L, 296E, 296Q, 296D, 296N, 296S, 296T, 296L, 296I, 296H, 269G, 297S, 297D, 297E, 298H, 298I, 298T, 298F, 299I, 299L, 299A, 299S, 299V, 299H, 299F, 299E, 305I, 313F, 316D, 325Q, 325L, 325I, 325D, 325E, 325A, 325T, 325V, 325H, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 330I, 330F, 330R, 330H, 331G, 331A, 331L, 331M, 331F, 331W, 331K, 331Q, 331E, 331S, 331V, 331I, 331C, 331Y, 331H, 331R, 331N, 331D, 331T, 332D, 332S, 332W, 332F, 332E, 332N, 332Q, 332T, 332H, 332Y, 332A, 339T, 370E, 370N, 378D, 392T, 396L, 416G, 419H, 421K, 440Y and 434W as numbered by the EU index as set forth in Kabat. Optionally, the Fc region may comprise additional and/or alternative non naturally occurring amino acid residues known to one skilled in the art (see, e.g., U.S. Pat. Nos. 5,624,821; 6,277,375; 6,737,056; PCT Patent Publications WO 01/58957; WO 02/06919; WO 04/016750; WO 04/029207; WO 04/035752 and WO 05/040217).

In a specific embodiment, the present invention provides an Fc variant protein formulation, wherein the Fc region comprises at least one non naturally occurring amino acid residue selected from the group consisting of 234D, 234E, 234N, 234Q, 234T, 234H, 234Y, 234I, 234V, 234F, 235A, 235D, 235R, 235W, 235P, 235S, 235N, 235Q, 235T, 235H, 235Y, 235I, 235V, 235F, 236E, 239D, 239E, 239N, 239Q, 239F, 239T, 239H, 239Y, 240I, 240A, 240T, 240M, 241W, 241 L, 241Y, 241E, 241R, 243W, 243L 243Y, 243R, 243Q, 244H, 245A, 247L, 247V, 247G, 251F, 252Y, 254T, 255L, 256E, 256M, 262I, 262A, 262T, 262E, 263I, 263A, 263T, 263M, 264L, 264I, 264W, 264T, 264R, 264F, 264M, 264Y, 264E, 265G, 265N, 265Q, 265Y, 265F, 265V, 265I, 265L, 265H, 265T, 266I, 266A, 266T, 266M, 267Q, 267L, 268E, 269H, 269Y, 269F, 269R, 270E, 280A, 284M, 292P, 292L, 296E, 296Q, 296D, 296N, 296S, 296T, 296L, 296I, 296H, 269G, 297S, 297D, 297E, 298H, 298I, 298T, 298F, 299I, 299L, 299A, 299S, 299V, 299H, 299F, 299E, 305I, 313F, 316D, 325Q, 325L, 325I, 325D, 325E, 325A, 325T, 325V, 325H, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 330I, 330F, 330R, 330H, 331G, 331A, 331L, 331M, 331F, 331W, 331K, 331Q, 331E, 331S, 331V, 331I, 331C, 331Y, 331H, 331R, 331N, 331D, 331T, 332D, 332S, 332W, 332F, 332E, 332N, 332Q, 332T, 332H, 332Y, 332A, 339T, 370E, 370N, 378D, 392T, 396L, 416G, 419H, 421K, 440Y and 434W as numbered by the EU index as set forth in Kabat. Optionally, the Fc region may comprise additional and/or alternative non naturally occurring amino acid residues known to one skilled in the art (see, e.g., U.S. Pat. Nos. 5,624,821; 6,277,375; 6,737,056; PCT Patent Publications WO 01/58957; WO 02/06919; WO 04/016750; WO 04/029207; WO 04/035752 and WO 05/040217).

In another embodiment, the present invention provides an Fc variant, wherein the Fc region comprises at least one non naturally occurring amino acid at one or more positions selected from the group consisting of 239, 330 and 332, as numbered by the EU index as set forth in Kabat. In a specific embodiment, the present invention provides an Fc variant, wherein the Fc region comprises at least one non naturally occurring amino acid selected from the group consisting of 239D, 330L and 332E, as numbered by the EU index as set forth in Kabat. Optionally, the Fc region may further comprise additional non naturally occurring amino acid at one or more positions selected from the group consisting of 252, 254, and 256, as numbered by the EU index as set forth in Kabat. In a specific embodiment, the present invention provides an Fc variant, wherein the Fc region comprises at least one non naturally occurring amino acid selected from the group consisting of 239D, 330L and 332E, as numbered by the EU index as set forth in Kabat and at least one non naturally occurring amino acid at one or more positions selected from the group consisting of 252Y, 254T and 256E, as numbered by the EU index as set forth in Kabat.

In another embodiment, the present invention provides an Fc variant, wherein the Fc region comprises at least one non naturally occurring amino acid at one or more positions selected from the group consisting of 234, 235 and 331, as numbered by the EU index as set forth in Kabat. In a specific embodiment, the present invention provides an Fc variant, wherein the Fc region comprises at least one non naturally occurring amino acid selected from the group consisting of 234F, 235F, 235Y, and 331S, as numbered by the EU index as set forth in Kabat. In a further specific embodiment, an Fc variant of the invention comprises the 234F, 235F, and 331S non naturally occurring amino acid residues, as numbered by the EU index as set forth in Kabat. In another specific embodiment, an Fc variant of the invention comprises the 234F, 235Y, and 331S non naturally occurring amino acid residues, as numbered by the EU index as set forth in Kabat. Optionally, the Fc region may further comprise additional non naturally occurring amino acid at one or more positions selected from the group consisting of 252, 254, and 256, as numbered by the EU index as set forth in Kabat. In a specific embodiment, the present invention provides an Fc variant, wherein the Fc region comprises at least one non naturally occurring amino acid selected from the group consisting of 234F, 235F, 235Y, and 331S, as numbered by the EU index as set forth in Kabat; and at least one non naturally occurring amino acid at one or more positions are selected from the group consisting of 252Y, 254T and 256E, as numbered by the EU index as set forth in Kabat.

In another embodiment, the present invention provides an Fc variant protein formulation, wherein the Fc region comprises at least a non naturally occurring amino acid at one or more positions selected from the group consisting of 239, 330 and 332, as numbered by the EU index as set forth in Kabat. In a specific embodiment, the present invention provides an Fc variant protein formulation, wherein the Fc region comprises at least one non naturally occurring amino acid selected from the group consisting of 239D, 330L and 332E, as numbered by the EU index as set forth in Kabat. Optionally, the Fc region may further comprise additional non naturally occurring amino acid at one or more positions selected from the group consisting of 252, 254, and 256, as numbered by the EU index as set forth in Kabat. In a specific embodiment, the present invention provides an Fc variant protein formulation, wherein the Fc region comprises at least one non naturally occurring amino acid selected from the group consisting of 239D, 330L and 332E, as numbered by the EU index as set forth in Kabat and at least one non naturally occurring amino acid at one or more positions are selected from the group consisting of 252Y, 254T and 256E, as numbered by the EU index as set forth in Kabat.

In another embodiment, the present invention provides an Fc variant protein formulation, wherein the Fc region comprises at least one non naturally occurring amino acid at one or more positions selected from the group consisting of 234, 235 and 331, as numbered by the EU index as set forth in Kabat. In a specific embodiment, the present invention provides an Fc variant protein formulation, wherein the Fc region comprises at least one non naturally occurring amino acid selected from the group consisting of 234F, 235F, 235Y, and 331S, as numbered by the EU index as set forth in Kabat. Optionally, the Fc region may further comprise additional non naturally occurring amino acid at one or more positions selected from the group consisting of 252, 254, and 256, as numbered by the EU index as set forth in Kabat. In a specific embodiment, the present invention provides an Fc variant protein formulation, wherein the Fc region comprises at least one non naturally occurring amino acid selected from the group consisting of 234F, 235F, 235Y, and 331S, as numbered by the EU index as set forth in Kabat; and at least one non naturally occurring amino acid at one or more positions are selected from the group consisting of 252Y, 254T and 256E, as numbered by the EU index as set forth in Kabat.

In one embodiment, the Fc variants of the present invention may be combined with other known Fc variants such as those disclosed in Ghetie et al., 1997, Nat. Biotech. 15:637-40; Duncan et al, 1988, Nature 332:563-564; Lund et al., 1991, J. Immunol. 147:2657-2662; Lund et al, 1992, Mol Immunol 29:53-59; Alegre et al, 1994, Transplantation 57:1537-1543; Hutchins et al., 1995, Proc Natl. Acad Sci USA 92:11980-11984; Jefferis et al, 1995, Immunol Lett. 44:111-117; Lund et al., 1995, Faseb J 9:115-119; Jefferis et al, 1996, Immunol Lett 54:101-104; Lund et al, 1996, J Immunol 157:4963-4969; Armour et al., 1999, Eur J Immunol 29:2613-2624; Idusogie et al, 2000, J Immunol 164:4178-4184; Reddy et al, 2000, J Immunol 164:1925-1933; Xu et al., 2000, Cell Immunol 200:16-26; Idusogie et al, 2001, J Immunol 166:2571-2575; Shields et al., 2001, J Biol Chem 276:6591-6604; Jefferis et al, 2002, Immunol Lett 82:57-65; Presta et al., 2002, Biochem Soc Trans 30:487-490; U.S. Pat. Nos. 5,624,821; 5,885,573; 5,677,425; 6,165,745; 6,277,375; 5,869,046; 6,121,022; 5,624,821; 5,648,260; 6,528,624; 6,194,551; 6,737,056; 6,821,505; 6,277,375; U.S. Patent Publication Nos. 2004/0002587 and PCT Publications WO 94/29351; WO 99/58572; WO 00/42072; WO 02/060919; WO 04/029207; WO 04/099249; WO 04/063351. Also encompassed by the present invention are Fc regions which comprise deletions, additions and/or modifications. Still other modifications/substitutions/additions/deletions of the Fc domain will be readily apparent to one skilled in the art.

Methods for generating non naturally occurring Fc regions are known in the art. For example, amino acid substitutions and/or deletions can be generated by mutagenesis methods, including, but not limited to, site-directed mutagenesis (Kunkel, Proc. Natl. Acad. Sci. USA 82:488-492 (1985)), PCR mutagenesis (Higuchi, in “PCR Protocols: A Guide to Methods and Applications”, Academic Press, San Diego, pp. 177-183 (1990)), and cassette mutagenesis (Wells et al., Gene 34:315-323 (1985)). Site-directed mutagenesis may be performed by the overlap-extension PCR method (Higuchi, in “PCR Technology: Principles and Applications for DNA Amplification”, Stockton Press, New York, pp. 61-70 (1989)). The technique of overlap-extension PCR (Higuchi, ibid.) can also be used to introduce any desired mutation(s) into a target sequence (the starting DNA). For example, the first round of PCR in the overlap-extension method involves amplifying the target sequence with an outside primer (primer 1) and an internal mutagenesis primer (primer 3), and separately with a second outside primer (primer 4) and an internal primer (primer 2), yielding two PCR segments (segments A and B). The internal mutagenesis primer (primer 3) is designed to contain mismatches to the target sequence specifying the desired mutation(s). In the second round of PCR, the products of the first round of PCR (segments A and B) are amplified by PCR using the two outside primers (primers 1 and 4). The resulting full-length PCR segment (segment C) is digested with restriction enzymes and the resulting restriction fragment is cloned into an appropriate vector. As the first step of mutagenesis, the starting DNA (e.g., encoding an Fc fusion protein, an antibody or simply an Fc region), is operably cloned into a mutagenesis vector. The primers are designed to reflect the desired amino acid substitution. Other methods useful for the generation of variant Fc regions are known in the art (see, e.g., U.S. Pat. Nos. 5,624,821; 5,885,573; 5,677,425; 6,165,745; 6,277,375; 5,869,046; 6,121,022; 5,624,821; 5,648,260; 6,528,624; 6,194,551; 6,737,056; 6,821,505; 6,277,375; U.S. Patent Publication Nos. 2004/0002587 and PCT Publications WO 94/29351; WO 99/58572; WO 00/42072; WO 02/060919; WO 04/029207; WO 04/099249; WO 04/063351).

In some embodiments, an Fc variant protein comprises one or more engineered glycoforms, i.e., a carbohydrate composition that is covalently attached to the molecule comprising an Fc region. Engineered glycoforms may be useful for a variety of purposes, including but not limited to enhancing or reducing effector function. Engineered glycoforms may be generated by any method known to one skilled in the art, for example by using engineered or variant expression strains, by co-expression with one or more enzymes, for example DI N-acetylglucosaminyltransferase III (GnTI11), by expressing a molecule comprising an Fc region in various organisms or cell lines from various organisms, or by modifying carbohydrate(s) after the molecule comprising Fc region has been expressed. Methods for generating engineered glycoforms are known in the art, and include but are not limited to those described in Umana et al, 1999, Nat. Biotechnol 17:176-180; Davies et al., 20017 Biotechnol Bioeng 74:288-294; Shields et al, 2002, J Biol Chem 277:26733-26740; Shinkawa et al., 2003, J Biol Chem 278:3466-3473; U.S. Pat. No. 6,602,684; U.S. Ser. No. 10/277,370; U.S. Ser. No. 10/113,929; PCT WO 00/61739A1; PCT WO 01/292246A1; PCT WO 02/311140A1; PCT WO 02/30954A1; Potillegent™ technology (Biowa, Inc. Princeton, N.J.); GlycoMAb™ glycosylation engineering technology (GLYCART biotechnology AG, Zurich, Switzerland). See, e.g., WO 00061739; EA01229125; US 20030115614; Okazaki et al., 2004, JMB, 336: 1239-49.

It is contemplated that an Fc variant described herein may be generated from, or a variant Fc region described herein may be introduced into any antibody described in the art including but not limited to anti-fluorescein monoclonal antibody, 4-4-20 (Kranz et al., 1982 J. Biol. Chem. 257(12): 6987-6995), a humanized anti-TAG72 antibody (CC49) (Sha et al., 1994 Cancer Biother. 9(4): 341-9), an antibody that specifically bind an Eph Receptor including, but not limited to those disclosed in PCT Publication Nos. WO 04/014292, WO 03/094859 and U.S. patent application Ser. No. 10/863,729, antibodies that specifically bind Integrin αVβ3 including, but not limited to, LM609 (Scripps), the murine monoclonal LM609 (PCT Publication WO 89/015155 and U.S. Pat. No. 5,753,230); the humanized monoclonal antibody MEDI-522 (a.k.a. VITAXIN®, MedImmune, Inc., Gaithersburg, Md.; Wu et al., 1998, PNAS USA 95(11): 6037-6042; PCT Publications WO 90/33919 and WO 00/78815), an antibody against interferon alpha as disclosed in WO/2005/05059106, an antibody against the interferon receptor 1 as disclosed in WO/2006/059106, Erbitux™ (also known as IMC-C225) (ImClone Systems Inc.), a chimerized monoclonal antibody against EGFR; HERCEPTIN® (Trastuzumab) (Genentech, CA) which is a humanized anti-HER2 monoclonal antibody for the treatment of patients with metastatic breast cancer; REOPRO® (abciximab) (Centocor) which is an anti-glycoprotein IIb/IIIa receptor on the platelets for the prevention of clot formation; ZENAPAX® (daclizumab) (Roche Pharmaceuticals, Switzerland) which is an immunosuppressive, humanized anti-CD25 monoclonal antibody for the prevention of acute renal allograft rejection. Other examples are a humanized anti-CD18 F(ab′)2 (Genentech); CDP860 which is a humanized anti-CD18 F(ab′)2 (Celltech, UK); PRO542 which is an anti-HIV gp120 antibody fused with CD4 (Progenics/Genzyme Transgenics); C14 which is an anti-CD14 antibody (ICOS Pharm); a humanized anti-VEGF IgG1 antibody (Genentech); OVAREX™ which is a murine anti-CA 125 antibody (Altarex); PANOREX™ which is a murine anti-17-IA cell surface antigen IgG2a antibody (Glaxo Wellcome/Centocor); IMC-C225 which is a chimeric anti-EGFR IgG antibody (ImClone System); VITAXIN™ which is a humanized anti-αVβ3 integrin antibody (Applied Molecular Evolution/MedImmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG1 antibody (IDEC Pharm/Genentech, Roche/Zettyaku); LYMPHOCIDE™ which is a humanized anti-CD22 IgG antibody (Immunomedics); Smart ID10 which is a humanized anti-HLA antibody (Protein Design Lab); ONCOLYM™ (Lym-1) is a radiolabelled murine anti-HLA DR antibody (Techniclone); anti-CD11a is a humanized IgG1 antibody (Genetech/Xoma); ICM3 is a humanized anti-ICAM3 antibody (ICOS Pharm); IDEC-114 is a primatized anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); IDEC-151 is a primatized anti-CD4 IgG1 antibody (IDEC Pharm/SmithKline Beecham); MDX-CD4 is a human anti-CD4 IgG antibody (Medarex/Eisai/Genmab); CDP571 is a humanized anti-TNF-α IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); MDX-33 is a human anti-CD64 (FcγR) antibody (Medarex/Centeon); rhuMab-E25 is a humanized anti-IgE IgG1 antibody (Genentech/Norvartis/Tanox Biosystems); IDEC-152 is a primatized anti-CD23 antibody (IDEC Pharm); ABX-CBL is a murine anti CD-147 IgM antibody (Abgenix); BTI-322 is a rat anti-CD2 IgG antibody (Medimmune/Bio Transplant); Orthoclone/OKT3 is a murine anti-CD3 IgG2a antibody (ortho Biotech); SIMULECT™ is a chimeric anti-CD25 IgG1 antibody (Novartis Pharm); LDP-01 is a humanized anti-β2-integrin IgG antibody (LeukoSite); Anti-LFA-1 is a murine anti CD18 F(ab′)2 (Pasteur-Merieux/Immunotech); CAT-152 is a human anti-TGF-β2 antibody (Cambridge Ab Tech); and Corsevin M is a chimeric anti-Factor VII antibody (Centocor).

Additional antibodies which may comprise an Fc variant region described herein may specifically bind a cancer or tumor antigen for example, including, but not limited to, KS 1/4 pan-carcinoma antigen (Perez and Walker, 1990, J. Immunol. 142: 3662-3667; Bumal, 1988, Hybridoma 7(4): 407-415), ovarian carcinoma antigen (CA 125) (Yu et al., 1991, Cancer Res. 51(2): 468-475), prostatic acid phosphate (Tailor et al., 1990, Nucl. Acids Res. 18(16): 4928), prostate specific antigen (Henttu and Vihko, 1989, Biochem. Biophys. Res. Comm. 160(2): 903-910; Israeli et al., 1993, Cancer Res. 53: 227-230), melanoma-associated antigen p97 (Estin et al., 1989, J. Natl. Cancer Instit. 81(6): 445-446), melanoma antigen gp75 (Vijayasardahl et al., 1990, J. Exp. Med. 171(4): 1375-1380), high molecular weight melanoma antigen (HMW-MAA) (Natali et al., 1987, Cancer 59: 55-63; Mittelman et al., 1990, J. Clin. Invest. 86: 2136-2144), prostate specific membrane antigen, carcinoembryonic antigen (CEA) (Foon et al., 1994, Proc. Am. Soc. Clin. Oncol. 13: 294), polymorphic epithelial mucin antigen, human milk fat globule antigen, colorectal tumor-associated antigens such as: CEA, TAG-72 (Yokata et al., 1992, Cancer Res. 52: 3402-3408), C017-1A (Ragnhammar et al., 1993, Int. J. Cancer 53: 751-758); GICA 19-9 (Herlyn et al., 1982, J. Clin. Immunol. 2: 135), CTA-1 and LEA, Burkitt's lymphoma antigen-38.13, CD19 (Ghetie et al., 1994, Blood 83: 1329-1336), human B-lymphoma antigen-CD20 (Reff et al., 1994, Blood 83:435-445), CD33 (Sgouros et al., 1993, J. Nucl. Med. 34:422-430), melanoma specific antigens such as ganglioside GD2 (Saleh et al., 1993, J. Immunol., 151, 3390-3398), ganglioside GD3 (Shitara et al., 1993, Cancer Immunol. Immunother. 36:373-380), ganglioside GM2 (Livingston et al., 1994, J. Clin. Oncol. 12: 1036-1044), ganglioside GM3 (Hoon et al., 1993, Cancer Res. 53: 5244-5250), tumor-specific transplantation type of cell-surface antigen (TSTA) such as virally-induced tumor antigens including T-antigen DNA tumor viruses and Envelope antigens of RNA tumor viruses, oncofetal antigen-alpha-fetoprotein such as CEA of colon, bladder tumor oncofetal antigen (Hellstrom et al., 1985, Cancer. Res. 45:2210-2188), differentiation antigen such as human lung carcinoma antigen L6, L20 (Hellstrom et al., 1986, Cancer Res. 46: 3917-3923), antigens of fibrosarcoma, human leukemia T cell antigen-Gp37 (Bhattacharya-Chatterjee et al., 1988, J. of Immun. 141:1398-1403), neoglycoprotein, sphingolipids, breast cancer antigen such as EGFR (Epidermal growth factor receptor), HER2 antigen (p185HER2), polymorphic epithelial mucin (PEM) (Hilkens et al., 1992, Trends in Bio. Chem. Sci. 17:359), malignant human lymphocyte antigen-APO-1 (Bernhard et al., 1989, Science 245: 301-304), differentiation antigen (Feizi, 1985, Nature 314: 53-57) such as I antigen found in fetal erythrocytes, primary endoderm I antigen found in adult erythrocytes, preimplantation embryos, I(Ma) found in gastric adenocarcinomas, M18, M39 found in breast epithelium, SSEA-1 found in myeloid cells, VEP8, VEP9, My1, VIM-D5, D156-22 found in colorectal cancer, TRA-1-85 (blood group H), C14 found in colonic adenocarcinoma, F3 found in lung adenocarcinoma, AH6 found in gastric cancer, Y hapten, Ley found in embryonal carcinoma cells, TL5 (blood group A), EGF receptor found in A431 cells, E1 series (blood group B) found in pancreatic cancer, FC10.2 found in embryonal carcinoma cells, gastric adenocarcinoma antigen, CO-514 (blood group Lea) found in Adenocarcinoma, NS-10 found in adenocarcinomas, CO-43 (blood group Leb), G49 found in EGF receptor of A431 cells, MH2 (blood group ALeb/Ley) found in colonic adenocarcinoma, 19.9 found in colon cancer, gastric cancer mucins, T5A7 found in myeloid cells, R24 found in melanoma, 4.2, GD3, D1.1, OFA-1, GM2, OFA-2, GD2, and M1:22:25:8 found in embryonal carcinoma cells, and SSEA-3 and SSEA-4 found in 4 to 8-cell stage embryos. In one embodiment, the antigen is a T cell receptor derived peptide from a Cutaneous Tcell Lymphoma (see, Edelson, 1998, The Cancer Journal 4:62).

An Fc variant described herein may be generated from, or a variant Fc region described herein may be introduced into any antibody. Furthermore, a variant Fc region described herein may be utilized to generate an Fc fusion protein. Accordingly, virtually any molecule may be targeted by and/or incorporated into an antibody and/or Fc fusion protein comprising an Fc variant described herein including, but not limited to, the following list of proteins, as well as subunits, domains, motifs and epitopes belonging to the following list of proteins: renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VII, factor VIIIC, factor IX, tissue factor (TF), and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors such as, for example, EGFR, VEGFR; interferons such as alpha interferon (α-IFN), beta interferon (β-IFN) and gamma interferon (γ-IFN); interferon receptor components such as interferon receptor 1; protein A or D; rheumatoid factors; a neurotrophic factor such as bone-derived neurotrophic factor (BDNF), neurotrophin-3,-4,-5, or -6 (NT-3, NT-4, NT-5, or NT-6), or a nerve growth factor; platelet-derived growth factor (PDGF); fibroblast growth factor such as αFGF and βFGF; epidermal growth factor (EGF); transforming growth factor (TGF) such as TGF-alpha and TGF-beta, including TGF-1, TGF-2, TGF-3, TGF-4, or TGF-5; insulin-like growth factor-I and -II (IGF-I and IGF-II); des (1-3)-IGF-I (brain IGF-I), insulin-like growth factor binding proteins; CD proteins such as CD2, CD3, CD4, CD 8, CD11a, CD14, CD18, CD19, CD20, CD22, CD23, CD25, CD33, CD34, CD40, CD40L, CD52, CD63, CD64, CD80 and CD147; erythropoietin; osteoinductive factors; immunotoxins; a bone morphogenetic protein (BMP); an interferon such as interferon-alpha,-beta, and -gamma; colony stimulating factors (CSFs), such as M-CSF, GM-CSF, and G-CSF; interleukins (ILs), e.g., IL-1 to IL-13; TNFα, HMGB1; HMGB2; superoxide dismutase; T-cell receptors; surface membrane proteins; decay accelerating factor; viral antigen such as, for example, a portion of the AIDS envelope, e.g., gp120; transport proteins; homing receptors; addressins; regulatory proteins; cell adhesion molecules such as LFA-1, Mac 1, p150.95, VLA-4, ICAM-1, ICAM-3 and VCAM, a4/p7 integrin, and (Xv/p3 integrin including either a or subunits thereof, integrin alpha subunits such as CD49a, CD49b, CD49c, CD49d, CD49e, CD49f, alpha7, alpha8, alpha9, alphaD, CD11a, CD11b, CD51, CD11c, CD41, alphaIIb, alphaIELb; integrin beta subunits such as, CD29, CD18, CD61, CD104, beta5, beta6, beta7 and beta8; Integrin subunit combinations including but not limited to, αVβ3, αVβ5 and αVβ7; a member of an apoptosis pathway; IgE; blood group antigens; flk2/flt3 receptor; obesity (OB) receptor; mp1 receptor; CTLA-4; protein C; a chitinase or chitinase-like molecule such as YKL-40 and AMCase; an Eph receptor such as EphA2, EphA4, EphB2, etc.; a Human Leukocyte Antigen (HLA) such as HLA-DR; complement proteins such as complement receptor CR1, C1Rq and other complement factors such as C3, and C5; a glycoprotein receptor such as GpIbα, GPIIb/IIIa and CD200; co-stimulatory molecules such as CD28/CTLA-4, ICOS/AILIM, PD-1.

Additional molecules which may comprise a variant Fc region described herein are those that specifically bind cancer antigens including, but not limited to, ALK receptor (pleiotrophin receptor), pleiotrophin, KS 1/4 pan-carcinoma antigen; ovarian carcinoma antigen (CA 125); prostatic acid phosphate; prostate specific antigen (PSA); melanoma-associated antigen p97; melanoma antigen gp75; high molecular weight melanoma antigen (HMW-MAA); prostate specific membrane antigen; carcinoembryonic antigen (CEA); polymorphic epithelial mucin antigen; human milk fat globule antigen; colorectal tumor-associated antigens such as: CEA, TAG-72, CO17-1A, GICA 19-9, CTA-1 and LEA; Burkitt's lymphoma antigen-38.13; CD19; human B-lymphoma antigen-CD20; CD33; melanoma specific antigens such as ganglioside GD2, ganglioside GD3, ganglioside GM2 and ganglioside GM3; tumor-specific transplantation type cell-surface antigen (TSTA); virally-induced tumor antigens including T-antigen, DNA tumor viruses and Envelope antigens of RNA tumor viruses; oncofetal antigen-alpha-fetoprotein such as CEA of colon, 5T4 oncofetal trophoblast glycoprotein and bladder tumor oncofetal antigen; differentiation antigen such as human lung carcinoma antigens L6 and L20; antigens of fibrosarcoma; human leukemia T cell antigen-Gp37; neoglycoprotein; sphingolipids; breast cancer antigens such as EGFR (Epidermal growth factor receptor); NY-BR-16; NY-BR-16 and HER2 antigen (p185HER2); polymorphic epithelial mucin (PEM); malignant human lymphocyte antigen-APO-1; differentiation antigen such as I antigen found in fetal erythrocytes; primary endoderm I antigen found in adult erythrocytes; preimplantation embryos; I(Ma) found in gastric adenocarcinomas; M18, M39 found in breast epithelium; SSEA-1 found in myeloid cells; VEP8; VEP9; Myl; VIM-D5; D156-22 found in colorectal cancer; TRA-1-85 (blood group H); SCP-1 found in testis and ovarian cancer; C14 found in colonic adenocarcinoma; F3 found in lung adenocarcinoma; AH6 found in gastric cancer; Y hapten; Ley found in embryonal carcinoma cells; TL5 (blood group A); EGF receptor found in A431 cells; E1 series (blood group B) found in pancreatic cancer; FC10.2 found in embryonal carcinoma cells; gastric adenocarcinoma antigen; CO-514 (blood group Lea) found in Adenocarcinoma; NS-10 found in adenocarcinomas; CO-43 (blood group Leb); G49 found in EGF receptor of A431 cells; MH2 (blood group ALeb/Ley) found in colonic adenocarcinoma; 19.9 found in colon cancer; gastric cancer mucins; T5A7 found in myeloid cells; R24 found in melanoma; 4.2, GD3, D1.1, OFA-1, GM2, OFA-2, GD2, and M1:22:25:8 found in embryonal carcinoma cells and SSEA-3 and SSEA-4 found in 4 to 8-cell stage embryos; Cutaneous Tcell Lymphoma antigen; MART-1 antigen; Sialy Tn (STn) antigen; Colon cancer antigen NY-CO-45; Lung cancer antigen NY-LU-12 variant A; Adenocarcinoma antigen ART1; Paraneoplastic associated brain-testis-cancer antigen (onconeuronal antigen MA2; paraneoplastic neuronal antigen); Neuro-oncological ventral antigen 2 (NOVA2); Hepatocellular carcinoma antigen gene 520; TUMOR-ASSOCIATED ANTIGEN CO-029; Tumor-associated antigens MAGE-C1 (cancer/testis antigen CT7), MAGE-B1 (MAGE-XP antigen), MAGE-B2 (DAM6), MAGE-2, MAGE-4a, MAGE-4-b and MAGE-X2; Cancer-Testis Antigen (NY-EOS-1); YKL-40 and fragments of any of the above-listed polypeptides.

### 6.10. Glycosylation of Antibodies

In still another embodiment, the glycosylation of antibodies utilized in accordance with the invention is modified. For example, an aglycoslated antibody can be made (i.e., the antibody lacks glycosylation). Glycosylation can be altered to, for example, increase the affinity of the antibody for a target antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861. One or more amino acid substitutions can also be made that result in elimination of a glycosylation site present in the Fc region (e.g., Asparagine 297 of IgG). Furthermore, aglycosylated antibodies may be produced in bacterial cells which lack the necessary glycosylation machinery.

An antibody can also be made that has an altered type of glycosylation, such as a hypofucosylated antibody having reduced amounts of fucosyl residues or an antibody having increased bisecting GlcNAc structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. See, for example, Shields, R. L. et al. (2002) J. Biol. Chem. 277:26733-26740; Umana et al. (1999) Nat. Biotech. 17:176-1, as well as, U.S. Pat. No. 6,946,292; European Patent No: EP 1,176,195; PCT Publications WO 03/035835; WO 99/54342 each of which is incorporated herein by reference in its entirety.

### 6.11. Engineering Effector Function

It may be desirable to modify an anti-CD19 antibody of the invention with respect to effector function, so as to enhance the effectiveness of the antibody in treating B cell malignancies, for example. For example, cysteine residue(s) may be introduced in the Fc region, thereby allowing interchain disulfide bond formation in this region. The homodimeric antibody thus generated may have improved internalization capability and/or increased complement-mediated cell killing and/or antibody-dependent cellular cytotoxicity (ADCC). See, Caron et al., J. Exp Med., 176:1191-1195 (1992) and Shopes, B., J. Immunol., 148:2918-2922 (1992). Homodimeric antibodies with enhanced anti-tumor activity may also be prepared using heterobifunctional cross-linkers as described in Wolff et al., Cancer Research, 53:2560-2565 (1993). An antibody can also be engineered which has dual Fc regions and may thereby have enhanced complement lysis and ADCC capabilities. See, Stevenson et al., Anti-Cancer Drug Design, 3:219-230 (1989).

Other methods of engineering Fc regions of antibodies so as to alter effector functions are known in the art (e.g., U.S. Patent Publication No. 20040185045 and PCT Publication No. WO 2004/016750, both to Koenig et al., which describe altering the Fc region to enhance the binding affinity for FcγRIIB as compared with the binding affinity for FCγRIIA; see, also, PCT Publication Nos. WO 99/58572 to Armour et al., WO 99/51642 to Idusogie et al., and U.S. Pat. No. 6,395,272 to Deo et al.; the disclosures of which are incorporated herein in their entireties). Methods of modifying the Fc region to decrease binding affinity to FcγRIIB are also known in the art (e.g., U.S. Patent Publication No. 20010036459 and PCT Publication No. WO 01/79299, both to Ravetch et al., the disclosures of which are incorporated herein in their entireties). Modified antibodies having variant Fc regions with enhanced binding affinity for FcγRIIIA and/or FcγRIIA as compared with a wildtype Fc region have also been described (e.g., PCT Publication Nos. WO 2004/063351, to Stavenhagen et al., the disclosure of which is incorporated herein in its entirety).

In vitro assays known in the art can be used to determine whether anti-CD19 antibodies used in compositions and methods of the invention are capable of mediating ADCC, such as those described herein.

### 6.12. Manufacture/Production of Anti-CD19 Antibodies

Once a desired anti-CD19 antibody is engineered, the anti-CD19 antibody can be produced on a commercial scale using methods that are well-known in the art for large scale manufacturing of antibodies. For example, this can be accomplished using recombinant expressing systems such as, but not limited to, those described below.

### 6.13. Recombinant Expression Systems

Recombinant expression of an antibody or variant thereof, generally requires construction of an expression vector containing a polynucleotide that encodes the antibody. Once a polynucleotide encoding an antibody molecule or a heavy or light chain of an antibody, or portion thereof, has been obtained, the vector for the production of the antibody molecule may be produced by recombinant DNA technology using techniques well-known in the art. See, e.g., U.S. Pat. No. 6,331,415, which is incorporated herein by reference in its entirety. Thus, methods for preparing a protein by expressing a polynucleotide containing an antibody encoding nucleotide sequence are described herein. Methods which are well-known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule, a heavy or light chain of an antibody, a heavy or light chain variable domain of an antibody or a portion thereof, or a heavy or light chain CDR, operably linked to a promoter. Such vectors may include the nucleotide sequence encoding the constant region of the antibody molecule (see, e.g., International Publication Nos. WO 86/05807 and WO 89/01036; and U.S. Pat. No. 5,122,464) and the variable domain of the antibody may be cloned into such a vector for expression of the entire heavy, the entire light chain, or both the entire heavy and light chains.

In another embodiment, anti-CD19 antibodies can be made using targeted homologous recombination to produce all or portions of the anti-CD19 antibodies (see, U.S. Pat. Nos. 6,063,630, 6,187,305, and 6,692,737). In certain embodiments, anti-CD19 antibodies can be made using random recombination techniques to produce all or portions of the anti-CD19 antibodies (see, U.S. Pat. Nos. 6,361,972, 6,524,818, 6,541,221, and 6,623,958). Anti-CD19 antibodies can also be produced in cells expressing an antibody from a genomic sequence of the cell comprising a modified immunoglobulin locus using Cre-mediated site-specific homologous recombination (see, U.S. Pat. No. 6,091,001). The host cell line may be derived from human or nonhuman species including but not limited to mouse, and Chinese hamster. Where human or humanized antibody production is desired, the host cell line should be a human cell line. These methods may advantageously be used to engineer stable cell lines which permanently express the antibody molecule.

Once the expression vector is transferred to a host cell by conventional techniques, the transfected cells are then cultured by conventional techniques to produce an antibody. Thus, the invention includes host cells containing a polynucleotide encoding an antibody of the invention or fragments thereof, or a heavy or light chain thereof, or portion thereof, or a single-chain antibody of the invention, operably linked to a heterologous promoter. In certain embodiments for the expression of double-chained antibodies, vectors encoding both the heavy and light chains may be co-expressed in the host cell for expression of the entire immunoglobulin molecule, as detailed below.

A variety of host-expression vector systems may be utilized to express an anti-CD19 antibody or portions thereof that can be used in the engineering and generation of anti-CD19 antibodies (see, e.g., U.S. Pat. No. 5,807,715). For example, mammalian cells such as Chinese hamster ovary cells (CHO), in conjunction with a vector such as the major intermediate early gene promoter element from human cytomegalovirus is an effective expression system for antibodies (Foecking et al., Gene, 45:101 (1986); and Cockett et al., Bio/Technology, 8:2 (1990)). In addition, a host cell strain may be chosen which modulates the expression of inserted antibody sequences, or modifies and processes the antibody gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the antibody or portion thereof expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT2O and T47D, NS0 (a murine myeloma cell line that does not endogenously produce any functional immunoglobulin chains), CRL7O3O and HsS78Bst cells.

In one embodiment, human cell lines developed by immortalizing human lymphocytes can be used to recombinantly produce monoclonal human anti-CD19 antibodies. In one embodiment, the human cell line PER.C6. (Crucell, Netherlands) can be used to recombinantly produce monoclonal human anti-CD19 antibodies.

In bacterial systems, a number of expression vectors may be advantageously selected depending upon the use intended for the antibody molecule being expressed. For example, when a large quantity of such an antibody is to be produced, for the generation of pharmaceutical compositions comprising an anti-CD19 antibody, vectors which direct the expression of high levels of fusion protein products that are readily purified may be desirable. Such vectors include, but are not limited to, the E. coli expression vector pUR278 (Ruther et al., EMBO, 12:1791 (1983)), in which the antibody coding sequence may be ligated individually into the vector in frame with the lac Z coding region so that a fusion protein is produced; pIN vectors (Inouye & Inouye, 1985, Nucleic Acids Res. 13:3101-3109 (1985); Van Heeke & Schuster, 1989, J. Biol. Chem., 24:5503-5509 (1989)); and the like. pGEX vectors may also be used to express foreign polypeptides as fusion proteins with glutathione-S-transferase (GST). In general, such fusion proteins are soluble and can easily be purified from lysed cells by adsorption and binding to glutathione-agarose affinity matrix followed by elution in the presence of free glutathione. The pGEX vectors are designed to introduce athrombin and/or factor Xa protease cleavage sites into the expressed polypeptide so that the cloned target gene product can be released from the GST moiety.

In an insect system, Autographa califormica nuclear polyhedrosis virus (AcNPV) is used as a vector to express foreign genes. The virus grows in Spodoptera frugiperda cells. The antibody coding sequence may be cloned individually into non-essential regions (for example, the polyhedrin gene) of the virus and placed under control of an AcNPV promoter (for example, the polyhedrin promoter).

In mammalian host cells, a number of virus based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion into a non-essential region of the viral genome (e.g., region E1 or E3) will result in a recombinant virus that is viable and capable of expressing the antibody molecule in infected hosts (e.g., see, Logan & Shenk, Proc. Natl. Acad. Sci. USA, 81:355-359 (1984)). Specific initiation signals may also be required for efficient translation of inserted antibody coding sequences. These signals include the ATG initiation codon and adjacent sequences. Furthermore, the initiation codon should generally be in frame with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., Methods in Enzymol., 153:51-544 (1987)).

Stable expression can be used for long-term, high-yield production of recombinant proteins. For example, cell lines which stably express the antibody molecule may be generated. Host cells can be transformed with an appropriately engineered vector comprising expression control elements (e.g., promoter, enhancer, transcription terminators, polyadenylation sites, etc.), and a selectable marker gene. Following the introduction of the foreign DNA, cells may be allowed to grow for 1-2 days in an enriched media, and then are switched to a selective media. The selectable marker in the recombinant plasmid confers resistance to the selection and allows cells that stably integrated the plasmid into their chromosomes to grow and form foci which in turn can be cloned and expanded into cell lines. Plasmids that encode an anti-CD19 antibody can be used to introduce the gene/cDNA into any cell line suitable for production in culture.

A number of selection systems may be used, including, but not limited to, the herpes simplex virus thymidine kinase (Wigler et al., Cell, 11:223 (1977)), hypoxanthineguanine phosphoribosyltransferase (Szybalska & Szybalski, Proc. Natl. Acad. Sci. USA, 48:202 (1992)), and adenine phosphoribosyltransferase (Lowy et al., Cell, 22:8-17 (1980)) genes can be employed in tk⁻, hgprt⁻ or aprT⁻ cells, respectively. Also, antimetabolite resistance can be used as the basis of selection for the following genes: dhfr, which confers resistance to methotrexate (Wigler et al., Natl. Acad. Sci. USA, 77:357 (1980); O'Hare et al., Proc. Natl. Acad. Sci. USA, 78:1527 (1981)); gpt, which confers resistance to mycophenolic acid (Mulligan & Berg, Proc. Natl. Acad. Sci. USA, 78:2072 (1981)); neo, which confers resistance to the aminoglycoside G-418 (Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIB TECH 11(5):155-2 15 (1993)); and hygro, which confers resistance to hygromycin (Santerre et al., Gene, 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds.), Current Protocols in Human Genetics, John Wiley & Sons, NY (1994); Colberre-Garapin et al., 1981, J. Mol. Biol., 150:1, which are incorporated by reference herein in their entireties.

The expression levels of an antibody molecule can be increased by vector amplification (for a review, see, Bebbington and Hentschel, The use of vectors based on gene amplification for the expression of cloned genes in mammalian cells in DNA cloning, Vol. 3. Academic Press, New York (1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol., 3:257 (1983)). Antibody expression levels may be amplified through the use recombinant methods and tools known to those skilled in the art of recombinant protein production, including technologies that remodel surrounding chromatin and enhance transgene expression in the form of an active artificial transcriptional domain.

The host cell may be co-transfected with two expression vectors, the first vector encoding a heavy chain derived polypeptide and the second vector encoding a light chain derived polypeptide. The two vectors may contain identical or different selectable markers. A single vector which encodes, and is capable of expressing, both heavy and light chain polypeptides may also be used. In such situations, the light chain should be placed 5′ to the heavy chain to avoid an excess of toxic free heavy chain (Proudfoot, Nature 322:562-65 (1986); and Kohler, 1980, Proc. Natl. Acad. Sci. USA, 77:2197 (1980)). The coding sequences for the heavy and light chains may comprise cDNA or genomic DNA.

Once an antibody molecule has been produced by recombinant expression, it may be purified by any method known in the art for purification of an immunoglobulin molecule, for example, by chromatography (e.g., ion exchange, affinity, particularly by affinity for the specific antigens Protein A or Protein G, and sizing column chromatography), centrifugation, differential solubility, or by any other standard technique for the purification of proteins. Further, the antibodies of the present invention or fragments thereof may be fused to heterologous polypeptide sequences described herein or otherwise known in the art to facilitate purification.

### 6.14. Antibody Purification and Isolation

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology, 10:163-167 (1992) describe a procedure for isolating antibodies which are secreted into the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody mutant is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, hydrophobic interaction chromatography, ion exchange chromatography, gel electrophoresis, dialysis, and/or affinity chromatography either alone or in combination with other purification steps. The suitability of protein A as an affinity ligand depends on the species and isotype of any immunoglobulin Fc domain that is present in the antibody mutant. Protein A can be used to purify antibodies that are based on human γ1, γ2, or γ4 heavy chains (Lindmark et al., J. Immunol. Methods, 62:1-13 (1983)). Protein G is recommended for all mouse isotypes and for human γ3 (Guss et al., EMBO J., 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH₃ domain, the Bakerbond ABX resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin, SEPHAROSE chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, and performed at low salt concentrations (e.g., from about 0-0.25 M salt).

### 6.15. Methods of Preparing the Antibody Formulations

The present invention provides methods for preparing liquid formulations of antibodies or derivatives, analogues, or fragments thereof that specifically bind to an antigen of interest (e.g., CD19 antigen). The methods for preparing liquid formulations of the present invention may comprise: purifying the antibody (including antibody fragment thereof) from conditioned medium (either single lots or pooled lots of medium) and concentrating a fraction of the purified antibody (including antibody fragment thereof) to a final concentration of about 10 mg/ml, about 15 mg/ml, about 20 mg/ml, about 30 mg/ml, about 40 mg/ml, about 50 mg/ml, about 60 mg/ml, about 70 mg/ml, about 80 mg/ml, about 90 mg/ml, about 100 mg/ml, about 150 mg/ml, about 175 mg/ml, about 200 mg/ml, about 250 mg/ml, or about 300 mg/ml. Conditioned medium containing the antibody (including antibody fragment thereof), for example, an antibody that specifically binds to CD19 may be subjected to CUNO filtration and the filtered antibody is subjected to HS50 cation exchange chromatography. The fraction from the HS50 cation exchange chromatography is then subjected to low pH treatment followed by MEP Hypercel chromatography. The fraction from the MEP Hypercel chromatography is subject to nanofiltration. The purified antibody or a fragment thereof obtained after nanofiltration is then subjected to diafiltration and ultrafiltration to buffer exchange and concentrate into the formulation buffer using the same membrane.

The liquid formulations of the present invention can be prepared as unit dosage forms by preparing a vial containing an aliquot of the liquid formulation for a one-time use. For example, a unit dosage per vial may contain 1 ml, 2 ml, 3 ml, 4 ml, 5 ml, 6 ml, 7 ml, 8 ml, 9 ml, 10 ml, 15 ml, or 20 ml of different concentrations of an antibody (including antibody fragment thereof) that specifically binds to CD19 ranging from about 5 mg/ml to about 300 mg/ml. If necessary, these preparations can be adjusted to a desired concentration by adding a sterile diluent to each vial. In a specific embodiment, the liquid formulations of the present invention are formulated into single dose vials as a sterile liquid that contains 10 mM histidine buffer at pH 6.0, 75 mM NaCl, and 4% trehalose. In another embodiment, the liquid formulations of the present invention are formulated into single dose vials as a sterile liquid that contains 10 mM histidine buffer at pH 6.0, 75 mM NaCl, 4% trehalose, and 0.02% Polysorbate 80. In another embodiment, the liquid formulations of the present invention are formulated into single dose vials as a sterile liquid that contains 20 mM histidine buffer at pH 6.0, 75 mM NaCl, 4% trehalose, and 0.02% Polysorbate 80. Each 1.0 mL of solution contains 10 mg of the antibody (including antibody fragment thereof). In one embodiment, the antibody (including antibody fragment thereof) of the invention is supplied at 100 mg/ml in 3 cc USP Type I borosilicate amber vials (West Pharmaceutical Services—Part No. 6800-0675). The target fill volume is 1.2 mL. In another embodiment, the antibody (including antibody fragment thereof) of the invention is supplied at 100 mg/ml in 3 cc vials in 10 mM histidine buffer at pH 6.0, 75 mM NaCl, 4% trehalose. In another embodiment, the antibody (including antibody fragment thereof) of the invention is supplied at 100 mg/ml in 3 cc vials in 10 mM histidine buffer at pH 6.0, 75 mM NaCl, 4% trehalose, and 0.02% Polysorbate 80. In another embodiment, the liquid formulations of the present invention are formulated into single dose vials as a sterile liquid that contains 10 mM histidine buffer at pH 6.0, 75 mM NaCl, 4% trehalose, and 0.02% Polysorbate 80, in which formulations each 1.0 mL of solution contains 25, 50 or 100 mg of the antibody (including antibody fragment thereof).

The liquid formulations of the present invention can be prepared as unit dosage forms by preparing a pre-filled syringe containing an aliquot of the liquid formulation for a one-time use. For example, a unit dosage per pre-filled syringe may contain 0.1 ml, 0.2 ml, 0.3 ml, 0.4 ml, 0.5 ml, 0.6 ml, 0.7 ml, 0.8 ml, 0.9 ml, 1 ml, 2 ml, 3 ml, 4 ml, 5 ml, 6 ml, 7 ml, 8 ml, 9 ml, 10 ml, 15 ml, or 20 ml of different concentrations of an antibody (including antibody fragment thereof) that specifically binds to CD19 ranging from about 5 mg/ml to about 300 mg/ml. In a specific embodiment, the liquid formulations of the present invention are formulated into single dose pre-filled syringes as a sterile liquid that contains 10 mM histidine buffer at pH 6.0, 75 mM NaCl, and 4% trehalose. In another embodiment, the liquid formulations of the present invention are formulated into single dose pre-filled syringes as a sterile liquid that contains 10 mM histidine buffer at pH 6.0, 75 mM NaCl, 4% trehalose, and 0.02% Polysorbate 80. Each 1.0 mL of solution contains 10 mg of the antibody (including antibody fragment thereof). In another embodiment, the liquid formulations of the present invention are formulated into single dose pre-filled syringes as a sterile liquid that contains 10 mM histidine buffer at pH 6.0, 75 mM NaCl, 4% trehalose, and 0.02% Polysorbate 80, in which each 1.0 mL of solution contains 25, 50 or 100 mg of the antibody (including antibody fragment thereof). In another embodiment, the liquid formulations of the present invention are formulated into single dose pre-filled syringes as a sterile liquid that contains 20 mM histidine buffer at pH 6.0, 75 mM NaCl, 4% trehalose, and 0.02% Polysorbate 80. Each 1.0 mL of solution contains 10 mg of the antibody (including antibody fragment thereof). In another embodiment, the liquid formulations of the present invention are formulated into single dose pre-filled syringes as a sterile liquid that contains 20 mM histidine buffer at pH 6.0, 75 mM NaCl, 4% trehalose, and 0.02% Polysorbate 80, in which each 1.0 mL of solution contains 25, 50 or 100 mg of the antibody (including antibody fragment thereof). In yet another embodiment, the liquid formulations may be formulated into pre-filled syringes or auto-injectors for subcutaneous injection in any suitable concentration levels, including ranging from about 5 mg/ml to about 300 mg/ml. In another embodiment, the liquid antibody formulations are in pre-filled syringes or auto-injectors for subcutaneous injection in a concentration of up to 100 mg/mL. In another embodiment, the liquid antibody formulations are in pre-filled tungsten-free syringes, such as “ultra-100”. In another embodiment the syringe is a Clearject™ (Geresheimer, AG, Germany) or InJentle™ (SCHOTT Pharmaceutical Packaging, Germany) syringe.

The liquid formulations of the present invention may be sterilized by various sterilization methods, including sterile filtration, radiation, etc. In a specific embodiment, the difiltrated antibody formulation is filter-sterilized with a presterilized 0.2 micron filter. In certain embodiments where a surfactant is added, the antibody formulation may be filter-sterilized prior to the addition of the surfactant and/or subsequent to the addition of the surfactant. In some embodiments, the antibody formulation is filter-sterilized prior to the addition of the surfactant. Moreover, the formulation may be filter-sterilized one or more times. In some embodiments, Polysorbate 80 is added to the antibody formulation and then filter-sterilized. In another embodiment, the antibody formulation is filter-sterilized and then Polysorbate 80 is added for the formulation. In another embodiment, the antibody formulation is first filter-sterilized, Polysorbate 80 is then added to the formulation, and then the formulation undergoes a second filter-sterilization. Sterilized liquid formulations of the present invention may be administered to a subject to prevent, treat and/or manage a B cell mediated disease or disorder, or one or more symptoms thereof.

Although the invention is directed to liquid non-lyophilized formulations, it should be noted for the purpose of equivalents that the formulations of the invention may be lyophilized if desired. Thus, the invention encompasses lyophilized forms of the formulations of the invention.

### 6.15.1. Immunoconjugates and Fusion Proteins

According to certain aspects of the invention, therapeutic agents or toxins can be conjugated to chimerized, human, or humanized anti-CD19 antibodies for use in compositions and methods of the invention. In certain embodiments, these conjugates can be generated as fusion proteins. Examples of therapeutic agents and toxins include, but are not limited to, members of the enediyne family of molecules, such as calicheamicin and esperamicin. Chemical toxins can also be taken from the group consisting of duocarmycin (see, e.g., U.S. Pat. No. 5,703,080 and U.S. Pat. No. 4,923,990), methotrexate, doxorubicin, melphalan, chlorambucil, ARA-C, vindesine, mitomycin C, cis-platinum, etoposide, bleomycin and 5-fluorouracil. Examples of chemotherapeutic agents also include Adriamycin, Doxorubicin, 5-Fluorouracil, Cytosine arabinoside (Ara-C), Cyclophosphamide, Thiotepa, Taxotere (docetaxel), Busulfan, Cytoxin, Taxol, Methotrexate, Cisplatin, Melphalan, Vinblastine, Bleomycin, Etoposide, Ifosfamide, Mitomycin C, Mitoxantrone, Vincreistine, Vinorelbine, Carboplatin, Teniposide, Daunomycin, Caminomycin, Aminopterin, Dactinomycin, Mitomycins, Esperamicins (see, U.S. Pat. No. 4,675,187), Melphalan, and other related nitrogen mustards.

In certain embodiments, anti-CD19 antibodies are conjugated to a cytostatic, cytotoxic or immunosuppressive agent wherein the cytotoxic agent is selected from the group consisting of an enediyne, a lexitropsin, a duocarmycin, a taxane, a puromycin, a dolastatin, a maytansinoid, and a vinca alkaloid. In certain, more specific embodiments, the cytotoxic agent is paclitaxel, docetaxel, CC-1065, SN-38, topotecan, morpholino-doxorubicin, rhizoxin, cyanomorpholino-doxorubicin, dolastatin-10, echinomycin, combretastatin, calicheamicin, maytansine, DM-1, auristatin E, AEB, AEVB, AEFP, MMAE (see, U.S. patent application Ser. No. 10/983,340), or netropsin.

In certain embodiments, the cytotoxic agent of an anti-CD19 antibody-cytotoxic agent conjugate of the invention is an anti-tubulin agent. In specific embodiments, the cytotoxic agent is selected from the group consisting of a vinca alkaloid, a podophyllotoxin, a taxane, a baccatin derivative, a cryptophysin, a maytansinoid, a combretastatin, and a dolastatin. In other embodiments, the cytotoxic agent is vincristine, vinblastine, vindesine, vinorelbine, VP-16, camptothecin, paclitaxel, docetaxel, epithilone A, epithilone B, nocodazole, coichicine, colcimid, estramustine, cemadotin, discodermolide, maytansine, DM-1, AEFP, auristatin E, AEB, AEVB, AEFP, MMAE or eleutherobin.

In specific embodiments, an anti-CD19 antibody is conjugated to the cytotoxic agent via a linker, wherein the linker is peptide linker. In other embodiments, an anti-CD19 antibody is conjugated to the cytotoxic agent via a linker, wherein the linker is a val-cit linker, a phe-lys linker, a hydrazone linker, or a disulfide linker.

In certain embodiments, the anti-CD19 antibody of an anti-CD19 antibody-cytotoxic agent conjugate is conjugated to the cytotoxic agent via a linker, wherein the linker is hydrolysable at a pH of less than 5.5. In a specific embodiment the linker is hydrolyzable at a pH of less than 5.0.

In certain embodiments, the anti-CD19 antibody of an anti-CD19 antibody-cytotoxic agent conjugate is conjugated to the cytotoxic agent via a linker, wherein the linker is cleavable by a protease. In a specific embodiment, the protease is a lysosomal protease. In other embodiments, the protease is, inter alia, a membrane-associated protease, an intracellular protease, or an endosomal protease.

Other toxins that can be used in immunoconjugates of the invention include poisonous lectins, plant toxins such as ricin, abrin, modeccin, botulina, and diphtheria toxins. Of course, combinations of the various toxins could also be coupled to one antibody molecule thereby accommodating variable cytotoxicity. Illustrative of toxins which are suitably employed in combination therapies of the invention are ricin, abrin, ribonuclease, DNase I, Staphylococcal enterotoxin-A, pokeweed anti-viral protein, gelonin, diphtherin toxin, Pseudomonas exotoxin, and Pseudomonas endotoxin. See, for example, Pastan et al., Cell, 47:641 (1986), and Goldenberg et al., Cancer Journal for Clinicians, 44:43 (1994). Enzymatically active toxins and fragments thereof which can be used include diphtheria A chain, non-binding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes. See, for example, WO 93/21232 published Oct. 28, 1993.

Suitable toxins and chemotherapeutic agents are described in Remington's Pharmaceutical Sciences, 19th Ed. (Mack Publishing Co. 1995), and in Goodman And Gilman's The Pharmacological Basis of Therapeutics, 7th Ed. (MacMillan Publishing Co. 1985). Other suitable toxins and/or chemotherapeutic agents are known to those of skill in the art.

The present invention further encompasses antibodies (including antibody fragments or variants thereof) comprising or conjugated to a radioactive agent suitable for diagnostic purposes. Examples of suitable radioactive materials include, but are not limited to, iodine (¹²¹I, ¹²³I, ¹²⁵I, ¹³¹I), carbon (¹⁴C), sulfur (³⁵S), tritium (³H), indium (¹¹¹In, ¹¹²In, ^{113m}In, ^{115m}In) technetium (⁹⁹Tc, ^{99m}Tc), thallium (²⁰¹Ti), gallium (⁶⁸Ga, ⁶⁷Ga), palladium (¹⁰³Pd), molybdenum (⁹⁹Mo), xenon (¹³⁵Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, and ⁹⁷Ru.

Further, an anti-CD19 antibody of the invention (including an scFv or other molecule comprising, or alternatively consisting of, antibody fragments or variants thereof), may be coupled or conjugated to a radioactive metal ion utilized for therapeutic purposes. Examples of suitable radioactive ions include, but are not limited to, alpha-emitters such as ²¹³Bi, or other radioisotopes such as ¹⁰³Pd, ¹³⁵Xe, ¹³¹I, ⁶⁸Ge, ⁵⁷Co, ⁶⁵Zn, ⁸⁵Sr, ³²P, ³⁵S, ⁹⁰Y, ¹⁵³Sm, ¹⁵³Gd, ¹⁶⁹Yb, ⁵¹Cr, ⁵⁴Mn, ⁷⁵Se, ¹¹³Sn, ⁹⁰Y, ¹¹⁷Tin, ¹⁸⁶Re, ¹⁸⁸Re and ¹⁶⁶Ho. In specific embodiments, an antibody or fragment thereof is attached to macrocyclic chelators that chelate radiometal ions, including but not limited to, ¹⁷⁷Lu, ⁹⁰Y, ¹⁶⁶Ho, and ¹⁵³Sm, to polypeptides. In specific embodiments, the macrocyclic chelator is 1,4,7,10-tetraazacyclod-odecane-N,N′, N″,N′″-tetraacetic acid (DOTA). In other specific embodiments, the DOTA is attached to the an antibody of the invention or fragment thereof via a linker molecule. Examples of linker molecules useful for conjugating DOTA to a polypeptide are commonly known in the art—see, for example, DeNardo et al., Clin Cancer Res 4(10):2483-90, 1998; Peterson et al., Bioconjug Chem 10(4):553-7, 1999; and Zimmerman et al., Nucl Med Biol 26(8):943-50, 1999 which are hereby incorporated by reference in their entirety.

An anti-CD19 antibody of the present invention may also be used in ADEPT by conjugating the antibody to a prodrug-activating enzyme which converts a prodrug (e.g., a peptidyl chemotherapeutic agent, see, WO81/01145) to an active anti-cancer drug. See, for example, WO 88/07378 and U.S. Pat. No. 4,975,278. The enzyme component of the immunoconjugate useful for ADEPT includes any enzyme capable of acting on a prodrug in such a way so as to convert it into its more active, cytotoxic form.

Enzymes that are useful in the method of this invention include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as β-galactosidase and neuraminidase useful for converting glycosylated prodrugs into free drugs; β-lactamase useful for converting drugs derivatized with α-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Antibodies with enzymatic activity, also known in the art as “abzymes,” can be used as well to convert the prodrugs into free active drugs (see, e.g., Massey, Nature 328:457-458 (1987)). Antibody-abzyme conjugates can be prepared as described herein for delivery of the abzyme as desired to portions of a human affected by a B cell malignancy.

Antibodies of this invention may be covalently bound to the enzymes by techniques well-known in the art such as the use of the heterobifunctional crosslinking reagents discussed above.

Fusion proteins comprising at least the antigen-binding region of an anti-CD19 antibody linked to at least a functionally active portion of an enzyme may also be constructed using recombinant DNA techniques well-known in the art (see, e.g., Neuberger et al., Nature, 312:604-608 (1984)).

Covalent modifications of an anti-CD19 antibody are included within the scope of this invention. They may be made by chemical synthesis or by enzymatic or chemical cleavage of the antibody, if applicable. Other types of covalent modifications of an anti-CD19 antibody are introduced into the molecule by reacting targeted amino acid residues of the antibody with an organic derivatizing agent that is capable of reacting with selected side chains or the N- or C-terminal residues.

Cysteinyl residues most commonly are reacted with α-haloacetates (and corresponding amines), such as chloroacetic acid or chloroacetamide, to give carboxymethyl or carboxyamidomethyl derivatives. Similarly, iodo-reagents may also be used. Cysteinyl residues also are derivatized by reaction with bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide also is useful; the reaction can be performed in 0.1 M sodium cacodylate at pH 6.0.

Lysyl and amino-terminal residues are reacted with succinic or other carboxylic acid anhydrides. Derivatization with these agents has the effect of reversing the charge of the lysinyl residues. Other suitable reagents for derivatizing α-amino-containing residues and/or ε-amino-containing residues include imidoesters such as methyl picolinimidate, pyridoxal phosphate, pyridoxal, chloroborohydride, trinitrobenzenesulfonic acid, 0-methylisourea, 2,4-pentanedione, and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginyl residues generally requires that the reaction be performed in alkaline conditions because of the high pKa of the guanidine functional group. Furthermore, these reagents may react with the ε-amino groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively. Tyrosyl residues are iodinated using ¹²⁵I or ¹³¹I to prepare labeled proteins for use in radioimmunoassay.

Carboxyl side groups (aspartyl or glutamyl) are selectively modified by reaction with carbodiimides (R—N═C═N—R′), where R and R′ are different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Glutaminyl and asparaginyl residues are frequently deamidated to the corresponding glutamyl and aspartyl residues, respectively. These residues are deamidated under neutral or basic conditions. The deamidated form of these residues falls within the scope of this invention.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W.H. Freeman & Co., San Francisco, pp. 79-86 (1983)), acetylation of the N-terminal amine, and amidation of any C-terminal carboxyl group.

Another type of covalent modification involves chemically or enzymatically coupling glycosides to the antibody. These procedures are advantageous in that they do not require production of the antibody in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330 published 11 Sep. 1987, and in Aplin and Wriston, CRC Crit. Rev. Biochem., pp. 259-306 (1981).

### 6.16. Chemotherapeutic Combinations

In other embodiments, an anti-CD19 mAb can be administered in combination with one or more additional chemotherapeutic agents. For example, “CVB” (1.5 g/m² cyclophosphamide, 200-400 mg/m² etoposide, and 150-200 mg/m² carmustine) can be used in combination therapies of the invention. CVB is a regimen used to treat non-Hodgkin's lymphoma (Patti et al., Eur. J. Haematol., 51:18 (1993)). Other suitable combination chemotherapeutic regimens are well-known to those of skill in the art. See, for example, Freedman et al., “Non-Hodgkin's Lymphomas,” in Cancer Medicine, Volume 2, 3rd Edition, Holland et al. (eds.), pp. 2028-2068 (Lea & Febiger 1993). As an illustration, first generation chemotherapeutic regimens for treatment of intermediate-grade non-Hodgkin's lymphoma include C-MOPP (cyclophosphamide, vincristine, procarbazine and prednisone) and CHOP (cyclophosphamide, doxorubicin, vincristine, and prednisone). A useful second generation chemotherapeutic regimen is m-BACOD (methotrexate, bleomycin, doxorubicin, cyclophosphamide, vincristine, dexamethasone, and leucovorin), while a suitable third generation regimen is MACOP-B (methotrexate, doxorubicin, cyclophosphamide, vincristine, prednisone, bleomycin, and leucovorin). Additional useful drugs include phenyl butyrate and brostatin-1.

According to the invention, cancer or one or more symptoms thereof may be prevented, treated, managed or ameliorated by the administration of an anti-CD19 mAb in combination with the administration of one or more therapies such as, but not limited to, chemotherapies, radiation therapies, hormonal therapies, and/or biological therapies/immunotherapies.

In a specific embodiment, methods of the invention encompass the administration of one or more angiogenesis antagonists such as but not limited to: Angiostatin (plasminogen fragment); antiangiogenic antithrombin III; Angiozyme; ABT-627; Bay 12-9566; Benefin; Bevacizumab; BMS-275291; cartilage-derived inhibitor (CDI); CAI; CD59 complement fragment; CEP-7055; Col 3; Combretastatin A-4; Endostatin (collagen XVIII fragment); Fibronectin fragment; Gro-beta; Halofuginone; Heparinases; Heparin hexasaccharide fragment; HMV833; Human chorionic gonadotropin (hCG); IM-862; Interferon alpha/beta/gamma; Interferon inducible protein (IP-10); Interleukin-12; Kringle 5 (plasminogen fragment); Marimastat; Metalloproteinase inhibitors (TIMPs); 2-Methoxyestradiol; MMI 270 (CGS 27023A); MoAb IMC-1C11; Neovastat; NM-3; Panzem; PI-88; Placental ribonuclease inhibitor; Plasminogen activator inhibitor; Platelet factor-4 (PF4); Prinomastat; Prolactin 16 kD fragment; Proliferin-related protein (PRP); PTK 787/ZK 222594; Retinoids; Solimastat; Squalamine; SS 3304; SU 5416; SU6668; SU11248; Tetrahydrocortisol-S; tetrathiomolybdate; thalidomide; Thrombospondin-1 (TSP-1); TNP-470; Transforming growth factor-beta (TGF-b); Vasculostatin; Vasostatin (calreticulin fragment); ZD6126; ZD 6474; farnesyl transferase inhibitors (FTI); and bisphosphonates (such as but are not limited to, alendronate, clodronate, etidronate, ibandronate, pamidronate, risedronate, tiludronate, and zoledronate).

In a specific embodiment, methods of the invention encompass the administration of one or more immunomodulatory agents, such as but not limited to, chemotherapeutic agents and non-chemotherapeutic immunomodulatory agents. Non-limiting examples of chemotherapeutic agents include methotrexate, cyclosporin A, leflunomide, cisplatin, ifosfamide, taxanes such as taxol and paclitaxol, topoisomerase I inhibitors (e.g., CPT-11, topotecan, 9-AC, and GG-211), gemcitabine, vinorelbine, oxaliplatin, 5-fluorouracil (5-FU), leucovorin, vinorelbine, temodal, cytochalasin B, gramicidin D, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin homologues, and cytoxan. Examples of non-chemotherapeutic immunomodulatory agents include, but are not limited to, anti-T cell receptor antibodies (e.g., anti-CD4 antibodies (e.g., cM-T412 (Boeringer), IDEC-CE9.1® (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD2 antibodies (e.g., MEDI-507 (MedImmune, Inc., International Publication Nos. WO 02/098370 and WO 02/069904), anti-CD11a antibodies (e.g., Xanelim (Genentech)), and anti-B7 antibodies (e.g., IDEC-114) (IDEC)); anti-cytokine receptor antibodies (e.g., anti-IFN receptor antibodies, anti-IL-2 receptor antibodies (e.g., Zenapax (Protein Design Labs)), anti-IL-4 receptor antibodies, anti-IL-6 receptor antibodies, anti-IL-10 receptor antibodies, and anti-IL-12 receptor antibodies), anti-cytokine antibodies (e.g., anti-IFN antibodies, anti-TNF-α antibodies, anti-IL-1β antibodies, anti-IL-6 antibodies, anti-IL-8 antibodies (e.g., ABX-IL-8 (Abgenix)), anti-IL-12 antibodies and anti-IL-23 antibodies)); CTLA4-immunoglobulin; LFA-3TIP (Biogen, International Publication No. WO 93/08656 and U.S. Pat. No. 6,162,432); soluble cytokine receptors (e.g., the extracellular domain of a TNF-α receptor or a fragment thereof, the extracellular domain of an IL-1β receptor or a fragment thereof, and the extracellular domain of an IL-6 receptor or a fragment thereof); cytokines or fragments thereof (e.g., interleukin (IL)-2, IL-3, IL-4, IL-5, IL-6, IL-7, IL-8, IL-9, IL-10, IL-11, IL-12, IL-15, IL-23, TNF-α, TNF-β, interferon (IFN)-α, IFN-β, IFN-γ, and GM-CSF); and anti-cytokine antibodies (e.g., anti-IL-2 antibodies, anti-IL-4 antibodies, anti-IL-6 antibodies, anti-IL-10 antibodies, anti-IL-12 antibodies, anti-IL-15 antibodies, anti-TNF-α antibodies, and anti-IFN-γ antibodies), and antibodies that immunospecifically bind to tumor-associated antigens (e.g., Herceptin®). In certain embodiments, an immunomodulatory agent is an immunomodulatory agent other than a chemotherapeutic agent. In other embodiments an immunomodulatory agent is an immunomodulatory agent other than a cytokine or hemapoietic such as IL-1, IL-2, IL-4, IL-12, IL-15, TNF, IFN-α, IFN-β, IFN-γ, M-CSF, G-CSF, IL-3 or erythropoietin. In yet other embodiments, an immunomodulatory agent is an agent other than a chemotherapeutic agent and a cytokine or hemapoietic factor.

In a specific embodiment, methods of the invention encompass the administration of one or more anti-inflammatory agents, such as but not limited to, non-steroidal anti-inflammatory drugs (NSAIDs), steroidal anti-inflammatory drugs, beta-agonists, anticholingeric agents, and methyl xanthines. Examples of NSAIDs include, but are not limited to, aspirin, ibuprofen, celecoxib (CELEBREX™), diclofenac (VOLTAREN™), etodolac (LODINE™), fenoprofen (NALFON™) indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxygenase enzyme (e.g., COX-1 and/or COX-2). Examples of steroidal anti-inflammatory drugs include, but are not limited to, glucocorticoids, dexamethasone (DECADRON™), cortisone, hydrocortisone, prednisone (DELTASONE™), prednisolone, triamcinolone, azulfidine, and eicosanoids such as prostaglandins, thromboxanes, and leukotrienes.

In another specific embodiment, methods of the invention encompass the administration of one or more antiviral agents (e.g., amantadine, ribavirin, rimantadine, acyclovir, famciclovir, foscarnet, ganciclovir, trifluridine, vidarabine, didanosine, stavudine, zalcitabine, zidovudine, interferon), antibiotics (e.g., dactinomycin (formerly actinomycin), bleomycin, mithramycin, and anthramycin (AMC)), anti-emetics (e.g., alprazolam, dexamethoasone, domperidone, dronabinol, droperidol, granisetron, haloperidol, haloperidol, iorazepam, methylprednisolone, metoclopramide, nabilone, ondansetron, prochlorperazine), anti-fungal agents (e.g., amphotericin, clotrimazole, econazole, fluconazole, flucytosine, griseofulvin, itraconazole, ketoconazole, miconazole and nystatin), anti-parasite agents (e.g., dehydroemetine, diloxanide furoate, emetine, mefloquine, melarsoprol, metronidazole, nifurtimox, paromomycin, pentabidine, pentamidine isethionate, primaquine, quinacrine, quinidine) or a combination thereof.

Specific examples of anti-cancer agents that can be used in various embodiments of the invention, including pharmaceutical compositions and dosage forms and kits, include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alpha-2a; interferon alpha-2b; interferon alpha-n1; interferon alpha-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; HMG-CoA reductase inhibitor (such as but not limited to, Lovastatin, Pravastatin, Fluvastatin, Statin, Simvastatin, and Atorvastatin); loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; Vitaxin®; vorozole; zanoterone; zeniplatin; zilascorb; and zinostatin stimalamer. Additional anti-cancer drugs are 5-fluorouracil and leucovorin. These two agents may be useful when used in methods employing thalidomide and a topoisomerase inhibitor. In specific embodiments, an anti-cancer agent is not a chemotherapeutic agent.

In more particular embodiments, the present invention also comprises the administration of an anti-CD19 mAb in combination with the administration of one or more therapies such as, but not limited to, anti-cancer agents such as those disclosed in Table 1, for the treatment of breast, ovary, melanoma, prostate, colon and lung cancers as described above. When used in a combination therapy, the dosages and/or the frequency of administration listed in Table 2 may be decreased.

TABLE 2
Anti-cancer agents

| Therapeutic | | | |
|---|---|---|---|
| Agent | Dose/Administration/Formulation | | |
| doxorubicin | Intravenous | 60-75 mg/m² on Day 1 | 21 day intervals |
| hydrochloride | | | |
| (Adriamycin | | | |
| RDF ® and | | | |
| Adriamycin PFS ® | | | |
| epirubicin | Intravenous | 100-120 mg/m² on Day 1 of | 3-4 week cycles |
| hydrochloride | | each cycle or | |
| (Ellence ™) | | divided equally and given on | |
| | | Days 1-8 of the cycle | |
| fluorousacil | Intravenous | How supplied: | |
| | | 5 mL and 10 mL vials | |
| | | (containing 250 and 500 mg | |
| | | flourouracil respectively) | |
| docetaxel | Intravenous | 60-100 mg/m² over 1 hour | Once every 3 weeks |
| (Taxotere ®) | | | |
| paclitaxel | Intravenous | 175 mg/m² over 3 hours | Every 3 weeks for |
| (Taxol ®) | | | 4 courses (administered |
| | | | sequentially to doxorubicin- |
| | | | containing combination |
| | | | chemotherapy) |
| tamoxifen citrate | Oral | 20-40 mg | Daily |
| (Nolvadex ®) | (tablet) | Dosages greater than 20 mg | |
| | | should be given in divided | |
| | | doses (morning and evening) | |
| leucovorin calcium | intravenous | How supplied: | Dosage is unclear from text. |
| for injection | or | 350 mg vial | PDR 3610 |
| | intramuscular | | |
| | injection | | |
| luprolide acetate | single | 1 mg (0.2 mL or 20 unit mark) | Once a day |
| Lupron ®) | subcutaneous | | |
| | injection | | |
| flutamide | Oral | 50 mg | 3 times a day at 8 hour |
| (Eulexin ®) | (capsule) | (capsules contain 125 mg | intervals (total daily dosage |
| | | flutamide each) | 750 mg) |
| nilutamide | Oral | 300 mg or 150 mg | 300 mg once a day for 30 |
| (Nilandron ®) | (tablet) | (tablets contain 50 or 150 mg | days followed by 150 mg |
| | | nilutamide each) | once a day |
| bicalutamide | Oral | 50 mg | Once a day |
| (Casodex ®) | (tablet) | (tablets contain 50 mg | |
| | | bicalutamide each) | |
| progesterone | Injection | USP in sesame oil 50 mg/mL | |
| ketoconazole | Cream | 2% cream applied once or twice | |
| (Nizoral ®) | | daily depending on symptoms | |
| prednisone | Oral | Initial dosage may vary from 5 | |
| | (tablet) | mg to 60 mg per day depending | |
| | | on the specific disease entity | |
| | | being treated. | |
| estramustine | Oral | 14 mg/kg of body weight (i.e. | Daily given in 3 or 4 divided |
| phosphate sodium | (capsule) | one 140 mg capsule for each 10 | doses |
| (Emcyt ®) | | kg or 22 lb of body weight) | |
| etoposide or | Intravenous | 5 mL of 20 mg/mL solution | |
| VP-16 | | (100 mg) | |
| dacarbazine | Intravenous | 2-4.5 mg/kg | Once a day for 10 days. |
| (DTIC-Dome ®) | | | May be repeated at 4 week |
| | | | intervals |
| polifeprosan 20 | wafer placed | 8 wafers, each containing 7.7 | |
| with carmustine | in resection | mg of carmustine, for a total of | |
| implant (BCNU) | cavity | 61.6 mg, if size and shape of | |
| (nitrosourea) | | resection cavity allows | |
| (Gliadel ®) | | | |
| cisplatin | Injection | [n/a in PDR 861] | |
| | | How supplied: | |
| | | solution of 1 mg/mL in multi- | |
| | | dose vials of 50 mL and 100 mL | |
| mitomycin | Injection | supplied in 5 mg and 20 mg | |
| | | vials (containing 5 mg and 20 | |
| | | mg mitomycin) | |
| gemcitabine HCl | Intravenous | For NSCLC—2 schedules have | 4 week schedule— |
| (Gemzar ®) | | been investigated and the | Days 1, 8 and 15 of each 28- |
| | | optimum schedule has not been | day cycle. Cisplatin |
| | | determined | intravenously at 100 mg/m² |
| | | 4 week schedule— | on day 1 after the infusion of |
| | | administration intravenously at | Gemzar. |
| | | 1000 mg/m² over 30 minutes on | 3 week schedule— |
| | | 3 week schedule— | Days 1 and 8 of each 21 day |
| | | Gemzar administered | cycle. Cisplatin at dosage of |
| | | intravenously at 1250 mg/m² | 100 mg/m² administered |
| | | over 30 minutes | intravenously after |
| | | | administration of Gemzar on |
| | | | day 1. |
| carboplatin | Intravenous | Single agent therapy: | Every 4 weeks |
| (Paraplatin ®) | | 360 mg/m² I.V. on day 1 | |
| | | (infusion lasting 15 minutes or | |
| | | longer) | |
| | | Other dosage calculations: | |
| | | Combination therapy with | |
| | | cyclophosphamide, Dose | |
| | | adjustment recommendations, | |
| | | Formula dosing, etc. | |
| ifosamide | Intravenous | 1.2 g/m² daily | 5 consecutive days |
| (Ifex ®) | | | Repeat every 3 weeks or |
| | | | after recovery from |
| | | | hematologic toxicity |
| topotecan | Intravenous | 1.5 mg/m² by intravenous | 5 consecutive days, starting |
| hydrochloride | | infusion over 30 minutes daily | on day 1 of 21 day course |
| (Hycamtin ®) | | | |
| Bisphosphonates | Intravenous | 60 mg or 90 mg single infusion | |
| Pamidronate | or Oral | over 4-24 hours to correct | |
| Alendronate | take with 6-8 | hypercalcemia in cancer | |
| Risedronate | oz water. | patients | |
| | | 5 mg/d daily for 2 years and | |
| | | then 10 mg/d for 9 month to | |
| | | prevent or control bone | |
| | | resorption. | |
| | | 5.0 mg to prevent or control | |
| | | bone resorption. | |
| Lovastatin | Oral | 10-80 mg/day in single or two | |
| (Mevacor ™) | | divided dose. | |

The invention also encompasses administration of an anti-CD19 mAb in combination with radiation therapy comprising the use of x-rays, gamma rays and other sources of radiation to destroy the cancer cells. In particular embodiments, the radiation treatment is administered as external beam radiation or teletherapy wherein the radiation is directed from a remote source. In other embodiments, the radiation treatment is administered as internal therapy or brachytherapy wherein a radiaoactive source is placed inside the body close to cancer cells or a tumor mass.

Cancer therapies and their dosages, routes of administration and recommended usage are known in the art and have been described in such literature as the Physician's Desk Reference (56^{th} ed., 2002).

### 6.17. Pharmaceutical Compositions

The invention also relates to immunotherapeutic compositions and methods for the treatment of B cell diseases and disorders in human subjects, such as, but not limited to, B cell malignancies, to immunotherapeutic compositions and methods for the treatment and prevention of GVHD, graft rejection, and post-transplant lymphocyte proliferative disorder in human transplant recipients, and to immunotherapeutic compositions and methods for the treatment of autoimmune diseases and disorders in human subjects, using therapeutic antibodies that bind to the CD19 antigen and may mediate human ADCC.

The present invention relates to pharmaceutical compositions comprising human, humanized, or chimeric anti-CD19 antibodies of the IgG1 or IgG3 human isotype. The present invention also relates to pharmaceutical compositions comprising human or humanized anti-CD19 antibodies of the IgG2 or IgG4 human isotype that may mediate human ADCC. In certain embodiments, the present invention also relates to pharmaceutical compositions comprising monoclonal human, humanized, or chimerized anti-CD19 antibodies that can be produced by means known in the art.

Therapeutic formulations and regimens are described for treating human subjects diagnosed with B cell malignancies that derive from B cells and their precursors, including but not limited to, acute lymphoblastic leukemias (ALL), Hodgkin's lymphomas, non-Hodgkin's lymphomas, B cell chronic lymphocytic leukemias (CLL), multiple myeloma, follicular lymphoma, mantle cell lymphoma, pro-lymphocytic leukemias, hairy cell leukemias, common acute lymphocytic leukemias and some Null-acute lymphoblastic leukemias.

In other particular embodiments, anti-CD19 antibodies may mediate ADCC, complement-dependent cellular cytoxicity, or apoptosis. Compositions and methods of the present invention also have the advantage of targeting a wider population of B cells than other B cell directed immunotherapies. For example, anti-CD19 antibodies of the present invention may be effective to target bone marrow cells, circulating B cells, and mature, antibody-secreting B cells. Accordingly, methods and compositions of the invention may be effective to reduce or deplete circulating B cells as well as circulating immunoglobulin.

Accordingly, in one aspect, the invention provides compositions and methods for the treatment and prevention of GVHD, graft rejection, and post-transplantation lymphoproliferative disorder, which are associated with fewer and/or less severe complications than less-targeted therapeutic agents and regimens. In one embodiment, compositions and methods of the invention are used with lower doses of traditional therapeutic agents than would be possible in the absence of the methods and compositions of the invention. In another embodiment, compositions and methods of the invention obviate the need for a more severe form of therapy, such as radiation therapy, high-dose chemotherapy, or splenectomy.

In certain embodiments, anti-CD19 antibodies and compositions may be administered to a transplant recipient patient prior to or following transplantation, alone or in combination with other therapeutic agents or regimens for the treatment or prevention of GVHD and graft rejection. For example, anti-CD19 antibodies and compositions may be used to deplete alloantibodies from a transplant recipient prior to or following transplantation of an allogeneic graft. Anti-CD19 antibodies and compositions may also be used to deplete antibody-producing cells from the graft ex vivo, prior to transplantation, or in the donor, or as prophylaxis against GVHD and graft rejection.

### 6.18. Pharmaceutical Formulations, Administration and Dosing

Pharmaceutical formulations of the invention contain as the active ingredient human, humanized, or chimeric anti-CD19 antibodies. The formulations contain naked antibody, immunoconjugate, or fusion protein in an amount effective for producing the desired response in a unit of weight or volume suitable for administration to a human patient, and are preferably sterile. The response can, for example, be measured by determining the physiological effects of the anti-CD19 antibody composition, such as, but not limited to, circulating B cell depletion, tissue B cell depletion, regression of a B cell malignancy, or decrease of disease symptoms. Other assays will be known to one of ordinary skill in the art and can be employed for measuring the level of the response.

### 6.18.1. Pharmaceutical Formulations

An anti-CD19 antibody composition may be formulated with a pharmaceutically acceptable carrier. The term “pharmaceutically acceptable” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also routinely contain compatible solid or liquid fillers, diluents or encapsulating substances which are suitable for administration into a human. When used in medicine, the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulfuric, nitric, phosphoric, maleic, acetic, salicylic, citric, boric, formic, malonic, succinic, and the like. Also, pharmaceutically acceptable salts can be prepared as alkaline metal or alkaline earth salts, such as sodium, potassium or calcium salts. The term “carrier” denotes an organic or inorganic ingredient, natural or synthetic, with which the active ingredient is combined to facilitate the application. The components of the pharmaceutical compositions also are capable of being co-mingled with the antibodies of the present invention, and with each other, in a manner such that there is no interaction which would substantially impair the desired pharmaceutical efficacy.

According to certain aspects of the invention, anti-CD19 antibody compositions can be prepared for storage by mixing the antibody or immunoconjugate having the desired degree of purity with optional physiologically acceptable carriers, excipients or stabilizers (Remington's Pharmaceutical Sciences, 16th edition, Osol, A. Ed. (1999)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN, PLURONICS™ or polyethylene glycol (PEG).

Anti-CD19 antibody compositions also may contain, optionally, suitable preservatives, such as: benzalkonium chloride; chlorobutanol; parabens and thimerosal.

Anti-CD19 antibody compositions may conveniently be presented in unit dosage form and may be prepared by any of the methods well-known in the art of pharmacy. All methods include the step of bringing the active agent into association with a carrier which constitutes one or more accessory ingredients. In general, anti-CD19 antibody compositions are prepared by uniformly and intimately bringing the active compound into association with a liquid carrier, a finely divided solid carrier, or both, and then, if necessary, shaping the product.

Compositions suitable for parenteral administration conveniently comprise a sterile aqueous or non-aqueous preparation of anti-CD19 antibody, which may be isotonic with the blood of the recipient. This preparation may be formulated according to known methods using suitable dispersing or wetting agents and suspending agents. The sterile injectable preparation also may be a sterile injectable solution or suspension in a non-toxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administration can be found in Remington's Pharmaceutical Sciences, Mack Publishing Co., Easton, Pa. In certain embodiments, carrier formulation suitable for various routes of administration can be the same or similar to that described for RITUXAN™. See, Physicians' Desk Reference (Medical Economics Company, Inc., Montvale, N.J., 2005), pp. 958-960 and 1354-1357, which is incorporated herein by reference in its entirety. In certain embodiments of the invention, anti-CD19 antibody compositions are formulated for intravenous administration with sodium chloride, sodium citrate dihydrate, polysorbate 80, and sterile water where the pH of the composition is adjusted to approximately 6.5. Those of skill in the art are aware that intravenous injection provides a useful mode of administration due to the thoroughness of the circulation in rapidly distributing antibodies. Intravenous administration, however, is subject to limitation by a vascular barrier comprising endothelial cells of the vasculature and the subendothelial matrix. Still, the vascular barrier is a more notable problem for the uptake of therapeutic antibodies by solid tumors. Lymphomas have relatively high blood flow rates, contributing to effective antibody delivery. Intralymphatic routes of administration, such as subcutaneous or intramuscular injection, or by catheterization of lymphatic vessels, also provide a useful means of treating B cell lymphomas. In certain embodiments, anti-CD19 antibodies of compositions and methods of the invention are self-administered subcutaneously. In such embodiments, the composition is formulated as a lyophilized drug or in a liquid buffer (e.g., PBS and/or citrate) at about 50 mg/mL.

The formulation herein may also contain more than one active compound as necessary for the particular indication being treated, such as those with complementary activities that do not adversely affect each other. For example, it may be desirable to further provide an immunosuppressive agent. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration are typically sterile. This is readily accomplished by filtration through sterile filtration membranes.

Sustained-release preparations may be prepared. Suitable examples of sustained-release preparations include semipermeable matrices of solid hydrophobic polymers containing an anti-CD19 antibody, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions. In certain embodiments, the pharmaceutically acceptable carriers used in compositions of the invention do not affect human ADCC or CDC.

Anti-CD19 antibody compositions disclosed herein may also be formulated as immunoliposomes. A “liposome” is a small vesicle composed of various types of lipids, phospholipids and/or surfactant which is useful for delivery of a drug (such as anti-CD19 antibodies disclosed herein) to a human. The components of the liposome are commonly arranged in a bilayer formation, similar to the lipid arrangement of biological membranes. Liposomes containing antibodies of the invention are prepared by methods known in the art, such as described in Epstein et al., Proc. Natl. Acad. Sci. USA, 82:3688 (1985); Hwang et al., Proc. Natl. Acad. Sci. USA, 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. The antibody of the present invention can be conjugated to the liposomes as described in Martin et al., J. Biol. Chem., 257:286-288 (1982) via a disulfide interchange reaction. A therapeutic agent can also be contained within the liposome. See, Gabizon et al., J. National Cancer Inst., (19)1484 (1989).

Some of the pharmaceutical formulations include, but are not limited to:

(a) a sterile, preservative-free liquid concentrate for intravenous (i.v.) administration of anti-CD19 antibody, supplied at a concentration of 10 mg/ml in either 100 mg (10 mL) or 500 mg (50 mL) single-use vials. The product can be formulated for i.v. administration using sodium chloride, sodium citrate dihydrate, polysorbate and sterile water for injection. For example, the product can be formulated in 9.0 mg/mL sodium chloride, 7.35 mg/mL sodium citrate dihydrate, 0.7 mg/mL polysorbate 80, and sterile water for injection. The pH is adjusted to 6.5.

(b) A sterile, lyophilized powder in single-use glass vials for subcutaneous (s.c.) injection. The product can be formulated with sucrose, L-histidine hydrochloride monohydrate, L-histidine and polysorbate 20. For example, each single-use vial can contain 150 mg anti-CD19 antibody, 123.2 mg sucrose, 6.8 mg L-histidine hydrochloride monohydrate, 4.3 mg L-histidine, and 3 mg polysorbate 20. Reconstitution of the single-use vial with 1.3 ml sterile water for injection yields approximately 1.5 ml solution to deliver 125 mg per 1.25 ml (100 mg/ml) of antibody.

(c) A sterile, preservative-free lyophilized powder for intravenous (i.v.) administration. The product can be formulated with α-trehalose dihydrate, L-histidine HCl, histidine and polysorbate 20 USP. For example, each vial can contain 440 mg anti-CD19 antibody, 400 mg α,α-trehalose dihydrate, 9.9 mg L-histidine HCl, 6.4 mg L-histidine, and 1.8 mg polysorbate 20, USP. Reconstitution with 20 ml of bacteriostatic water for injection (BWFI), USP, containing 1.1% benzyl alcohol as a preservative, yields a multi-dose solution containing 21 mg/ml antibody at a pH of approximately 6.

(d) A sterile, lyophilized powder for intravenous infusion in which an anti-CD19 antibody is formulated with sucrose, polysorbate, monobasic sodium phosphate monohydrate, and dibasic sodium phosphate dihydrate. For example, each single-use vial can contain 100 mg antibody, 500 mg sucrose, 0.5 mg polysorbate 80, 2.2 mg monobasic sodium phosphate monohydrate, and 6.1 mg dibasic sodium phosphate dihydrate. No preservatives are present. Following reconstitution with 10 ml sterile water for injection, USP, the resulting pH is approximately 7.2.

(e) A sterile, preservative-free solution for subcutaneous administration supplied in a single-use, 1 ml pre-filled syringe. The product can be formulated with sodium chloride, monobasic sodium phosphate dihydrate, dibasic sodium phosphate dihydrate, sodium citrate, citric acid monohydrate, mannitol, polysorbate 80 and water for injection, USP. Sodium hydroxide may be added to adjust pH to about 5.2.

For example, each syringe can be formulated to deliver 0.8 ml (40 mg) of drug product. Each 0.8 ml contains 40 mg anti-CD19 antibody, 4.93 mg sodium chloride, 0.69 mg monobasic sodium phosphate dihydrate, 1.22 mg dibasic sodium phosphate dihydrate, 0.24 mg sodium citrate, 1.04 citric acid monohydrate, 9.6 mg mannitol, 0.8 mg polysorbate 80 and water for injection, USP.

(f) A sterile, preservative-free, lyophilized powder contained in a single-use vial that is reconstituted with sterile water for injection (SWFI), USP, and administered as a subcutaneous (s.c.) injection. The product can be formulated with sucrose, histidine hydrochloride monohydrate, L-histidine, and polysorbate. For example, a 75 mg vial can contain 129.6 mg or 112.5 mg of an anti-CD19 antibody, 93.1 mg sucrose, 1.8 mg L-histidine hydrochloride monohydrate, 1.2 mg L-histidine, and 0.3 mg polysorbate 20, and is designed to deliver 75 mg of the antibody in 0.6 ml after reconstitution with 0.9 ml SWFI, USP. A 150 mg vial can contain 202.5 mg or 175 mg anti-CD19 antibody, 145.5 mg sucrose, 2.8 mg L-histidine hydrochloride monohydrate, 1.8 mg L-histidine, and 0.5 mg polysorbate 20, and is designed to deliver 150 mg of the antibody in 1.2 ml after reconstitution with 1.4 ml SWFI, USP.

(g) A sterile, hyophilized product for reconstitution with sterile water for injection. The product can be formulated as single-use vials for intramuscular (IM) injection using mannitol, histidine and glycine. For example, each single-use vial can contain 100 mg anti-CD19 antibody, 67.5 mg of mannitol, 8.7 mg histidine and 0.3 mg glycine, and is designed to deliver 100 mg antibody in 1.0 ml when reconstituted with 1.0 ml sterile water for injection. As another example, each single-use vial can contain 50 mg anti-CD19 antibody, 40.5 mg mannitol, 5.2 mg histidine and 0.2 mg glycine, and is designed to deliver 50 mg of antibody when reconstituted with 0.6 ml sterile water for injection.

(h) A sterile, preservative-free solution for intramuscular (IM) injection, supplied at a concentration of 100 mg/ml. The product can be formulated in single-use vials with histidine, glycine, and sterile water for injection. For example, each single-use vial can be formulated with 100 mg antibody, 4.7 mg histidine, and 0.1 mg glycine in a volume of 1.2 ml designed to deliver 100 mg of antibody in 1 ml. As another example, each single-use vial can be formulated with 50 mg antibody, 2.7 mg histidine and 0.08 mg glycine in a volume of 0.7 ml or 0.5 ml designed to deliver 50 mg of antibody in 0.5 ml.

(i) A sterile, preservative-free solution for subcutaneous administration of anti-CD19 antibody, supplied at a concentration of 100 mg/mL in a single-use, 1 ml pre-filled syringe, such as tungsten-free ultra-100 syringe, or auto-injector. The product can be formulated with sodium chloride, histidine, trehalose, polysorbate and sterile water for injection. For example, the product can be formulated in 10 mM histidine, 75 mM sodium chloride, and 4% trehalose, wherein said formulation has a pH of 6.0.

(j) A sterile, preservative-free solution for subcutaneous administration of anti-CD19 antibody, supplied at a concentration of 50 mg/mL in a single-use, 1 ml pre-filled syringe, such as tungsten-free ultra-100 syringe, or auto-injector. The product can be formulated with sodium chloride, histidine, trehalose, polysorbate and sterile water for injection. For example, the product can be formulated in 10 mM histidine, 75 mM sodium chloride, and 4% trehalose, wherein said formulation has a pH of 6.0.

(k) A sterile, preservative-free solution for subcutaneous administration of anti-CD19 antibody, supplied at a concentration of 25 mg/mL in a single-use, 1 ml pre-filled syringe, such as tungsten-free ultra-100 syringe, or auto-injector. The product can be formulated with sodium chloride, histidine, trehalose, polysorbate and sterile water for injection. For example, the product can be formulated in 10 mM histidine, 75 mM sodium chloride, and 4% trehalose, wherein said formulation has a pH of 6.0.

(l) A sterile, preservative-free solution for subcutaneous administration of anti-CD19 antibody, supplied at a concentration of 10 mg/mL in a single-use, 1 ml pre-filled syringe, such as tungsten-free ultra-100 syringe, or auto-injector. The product can be formulated with sodium chloride, histidine, trehalose, polysorbate and sterile water for injection. For example, the product can be formulated in 10 mM histidine, 75 mM sodium chloride, and 4% trehalose, wherein said formulation has a pH of 6.0.

(m) A sterile, preservative-free liquid concentrate for intravenous (i.v.) administration of anti-CD19 antibody, supplied at a concentration of 10 mg/ml in either 100 mg (10 mL) or 500 mg (50 mL) single-use vials. The product can be formulated for i.v. administration using sodium chloride, histidine, trehalose, polysorbate and sterile water for injection. For example, the product can be formulated in 10 mM histidine, 75 mM sodium chloride, and 4% trehalose, wherein said formulation has a pH of 6.0.

In certain embodiments, apharmaceutical composition of the invention is stable at 4° C. In certain embodiments, a pharmaceutical composition of the invention is stable at room temperature.

### 6.18.2. Antibody Half-Life

In certain embodiments, the half-life of an anti-CD19 antibody of compositions and methods of the invention is at least about 4 to 7 days. In certain embodiments, the mean half-life of an anti-CD19 antibody of compositions and methods of the invention is at least about 2 to 5 days, 3 to 6 days, 4 to 7 days, 5 to 8 days, 6 to 9 days, 7 to 10 days, 8 to 11 days, 8 to 12, 9 to 13, 10 to 14, 11 to 15, 12 to 16, 13 to 17, 14 to 18, 15 to 19, or 16 to 20 days. In other embodiments, the mean half-life of an anti-CD19 antibody of compositions and methods of the invention is at least about 17 to 21 days, 18 to 22 days, 19 to 23 days, 20 to 24 days, 21 to 25, days, 22 to 26 days, 23 to 27 days, 24 to 28 days, 25 to 29 days, or 26 to 30 days. In still further embodiments the half-life of an anti-CD19 antibody of compositions and methods of the invention can be up to about 50 days. In certain embodiments, the half-lives of antibodies of compositions and methods of the invention can be prolonged by methods known in the art. Such prolongation can in turn reduce the amount and/or frequency of dosing of the antibody compositions. Antibodies with improved in vivo half-lives and methods for preparing them are disclosed in U.S. Pat. No. 6,277,375; and International Publication Nos. WO 98/23289 and WO 97/3461.

The serum circulation of anti-CD19 antibodies in vivo may also be prolonged by attaching inert polymer molecules such as high molecular weight polyethyleneglycol (PEG) to the antibodies with or without a multifunctional linker either through site-specific conjugation of the PEG to the N- or C-terminus of the antibodies or via epsilon-amino groups present on lysyl residues. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can be tested for binding activity as well as for in vivo efficacy using methods known to those of skill in the art, for example, by immunoassays described herein.

Further, the antibodies of compositions and methods of the invention can be conjugated to albumin in order to make the antibody more stable in vivo or have a longer half-life in vivo. The techniques are well known in the art, see, e.g., International Publication Nos. WO 93/15199, WO 93/15200, and WO 01/77137; and European Patent No. EP 413, 622, all of which are incorporated herein by reference.

### 6.18.3. Administration and Dosing

Administration of compositions of the invention to a human patient can be by any route, including but not limited to intravenous, intradermal, transdermal, subcutaneous, intramuscular, inhalation (e.g., via an aerosol), buccal (e.g., sub-lingual), topical (i.e., both skin and mucosal surfaces, including airway surfaces), intrathecal, intraarticular, intraplural, intracerebral, intra-arterial, intraperitoneal, oral, intralymphatic, intranasal, rectal or vaginal administration, by perfusion through a regional catheter, or by direct intralesional injection. In one embodiment, compositions of the invention are administered by intravenous push or intravenous infusion given over defined period (e.g., 0.5 to 2 hours). Compositions of the invention can be delivered by peristaltic means or in the form of a depot, although the most suitable route in any given case will depend, as is well known in the art, on such factors as the species, age, gender and overall condition of the subject, the nature and severity of the condition being treated and/or on the nature of the particular composition (i.e., dosage, formulation) that is being administered. In particular embodiments, the route of administration is via bolus or continuous infusion over a period of time, once or twice a week. In other particular embodiments, the route of administration is by subcutaneous injection, optionally once or twice weekly. In one embodiment, compositions, and/or methods of the invention are administered on an outpatient basis.

In certain embodiments, the dose of a composition comprising anti-CD19 antibody is measured in units of mg/kg of patient body weight. In other embodiments, the dose of a composition comprising anti-CD19 antibody is measured in units of mg/kg of patient lean body weight (i.e., body weight minus body fat content). In yet other embodiments, the dose of a composition comprising anti-CD19 antibody is measured in units of mg/m² of patient body surface area. In yet other embodiments, the dose of a composition comprising anti-CD19 antibody is measured in units of mg per dose administered to a patient. Any measurement of dose can be used in conjunction with compositions and methods of the invention and dosage units can be converted by means standard in the art.

Those skilled in the art will appreciate that dosages can be selected based on a number of factors including the age, sex, species and condition of the subject (e.g., stage of B cell malignancy), the desired degree of cellular depletion, the disease to be treated and/or the particular antibody or antigen-binding fragment being used and can be determined by one of skill in the art. For example, effective amounts of compositions of the invention may be extrapolated from dose-response curves derived in vitro test systems or from animal model (e.g., the cotton rat or monkey) test systems. Models and methods for evaluation of the effects of antibodies are known in the art (Wooldridge et al., Blood, 89(8): 2994-2998 (1997)), incorporated by reference herein in its entirety). In certain embodiments, for particular B cell malignancies, therapeutic regimens standard in the art for antibody therapy can be used with compositions and methods of the invention.

Examples of dosing regimens that can be used in methods of the invention include, but are not limited to, daily, three times weekly (intermittent), weekly, or every 14 days. In certain embodiments, dosing regimens include, but are not limited to, monthly dosing or dosing every 6-8 weeks.

Those skilled in the art will appreciate that dosages are generally higher and/or frequency of administration greater for initial treatment as compared with maintenance regimens.

In some embodiments of the invention, anti-CD19 antibodies bind to B cells and may result in efficient (i.e., at low dosage) depletion of B cells (as described herein). Higher degrees of binding may be achieved where the density of human CD19 on the surface of a patient's B cells is high. In certain embodiments, dosages of the antibody (optionally in a pharmaceutically acceptable carrier as part of a pharmaceutical composition) are at least about 0.0005, 0.001, 0.05, 0.075, 0.1, 0.25, 0.375, 0.5, 1, 2.5, 5, 10, 20, 37.5, or 50 mg/m² and/or less than about 500, 475, 450, 425, 400, 375, 350, 325, 300, 275, 250, 225, 200, 175, 150, 125, 100, 75, 60, 50, 37.5, 20, 15, 10, 5, 2.5, 1, 0.5, 0.375, 0.1, 0.075 or 0.01 mg/m². In certain embodiments, the dosage is between about 0.0005 to about 200 mg/m², between about 0.001 and 150 mg/m², between about 0.075 and 125 mg/m², between about 0.375 and 100 mg/m², between about 2.5 and 75 mg/m², between about 10 and 75 mg/m², and between about 20 and 50 mg/m². In related embodiments, the dosage of anti-CD19 antibody used is at least about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5 mg/kg of body weight of a patient. In certain embodiments, the dose of naked anti-CD19 antibody used is at least about 1 to 10, 5 to 15, 10 to 20, or 15 to 25 mg/kg of body weight of a patient. In certain embodiments, the dose of anti-CD19 antibody used is at least about 1 to 20, 3 to 15, or 5 to 10 mg/kg of body weight of a patient. In other embodiments, the dose of anti-CD19 antibody used is at least about 5, 6, 7, 8, 9, or 10 mg/kg of body weight of a patient. In certain embodiments, a single dosage unit of the antibody (optionally in a pharmaceutically acceptable carrier as part of a pharmaceutical composition) can be at least about 0.5, 1, 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 204, 206, 208, 210, 212, 214, 216, 218, 220, 222, 224, 226, 228, 230, 232, 234, 236, 238, 240, 242, 244, 246, 248, or 250 micrograms/m². In other embodiments, dose is up to 1 g per single dosage unit.

All of the above doses are exemplary and can be used in conjunction with compositions and methods of the invention, however where an anti-CD19 antibody is used in conjunction with a toxin or radiotherapeutic agent the lower doses described above may be preferred. In certain embodiments, where the patient has low levels of CD19 density, the lower doses described above may be preferred.

In certain embodiments of the invention where chimeric anti-CD19 antibodies are used, the dose or amount of the chimeric antibody is greater than about 2, 3, 4, 5, 6, 7, 8, 9, 10, 11, 12, 13, 14, 15, or 16 mg/kg of patient body weight. In other embodiments of the invention where chimeric anti-CD19 antibodies are used, the dose or amount of the chimeric antibody is less than about 1, 0.9, 0.8, 0.7, 0.6, 0.5, 0.4, 0.3, 0.2, or 0.1 mg/kg of patient body weight.

In some embodiments of methods of this invention, antibodies and/or compositions of this invention can be administered at a dose lower than about 375 mg/m²; at a dose lower than about 37.5 mg/m²; at a dose lower than about 0.375 mg/m²; and/or at a dose between about 0.075 mg/m² and about 125 mg/m². In certain embodiments of methods of the invention, dosage regimens comprise low doses, administered at repeated intervals. For example, in one embodiment, compositions of the invention can be administered at a dose lower than about 375 mg/m² at intervals of approximately every 1, 2, 3, 4, 5, 6, 7, 8, 9, 10, 15, 20, 25, 30, 35, 40, 45, 50, 60, 70, 80, 90, 100, 125, 150, 175, or 200 days.

The specified dosage can result in B cell depletion in the human treated using compositions and methods of the invention for a period of at least about 1, 2, 3, 5, 7, 10, 14, 20, 30, 45, 60, 75, 90, 120, 150 or 180 days or longer. In certain embodiments, pre-B cells (not expressing surface immunoglobulin) are depleted. In certain embodiments, mature B cells (expressing surface immunoglobulin) are depleted. In other embodiments, all non-malignant types of B cells can exhibit depletion. Any of these types of B cells can be used to measure B cell depletion. B cell depletion can be measured in bodily fluids such as blood serum, or in tissues such as bone marrow. In certain embodiments of methods of the invention, B cells are depleted by at least 30%, 40%, 50%, 60%, 70%, 80%, 90%, or 100% in comparison to B cell levels in the patient being treated before use of compositions and methods of the invention. In other embodiments of methods of the invention, B cells are depleted by at least 30%, 40%, 50%, 60%, 70%, 80%, 90%, or 100% in comparison to typical standard B cell levels for humans. In related embodiments, the typical standard B cell levels for humans are determined using patients comparable to the patient being treated with respect to age, sex, weight, and other factors.

In certain embodiments of the invention, a dosage of about 125 mg/m² or less of an antibody or antigen-binding fragment results in B cell depletion for a period of at least about 7, 14, 21, 30, 45, 60, 90, 120, 150, or 200 days. In another representative embodiment, a dosage of about 37.5 mg/m² or less depletes B cells for a period of at least about 7, 14, 21, 30, 45, 60, 90, 120, 150, or 200 days. In still other embodiments, a dosage of about 0.375 mg/m² or less results in depletion of B cells for at least about 7, 14, 21, 30, 45 or 60 days. In another embodiment, a dosage of about 0.075 mg/m² or less results in depletion of B cells for a period of at least about 7, 14, 21, 30, 45, 60, 90, 120, 150, or 200 days. In yet other embodiments, a dosage of about 0.01 mg/m², 0.005 mg/m² or even 0.001 mg/m² or less results in depletion of B cells for at least about 3, 5, 7, 10, 14, 21, 30, 45, 60, 90, 120, 150, or 200 days. According to these embodiments, the dosage can be administered by any suitable route, but is optionally administered by a subcutaneous route or intravenous route.

As another aspect, the invention provides the discovery that B cell depletion and/or treatment of B cell disorders can be achieved at lower dosages of antibody or antibody fragments than employed in currently available methods. Thus, in another embodiment, the invention provides a method of depleting B cells and/or treating a B cell disorder, comprising administering to a human, an effective amount of an antibody that specifically binds to CD19, wherein a dosage of about 500, 475, 450, 425, 400, 375, 350, 325, 300, 275, 250, 225, 200, 175, 150, 125, 100, 75, 60, 50, 37.5, 20, 10, 5, 2.5, 1, 0.5, 0.375, 0.25, 0.1, 0.075, 0.05, 0.001, 0.0005 mg/m² or less results in a depletion of B cells (circulating and/or tissue B cells) of 25%, 35%, 50%, 60%, 75%, 80%, 85%, 90%, 95%, 98% or more for a period at least about 3, 5, 7, 10, 14, 21, 30, 45, 60, 75, 90, 120, 150, 180, or 200 days or longer. In representative embodiments, a dosage of about 125 mg/m² or 75 mg/m² or less results in at least about 50%, 75%, 85% or 90% depletion of B cells for at least about 7, 14, 21, 30, 60, 75, 90, 120, 150 or 180 days. In other embodiments, a dosage of about 50, 37.5 or 10 mg/m² results in at least about a 50%, 75%, 85% or 90% depletion of B cells for at least about 7, 14, 21, 30, 60, 75, 90, 120 or 180 days. In still other embodiments, a dosage of about 0.375 or 0.1 mg/m² results in at least about a 50%, 75%, 85% or 90% depletion of B cells for at least about 7, 14, 21, 30, 60, 75 or 90 days. In further embodiments, a dosage of about 0.075, 0.01, 0.001, or 0.0005 mg/m² results in at least about a 50%, 75%, 85% or 90% depletion of B cells for at least about 7, 14, 21, 30 or 60 days.

In certain embodiments of the invention, the dose can be escalated or reduced to maintain a constant dose in the blood or in a tissue, such as, but not limited to, bone marrow. In related embodiments, the dose is escalated or reduced by about 2%, 5%, 8%, 10%, 15%, 20%, 30%, 40%, 50%, 60%, 70%, 80%, 90%, and 95% in order to maintain a desired level of an antibody of compositions and methods of the invention.

In certain embodiments, the dosage can be adjusted and/or the infusion rate can be reduced based on patient's immunogenic response to compositions and methods of the invention.

According to one aspect of methods of the invention, a loading dose of an anti-CD19 antibody and/or composition of the invention can be administered first followed by a maintenance dose until the B cell malignancy being treated progresses or followed by a defined treatment course (e.g., CAMPATH™, MYLOTARG™, or RITUXAN™, the latter of which allow patients to be treated for a defined number of doses that has increased as additional data have been generated).

According to another aspect of methods of the invention, a patient may be pretreated with compositions and methods of the invention to detect, minimize immunogenic response, or minimize adverse effects of compositions and methods of the invention.

### 6.18.4. Toxicity Testing

The tolerance, toxicity and/or efficacy of the compositions and/or treatment regimens of the present invention can be determined by standard pharmaceutical procedures in cell cultures or experimental animals, e.g., for determining the LD50 (the dose lethal to 50% of the population), the ED50 (the dose therapeutically effective in 50% of the population), and IC50 (the dose effective to achieve a 50% inhibition). In one embodiment, the dose is a dose effective to achieve at least a 60%, 70%, 80%, 90%, 95%, or 99% depletion of circulating B cells or circulating immunogloblulin, or both. The dose ratio between toxic and therapeutic effects is the therapeutic index and it can be expressed as the ratio LD50/ED50. Therapies that exhibit large therapeutic indices may be preferred. While therapies that exhibit toxic side effects may be used, care should be taken to design a delivery system that targets such agents to CD19-expressing cells in order to minimize potential damage to CD19 negative cells and, thereby, reduce side effects.

Data obtained from the cell culture assays and animal studies can be used in formulating a range of dosages of the compositions and/or treatment regimens for use in humans. The dosage of such agents may lie within a range of circulating concentrations that include the ED50 with little or no toxicity. The dosage may vary within this range depending upon the dosage form employed and the route of administration utilized. For any therapy used in methods of the invention, a therapeutically effective dose can be estimated by appropriate animal models. Depending on the species of the animal model, the dose can be scaled for human use according to art-accepted formulas, for example, as provided by Freireich et al., Quantitative comparison of toxicity of anticancer agents in mouse, rat, monkey, dog, and human, Cancer Chemotherapy Reports, NCI 1966 40:219-244. Data obtained from cell culture assays can be useful for predicting potential toxicity Animal studies can be used to formulate a specific dose to achieve a circulating plasma concentration range that includes the IC50 (i.e., the concentration of the test compound that achieves a half-maximal inhibition of symptoms) as determined in cell culture. Such information can be used to more accurately determine useful doses in humans. Plasma drug levels may be measured, for example, by high performance liquid chromatography, ELISA, or by cell based assays.

### 6.19. Patient Diagnosis, Staging and Therapeutic Regimens Oncology

According to certain aspects of the invention, the treatment regimen and dose used with compositions and methods of the invention is chosen based on a number of factors including, but not limited to, the stage of the B cell disease or disorder being treated. Appropriate treatment regimens can be determined by one of skill in the art for particular stages of a B cell disease or disorder in a patient or patient population. Dose response curves can be generated using standard protocols in the art in order to determine the effective amount of compositions of the invention for treating patients having different stages of a B cell disease or disorder. In general, patients having more advanced stages of a B cell disease or disorder will require higher doses and/or more frequent doses which may be administered over longer periods of time in comparison to patients having an early stage B cell disease or disorder.

Anti-CD19 antibodies, compositions and methods of the invention may be practiced to treat B cell diseases, including B cell malignancies. The term “B cell malignancy” includes any malignancy that is derived from a cell of the B cell lineage. Exemplary B cell malignancies include, but are not limited to: B cell subtype non-Hodgkin's lymphoma (NHL) including low grade/follicular NHL, small lymphocytic (SL) NHL, intermediate grade/follicular NHL, intermediate grade diffuse NHL, high grade immunoblastic NHL, high grade lymphoblastic NHL, high grade small non-cleaved cell NHL; mantle-cell lymphoma, and bulky disease NHL; Burkitt's lymphoma; multiple myeloma; pre-B acute lymphoblastic leukemia and other malignancies that derive from early B cell precursors; common acute lymphocytic leukemia (ALL); chronic lymphocytic leukemia (CLL) including immunoglobulin-mutated CLL and immunoglobulin-unmutated CLL; hairy cell leukemia; Null-acute lymphoblastic leukemia; Waldenstrom's Macroglobulinemia; diffuse large B cell lymphoma (DLBCL) including germinal center B cell-like (GCB) DLBCL, activated B cell-like (ABC) DLBCL, and type 3 DLBCL; pro-lymphocytic leukemia; light chain disease; plasmacytoma; osteosclerotic myeloma; plasma cell leukemia; monoclonal gammopathy of undetermined significance (MGUS); smoldering multiple myeloma (SMM); indolent multiple myeloma (IMM); Hodgkin's lymphoma including classical and nodular lymphocyte pre-dominant type; lymphoplasmacytic lymphoma (LPL); and marginal-zone lymphoma including gastric mucosal-associated lymphoid tissue (MALT) lymphoma.

In a further embodiment the invention can be employed to treat mature B cell malignancies (i.e., express Ig on the cell surface) including but not limited to follicular lymphoma, mantle-cell lymphoma, Burkitt's lymphoma, multiple myeloma, diffuse large B-cell lymphoma (DLBCL) including germinal center B cell-like (GCB) DLBCL, activated B cell-like (ABC) DLBCL, and type 3 DLBCL, Hodgkin's lymphoma including classical and nodular lymphocyte pre-dominant type, lymphoplasmacytic lymphoma (LPL), marginal-zone lymphoma including gastric mucosal-associated lymphoid tissue (MALT) lymphoma, and chronic lymphocytic leukemia (CLL) including immunoglobulin-mutated CLL and immunoglobulin-unmutated CLL.

Further, CD19 is expressed earlier in B cell development than, for example, CD20, and is therefore particularly suited for treating pre-B cell and immature B cell malignancies (i.e., do not express Ig on the cell surface), for example, in the bone marrow. Illustrative pre-B cell and immature B cell malignancies include, but are not limited to, acute lymphoblastic leukemia.

In other particular embodiments, the invention can be practiced to treat extranodal tumors.

### 6.19.1. Diagnosis and Staging of B Cell Malignancies

The progression of cancer, such as a B cell disease or disorder capable of tumor formation (e.g., non-Hodgkin lymphoma, diffuse large B cell lymphoma, follicular lymphoma, and Burkitt lymphoma) is typically characterized by the degree to which the cancer has spread through the body and is often broken into the following four stages which are prognostic of outcome. Stage I: The cancer is localized to a particular tissue and has not spread to the lymph nodes. Stage 11: The cancer has spread to the nearby lymph nodes, i.e., metastasis. Stage III: The cancer is found in the lymph nodes in regions of the body away from the tissue of origin and may comprise a mass or multiple tumors as opposed to one. Stage IV: The cancer has spread to a distant part of the body. The stage of a cancer can be determined by clinical observations and testing methods that are well known to those of skill in the art. The stages of cancer described above are traditionally used in conjunction with clinical diagnosis of cancers characterized by tumor formation, and can be used in conjunction with the compositions and methods of the present invention to treat B cell diseases and disorders. Typically early stage disease means that the disease remains localized to a portion of a patient's body or has not metastasized.

With respect to non-tumor forming B cell diseases and disorders such as, but not limited to, multiple myeloma, the criteria for determining the stage of disease differs. The Durie-Salmon Staging System has been widely used. In this staging system, clinical stage of disease (stage I, II, or III) is based on several measurements, including levels of M protein, the number of lytic bone lesions, hemoglobin values, and serum calcium levels. Stages are further divided according to renal (kidney) function (classified as A or B). According to the Durie-Salmon Staging System Stage I (low cell mass) is characterized by all of the following: Hemoglobin value>10 g/dL; Serum calcium value normal or ≦12 mg/dL; Bone x-ray, normal bone structure (scale 0) or solitary bone plasmacytoma only; and Low M-component production rate: IgG value<5 g/dL, IgA value<3 g/d, Bence Jones protein<4 g/24 h. Stage I patients typically have no related organ or tissue impairment or symptoms. Stage II (intermediate cell mass) is characterized by fitting neither stage I nor stage III. Stage III (high cell mass) is characterized by one or more of the following: Hemoglobin value<8.5 g/dL; Serum calcium value>12 mg/dL; Advanced lytic bone lesions (scale 3); High M-component production rate: IgG value>7 g/dL, IgA value>5 g/dL, Bence Jones protein>12 g/24 h Subclassification (either A or B), where A is Relatively normal renal function (serum creatinine value<2.0 mg/dL) and B is Abnormal renal function (serum creatinine value≧2.0 mg/dL).

Another staging system for myeloma is the International Staging System (ISS) for myeloma. This system can more effectively discriminate between staging groups and is based on easily measured serum levels of beta 2-microglobulin (β2-M) and albumin. According to the ISS for myeloma, Stage I is characterized by β2-M<3.5 and Albumin≧3.5, Stage II is characterized by β2-M<3.5 and albumin<3.5 or β2-M 3.5-5.5, and Stage III is characterized by β2-M>5.5 (Multiple Myeloma Research Foundation, New Canaan, Conn.).

The stage of a B cell malignancy in a patient is a clinical determination. As indicated above, with respect to solid tumors, the spread, location, and number of tumors are the primary factors in the clinical determination of stage. Determination of stage in patients with non-tumor forming B cell malignancies can be more complex requiring serum level measurements as described above.

The descriptions of stages of B cell diseases and disorders above are not limiting. Other characteristics known in the art for the diagnosis of B cell diseases and disorders can be used as criteria for patients to determine stages of B cell diseases or disorders.

### 6.19.1.1. Multiple Myeloma

Multiple myeloma is a malignancy of plasma cells. Neoplastic cells are located in the bone marrow, and osteolytic bone lesions are characteristic. Reciprocal chromosomal translocations between one of the immunoglobulin loci and a variety of other genes, e.g., cyclin D1, cyclin D3, c-MAF, MMSET (multiple myeloma SET-domain protein) or fibroblast growth factor receptor 3 are believed to be the primary oncogenic events. Multiple myeloma is characterized by SHM, and the putative cell of origin is a post-GC B cell. Multiple myeloma is typically first identified by symptoms such as recurrent infection, fatigue, pain, and kidney problems and is confirmed with clinical testing (see, for example, Cancer: Principles and Practice of Oncology. 6th edition. DeVita, V. T., Hellman, S, and Rosenberg, S. A. editors. 2001 Lippincott Williams and Wilkins Philadelphia, Pa. 19106 pp. 2465-2499).

In certain embodiments, patients who are candidates for treatment by compositions and methods of the invention can undergo further diagnostic tests on blood and/or urine to confirm the diagnosis or suspicion of multiple myeloma including, but not limited to, complete blood count (CBC) tests to determine if the types of cells reported in a CBC are within their normal ranges which are well known in the art, blood chemistry profile to determine whether levels of various blood components, such as albumin, blood urea nitrogen (BUN), calcium, creatinine, and lactate dehydrogenase (LDH), deviate from standard values. Serum levels of beta₂-microglobulin (β₂-M) can also be examined and surrogate markers for IL-6, a growth factor for myeloma cells. Urinalysis can be used to measure the levels of protein in the urine. Electrophoresis can be used to measure the levels of various proteins, including M protein in the blood (called serum protein electrophoresis, or SPEP) or urine (called urine electrophoresis, or UEP). An additional test, called immunofixation electrophoresis (IFE) or immunoelectrophoresis, may also be performed to provide more specific information about the type of abnormal antibody proteins present. Assessing changes and proportions of various proteins, particularly M protein, can be used to track the progression of myeloma disease and response to treatment regimens. Multiple myeloma is characterized by a large increase in M protein which is secreted by the myeloma tumor cells.

Diagnostic tests on bone can also be conducted to confirm the diagnosis or suspicion of multiple myeloma including, but not limited to, X-rays and other imaging tests—including a bone (skeletal) survey, magnetic resonance imaging (MRI), and computerized axial tomography (CAT), also known as computed tomography (CT)—can assess changes in the bone structure and determine the number and size of tumors in the bone. Bone marrow aspiration or bone marrow biopsy can be used to detect an increase in the number of plasma cells in the bone marrow. Aspiration requires a sample of liquid bone marrow, and biopsy requires a sample of solid bone tissue. In both tests, samples can be taken from the pelvis (hip bone). The sternum (breast bone) can also be used for aspiration of bone marrow.

Patients with multiple myeloma are typically categorized into the following three groups that help define effective treatment regimens. Monoclonal gammopathy of undetermined significance (MGUS) is typically characterized by a serum M protein level of less than 3 g/dL, bone marrow clonal plasma cells of less than 10%, no evidence of other B cell disorders, and no related organ or tissue impairment, such as hypercalcemia (increased serum calcium levels), impaired kidney function noted by increased serum creatinine, anemia, or bone lesions. Asymptomatic myelomas are typically stage I and includes smoldering multiple myeloma (SMM) and indolent multiple myeloma (IMM). SMM is characterized by serum M protein greater than or equal to 3 g/dL and IMM is characterized by bone marrow clonal plasma cells greater than or equal to 10% of the bone marrow cells. Symptomatic myeloma is characterized by M protein in serum and/or urine and includes Stage II multiple myeloma characterized by the presence of bone marrow clonal plasma cells or plasmacytoma and Stage III multiple myeloma characterized by related organ or tissue impairment.

Osteosclerotic myeloma is a component of the rare POEMS syndrome (polyneuropathy, organomegaly, endocrinopathy, monoclonal gammopathy and skin lesions). Peak incidence is at 40 to 50 years of age. Systemic features include skeletal lesions, marrow-plasma cells<5%, a normal CBC, increased platelets, and organomegaly. The CSF has a high protein with no cells present. The M-protein levels are low (<3 g/dl, median=1.1 g/dl); heavy chain class—usually α or γ; light chain class—usually λ; rare urine monoclonal and occasional cryoglobulinemia. Neuropathy occurs in 50% of the patients with weakness both proximal and distal, sensory loss is greater in larger than small fibers; and demyelination and long distal latency.

Smoldering multiple myeloma patients generally present with stable disease for months/years; no anemia, bone lesions, renal insufficiency or hypercalcemia; have>10% plasma cells in bone marrow and monoclonal serum protein. The criteria for smoldering multiple myeloma is compatible with the diagnosis of multiple myeloma; however, there is no evidence of progressive course. These are cases with a slow progression, the tumor cell mass is low at diagnosis and the percentage of bone marrow plasma cells in S phase is low (<0.5%). Characteristic clinical features include: serum M protein levels>3 g/dL and/or bone marrow plasma cells≧10%; absence of anemia, renal failure, hypercalcemia, lytic bone lesions.

Indolent (or asymptomatic) multiple myeloma is a multiple myeloma diagnosed by chance in the absence of symptoms, usually after screening laboratory studies. Indolent multiple myeloma is similar to smoldering myeloma but with few bone lesions and mild anemia. Most cases of indolent multiple myeloma develop overt multiple myeloma within 3 years. Diagnostic criteria are the same as for multiple myeloma except: no bone lesions or one asymptomatic lytic lesion (X-ray survey); M component level<3 g/dL for IgG, 2 g/dL for IgA urine light chain<4 g/24 h; hemoglobin>10 g/dl, serum calcium normal, serum creatinine<2 mg/dL, and no infections.

The identity of the cells responsible for the initiation and maintenance of multiple myeloma (MM) is still unclear because of the difficulty growing MM cells in vitro and in vivo. Many agents are active in multiple myeloma, but the majority of patients relapse. This clinical pattern suggests most cancer cells are eliminated, but cells with the clonogenic potential to mediate tumor regrowth are relatively chemoresistant. The cancer stem cell hypothesis is supported by the finding that human MM cell lines contained small (<5%) subpopulations that lacked CD138 expression and had greater clonogenic potential in vitro than corresponding CD138+ plasma cells (Matsui et al. Blood. 2004 Mar. 15; 103(6):2332-6). CD138− cells from clinical MM samples were similarly clonogenic both in vitro and in nonobese diabetic/severe combined immunodeficiency (NOD/SCID) mice, whereas CD138+ cells were not. Furthermore, CD138− cells from both cell lines and clinical samples phenotypically resembled postgerminal center B cells, and their clonogenic growth was inhibited by the anti-CD20 monoclonal antibody rituximab. Additionally, CD138− cells from both cell lines and clinical samples expressed the CD27 and CD19 B cell markers. These data suggest that MM “stem cells” are CD138− B cells with the ability to replicate and subsequently differentiate into malignant CD138+ plasma cells. Additional results supporting a cancer stem cell hypothesis in multiple myeloma were reviewed in Huff & Matsui (J Clin Oncol. (2008) 26(17):2895-900) and Matsui et al. (Cancer Res. (2008) 68(1):190-7).

The present invention provides for antibodies that efficiently deplete CD138-clonogenic B cells in a human multiple myeloma patient. In a specific embodiment, an anti-CD-19 antibody of the invention may achieve at least about 20%, at least about 30%, at least about 40%, at least about 50%, at least about 60%, at least about 70%, at least about 80%, at least about 90%, at least about 95%, or about 100% CD138-clonogenic B cell depletion in a human multiple myeloma patient. Depletion of CD138-clonogenic B cells may persist for extended periods of time. In one embodiment, CD138-clonogenic B cell depletion by an anti-CD19 antibody of the invention may persist for at least 1 day, at least 2 days, at least 3 days, at least 4 days, at least 5 days at least 6 days, at least 7 days, at least 8 days, at least 9 days, at least 10 days, at least 15 days, at least 20 days, at least 25 days, or at least 30 days. In another embodiment, CD138-clonogenic B cell depletion by an anti-CD19 antibody of the invention may persist for at least 1 week, at least 2 weeks, at least 3 weeks, at least 4 weeks, at least 5 weeks, at least 6 weeks, at least 7 weeks, at least 8 weeks, at least 9 weeks, or at least 10 weeks. In a further embodiment, CD138-clonogenic B cell depletion by an anti-CD19 antibody of the invention may persist for at least 1 month, at least 2 months, at least 3 months, at least 4 months, at least 5 months, at least 6 months, at least 7 months, at least 8 months, at least 9 months, at least 10 months, at least 11 months or at least 12 months.

The present invention provides for antibodies that efficiently deplete cancer stem cells in a human multiple myeloma patient. In a specific embodiment, an anti-CD-19 antibody of the invention may achieve at least about 20%, at least about 30%, at least about 40%, at least about 50%, at least about 60%, at least about 70%, at least about 80%, at least about 90%, at least about 95%, or about 100% cancer stem cell depletion in a human multiple myeloma patient. Depletion of cancer stem cells may persist for extended periods of time. In one embodiment, cancer stem cell depletion by an anti-CD19 antibody of the invention may persist for at least 1 day, at least 2 days, at least 3 days, at least 4 days, at least 5 days at least 6 days, at least 7 days, at least 8 days, at least 9 days, at least 10 days, at least 15 days, at least 20 days, at least 25 days, or at least 30 days. In another embodiment, cancer stem cell depletion by an anti-CD19 antibody of the invention may persist for at least 1 week, at least 2 weeks, at least 3 weeks, at least 4 weeks, at least 5 weeks, at least 6 weeks, at least 7 weeks, at least 8 weeks, at least 9 weeks, or at least 10 weeks. In a further embodiment, cancer stem cell depletion by an anti-CD19 antibody of the invention may persist for at least 1 month, at least 2 months, at least 3 months, at least 4 months, at least 5 months, at least 6 months, at least 7 months, at least 8 months, at least 9 months, at least 10 months, at least 11 months or at least 12 months.

The present invention also relates to a method of treating multiple myeloma in a human comprising administering to a human in need thereof, a human, humanized or chimeric anti-CD19 antibody that may mediate human antibody-dependent cellular cytotoxicity (ADCC), complement-dependent cell-mediated cytotoxicity (CDC), and/or apoptosis in an amount sufficient to deplete circulating B cells. In one embodiment, a method of treating multiple myeloma in a human comprises the depletion of CD138-clonogenic B cells. In another embodiment, a method of treating multiple myeloma in a human comprises the depletion of cancer stem cells. In a specific embodiment, the depletion may achieve at least about 20%, at least about 30%, at least about 40%, at least about 50%, at least about 60%, at least about 70%, at least about 80%, at least about 90%, at least about 95%, or about 100% reduction in CD138-clonogenic B cells or cancer stem cells. The depletion may persist for extended periods of time. In one embodiment, the depletion of CD138-clonogenic B cells or cancer stem cells may persist for at least 1 day, at least 2 days, at least 3 days, at least 4 days, at least 5 days at least 6 days, at least 7 days, at least 8 days, at least 9 days, at least 10 days, at least 15 days, at least 20 days, at least 25 days, or at least 30 days. In another embodiment, the depletion may persist for at least 1 week, at least 2 weeks, at least 3 weeks, at least 4 weeks, at least 5 weeks, at least 6 weeks, at least 7 weeks, at least 8 weeks, at least 9 weeks, or at least 10 weeks. In a further embodiment, the depletion may persist for at least 1 month, at least 2 months, at least 3 months, at least 4 months, at least 5 months, at least 6 months, at least 7 months, at least 8 months, at least 9 months, at least 10 months, at least 11 months or at least 12 months.

### 6.20. Patient Diagnosis and Therapeutic Regimens: Autoimmune Disease

According to certain aspects of the invention, the treatment regimen and dose used with compositions and methods of the invention is chosen based on a number of factors including, but not limited to, the stage of the autoimmune disease or disorder being treated. Appropriate treatment regimens can be determined by one of skill in the art for particular stages of an autoimmune disease or disorder in a patient or patient population. Dose response curves can be generated using standard protocols in the art in order to determine the effective amount of compositions of the invention for treating patients having different stages of an autoimmune disease or disorder. In general, patients having more activity of an autoimmune disease or disorder will require higher doses and/or more frequent doses which may be administered over longer periods of time in comparison to patients having less activity of an autoimmune disease or disorder.

Anti-CD19 antibodies, compositions and methods described herein may be practiced to treat an autoimmune disease or disorder. The term “autoimmune disease or disorder” refers to a condition in a subject characterized by cellular, tissue and/or organ injury caused by an immunologic reaction of the subject to its own cells, tissues and/or organs. The term “inflammatory disease” is used interchangeably with the term “inflammatory disorder” to refer to a condition in a subject characterized by inflammation, including, but not limited to chronic inflammation. Autoimmune disorders may or may not be associated with inflammation. Moreover, inflammation may or may not be caused by an autoimmune disorder. Thus, certain disorders may be characterized as both autoimmune and inflammatory disorders. Exemplary autoimmune diseases or disorders include, but are not limited to: alopecia greata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, diabetes, eosinophilic fascites, fibromyalgia-fibromyositis, glomerulonephritis, Graves' disease, Guillain-Barre, Hashimoto's thyroiditis, Henoch-Schönlein purpura, idiopathic pulmonary fibrosis, idiopathic/autoimmune thrombocytopenia purpura (ITP), IgA neuropathy, juvenile arthritis, lichen planus, lupus erthematosus, Ménière's disease, mixed connective tissue disease, multiple sclerosis, type 1 or immune-mediated diabetes mellitus, myasthenia gravis, pemphigus-related disorders (e.g., pemphigus vulgaris), pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjögren's syndrome, stiff-man syndrome, systemic lupus erythematosis (SLE), Sweet's syndrome, Still's disease, lupus erythematosus, takayasu arteritis, temporal arteristis/giant cell arteritis, ulcerative colitis, uveitis, vasculitides such as dermatitis herpetiformis vasculitis, vitiligo, and Wegener's granulomatosis. Examples of inflammatory disorders include, but are not limited to, asthma, encephilitis, inflammatory bowel disease, chronic obstructive pulmonary disease (COPD), allergic disorders, septic shock, pulmonary fibrosis, undifferentitated spondyloarthropathy, undifferentiated arthropathy, arthritis, inflammatory osteolysis, graft versus host disease, urticaria, Vogt-Koyanagi-Hareda syndrome and chronic inflammation resulting from chronic viral or bacteria infections.

Anti-CD19 immunotherapy encompasses the administration of an anti-CD19 antibody as a single agent therapeutic for the treatment of an autoimmune disease or disorder. In one embodiment, an anti-CD19 immunotherapy of the invention encompasses the administration of an anti-CD19 antibody capable of inhibiting in vitro stimulated B cell proliferation. In another embodiment, an anti-CD19 immunotherapy of the invention encompasses the administration of an Fc variant anti-CD19 antibody wherein said Fc variant has altered binding affinity to one or more Fc ligand relative to a comparable non-variant molecule. In a specific embodiment, an anti-CD19 immunotherapy of the invention encompasses the administration of an Fc variant anti-CD19 antibody wherein said Fc variant has enhanced binding to Fc gamma receptor JIB relative to a comparable non-variant Fc domain.

Anti-CD19 immunotherapy further encompasses the administration of an anti-CD19 bispecific antibody as a single agent therapeutic for the treatment of an autoimmune disease or disorder. In one embodiment, an anti-CD19 immunotherapy of the invention encompasses the administration of an anti-CD19 bispecific antibody capable to specifically bind to a first and second antigen, wherein said first antigen is human CD19 and said second antigen is an Fc gamma receptor selected from the group consisting of FcγRI, FcγRIIA, FcγRIIB, FcγRIIIA and/or FcγRIV. In a further embodiment, an anti-CD19 immunotherapy of the invention encompasses the administration of an anti-CD19 bispecific antibody capable of specifically binding to human CD19 and FcγRIIB.

Because CD19 is expressed on immature B cells, an anti-CD19 mAb may be particularly suited for depleting pre-B cells and immature B cells, e.g, in the bone marrow.

### 6.20.1. Clinical Criteria for Diagnosing Autoimmune Diseases or Disorders

Diagnostic criteria for different autoimmune diseases or disorders are known in the art. Historically, diagnosis is typically based on a combination of physical symptoms. More recently, molecular techniques such as gene-expression profiling have been applied to develop molecular definitions of autoimmune diseases or disorders. Exemplary methods for clinical diagnosis of particular autoimmune diseases or disorders are provided below. Other suitable methods will be apparent to those skilled in the art.

In certain embodiments, patients with low levels of autoimmune disease activity or patients with an early stage of an autoimmune disease (for diseases where stages are recognized) can be identified for treatment using anti-CD19 antibody compositions and methods. The early diagnosis of autoimmune disease is difficult due to the generality of its symptoms and overlap of symptoms among autoimmune diseases. In such embodiments, a patient treated at an early stage or with low levels of an autoimmune disease activity has symptoms comprising at least one symptom of an autoimmune disease or disorder. In related embodiments, a patient treated at an early stage or with low levels of an autoimmune disease has symptoms comprising at least 1, 2, 3, 4, 5, 6, 7, 8, 9, 10, 11, 12, 13, 14, or 15 symptoms of an autoimmune disease or disorder. The symptoms may be of any autoimmune diseases and disorders or a combination thereof. Examples of autoimmune disease and disorder symptoms are described below.

### 6.20.2. Systemic Lupus Erythematosis (SLE)

Systemic lupus erythematosis (SLE) is a chronic (long-lasting) rheumatic disease which affects joints, muscles and other parts of the body. Patients or patient populations in need of treatment for SLE can be identified by examining physical symptoms and/or laboratory test results. Physical symptoms vary widely among patients. For example, in SLE, typically 4 of the following 11 symptoms exist before a patient is diagnosed with SLE: 1) malar rash: rash over the cheeks; 2) discoid rash: red raised patches; 3) photosensitivity: reaction to sunlight, resulting in the development of or increase in skin rash; 4) oral ulcers: ulcers in the nose or mouth, usually painless; 5) arthritis: nonerosive arthritis involving two or more peripheral joints (arthritis in which the bones around the joints do not become destroyed); 6) serositis pleuritis or pericarditis: inflammation of the lining of the lung or heart; 7) renal disorder: excessive protein in the urine (greater than 0.5 gm/day or 3+ on test sticks) and/or cellular casts (abnormal elements the urine, derived from red and/or white cells and/or kidney tubule cells); 8) neurologic disorder: seizures (convulsions) and/or psychosis in the absence of drugs or metabolic disturbances which are known to cause such effects; 9) hematologic disorder: hemolytic anemia or leukopenia (white blood count below 4,000 cells per cubic millimeter) or lymphopenia (less than 1,500 lymphocytes per cubic millimeter) or thrombocytopenia (less than 100,000 platelets per cubic millimeter) (where the leukopenia or lymphopenia must be detected on two or more occasions. The thrombocytopenia must be detected in the absence of drugs known to induce it); 10) antinuclear antibody: positive test for antinuclear antibodies (ANA) in the absence of drugs known to induce it; and/or 11) immunologic disorder: positive anti-double stranded anti-DNA test, positive anti-sm test, positive antiphospholipid antibody such as anticardiolipin, or false positive syphilis test (vdrl).

Other physical symptoms that may be indicative of SLE include, but are not limited to, anemia, fatigue, fever, skin rash, muscle aches, nausea, vomiting and diarrhea, swollen glands, lack of appetite, sensitivity to cold (Raynaud's phenomenon), and weight loss.

Laboratory tests can also be used to identify patients or patient populations in need of treatment. For example, a blood test can be used to detect autoantibodies found in the blood of almost all people with SLE. Such tests may include but are not limited to tests for antinuclear antibodies (ANA) in the absence of drugs known to induce it (Rahman, A. and Hiepe, F. Lupus. (2002). 11(12):770-773), anti-double stranded anti-DNA (Keren, D. F. Clin. Lab. Med. (2002) 22(2):447-474), anti-Sm, antiphospholipid antibody such as anticardiolipin (Gezer, S. Dis. Mon. 2003. 49(12):696-741), or false positive syphilis tests (VDRL).

Other tests may include a complement test (C3, C4, CH50, CH100) which can be used to measure the amount of complement proteins circulating in the blood (Manzi et al. Lupus 2004. 13(5):298-303), a sedimentation rate (ESR) or C-reactive protein (CRP) may be used to measure inflammation levels, a urine analysis can be used to detect kidney problems, chest X-rays may be taken to detect lung damage, and an EKG can be used to detect heart problems.

Chronic SLE is associated with accumulating collateral damage to involved organ, particularly the kidney. Accordingly, early therapeutic intervention is desirable, i.e. prior to, for example, kidney failure. Available treatments for SLE are similar to those available for rheumatoid arthritis. These include initial treatments, either with an analgesic or a nonsteroidal anti-inflammatory (NSAID) compound. As the disease progresses and/or the symptoms increase in severity, SLE may be treated by the administration of steroids such as but not limited to dexamethasone and prednisone.

In more severe cases, a chemotherapeutic agent, such as but not limited to methotrexate or cytoxin may be administered to relieve the symptoms of SLE. However, this approach is not preferred where the patient is a female of child-bearing age. In such instances, those therapeutic approaches that do not interfere with the reproductive capacity of the patient are preferred.

In certain instances, SLE may be treated by administration of a biologic, such as an antibody or a receptor (or receptor analog). Examples of such therapeutic antibodies are Rituxin and Remicade. An illustrative example of a soluble receptor for an inflammatory cytokine that can be administered to treat SLE is Enbrel.

In certain embodiments of the methods of invention, a patient can be treated with an anti-CD19 antibody prior, concurrent, or subsequent to any of the therapies disclosed above that are used for the treatment of SLE. Moreover, anti-CD19 antibodies of the present invention may be administered in combination with any of the analgesic, NSAID, steroid, or chemotherapeutic agents noted above, as well as in combination with a biologic administered for the treatment of SLE.

### 6.20.3. Systemic Sclerosis (Scleroderma) and Related Disorders

Systemic sclerosis also known as Scleroderma encompasses a heterogeneous group of diseases including but not limited to, Limited cutaneous disease, Diffuse cutaneous disease, Sine scleroderma, Undifferentiated connective tissue disease, Overlap syndromes, Localized scleroderma, Morphea, Linear scleroderma, En coup de saber, Scleredema adultorum of Buschke, Scleromyxedema, Chronic graft-vs.-host disease, Eosinophilic fasciitis, Digital sclerosis in diabetes, and Primary anylooidosisand anyloidosis associated with multiple myeloma. (Reviewed in: Harrison's Principles of Internal Medicine, 16^{th} ed./editors, Dennis L. Kasper, et al. The McGraw-Hill Companies, Inc. 2005 New York, N.Y.).

Clinical features associated with scleroderma can include Raynaud's phenomenon, skin thickening, subcutaneious calcinosis, telangiectasia, arthralgias/arthritis, myopathy, esophageal dysmotility, pulmonary fibrosis, isolated pulmonary arterial hypertension, congestive heart failure and renal crisis. The extent to which an patient displays one or more of these disease manifestations can influence the diagnosis and potential treatment plan.

Autoantibodies include: Anti-topioisomerase 1, anticentromere, anti-RNA polymerase I, II, and/or III, anti-Th RNP, anti-U, RNP (anti-fibrillarin), anti-PM/Sci, anti-nuclear antibodies (ANA).

Identification of patients and patient populations in need of treatment of scleroderma can be based on clinical history and physical findings. Patients or patient populations in need of treatment for scleroderma can be identified by examining a patient's medical history, physical symptoms, and/or laboratory test results. Diagnosis may be delayed in patients without significant skin thickening. Laboratory, X-ray, pulmonary function tests, and skin or renal (kidney) biopsies can be used to determine the extent and severity of internal organ involvement.

In the early months or years of disease onset, scleroderma may resemble many other connective tissue diseases, such as, but not limited to, Systemic Lupus Erythematosus, Polymyositis, and Rheumatoid Arthritis.

The most classic symptom of systemic sclerosis (scleroderma) is sclerodactyly. Initial symptoms include swollen hands, which sometimes progress to this tapering and claw-like deformity. Not everyone with scleroderma develops this degree of skin hardening. Other symptoms can include morphea, linear sclerodactyly (hardened fingers), Raynaud's syndrome, calcinosis, and telangiectasia.

Blood tests such as antinuclear antibody (ANA) tests can be used in the diagnosis of both localized and systemic scleroderma. For example, anti-centromere antibodies (ACA) and anti-Scl-70 antibodies are indicative of patients in need of treatment for systemic sclerosis (Ho et al., 2003, Arthritis Res Ther. 5:80-93); anti-topo II alpha antibody are indicative of patients in need of treatment for local scleroderma; and anti-topo I alpha antibody are indicative of patients in need of treatment for systemic scleroderma. Several types of scleroderma and methods for diagnosing these types are recognized and well known in the art, including, but not limited to, juvenile scleroderma (Foeldvari, Curr Opin Rheumatol 14:699-703 (2002); Cefle et al., Int J Clin Pract. 58:635-638 (2004)); localized scleroderma; Nodular Scleroderma (Cannick, J. Rheumatol. 30:2500-2502 (2003)); and Systemic scleroderma, including, but not limited to, Calcinosis, Raynaud's, Esophagus, Sclerodactyly, and Telangiectasia (CREST), limited systemic scleroderma, and diffuse systemic scleroderma. Systemic scleroderma is also known as systemic sclerosis (SSc). It may also be referred to as Progressive Systemic Sclerosis (PSSc), or Familial Progressive Systemic Sclerosis (FPSSc) (Nadashkevich et al., Med Sci Monit. 10:CR615-621 (2004); Frances et al., Rev Prat. 52:1884-90 (2002)). Systemic sclerosis is a multisystem disorder characterized by the presence of connective tissue sclerosis, vascular abnormalities concerning small-sized arteries and the microcirculation, and autoimmune changes.

The type of systemic scleroderma known as CREST is not characterized by any skin tightening. CREST is characterized by Calcinosis (calcium deposits), usually in the fingers; Raynaud's; loss of muscle control of the Esophagus, which can cause difficulty swallowing; Sclerodactyly, a tapering deformity of the bones of the fingers; and Telangiectasia, small red spots on the skin of the fingers, face, or inside of the mouth. Typically two of these symptoms is sufficient for diagnosis of CREST. CREST may occur alone, or in combination with any other form of Scleroderma or with other autoimmune diseases.

Limited Scleroderma is characterized by tight skin limited to the fingers, along with either pitting digital ulcers (secondary to Raynaud's) and/or lung fibrosis. The skin of the face and neck may also be involved in limited scleroderma.

Diffuse Scleroderma is diagnosed whenever there is proximal tight skin. Proximal means located closest to the reference point. Proximal tight skin can be skin tightness above the wrists or above the elbows. Typically, a patient with skin tightness only between their elbows and their wrists will receive a diagnosis of either diffuse or limited systemic Scleroderma, depending on which meaning of proximal the diagnosing clinician uses.

The current therapies for scleroderma include extracorporeal photophoresis following 6-methoxypsoralen, and autologous stem cell transplant.

The current treatments for scleroderma include the administration of the following agents, penicillamine, cholchicine, interferon alpha, interpheron gamma, chlorambucil, cyclosporine, 5-fluorouracil, cyclophosphamide, minocycline, thalidomide, etanercept, or methotrexate.

In certain embodiments of the methods of invention, a patient can be treated with an anti-CD19 antibody prior, concurrent, or subsequent to any of the therapies disclosed above that are used for the treatment of autoimmune diabetes. Moreover, anti-CD19 antibodies of the present invention may be administered in combination with any of the agents noted above.

### 6.21. Immunotherapeutic Protocols

Anti-CD19 antibody compositions used in the therapeutic regimen/protocols, referred to herein as “anti-CD19 immunotherapy” can be naked antibodies, immunoconjugates and/or fusion proteins. Compositions of the invention can be used as a single agent therapy or in combination with other therapeutic agents or regimens. Anti-CD19 antibodies or immunoconjugates can be administered prior to, concurrently with, or following the administration of one or more therapeutic agents. Therapeutic agents that can be used in combination therapeutic regimens with compositions of the invention include any substance that inhibits or prevents the function of cells and/or causes destruction of cells. Examples include, but are not limited to, radioactive isotopes, chemotherapeutic agents, and toxins such as enzymatically active toxins of bacterial, fungal, plant or animal origin, or fragments thereof.

The therapeutic regimens described herein, or any desired treatment regimen can be tested for efficacy using a transgenic animal model which expresses human CD19 antigen in place of native CD19 antigen. Thus, an anti-CD19 antibody treatment regimen can be tested in an animal model to determine efficacy before administration to a human.

Anti-CD19 antibodies, compositions and methods may be practiced to treat B cell diseases, including B cell malignancies. The term “B cell malignancy” includes any malignancy that is derived from a cell of the B cell lineage. Exemplary B cell malignancies include, but are not limited to: B cell subtype non-Hodgkin's lymphoma (NHL) including low grade/follicular, NHL, small lymphocytic (SL) NHL, intermediate grade/follicular NHL, intermediate grade diffuse NHL, high grade immunoblastic NHL, high grade lymphoblastic NHL, high grade small non-cleaved cell NHL; mantle-cell lymphoma, and bulky disease NHL; Burkitt's lymphoma; multiple myeloma; pre-B acute lymphoblastic leukemia and other malignancies that derive from early B cell precursors; common acute lymphocytic leukemia (ALL); chronic lymphocytic leukemia (CLL) including including immunoglobulin-mutated CLL and immunoglobulin-unmutated CLL; hairy cell leukemia; Null-acute lymphoblastic leukemia; Waldenstrom's Macroglobulinemia; diffuse large B cell lymphoma (DLBCL) including germinal center B cell-like (GCB) DLBCL, activated B cell-like (ABC) DLBCL, and type 3 DLBCL; pro-lymphocytic leukemia; light chain disease; plasmacytoma; osteosclerotic myeloma; plasma cell leukemia; monoclonal gammopathy of undetermined significance (MGUS); smoldering multiple myeloma (SMM); indolent multiple myeloma (IMM); Hodgkin's lymphoma including classical and nodular lymphocyte pre-dominant type; lymphoplasmacytic lymphoma (LPL); and marginal-zone lymphoma including gastric mucosal-associated lymphoid tissue (MALT) lymphoma.

In a further embodiment the invention can be employed to treat mature B cell malignancies (i.e., express Ig on the cell surface) including but not limited to follicular lymphoma, mantle-cell lymphoma, Burkitt's lymphoma, multiple myeloma, diffuse large B-cell lymphoma (DLBCL) including germinal center B cell-like (GCB) DLBCL, activated B cell-like (ABC) DLBCL, and type 3 DLBCL, Hodgkin's lymphoma including classical and nodular lymphocyte pre-dominant type, lymphoplasmacytic lymphoma (LPL), marginal-zone lymphoma including gastric mucosal-associated lymphoid tissue (MALT) lymphoma, and chronic lymphocytic leukemia (CLL) including immunoglobulin-mutated CLL and immunoglobulin-unmutated CLL.

Further, CD19 is expressed earlier in B cell development than, for example, CD20, and is therefore particularly suited for treating pre-B cell and immature B cell malignancies (i.e., do not express Ig on the cell surface), for example, in the bone marrow. Illustrative pre-B cell and immature B cell malignancies include but are not limited to acute lymphoblastic leukemia.

In other particular embodiments, the invention can be practiced to treat extranodal tumors.

### 6.22. Anti-CD19 Immunotherapy

In accordance with the present invention “anti-CD19 immunotherapy” encompasses the administration of any of the anti-CD19 antibodies of the invention in accordance with any therapeutic regimen described herein. Anti-CD19 antibodies can be administered as naked antibodies, or immunoconjugates or fusion proteins.

Anti-CD19 immunotherapy encompasses the administration of an anti-CD19 antibody as a single agent therapeutic for the treatment of a B cell malignancy. Anti-CD19 immunotherapy encompasses methods of treating an early stage disease resulting from a B cell malignancy. Anti-CD19 immunotherapy encompasses methods of treating a B cell malignancy wherein an anti-CD19 antibody mediates ADCC. Anti-CD19 immunotherapy encompasses methods of treating a B cell malignancy wherein an anti-CD19 antibody is administered before the patient has received any treatment for the malignancy, whether that therapy is chemotherapy, radio chemical based therapy or surgical therapy.

In one embodiment, a human subject having a B cell malignancy can be treated by administering a human or humanized antibody that may be able to mediate human ADCC. In cases of early stage disease, or single agent therapies, any anti-CD19 antibody that may mediate ADCC can be used in the human subjects (including murine and chimeric antibodies); however, human and humanized antibodies may be preferred.

Antibodies of IgG1 or IgG3 human isotypes are in some cases preferred for therapy. However, the IgG2 or IgG4 human isotypes can be used as well, provided they have the relevant effector function, for example human ADCC. Such effector function can be assessed by measuring the ability of the antibody in question to mediate target cell lysis by effector cells in vitro or in vivo.

In one embodiment, the dose of antibody used should be sufficient to deplete circulating B cells. Progress of the therapy can be monitored in the patient by analyzing blood samples. Other signs of clinical improvement can be used to monitor therapy.

Methods for measuring depletion of B cells that can be used in connection with compositions and methods of the invention are well known in the art and include, but are not limited to the following embodiments. In one embodiment, circulating B cells depletion can be measured with flow cytometry using a reagent other than an anti-CD19 antibody that binds to B cells to define the amount of B cells. In other embodiments, B cell levels in the blood can be monitored using standard serum analysis. In such embodiments, B cell depletion is indirectly measured by defining the amount to an antibody known to be produced by B cells. The level of that antibody is then monitored to determine the depletion and/or functional depletion of B cells. In another embodiment, B cell depletion can be measured by immunochemical staining to identify B cells. In such embodiments, B cells or tissues or serum comprising B cells extracted from a patient can be placed on microscope slides, labeled and examined for presence or absence. In related embodiments, a comparison is made between B cells extracted prior to therapy and after therapy to determine differences in the presence of B cells.

Tumor burden can be measured and used in connection with compositions and methods of the invention. Methods for measuring tumor burden are well known in the art and include, but are not limited to the following embodiments. In certain embodiments, PET scans can be used to measure metabolic activity and identify areas of higher activity which are indicative of tumors. CT scans and MRI can also be used to examine soft tissue for the presence and size of tumors. In other embodiments, bone scan can be used to measure tumor volume and location. In yet other embodiments, tumor burden can be measured by examining the blood flow into and out of a tumor using doppler technology (e.g., ultrasound). In such embodiments, changes in blood flow over time or deviations from normal blood flow in the appropriate tissue of a patient can be used to calculate an estimate to tumor burden. Such methods for measuring tumor burden can be used prior to and following methods of treatment of the invention.

In certain embodiments of methods of the invention B cells are depleted and/or tumor burden is decreased while ADCC function is maintained.

In embodiments of the invention where an anti-CD19 antibody is administered as a single agent therapy, the invention contemplates use of different treatment regimens.

According to certain aspects of the invention, an anti-CD19 antibody used in compositions and methods of the invention, is a naked antibody. In related embodiments, the dose of naked anti-CD19 antibody used is at least about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, or 20.5 mg/kg of body weight of a patient. In certain embodiments, the dose of naked anti-CD19 antibody used is at least about 1 to 10, 5 to 15, 10 to 20, or 15 to 25 mg/kg of body weight of a patient. In certain embodiments, the dose of naked anti-CD19 antibody used is at least about 1 to 20, 3 to 15, or 5 to 10 mg/kg of body weight of a patient. In other embodiments, the dose of naked anti-CD19 antibody used is at least about 5, 6, 7, 8, 9, or 10 mg/kg of body weight of a patient.

In certain embodiments, the dose comprises about 375 mg/m² of anti-CD19 antibody administered weekly for 4 to 8 consecutive weeks. In certain embodiments, the dose is at least about 1, 2, 3, 4, 5, 6, 7, 8, 9, 10, 11, 12, 13, 14, or 15 mg/kg of body weight of the patient administered weekly for 4 to 8 consecutive weeks.

The exemplary doses of anti-CD19 antibody described above can be administered as described in Section 6.18.3. In one embodiment, the above doses are single dose injections. In other embodiments, the doses are administered over a period of time. In other embodiments, the doses are administered multiple times over a period of time. The period of time may be measured in days, weeks, or months. Multiple doses of an anti-CD19 antibody can be administered at intervals suitable to achieve a therapeutic benefit while balancing toxic side effects. For example, where multiple doses are used, it may be preferred to time the intervals to allow for recovery of the patient's monocyte count prior to the repeat treatment with antibody. This dosing regimen will optimize the efficiency of treatment, since the monocyte population reflects ADCC function in the patient.

In certain embodiments, compositions of the invention are administered to a human patient as long as the patient is responsive to therapy. In other embodiments, compositions of the invention are administered to a human patient as long as the patient's disease does not progress. In related embodiments, compositions of the invention are administered to a human patient until a patient's disease does not progress or has not progressed for a period of time, then the patient is not administered compositions of the invention unless the disease reoccurs or begins to progress again. For example, a patient can be treated with any of the above doses for about 4 to 8 weeks, during which time the patient is monitored for disease progression. If disease progression stops or reverses, then the patient will not be administered compositions of the invention until that patient relapses, i.e., the disease being treated reoccurs or progresses. Upon this reoccurrence or progression, the patient can be treated again with the same dosing regimen initially used or using other doses described above.

In certain embodiments, compositions of the invention can be administered as a loading dose followed by multiple lower doses (maintenance doses) over a period of time. In such embodiments, the doses may be timed and the amount adjusted to maintain effective B cell depletion. In certain embodiments, the loading dose is about 10, 11, 12, 13, 14, 15, 16, 17, or 18 mg/kg of patient body weight and the maintenance dose is at least about 5 to 10 mg/kg of patient body weight. In other embodiments, the maintenance dose is administered at intervals of every 7, 10, 14 or 21 days. The maintenance doses can be continued indefinitely, until toxicity is present, until platelet count decreases, until there is no disease progression, until the patient exhibits immunogenicity, or until disease progresses to a terminal state. In yet other embodiments, compositions of the invention are administered to a human patient until the disease progresses to a terminal stage.

In embodiments of the invention where circulating monocyte levels of a patient are monitored as part of a treatment regimen, doses of anti-CD19 antibody administered may be spaced to allow for recovery of monocyte count. For example, a composition of the invention may be administered at intervals of every 8, 9, 10, 11, 12, 13, 14, 15, 16, 17, 18, 19, 20, 21, 22, 23, 24, 25, 26, 27, 28, 29, or 30 days.

In embodiments of the invention where an anti-CD19 antibody is conjugated to or administered in conjunction with a toxin, one skilled in the art will appreciate that the dose of anti-CD19 antibody can be adjusted based on the toxin dose and that the toxin dose will depend on the specific type of toxin being used. Typically, where a toxin is used, the dose of anti-CD19 antibody will be less than the dose used with a naked anti-CD19 antibody. The appropriate dose can be determined for a particular toxin using techniques well known in the art. For example, a dose range study can be conducted to determine the maximum tolerated dose of anti-CD19 antibody when administered with or conjugated to a toxin.

In embodiments of the invention where an anti-CD19 antibody is conjugated to or administered in conjunction with a radiotherapeutic agent, the dose of the anti-CD19 antibody will vary depending on the radiotherapeutic used. In certain embodiments, a two step process is used. First, the human patient is administered a composition comprising a naked anti-CD19 antibody and about 6, 7, 8, 9, or 10 days later a small amount of the radiotherapeutic is administered. Second, once the tolerance, distribution, and clearance of the low dose therapy has been determined, the patient is administered a dose of the naked anti-CD19 antibody followed by a therapeutic amount of the radiotherapeutic is administered. Such treatment regimens are similar to those approved for treatment of non-Hodgkin's lymphoma using ZEVALIN™ (Indium labeled anti-CD20 mAb) (Biogen Idec) or BEXXAR™ (GSK, Coulter Pharmaceutical).

6.23. Combination with Chemotherapeutic Agents

Anti-CD19 immunotherapy (using naked antibody, immunoconjugates, or fusion proteins) can be used in conjunction with other therapies including but not limited to, chemotherapy, radioimmunotherapy (RIT), chemotherapy and external beam radiation (combined modality therapy, CMT), or combined modality radioimmunotherapy (CMRIT) alone or in combination, etc. In certain embodiments, an anti-CD19 antibody therapy of the present invention can be administered in conjunction with CHOP (Cyclophosphamide-Hydroxydoxorubicin-Oncovin (vincristine)-Prednisolone), the most common chemotherapy regimen for treating non-Hodgkin's lymphoma. As used herein, the term “administered in conjunction with” means that an anti-CD19 immunotherapy can be administered before, during, or subsequent to the other therapy employed.

In certain embodiments, an anti-CD19 immunotherapy is in conjunction with a cytotoxic radionuclide or radiotherapeutic isotope. For example, an alpha-emitting isotope such as ²²⁵Ac, ²²⁴Ac, ²¹¹At, ²¹²Bi, ²¹³Bi, ²¹²Pb, ²²⁴Ra, or ²²³Ra. The cytotoxic radionuclide may also be a beta-emitting isotope such as ¹⁸⁶Re, ¹⁸⁸Re, ⁹⁰Y, ¹³¹I, ⁶⁷Cu, ¹⁷⁷Lu, ¹⁵³Sm, ¹⁶⁶Ho, or ⁶⁴Cu. Further, the cytotoxic radionuclide may emit Auger and low energy electrons and include the isotopes ¹²⁵I, ¹²³I or ⁷⁷Br. In other embodiments the isotope may be ¹⁹⁸Au, ³²P, and the like. In certain embodiments, the amount of the radionuclide administered to the subject is between about 0.001 mCi/kg and about 10 mCi/kg.

In some embodiments, the amount of the radionuclide administered to the subject is between about 0.1 mCi/kg and about 1.0 mCi/kg. In other embodiments, the amount of the radionuclide administered to the subject is between about 0.005 mCi/kg and 0.1 mCi/kg.

In certain embodiments, an anti-CD19 immunotherapy is in conjunction with a chemical toxin or chemotherapeutic agent. The chemical toxin or chemotherapeutic agent may be selected from the group consisting of an enediyne such as calicheamicin and esperamicin; duocarmycin, methotrexate, doxorubicin, melphalan, chlorambucil, ARA-C, vindesine, mitomycin C, cis-platinum, etoposide, bleomycin and 5-fluorouracil.

Suitable chemical toxins or chemotherapeutic agents that can be used in combination therapies with an anti-CD19 immunotherapy include members of the enediyne family of molecules, such as calicheamicin and esperamicin. Chemical toxins can also be taken from the group consisting of duocarmycin (see, e.g., U.S. Pat. No. 5,703,080 and U.S. Pat. No. 4,923,990), methotrexate, doxorubicin, melphalan, chlorambucil, ARA-C, vindesine, mitomycin C, cis-platinum, etoposide, bleomycin and 5-fluorouracil. Examples of chemotherapeutic agents also include Adriamycin, Doxorubicin, 5-Fluorouracil, Cytosine arabinoside (“Ara-C”), Cyclophosphamide, Thiotepa, Taxotere (docetaxel), Busulfan, Cytoxin, Taxol, Methotrexate, Cisplatin, Melphalan, Vinblastine, Bleomycin, Etoposide, Ifosfamide, Mitomycin C, Mitoxantrone, Vincreistine, Vinorelbine, Carboplatin, Teniposide, Daunomycin, Caminomycin, Aminopterin, Dactinomycin, Mitomycins, Esperamicins (see, U.S. Pat. No. 4,675,187), Melphalan and other related nitrogen mustards.

In other embodiments, for example, “CVB” (1.5 g/m² cyclophosphamide, 200-400 mg/m² etoposide, and 150-200 mg/m² carmustine) can be used in combination therapies of the invention. CVB is a regimen used to treat non-Hodgkin's lymphoma. Patti et al., Eur. J. Haematol. 51:18 (1993). Other suitable combination chemotherapeutic regimens are well-known to those of skill in the art. See, for example, Freedman et al., “Non-Hodgkin's Lymphomas,” in CANCER MEDICINE, VOLUME 2, 3rd Edition, Holland et al. (eds.), pp. 2028-2068 (Lea & Febiger 1993). As an illustration, first generation chemotherapeutic regimens for treatment of intermediate-grade non-Hodgkin's lymphoma include C-MOPP (cyclophosphamide, vincristine, procarbazine and prednisone) and CHOP (cyclophosphamide, doxorubicin, vincristine, and prednisone). A useful second generation chemotherapeutic regimen is m-BACOD (methotrexate, bleomycin, doxorubicin, cyclophosphamide, vincristine, dexamethasone and leucovorin), while a suitable third generation regimen is MACOP-B (methotrexate, doxorubicin, cyclophosphamide, vincristine, prednisone, bleomycin and leucovorin). Additional useful drugs include phenyl butyrate and brostatin-1. In a multimodal therapy, both chemotherapeutic drugs and cytokines are co-administered with an antibody, immunoconjugate or fusion protein according to the present invention. The cytokines, chemotherapeutic drugs and antibody, immunoconjugate or fusion protein can be administered in any order, or together.

Other toxins that may be used in compositions and methods of the invention include poisonous lectins, plant toxins such as ricin, abrin, modeccin, botulina and diphtheria toxins. Of course, combinations of the various toxins could also be coupled to one antibody molecule thereby accommodating variable cytotoxicity. Illustrative of toxins which are suitably employed in combination therapies of the invention are ricin, abrin, ribonuclease, DNase I, Staphylococcal enterotoxin-A, pokeweed antiviral protein, gelonin, diphtherin toxin, Pseudomonas exotoxin, and Pseudomonas endotoxin. See, for example, Pastan et al., Cell 47:641 (1986), and Goldenberg et al., Cancer Journal for Clinicians 44:43 (1994). Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleuritesfordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232 published Oct. 28, 1993.

Suitable toxins and chemotherapeutic agents are described in REMINGTON′S PHARMACEUTICAL SCIENCES, 19th Ed. (Mack Publishing Co. 1995), and in GOODMAN AND GILMAN′S THE PHARMACOLOGICAL BASIS OF THERAPEUTICS, 7th Ed. (MacMillan Publishing Co. 1985). Other suitable toxins and/or chemotherapeutic agents are known to those of skill in the art.

An anti-CD19 immunotherapy of the present invention may also be in conjunction with a prodrug-activating enzyme which converts a prodrug (e.g., a peptidyl chemotherapeutic agent, see, WO81/01145) to an active anti-cancer drug. See, for example, WO 88/07378 and U.S. Pat. No. 4,975,278. The enzyme component of such combinations includes any enzyme capable of acting on a prodrug in such a way so as to convert it into its more active, cytotoxic form. The term “prodrug” as used in this application refers to a precursor or derivative form of a pharmaceutically active substance that is less cytotoxic to tumor cells compared to the parent drug and is capable of being enzymatically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al. (ed.), pp. 247-267, Humana Press (1985). Prodrugs that can be used in combination with anti-CD19 antibodies include, but are not limited to, phosphate-containing prodrugs, thiophosphate-containing prodrugs, sulfate-containing prodrugs, peptide-containing prodrugs, D-amino acid-modified prodrugs, glycosylated prodrugs, α-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

In certain embodiments, administration of compositions and methods of the invention may enable the postponement of toxic therapy and may help avoid unnecessary side effects and the risks of complications associated with chemotherapy and delay development of resistance to chemotherapy. In certain embodiments, toxic therapies and/or resistance to toxic therapies is delayed in patients administered compositions and methods of the invention delay for up to about 6 months, 1, 2, 3, 4, 5, 6, 7, 8, 9, or 10 years.

6.24. Combination with Therapeutic Antibodies

An anti-CD19 immunotherapy described herein may be administered in combination with other antibodies, including, but not limited to, anti-CD20 mAb, anti-CD52 mAb, anti-CD22 antibody, and anti-CD20 antibodies, such as RITUXAN™ (C2B8; RITUXIMAB™; IDEC Pharmaceuticals). Other examples of therapeutic antibodies that can be used in combination with antibodies of the invention or used in compositions of the invention include, but are not limited to, HERCEPTIN™ (Trastuzumab; Genentech), MYLOTARG™ (Gemtuzumab ozogamicin; Wyeth Pharmaceuticals), CAMPATH™ (Alemtuzumab; Berlex), ZEVALIN™ (Ipritumomab tiuxetan; Biogen Idec), BEXXAR™ (Tositumomab; GlaxoSmithKline Corixa), ERBITUX™ (Cetuximab; Imclone), and AVASTIN™ (Bevacizumab; Genentech).

An anti-CD19 immunotherapy described herein may be administered in combination with an antibody specific for an Fc receptor selected from the group consisting of FcγRI, FcγRIIA, FcγRIIB, FcγRIII and/or FcγRIV. In a specific embodiment, an anti-CD19 immunotherapy described herein may be administered in combination with an antibody specific for FcγRIIB. Anti-FcγRIIB antibodies suitable for this purpose have been described in US Patent Application Publication No. 2004185045, PCT Publication Nos. WO05051999A, WO05018669 and WO04016750.

In certain embodiments, an anti-CD19 and an anti-CD20 and/or an anti-CD22 mAb and/or an anti-CD52 mAb can be administered, optionally in the same pharmaceutical composition, in any suitable ratio. To illustrate, the ratio of the anti-CD19 and anti-CD20 antibody can be a ratio of about 1000:1, 500:1, 250:1, 100:1, 90:1, 80:1, 70:1, 60;1, 50:1, 40:1, 30:1. 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:30, 1:40, 1:50, 1:60, 1:70, 1:80, 1:90. 1:100, 1:250, 1:500 or 1:1000 or more. Likewise, the ratio of the anti-CD19 and anti-CD22 antibody can be a ratio of about 1000:1, 500:1, 250:1, 100:1, 90:1, 80:1, 70:1, 60;1, 50:1, 40:1, 30:1. 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:30, 1:40, 1:50, 1:60, 1:70, 1:80, 1:90. 1:100, 1:250, 1:500 or 1:1000 or more. Similarly, the ratio of the anti-CD19 and anti-CD52 antibody can be a ratio of about 1000:1, 500:1, 250:1, 100:1, 90:1, 80:1, 70:1, 60;1, 50:1, 40:1, 30:1. 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:30, 1:40, 1:50, 1:60, 1:70, 1:80, 1:90. 1:100, 1:250, 1:500 or 1:1000 or more.

6.25. Combination Compounds that Enhance Monocyte or Macrophage Function

In certain embodiments of methods of the invention, a compound that enhances monocyte or macrophage function (e.g., at least about 25%, 50%, 75%, 85%, 90%, 95% or more) can be used in conjunction with an anti-CD19 immunotherapy. Such compounds are known in the art and include, without limitation, cytokines such as interleukins (e.g., IL-12), and interferons (e.g., alpha or gamma interferon).

The compound that enhances monocyte or macrophage function or enhancement can be formulated in the same pharmaceutical composition as the antibody, immunoconjugate or antigen-binding fragment. When administered separately, the antibody/fragment and the compound can be administered concurrently (within a period of hours of each other), can be administered during the same course of therapy, or can be administered sequentially (i.e., the patient first receives a course of the antibody/fragment treatment and then a course of the compound that enhances macrophage/monocyte function or vice versa). In such embodiments, the compound that enhances monocyte or macrophage function is administered to the human subject prior to, concurrently with, or following treatment with other therapeutic regimens and/or compositions of the invention. In one embodiment, the human subject has a blood leukocyte, monocyte, neutrophil, lymphocyte, and/or basophil count that is within the normal range for humans. Normal ranges for human blood leukocytes (total) is about 3.5-about 10.5 (10⁹/L). Normal ranges for human blood neutrophils is about 1.7-about 7.0 (10⁹/L), monocytes is about 0.3-about 0.9 (10⁹/L), lymphocytes is about 0.9-about 2.9 (10⁹/L), basophils is about 0-about 0.3 (10⁹/L), and eosinophils is about 0.05-about 0.5 (10⁹/L). In other embodiments, the human subject has a blood leukocyte count that is less than the normal range for humans, for example at least about 0.01, 0.05, 0.1, 0.2, 0.3, 0.4, 0.5, 0.6, 0.7, or 0.8 (10⁹/L) leukocytes.

This embodiment of the invention can be practiced with the antibodies, immunoconjugates or antibody fragments of the invention or with other antibodies known in the art and is particularly suitable for subjects that are resistant to anti-CD22, anti-CD52 and/or anti-CD20 antibody therapy (for example, therapy with existing antibodies such as C2B8), subjects that are currently being or have previously been treated with chemotherapy, subjects that have had a relapse in a B cell disorder, subjects that are immunocompromised, or subjects that otherwise have an impairment in macrophage or monocyte function. The prevalence of patients that are resistant to therapy or have a relapse in a B cell disorder may be attributable, at least in part, to an impairment in macrophage or monocyte function. Thus, the invention provides methods of enhancing ADCC and/or macrophage and/or monocyte function to be used in conjunction with the methods of administering anti-CD19 antibodies and antigen-binding fragments.

6.26. Combination with Immunoregulatory Agents

An anti-CD19 immunotherapy of the invention may also be used in conjunction with an immunoregulatory agent. In this approach, a chimeric, human or humanized anti-CD19 antibody can be used. The term “immunoregulatory agent” as used herein for combination therapy refers to substances that act to suppress, mask, or enhance the immune system of the host. This would include substances that suppress cytokine production, downregulate or suppress self-antigen expression, or mask the MHC antigens. Examples of such agents include 2-amino-6-aryl-5-substituted pyrimidines (see, U.S. Pat. No. 4,665,077), azathioprine (or cyclophosphamide, if there is an adverse reaction to azathioprine); bromocryptine; glutaraldehyde (which masks the MHC antigens, as described in U.S. Pat. No. 4,120,649); anti-idiotypic antibodies for MHC antigens and MHC fragments; cyclosporin A; steroids such as glucocorticosteroids, e.g., prednisone, methylprednisolone, and dexamethasone; cytokine or cytokine receptor antagonists including anti-interferon-γ, -β, or -α antibodies; anti-tumor necrosis factor-α antibodies; anti-tumor necrosis factor-β antibodies; anti-interleukin-2 antibodies and anti-IL-2 receptor antibodies; anti-L3T4 antibodies; heterologous anti-lymphocyte globulin; pan-T antibodies, for example anti-CD3 or anti-CD4/CD4a antibodies; soluble peptide containing a LFA-3 binding domain (WO 90/08187 published Jul. 26, 1990); streptokinase; TGF-β; streptodornase; RNA or DNA from the host; FK506; RS-61443; deoxyspergualin; rapamycin; T-cell receptor (U.S. Pat. No. 5,114,721); T-cell receptor fragments (Offner et al., Science 251:430-432 (1991); WO 90/11294; and WO 91/01133); and T-cell receptor antibodies (EP 340,109) such as T10B9. Examples of cytokines include, but are not limited to lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α; mullerian-inhibiting substance; mouse gonadotropin-associated peptide; inhibin; activin; vascular endothelial growth factor; integrin; thrombopoiotin (TPO); nerve growth factors such as NGF-α; platelet-growth factor; transforming growth factors (TGFs) such as TGF-α and TGF-β; insulin-like growth factor-I and -II; erythropoietin (EPO); osteoinductive factors; interferons; colony stimulating factors (CSFs) such as macrophage-CSF (M-CSF); granulocyte-macrophage-CgP (GM-CSP); and granulocyte-CSF (G-CSF); interleukins (ILs) such as IL-1, IL-1a, IL-2, IL-3, IL-4, IL-5, IL-6, IL-7, IL-8, IL-9, IL-1 I, IL-12, IL-15; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native sequence cytokines. In certain embodiments, the methods further include administering to the subject one or more immunomodulatory agents, for example a cytokine. Suitable cytokines may be selected from the group consisting of interleukin-1 (IL-1), IL-2, IL-3, IL-12, IL-15, IL-18, G-CSF, GM-CSF, thrombopoietin, and y interferon.

These immunoregulatory agents are administered at the same time or at separate times from anti-CD19 antibodies. The preferred immunoregulatory agent will depend on many factors, including the type of disorder being treated, as well as the patient's history, but the agent frequently may be selected from cyclosporin A, a glucocorticosteroid (for example prednisone or methylprednisolone), OKT-3 monoclonal antibody, azathioprine, bromocryptine, heterologous anti-lymphocyte globulin, or a mixture thereof.

6.27. Combination with Other Therapeutic Agents

Agents that act on the tumor neovasculature can also be used in conjunction with anti-CD19 immunotherapy and include tubulin-binding agents such as combrestatin A4 (Griggs et al., Lancet Oncol. 2:82, (2001)) and angiostatin and endostatin (reviewed in Rosen, Oncologist 5:20 (2000), incorporated by reference herein) Immunomodulators suitable for use in combination with anti-CD19 antibodies include, but are not limited to, α-interferon, γ-interferon, and tumor necrosis factor alpha (TNFα). In certain embodiments, the therapeutic agents used in combination therapies using compositions and methods of the invention are peptides.

In certain embodiments, an anti-CD19 immunotherapy is in conjunction with one or more calicheamicin molecules. The calicheamicin family of antibiotics are capable of producing double-stranded DNA breaks at sub-picomolar concentrations. Structural analogues of calicheamicin which may be used include, but are not limited to, γ1^{I}, γ2^{I}, γ3^{I}, N-acetyl-γ1^{I}, PSAG and 011 Hinman et al., Cancer Research 53:3336-3342 (1993) and Lode et al., Cancer Research 58: 2925-2928 (1998)).

A fusion protein comprising an anti-CD19 antibody and a cytotoxic agent may also be made, e.g., by recombinant techniques or peptide synthesis.

In yet another embodiment, an anti-CD19 antibody may be conjugated to a “receptor” (such as streptavidin) for utilization in tumor pretargeting wherein the antagonist-receptor conjugate is administered to the patient, followed by removal of unbound conjugate from the circulation using a clearing agent and then administration of a “ligand” (e.g., avidin) which is conjugated to a therapeutic agent (e.g., a radionucleotide).

In certain embodiments, a treatment regimen includes compounds that mitigate the cytotoxic effects of an anti-CD19 antibody composition. Such compounds include analgesics (e.g., acetaminophen), bisphosphonates, antihistamines (e.g., chlorpheniramine maleate), and steroids (e.g., dexamethasone, retinoids, deltoids, betamethasone, cortisol, cortisone, prednisone, dehydrotestosterone, glucocorticoids, mineralocorticoids, estrogen, testosterone, progestins).

In certain embodiments, the therapeutic agent used in combination with an anti-CD19 immunotherapy is a small molecule (i.e., inorganic or organic compounds having a molecular weight of less than about 2500 daltons). For example, libraries of small molecules may be commercially obtained from Specs and BioSpecs B.V. (Rijswijk, The Netherlands), Chembridge Corporation (San Diego, Calif.), Comgenex USA Inc. (Princeton, N.J.), and Maybridge Chemicals Ltd. (Cornwall PL34 OHW, United Kingdom).

In certain embodiments an anti-CD19 immunotherapy can be administered in combination with an anti-bacterial agent. Non-limiting examples of anti-bacterial agents include proteins, polypeptides, peptides, fusion proteins, antibodies, nucleic acid molecules, organic molecules, inorganic molecules, and small molecules that inhibit and/or reduce a bacterial infection, inhibit and/or reduce the replication of bacteria, or inhibit and/or reduce the spread of bacteria to other cells or subjects. Specific examples of anti-bacterial agents include, but are not limited to, antibiotics such as penicillin, cephalosporin, imipenem, axtreonam, vancomycin, cycloserine, bacitracin, chloramphenicol, erythromycin, clindamycin, tetracycline, streptomycin, tobramycin, gentamicin, amikacin, kanamycin, neomycin, spectinomycin, trimethoprim, norfloxacin, rifampin, polymyxin, amphotericin B, nystatin, ketocanazole, isoniazid, metronidazole, and pentamidine.

In certain embodiments an anti-CD19 immunotherapy can be administered in combination with an anti-fungal agent. Specific examples of anti-fungal agents include, but are not limited to, azole drugs (e.g., miconazole, ketoconazole (NIZORAL®), caspofungin acetate (CANCIDAS®), imidazole, triazoles (e.g., fluconazole (DIFLUCAN®)), and itraconazole (SPORANOX®), polyene (e.g., nystatin, amphotericin B (FUNGIZON®, amphotericin B lipid complex (“ABLC”) (ABELCET®), amphotericin B colloidal dispersion (“ABCD”) (AMPHOTEC®), liposomal amphotericin B (AMBISONE®)), potassium iodide (KI), pyrimidine (e.g., flucytosine (ANCOBON®), and voriconazole (VFEND®)). Administration of anti-bacterial and anti-fungal agents can mitigate the effects or escalation of infectious disease that may occur in methods of the invention where a patient's B cells are significantly depleted.

In certain embodiments of the invention, an anti-CD19 immunotherapy can be administered in combination with one or more of the agents described above to mitigate the toxic side effects that may accompany administration of compositions of the invention. In other embodiments, an anti-CD19 immunotherapy can be administered in combination with one or more agents that are well known in the art for use in mitigating the side effects of antibody administration, chemotherapy, toxins, or drugs.

In certain embodiments of the invention where an anti-CD19 immunotherapy is administered to treat multiple myeloma, compositions of the invention may be administered in combination with or in treatment regimens with high-dose chemotherapy (melphalan, melphalan/prednisone (MP), vincristine/doxorubicin/dexamethasone (VAD), liposomal doxorubicin/vincristine, dexamethasone (DVd), cyclophosphamide, etoposide/dexamethasone/cytarabine, cisplatin (EDAP)), stem cell transplants (e.g., autologous stem cell transplantation or allogeneic stem cell transplantation, and/or mini-allogeneic (non-myeloablative) stem cell transplantation), radiation therapy, steroids (e.g., corticosteroids, dexamethasone, thalidomide/dexamethasone, prednisone, melphalan/prednisone), supportive therapy (e.g., bisphosphonates, growth factors, antibiotics, intravenous immunoglobulin, low-dose radiotherapy, and/or orthopedic interventions), THALOMID™ (thalidomide, Celgene), and/or VELCADE™ (bortezomib, Millennium).

In embodiments of the invention where an anti-CD19 immunotherapy is administered in combination with another antibody or antibodies and/or agent, the additional antibody or antibodies and/or agents can be administered in any sequence relative to the administration of the antibody of this invention. For example, the additional antibody or antibodies can be administered before, concurrently with, and/or subsequent to administration of an anti-CD19 antibody or immunoconjugate to the human subject. The additional antibody or antibodies can be present in the same pharmaceutical composition as an antibody of the invention, and/or present in a different pharmaceutical composition. The dose and mode of administration of an antibody of this invention and the dose of the additional antibody or antibodies can be the same or different, in accordance with any of the teachings of dosage amounts and modes of administration as provided in this application and as are well known in the art.

### 6.28. Kits

The invention provides a pharmaceutical pack or kit comprising one or more containers filled with a composition of the invention for the prevention, treatment, management or amelioration of a B cell malignancy, or one or more symptoms thereof, potentiated by or potentiating a B cell malignancy.

In one embodiment, a container filled with a liquid formulation of the invention is a pre-filled syringe. Any pre-filled syringe known to one of skill in the art may be used in combination with a liquid formulation of the invention. Pre-filled syringes that may be used are described in, for example, but not limited to, PCT Publications WO05032627, WO08094984, WO9945985, WO03077976, U.S. Pat. No. 6,792,743, U.S. Pat. No. 5,607,400, U.S. Pat. No. 5,893,842, U.S. Pat. No. 7,081,107, U.S. Pat. No. 7,041,087, U.S. Pat. No. 5,989,227, U.S. Pat. No. 6,807,797, U.S. Pat. No. 6,142,976, U.S. Pat. No. 5,899,889, US Patent Publications US20070161961A1, US20050075611A1, US20070092487A1, US20040267194A1, US20060129108A1. Pre-filled syringes may be made of various materials. In one embodiment a pre-filled syringe is a glass syringe. In another embodiment a pre-filled syringe is a plastic syringe. One of skill in the art understands that the nature and/or quality of the materials used for manufacturing the syringe may influence the stability of a protein formulation stored in the syringe. For example, it is understood that silicon based lubricants deposited on the inside surface of the syringe chamber may affect particle formation in the protein formulation. In one embodiment, a pre-filled syringe comprises a silicone based lubricant. In one embodiment, a pre-filled syringe comprises baked on silicone. In another embodiment, a pre-filled syringe is free from silicone based lubricants. One of skill in the art also understands that small amounts of contaminating elements leaching into the formulation from the syringe barrel, syringe tip cap, plunger or stopper may also influence stability of the formulation. For example, it is understood that tungsten introduced during the manufacturing process may adversely affect formulation stability. In one embodiment, a pre-filled syringe may comprise tungsten at a level above 500 ppb. In another embodiment, a pre-filled syringe is a low tungsten syringe. In another embodiment, a pre-filled syringe may comprise tungsten at a level between about 500 ppb and about 10 ppb, between about 400 ppb and about 10 ppb, between about 300 ppb and about 10 ppb, between about 200 ppb and about 10 ppb, between about 100 ppb and about 10 ppb, between about 50 ppb and about 10 ppb, between about 25 ppb and about 10 ppb. In another embodiment, the syringe may be a tungsten-free syringe. In another embodiment, the syringe is the tungsten-free “ultra-100” syringe. In another embodiment the syringe is a Clearject™ (Geresheimer, AG, Germany) or InJentle™ (SCHOTT Pharmaceutical Packaging, Germany) syringe.

The present invention provides kits that can be used in the above-described methods. In one embodiment, a kit comprises a composition of the invention, in one or more containers. In another embodiment, a kit comprises a composition of the invention, in one or more containers, and one or more other prophylactic or therapeutic agents useful for the prevention, management or treatment of a B cell malignancy, or one or more symptoms thereof, potentiated by or potentiating a B cell malignancy in one or more other containers. The kit may further comprise instructions for preventing, treating, managing or ameliorating a B cell malignancy, as well as side effects and dosage information for method of administration. Optionally associated with such container(s) can be a notice in the form prescribed by a governmental agency regulating the manufacture, use or sale of pharmaceuticals or biological products, which notice reflects approval by the agency of manufacture, use or sale for human administration.

### 7. EXAMPLES

The invention is now described with reference to the following examples. These examples are provided for the purpose of illustration only and the invention should in no way be construed as being limited to these examples but rather should be construed to encompass any and all variations which become evident as a result of the teachings provided herein.

### 7.1. Formulation Development

The following section describes the development of formulations comprising an anti-human CD19 antibody. Unless stated otherwise, experimental results presented here were generated using the 16C4 anti-human CD19 antibody comprising the heavy chain variable region having the amino acid sequence of SEQ ID NO:104, a light chain variable region having the amino acid sequence of SEQ ID NO:111 and an Fc region having complex N-glycoside-linked sugar chains in which fucose is not bound to N-acetylglucosamine in the reducing end in the sugar chain (see, U.S. patent application Ser. No. 11/852,106 filed on Sep. 7, 2007, the disclosure of which is incorporated herein in its entirety for all purposes).

### 7.1.1. Construction, Expression and Binding Characteristics of Humanized Anti-CD19 Antibodies

The construction, expression and characterization of humanized afucosylated anti-CD19 antibodies, e.g. 16C4-aFuc (also referred to as 551 antibody), was described in U.S. patent application Ser. No. 11/852,106 filed on Sep. 7, 2007.

### 7.1.2. Experimental Methods

Purified anti-human CD19 antibody may be generated following standard industrial scale protocols described herein. Protein concentration may be estimated from optical density measurement at 280 nm.

Purified anti-human CD19 antibody is nanofiltered using a Planova 20N filter to remove particulate matter. Anti-human CD19 antibody formulations is prepared using Tangential Flow Filtration (TFF). The nanofiltered anti-human CD19 antibody is concentrated to approximately 25 mg/ml on a Millipore Labscale TFF device. The anti-human CD19 antibody is then 5× diafiltered into the appropriate buffer (e.g., 10 mM histidine-HCL (pH 6.0), 75 mM NaCl). Once the buffer exchange is complete, the antibody may be further concentrated to approximately 150 mg/ml. Excipients are introduced by spiking the antibody preparation with the appropriate concentrated stock solutions. For example, a final concentration of 4% Trehalose is achieved by adding 11 ml of 10 mM histidine-HCl, 75 mM NaCl, 40% Trehalose (pH 6.0) to every 100 ml of antibody preparation. Multiple excipients may be introduced in consecutive steps. For example, a final concentration of 0.02% Polysorbate 80 may be introduced after the addition of Trehalose by diluting 100 fold a 10 mM histidine-HCl (pH 6.0), 75 mM NaCl, 4% Trehalose, 2% Polysorbate 80 stock solution with the Trehalose containing antibody preparation. The final antibody concentration is adjusted to the desired level (e.g. 10 mg/ml) with the final formulation buffer (e.g., 10 mM histidine-HCl (pH 6.0), 75 mM NaCl. 4% Trehalose).

The following section describes methods that may be used to characterize liquid antibody formulations, for example a formulation comprising 10 mg/ml anti-CD19 antibody in 10 mM Histidine (pH 6.0), 75 mM NaCl, 4% Trehalose in a sterile aqueous solution.

Formulation stability is tested by analyzing the physical properties of single dose aliquots stored for extended periods of time. Some aliquots are stored under temperatures recommended for clinical storage (5° C.). Other aliquots are stored under elevated temperature (25° C. or 40° C.) to simulate the effects of very long term storage. Test aliquots may be stored in vials (e.g., glass or plastic vials), syringes or any other suitable vessel. Test aliquots may also be subjected to stress conditions, for example, but not limited to shaking, freeze-thawing. Additional storage conditions that may affect stability of a formulation include, but are not limited to, light intensity, light wavelength, humidity, vial composition, and stopper composition. The effect of these parameters on formulation stability may also be determined using the methods described herein.

Reversed Phase High Performance Liquid Chromatography (RP-HPLC) may also be used to determine the amount of antibody fragments in the formulation. RP-HPLC is performed using the Agilent 1100 Series High Performance Liquid Chromatography (HPLC) system. Samples are analysed on a PLRP-S (8 um, 4000 A, 2.0×150 mm) column from Michrom Bioresources. Protein elution profile is determined by following the eluate's optical density at 280 nm. Data analysis may be performed using ChemStation (Agilent) auto integration parameters.

### 7.1.2.1. Size Exclusion Chromatography (SEC)

Size exclusion chromatography may be performed to analyze the antibody formulation for the presence of antibody aggregates and fragments. The test samples are injected onto a high resolution size exclusion column (e.g., G3000 SW_{XL} 5 μm, 300 Å, 7.8×300 mm, TosoHaas). The mobile phase is 0.1 M di-sodium phosphate, 0.1 M sodium sulphate and 0.05% sodium azide (pH 6.7), running isocratically at a flow rate of 0.25-1.0 mL/min. Eluted protein may be detected by UV absorbance at 280 nm and collected for further characterization. The relative amount of any protein species detected is reported as the area percent of the product peak as compared to the total area of all other detected peaks excluding the initial excluded volume peak. Peaks eluting earlier than the antibody monomer peak are recorded in the aggregate percentile, while peaks eluting later than the antibody monomer peak, but earlier than the buffer peak, are recorded in the fragment percentile. The hydrodynamic radius and molecular weight of the individual peaks may be obtained with a coupled multiangle light scattering detector.

SEC may be used to monitor antibody aggregate formation and antibody fragmentation in formulations stored for extended time periods (e.g., multiple measurements performed over 9 months). The formulation may be stored at different temperature ranges (e.g., 2-8° C., 20-24° C. and 38-42° C.). Temperature ranges above the proposed clinical storage temperature (2-8° C.) are used to stress the formulation with the goal of simulating the effects of storage beyond 9 months. The ratio of fragments and aggregates is expected to increase over time; this increase is likely to be accelerated at elevated temperatures. A finding that fragmentation and aggregation rates are constant within each temperature range would show that higher storage temperatures accurately simulate an accelerated time scale.

The logarithm of the estimated rates of fragmentation/aggregation (log(rate)) may also be determined A finding that the log(rate) shows a linear dependence to the reciprocal of the storage temperature (1/T (K⁻¹) would allow the investigator to predict the rate of aggregation/fragmentation of the formulation at any temperature or, more importantly, the formulation characteristics at any time at a given temperature.

In situations where the chromatography peaks corresponding to aggregates and fragments are not be sufficiently distinct from each other, or from the monomer peak (e.g., at low relative levels of aggregates/fragments), SEC may not serve as an accurate measure of fragmentation/aggregation.

Alternatively, SEC may be performed using the Agilent 1100 Series High Performance Liquid Chromatography (HPLC) system as follows. Samples are diluted to 10 mg/ml. 25 μl diluted sample containing 250 ug protein is injected onto a TSK-Gel 3000 column (size 7.8 mm×30.0 cm.; Tosoh Biosciences Corporation). Protein elution profile is determined by following the eluate's optical density at 280 nm. Data analysis may be performed using ChemStation (Agilent) auto integration parameters.

### 7.1.2.2. Analytical Ultracentrifugation

Analytical ultracentrifugation (AUC) may also be used to characterize the antibody formulation for the presence of antibody aggregates and fragments. AUC is an orthogonal technique which determines the sedimentation coefficients (reported in Svedberg, S) of macromolecules in a liquid sample. Like SEC, AUC is capable of separating and detecting antibody fragments/aggregates from monomers and is further able to provide information on molecular mass. Compared to SEC, AUC eliminates the possibility of aggregate loss due to solid-phase interaction and is better able to resolve differing species of a given macromolecule.

Sedimentation velocity experiments may be performed using an analytical ultracentrifuge, for example, Beckman Optima XL-A. Test samples are diluted to an antibody concentration of 0.5 mg/ml with reference buffer (e.g., 20 mM citric acid, 100 mM NaCl, 1.5% mannitol, 50 μM diethylenetriamine-pentaacetic acid, 0.02% Polysorbate 80, pH 6.0). 415 μl of the diluted antibody sample and 412 μl of the reference buffer is loaded into a 12 mm centrifuge cell in the sample and reference channels, respectively. Loaded cells are placed into an AN-50Ti analytical rotor and equilibrated to 25° C. Samples are scanned at 280 nm with a rotor speed of 42000 rpm at full vacuum. A total of 80 scans for each cell are collected for analysis. The first scan for each sample is excluded from downstream data processing to avoid artifacts caused by meniscus.

The data is analyzed using the c(s) method developed by Peter Shuck at N.I.H. and the SEDFIT (version 8.8) program with implemented c(s). Using the c(s) method, raw data scans are directly fit to a Lamm function of S in order to derive a distribution of sedimentation coefficients. The parameters used for the fitting procedure are resolution, 400; confidence interval, 0.75; grid size, 1000; partial specific volume, 0.7245; buffer density, 1.000; and buffer viscosity, 0.1002. Frictional ratio, meniscus and bottom positions are set as fitted parameters. Time independent noise is also fitted. The detected peaks are integrated and classified as follows: from 0 to 6 S, fragments; from 6 to 9 S, monomer; and from 9 to 20 S, aggregates.

AUC may be used to characterize antibody formulations with low relative levels of aggregation and fragmentation. AUC may be able to better resolve antibody fragments and aggregates from the monomer species in situations that are beyond the resolution capabilities of SEC. peaks. AUC estimates of the molecular mass of an aggregate peak may also be used as an indicator of its composition (e.g., dimers vs. higher multimers).

Compared to SEC, AUC may also be able to better resolve differing species of a given macromolecule. It is, however, necessary to establish first the proper sample dilution rate, as the noise/signal ratio of AUC is dependent on the antibody concentration in the sample.

### 7.1.2.3. Turbidity Measurement:

Protein aggregation in the antibody formulation may also be characterized by turbidity measurement. Turbidity is a measure of the amount by which the particles in a solution scatter light and, thus, may be used as a general indicator of protein aggregation or denaturation. Elevated turbidity may indicate a higher level of aggregation or an increased number/increased size of particles.

Turbidity measurement may be performed with a turbidimeter (e.g., 2100AN or 2100N, Hatch) following the manufacturer's instructions. Approximately 3 to 4 ml of formulation sample is transferred into a glass test tube and degassed for 2 minutes using an in-line vacuum system. The degassed sample is then placed into a turbidimeter (e.g., 2100AN or 2100N, Hatch) sample compartment at room temperature for analysis. The turbidimeter is calibrated with STABLCAL® Stabilized Formazin Turbidity standard (Hatch) at 40, 200, 1000 and 4000 NTU (nephelometric turbidity unit) and verified by analyzing control suspensions of formazin at 3, 6, 18, 30 and 60 NTU.

### 7.1.2.4. Particle Count

The number and size of particles in a particular formulation may be determined using a particle counter (e.g., Beckman Coulter Multisizer 3) according to the manufacturers instruction.

### 7.1.2.5. Viscosity Profile

Viscosities of antibody formulations may be measured using a viscometer (e.g., ViscoLab 4000 Viscometer System from Cambridge Applied Systems equipped with a ViscoLab Piston (0.3055″, 1-20 cP)). The viscometer is calibrated before use with the appropriate standards (e.g., S6S Reference Standard from Koehler Instrument Company, Inc.). The viscometer is connected to a water bath to equilibrate the system to 20° C. Piston is checked using S6S viscosity reference standard (8.530 cP @ 20.00° C.). Piston is also checked using RODI H₂O (1.00 cP @ 20.0° C.). The piston is cleaned and rinsed thoroughly with soap and water between measurements of each different solution type. Subsequently the system is cooled to ≦2° C. Once the system temperature is at or below 2° C., sample is loaded into the chamber and the piston is lowered into the sample. After sample is equilibrated to the temperature of the chamber, measurement is initiated. The temperature is increased at 1° C. increments every 7-10 minutes to a final temperature of ≧25° C. The viscosity result is recorded immediately prior to increasing the temperature. The piston remains in motion during measurements to minimize the need for re-equilibration. The Rheometer may be used to measure viscosities of formulations.

### 7.1.2.6. Differential Scanning Calorimetry

Differential Scanning calorimetry (DSC) may be used to ascertain changes over time in the thermal stability of an antibody in a particular formulation. Thermal melting temperatures (Tₘ) are determined with a differential scanning calorimeter (e.g., VP-DSC from MicroCal, LLC) following the manufacturer's instruction. In one example, VP-DSC is used at a scan rate of 1.0° C./min and with a temperature range of 25-120° C. A filter period of 8 seconds is used along with a 5 minute pre-scan thermostating. Samples are prepared by dialysis into 20 mM Histidine-HCl, pH 6 using Pierce dialysis cups (3.5 kD). Average Mab concentrations are 500 μg/mL as determined by A₂₈₀. Melting temperatures are determined following the manufacturer's instructions using software supplied with the system.

### 7.1.2.7. Liquid Chromatography Mass Spectrometry (LC-MS)

Liquid Chromatography Mass Spectrometry (LC-MS) may be used to characterize a degradation fragment detected by SEC or AUC in the antibody formulation.

Peak SEC column fractions containing the degradation fragment are collected and digested with N-Glycosidase F, also known as PNGase F, at 37° C. overnight. PNGase F is an amidase used to deglycosylate protein samples. The enzyme cleaves between the innermost GlcNAc and asparagine residues of high mannose, hybrid and complex oligosaccharides on N-linked glycoproteins. The deglycosylated samples mixed with a reducing buffer (e.g., 2.5 mg/mL DTT, 6.0 M guanindine HCl, pH 8.2) and kept at 56° C. in a water bath for 60 minutes. Neat 4-vinylpyridine (e.g., Aldrich Chem. Co., WI) is then added to the sample, and the reaction mixture is held at ambient temperature for 30 minutes. The deglycosylated, reduced and alkylated sample is immediately loaded onto a reversed phase column in order to separate the modified samples from the reactants.

Deglycosylated, reduced, and alkylated samples are fractionated using a reversed phase column (e.g., Jupiter 5 μm C4, 300 Å, 250×2.00 mm, Phenomenex) with a binary gradient HPLC system (Agilent 1100). Mobile phase A consists of 30% acetonitrile in water with 0.1% trifluoroacetic acid and mobile phase B consists of 50% acetonitrile in water with 0.1% trifluoroacetic acid. The samples are separated using a linear gradient of 30-50% acetonitrile in water, over 16 min. with a flow rate of approximately 200 mL/min. The column effluent is directed to a UV detector and then split 1:1, one half going through a switching valve on an Ion Trap mass spectrometer (e.g., LTQ, ThermoElectro, San Jose, Calif.), and the remaining half to waste.

The Ion Trap mass spectrometer is calibrated before the experimental run using a mixture of caffeine, L-methionyl-arginyl-phenylalanyl-alanine (SEQ ID NO: 239) acetate H₂O, and Ultramark 162. The Electrospray Ionisation Mass Spectrometry (ESI-MS) data is acquired in positive ESI full scan mode. The BioWork deconvolution program (ThermoFinnigan) may be used to reconstruct the mass spectra and obtain the molecular masses of the peptides/proteins from their original mass spectra. The mass data subsequently is used to determine the identity of the degradation fragment.

### 7.1.2.8. Binding Affinity Characterization

Binding affinity of monoclonal antibody recovered from the formulation following long term storage (e.g., 1 month at 40° C. or 6 months at 5° C.) may be determined by any assay know in the art, for example, but not limited to ELISA assay, flow cytometry assay. The functional properties of an antibody may also be analyzed using in vitro and in vivo assays (e.g., ADCC assay, in vivo depletion assay). Assays suitable for the functional characterization of anti-CD19 antibodies are provided in U.S. patent application Ser. No. 11/852,106 filed on Sep. 7, 2007.

### 7.1.3. Formulation Development

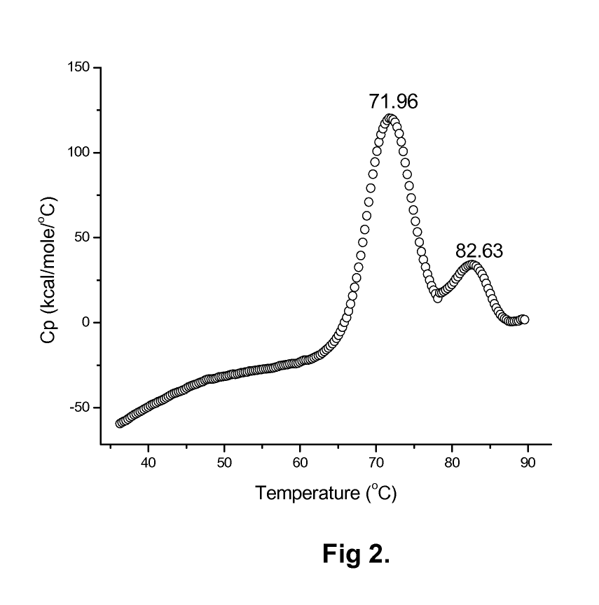

The physicochemical properties of the 551 antibody were characterized as follows. The DSC profile of the 551 antibody was determined as described above. DSC measurements were performed in 20 mM histidine (pH 6.0) buffer at 0.25 mg/ml antibody concentration. The DSC profile of the 551 antibody is shown in FIG. 2.

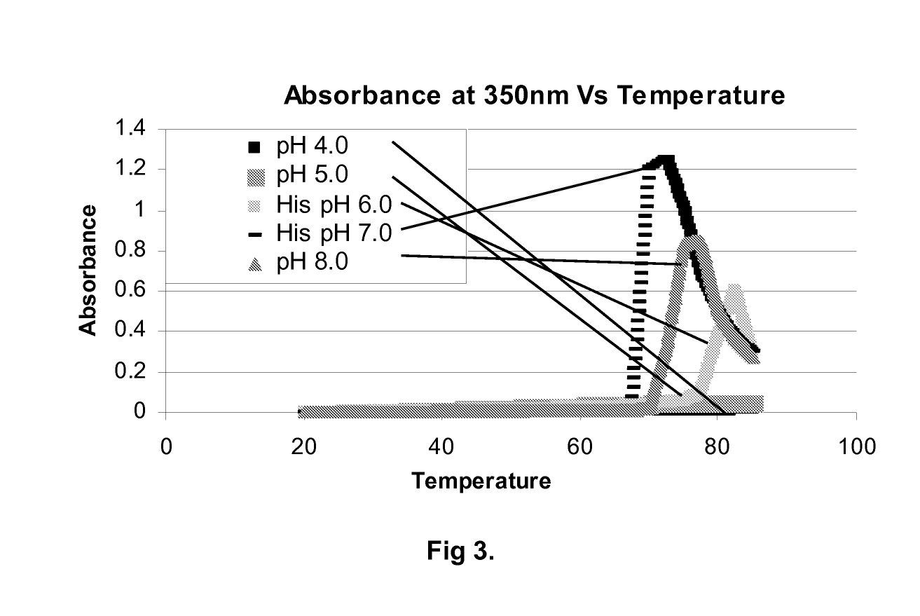

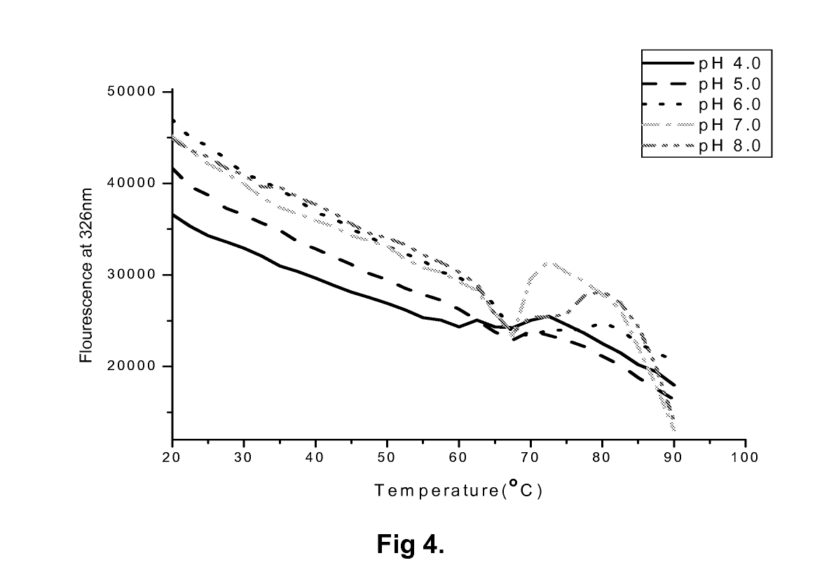

The effect of pH on the stability of the 551 antibody was ascertained by measuring colloidal stability (FIG. 3) and tryptophan fluorescence (FIG. 4) as a function of temperature at various pHs. Colloidal stability data suggested that 551 may be most stable at pH 4-5. 551 was colloidally most unstable at pH 7 within the range tested.

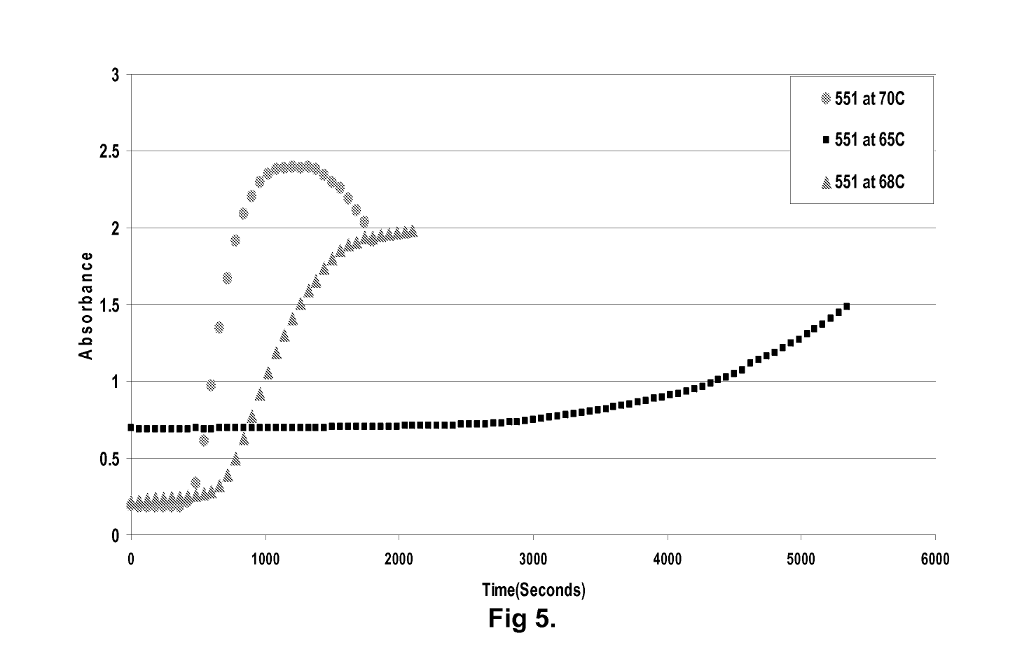

The colloidal destabilization rate of 551 at various temperatures was assessed to select the optimal temperature for high throughput excipient screening. Colloidal destabilization rate was assessed by measuring turbidity (A350 nm) of a 551 formulation at various temperatures as a function of time. A representative sample of the experimental results is shown in FIG. 5. Based on these results, 68° C. was selected as the temperature for high throughput excipient screening.

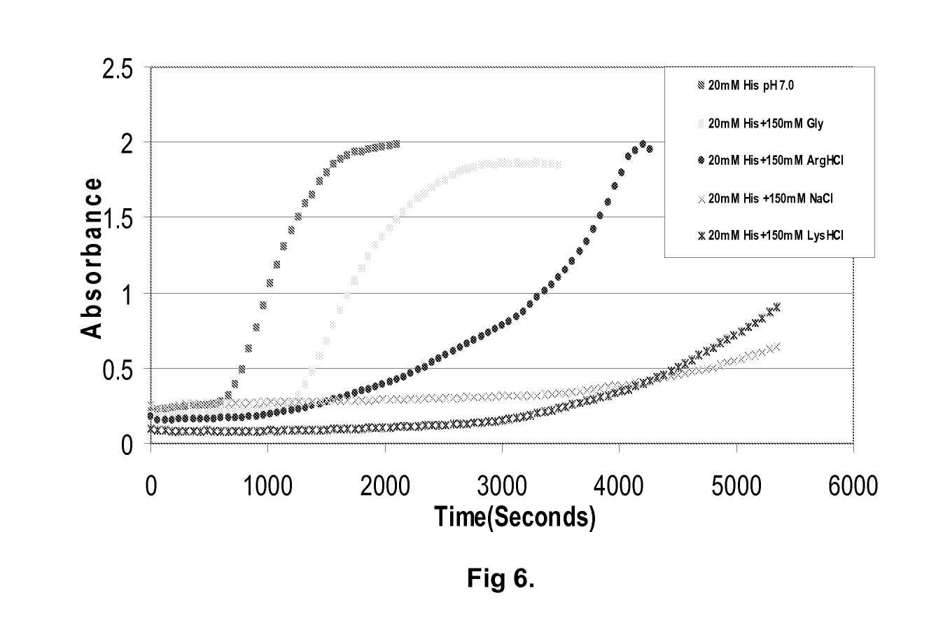

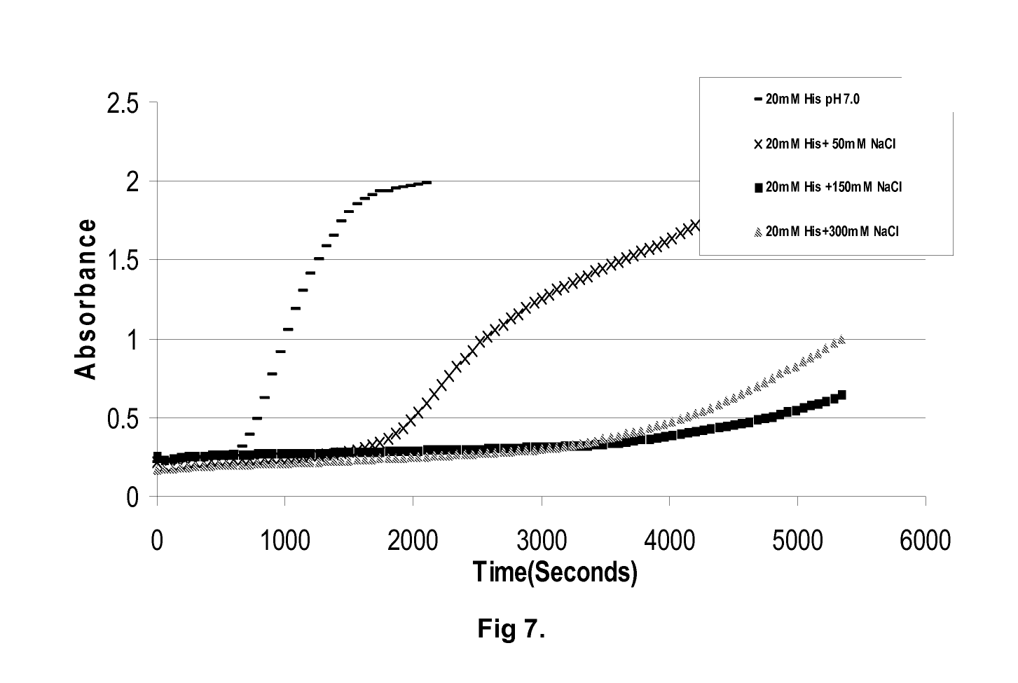

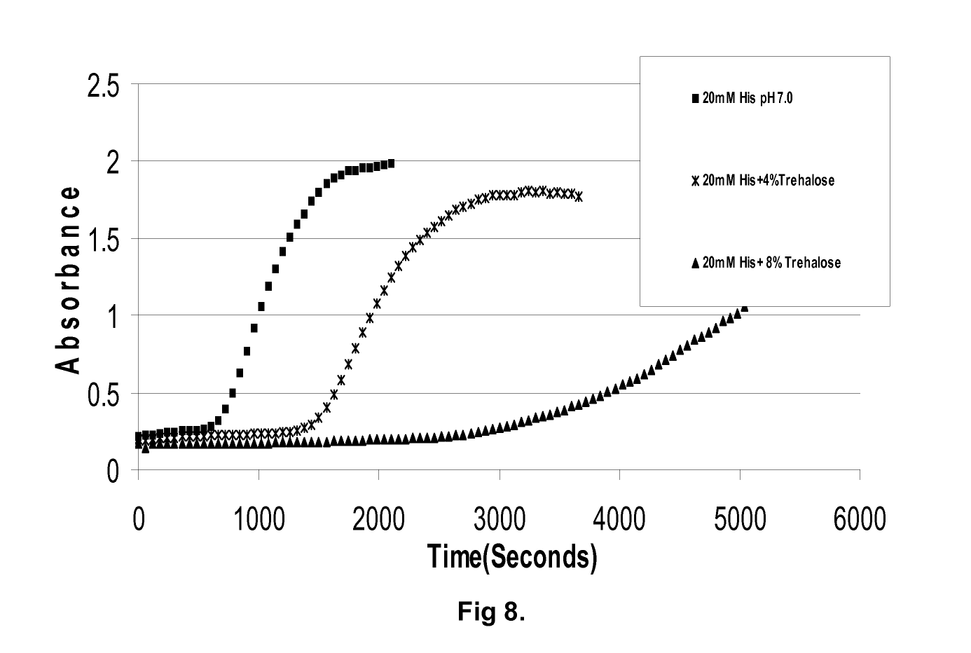

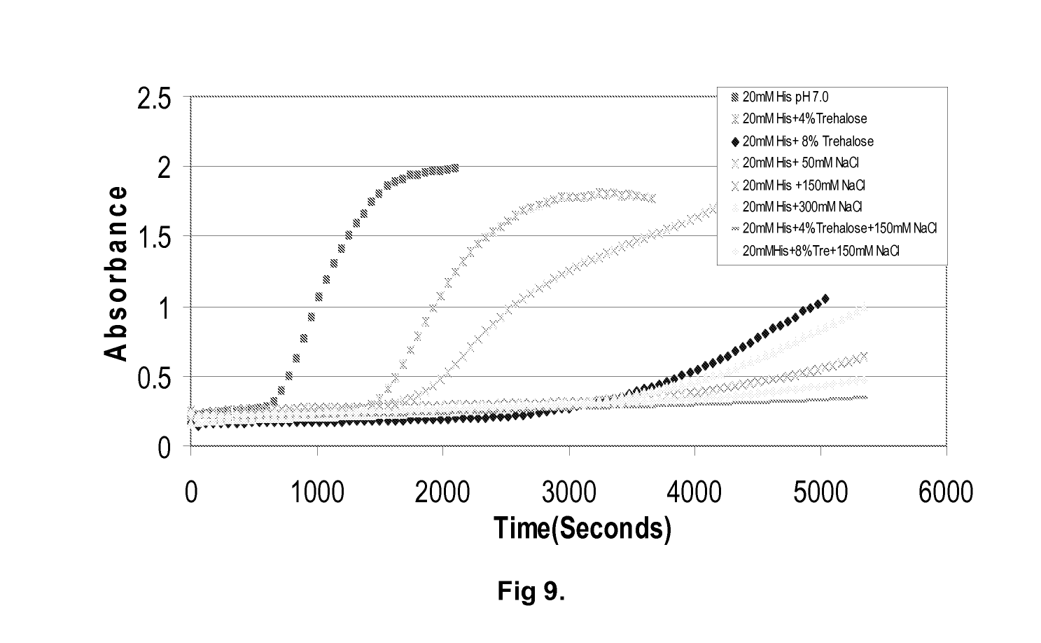

A high throughput screen was used to identify excipients with the potential to stabilize the 551 antibody. A metastable control buffer (20 mM histidine at pH 7.0) was selected for the screen based on the pH sensitivity of the 551 antibody. Excipients were screened by comparing the colloidal stability of 551 in the control buffer to the colloidal stability of 551 in a buffer comprising additional excipients either alone or in combination. Changes in colloidal stability were followed by assessing the colloidal destabilization rate of the various test formulations. Stabilizing excipients reduce the destabilization rate while destabilizing excipients increase it. A representative result is shown in FIG. 6. The destabilization rate was reduced by the addition of NaCl, lysine, arginine or glycine as shown by the turbidity curves' shift to the right of the tubidity curve of the formulation without salt or amino acids. Additional samples from the high throughput excipient screen are shown in FIGS. 7-9. The high throughput screen identified glycine, arginine, lysine, NaCl, and trehalose as stabilizing excipients for the 551 antibody. The screen also demonstrated that optimal stabilization may be achieved with a formulation whose ionic strength is equivalent that of 150 mM NaCl. Additional experiments were performed to ascertain the effect of combinations of these excipients on 551 stability (FIG. 9). Out of the combinations tested, the most pronounced stabilizing effect was observed with 150 mM NaCl in combination with 4% or 8% trehalose.

Based on the excipient screen, 6 formulations were chosen for real time accelerated stability study at 40° C.:

A: 20 mM Histidine, pH 6

B: 20 mM Histidine, 150 mM Glycine, pH 6.0

C: 20 mM Histidine, 4% Trehalose, pH 6.0

D: 20 mM Histidine, 150 mM NaCl, pH 6.0

E: 20 mM Histidine, 150 mM NaCl, 4% Trehalose, pH 6.0

F: 20 mM Histidine, 150 mM ArgHCl, pH 6.0

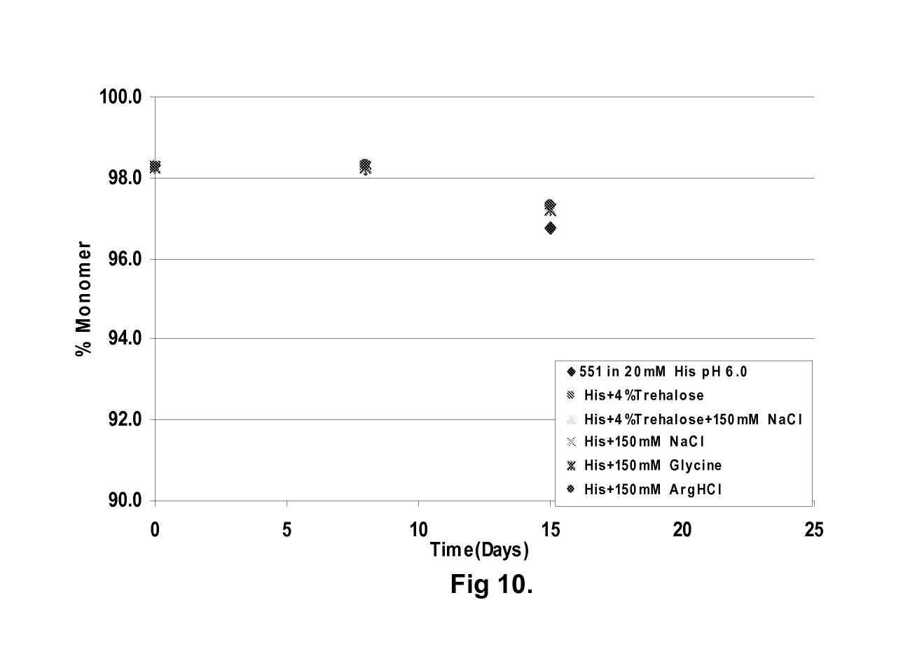

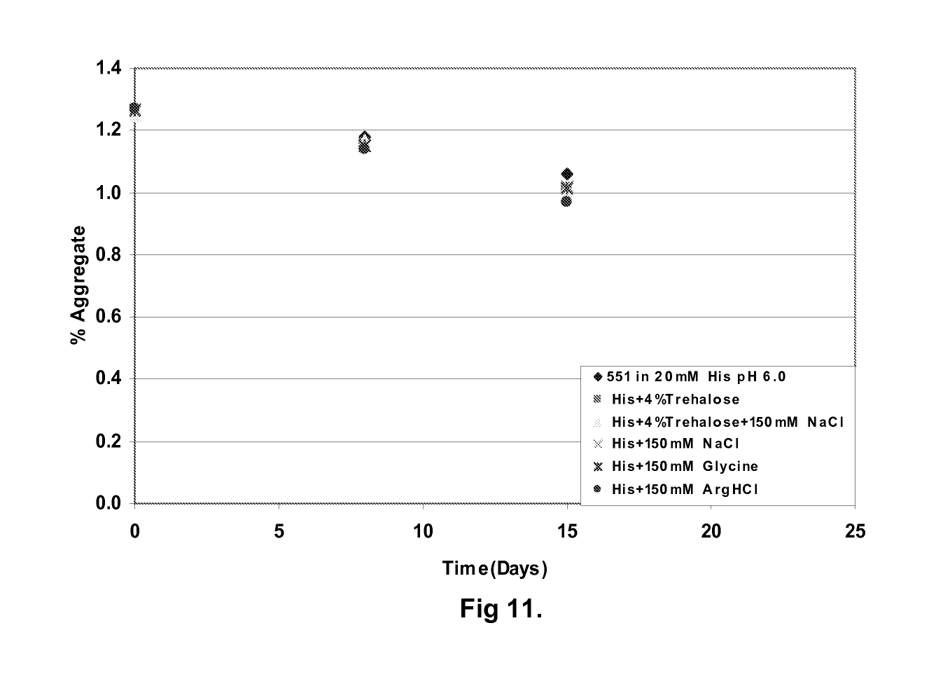

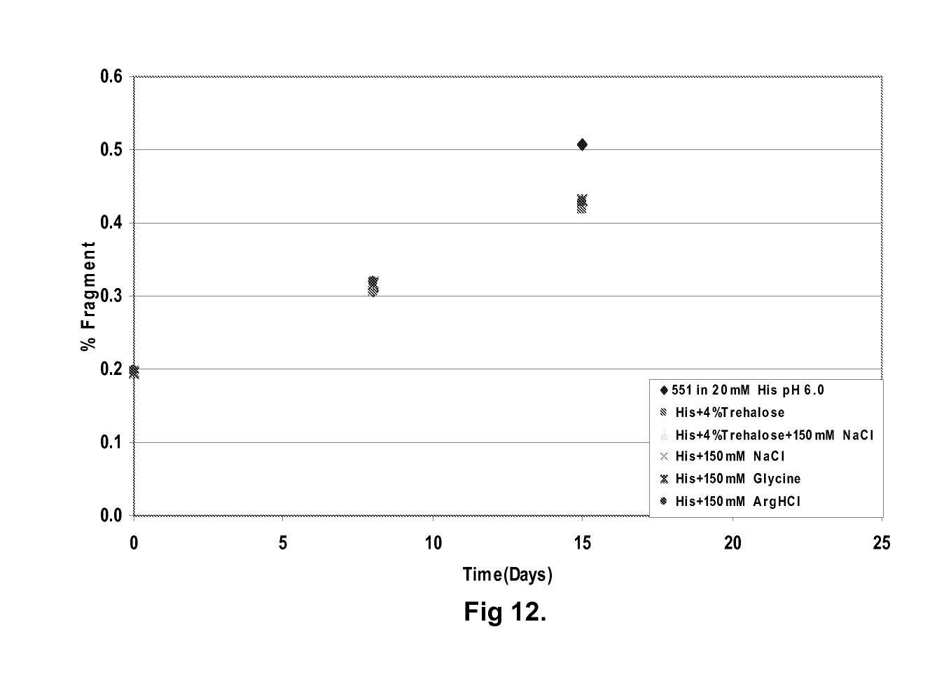

Each formulation comprised 15 mg/ml 551 antibody. Antibody aggregation and fragmentation was assessed using SEC. A sample of the experimental results is shown in FIGS. 10-12. A small increase in the higher order aggregates over time was seen in all formulations tested.

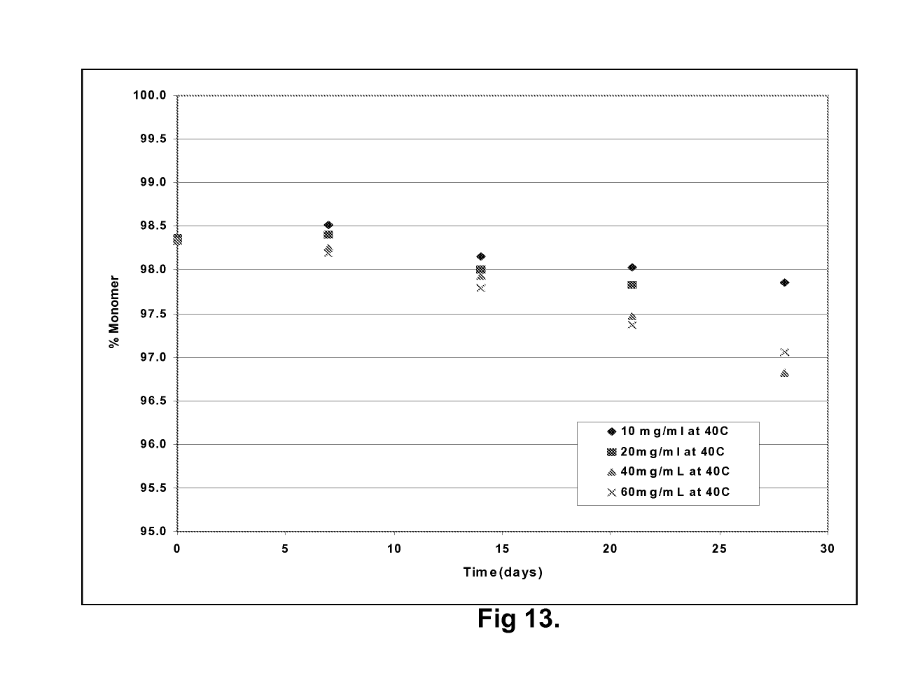

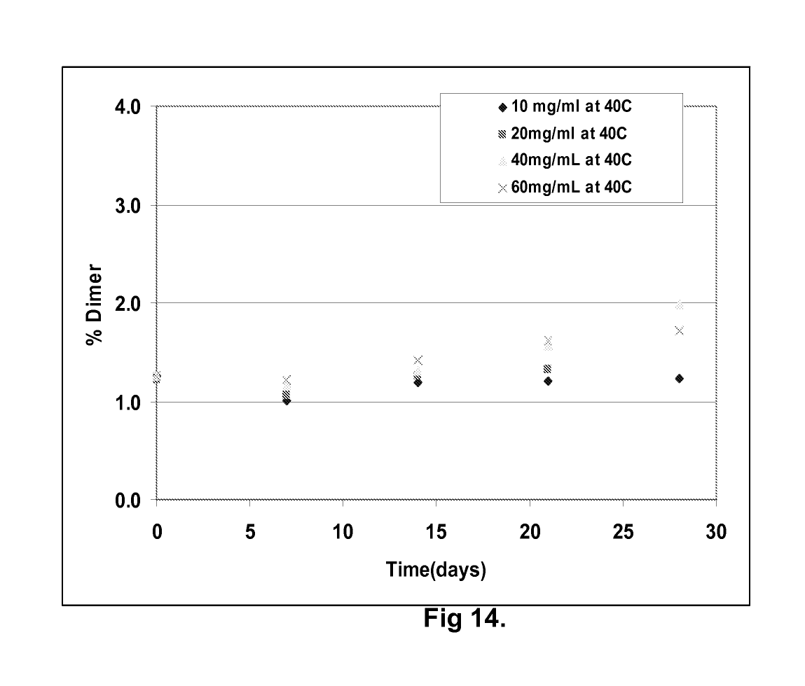

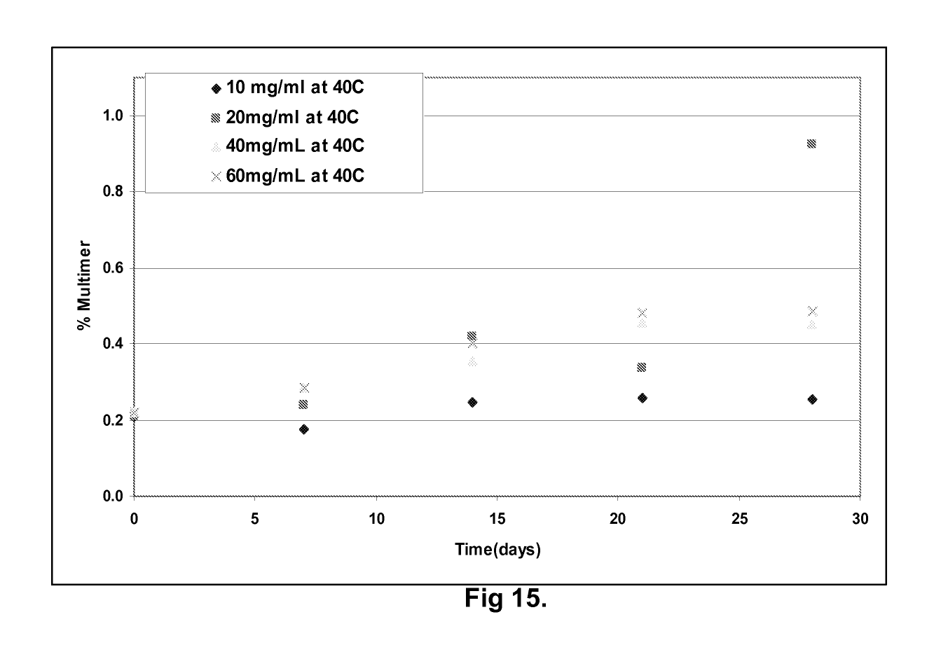

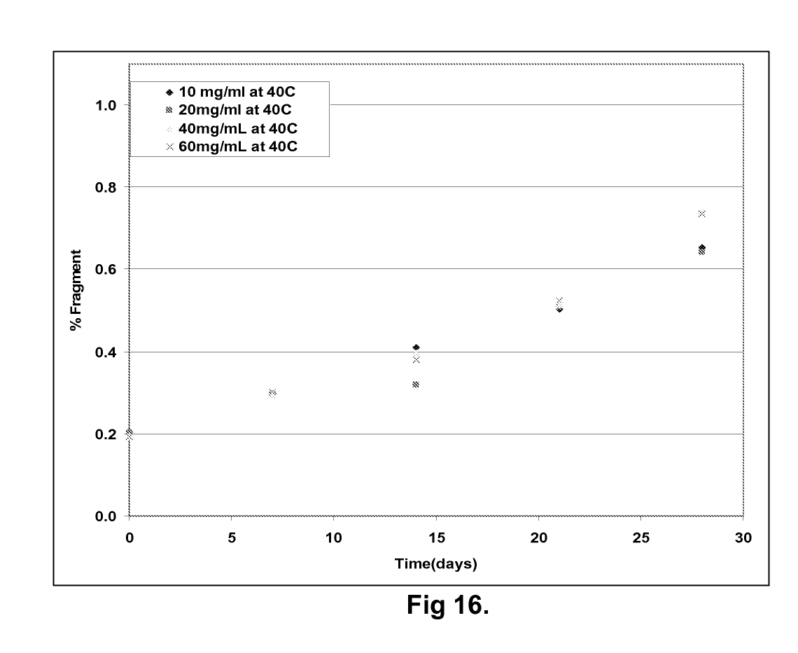

Additional real time stability studies were performed to ascertain the effect of antibody concentration on antibody aggregation. Formulations comprising 10 mg/ml, 20 mg/ml, 40 mg/ml or 60 mg/ml 551 and 10 mM histidine pH 6.0 were stored at 5° C. or 40° C. Antibody aggregation after extended storage was determined by SEC. A sample of the experimental results is shown in FIGS. 13-16. No change in monomer concentration was seen during 30 days of storage at 5° C. Rate of monomer loss at 40° C. increases with increased antibody concentration. Dimer formation was higher at 40° C. than 5° C. and it also increased with increased antibody concentration. Multimer concentration did not change over 30 days at 5° C. Multimer formation rate increased with higher antibody concentration at 40° C. Antibody fragmentation was higher at 40° C. than 5° C.; fragmentation also increased with increased antibody concentration.

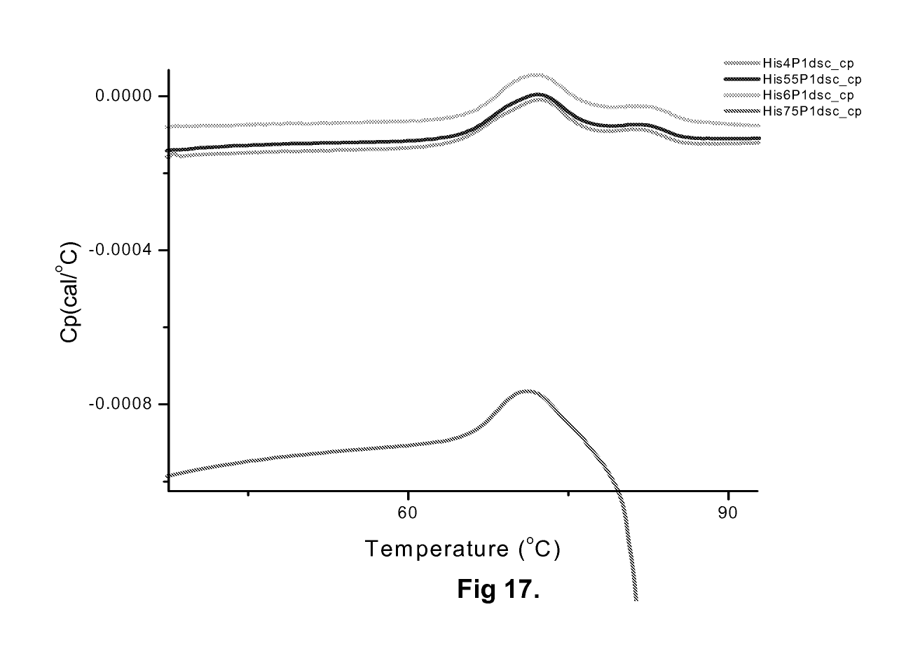

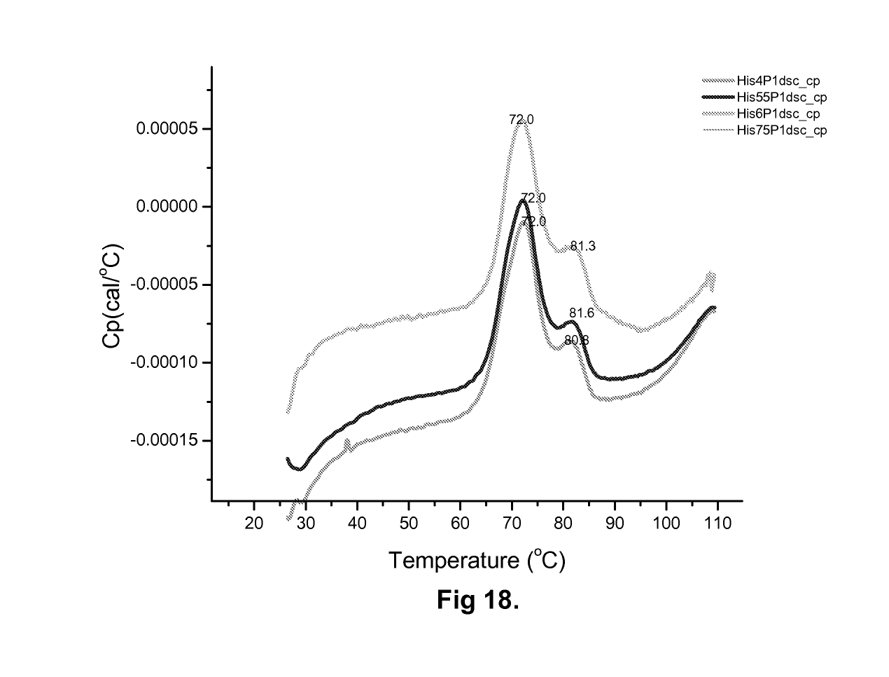

Additional short term studies were performed to study the effect of pH the stability of 551 formulations. DSC profiles measured at different pHs (FIGS. 17 and 18) show no pH dependent changes in Tm with the exception of pH 7.5. DSC profiles were determined at 0.25 mg/ml protein concentration in 20 mM histidine buffer. Aggregation studies involving formulations with various pHs (pH 4.0-7.5) showed multimer formation at all pHs except pH 6.5. Multimer formation in a pH 6.5 formulation was arrested at both 5° C. and 40° C. In other studies pH 6.0 also showed an effect similar to pH 6.5 with respect to aggregation and multimer formation. The highest multimer formation rate was seen at pH 4 and 7.5. The pH dependence of antibody aggregation was studied using formulations comprising 2.4 mg/ml 551 in 20 mM histidine. So based on this for the next set of studies pH 6.0 and pH 6.5 were considered for further evaluation.

7 formulations comprising 10 mg/mL 551 antibody were put on stability studies at 5° C., 25° C. and 40° C.:

Formulation 1. 10 mM His, 140 mM NaCl, pH 6.0

Formulation 2. 10 mM His, 75 mM NaCl, 4% Trehalose, pH 6.0

Formulation 3. 10 mM His, 100 mM NaCl, 2% Trehalose, pH 6.0

Formulation 4. 10 mM His, 9% Trehalose, pH 6.0

Formulation 5. 10 mM His, 75 mM NaCl, 4% Trehalose, pH 6.5

Formulation 6. 10 mM His, 140 mM NaCl, 9% Trehalose, pH 6.0

Formulation 7. 10 mM His, 150 mM LysHCl, 4% Trehalose, pH 6.0

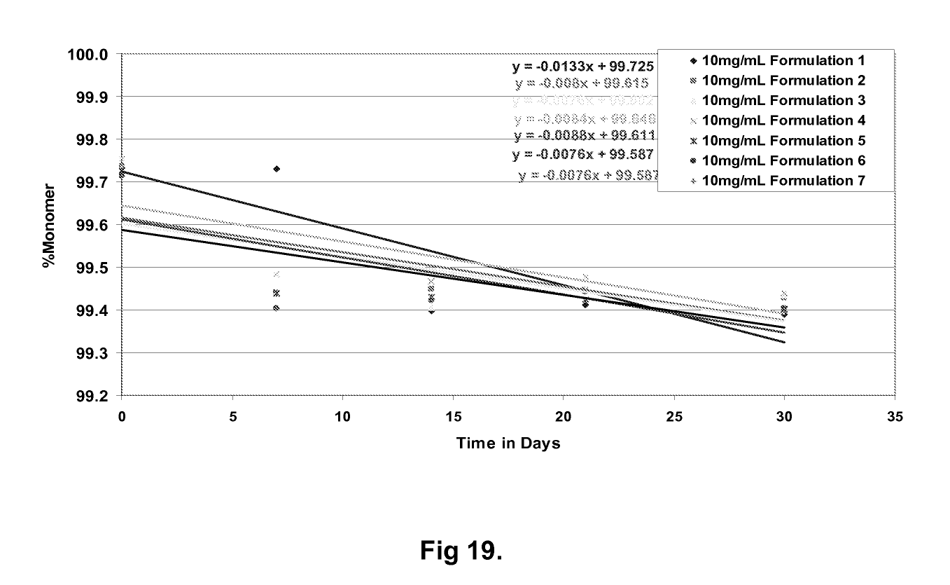

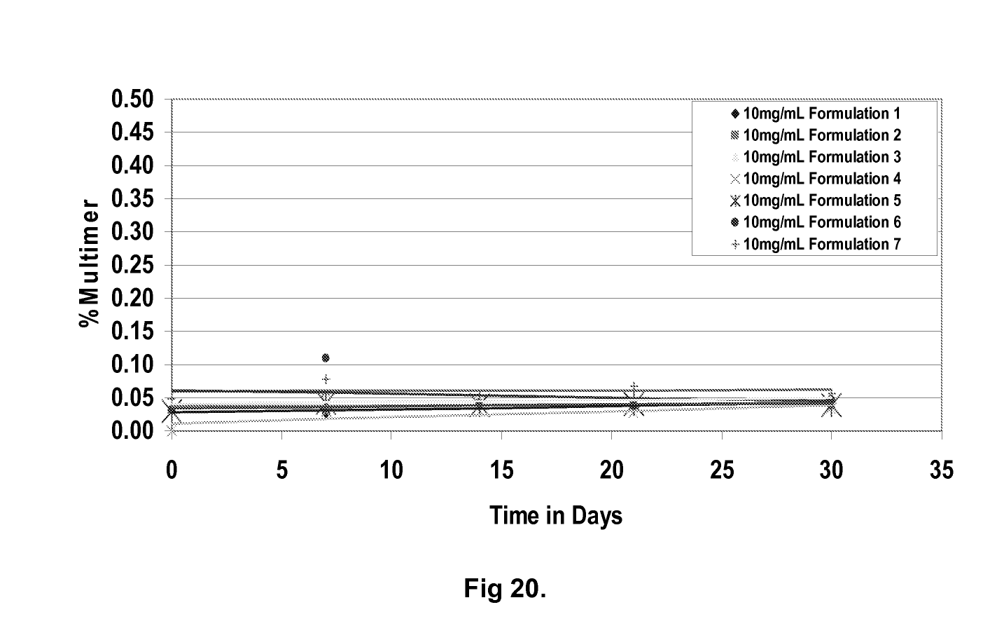

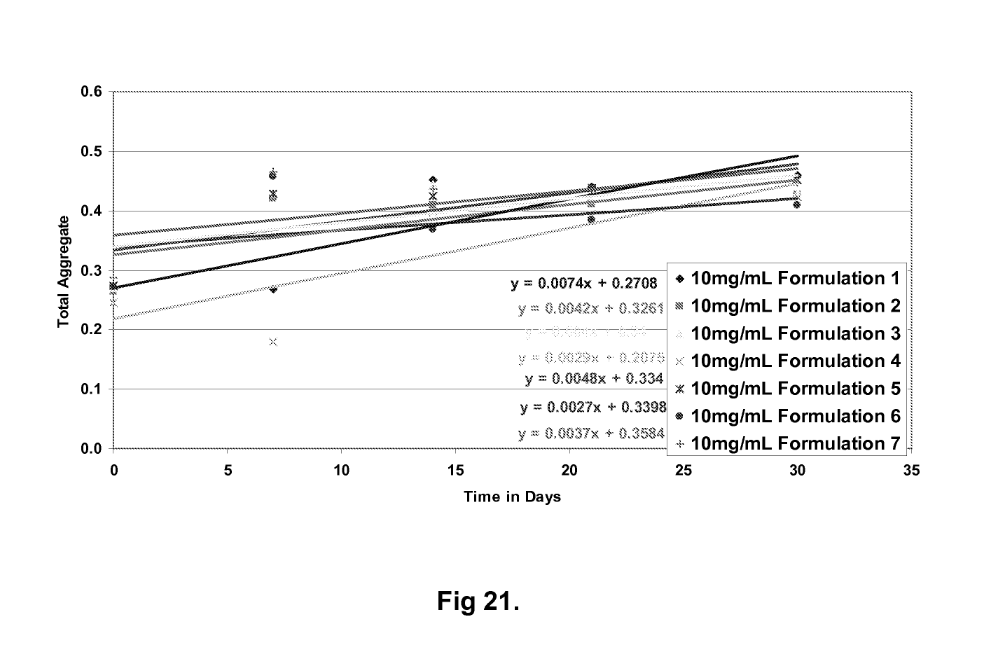

Monomer, multimer and total aggregate concentration was followed over time using SEC. Examples of the results are shown in FIGS. 19-21. All formulations showed similar stability. The two highest monomer loss rates were observed in Formulation 4 and 5.

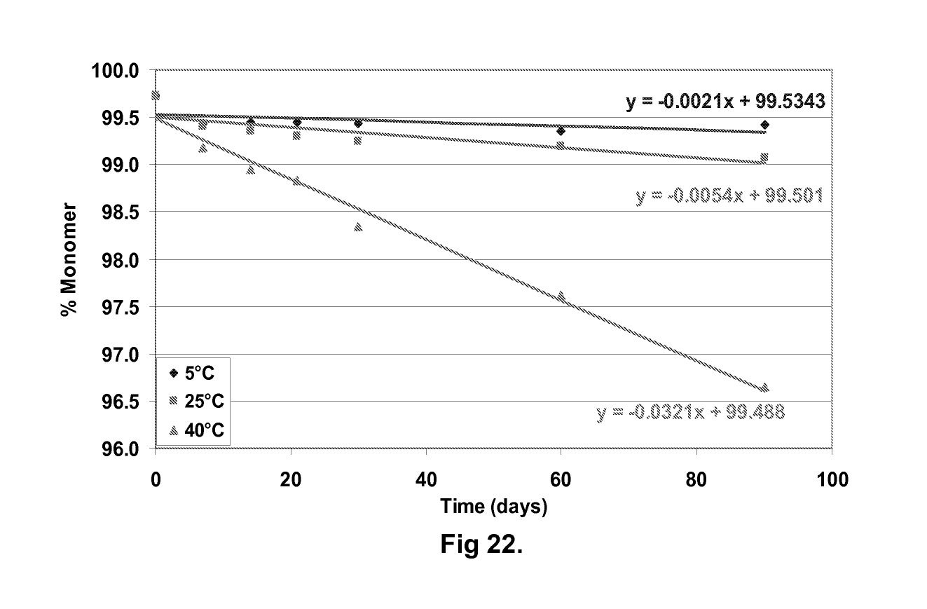

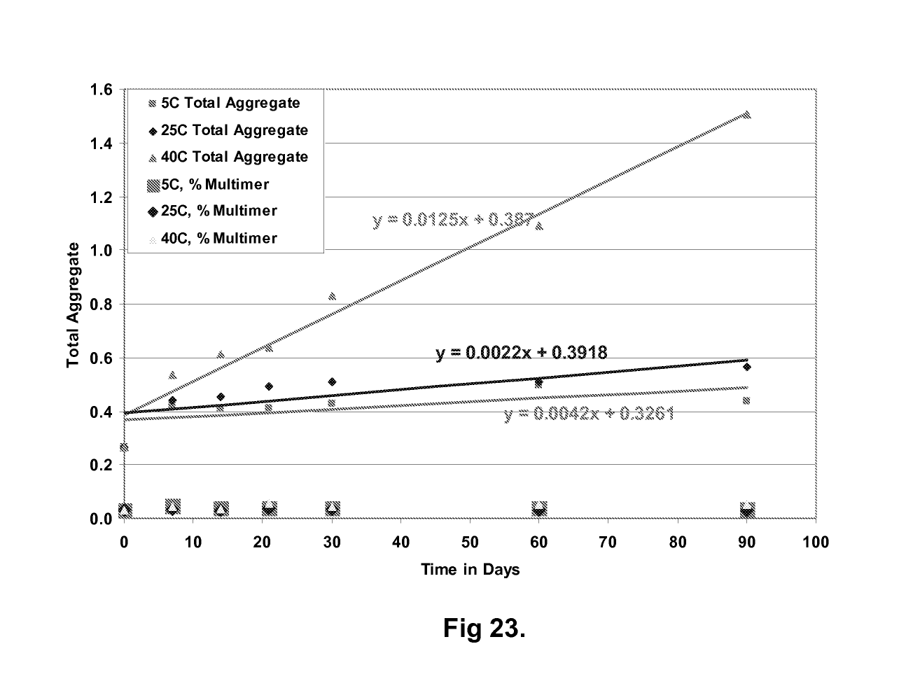

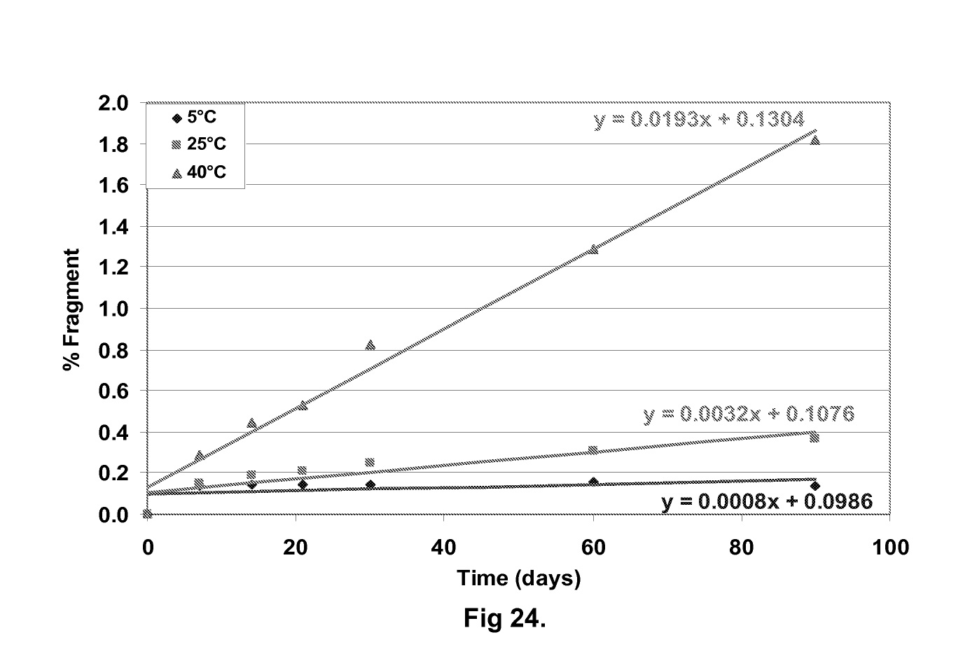

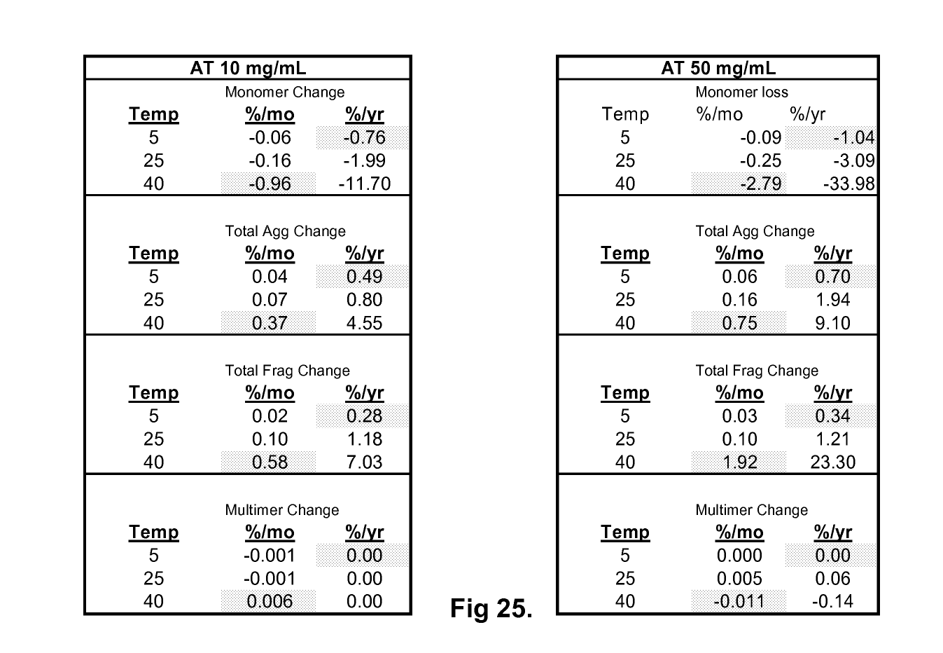

The formulation comprising 10 mM Histidine, 75 mM NaCl, 4% Trehalose, pH 6.0 and 10 mg/ml or 50 mg/ml 551 antibody was put on stability studies at 5° C., 25° C. and 40° C. Aggregation and fragmentation was followed using SEC. Results are displayed in FIGS. 22-24. A table summarizing aggregation and fragmentation in the formulation is presented in FIG. 25.

The formulation comprising 10 mM Histidine, 75 mM NaCl, 4% Trehalose, pH 6.0 and 10 mg/ml 551 antibody was subjected to sheer stress using multiple cycles of freeze-thawing. Nunc 2.0 mL Cryo vials filled with 1.7 ml of formulations were used for the stress studies. Samples were subjected to 5 freeze-thaw cycles (−80° C. to 25° C., each cycle includes 2 hrs freezing followed by 2 hrs at room temperature). Samples were analyzed by visual inspection, SEC and absorbance measurements at 280 nm and 340 nm. Starting material contained some multimers; multimer concentration was monitored during the course of the study. No change in visual appearance or multimer level was detected during the freeze-thaw study. Results are summarized in Table 3.

TABLE 3
Aggregation following 5 cycles of freeze-thaw.

| Sample Type | % Monomer | % Aggregate | Recovery |
|---|---|---|---|
| T = 0 | 98.0 | 1.6 | No loss in Area |
| After 1X F/T | 98.0 | 1.6 | after 5X F/T |
| After 2X F/T | 98.0 | 1.6 | |
| After 3X F/T | 98.0 | 1.6 | |
| After 4X F/T | 98.0 | 1.6 | |
| After 5X F/T | 97.9 | 1.7 | |

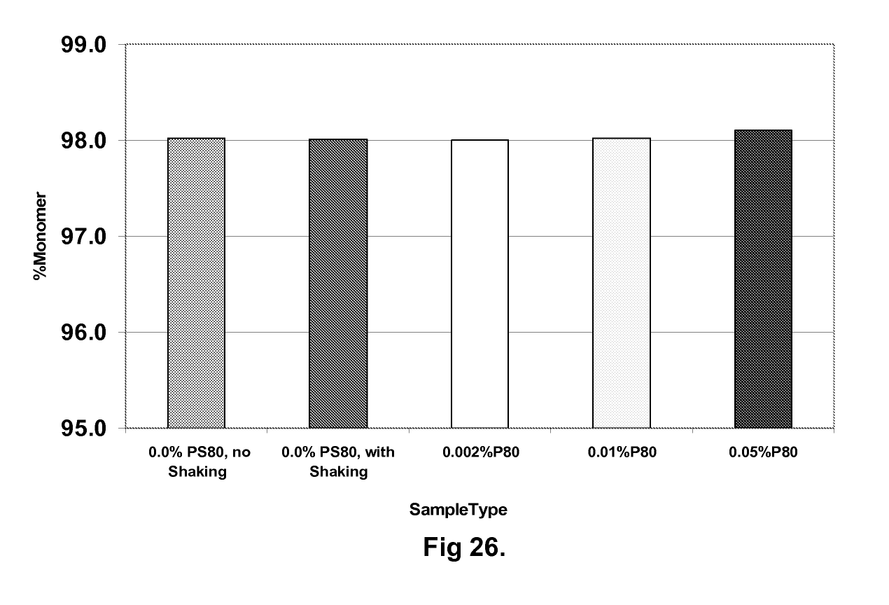

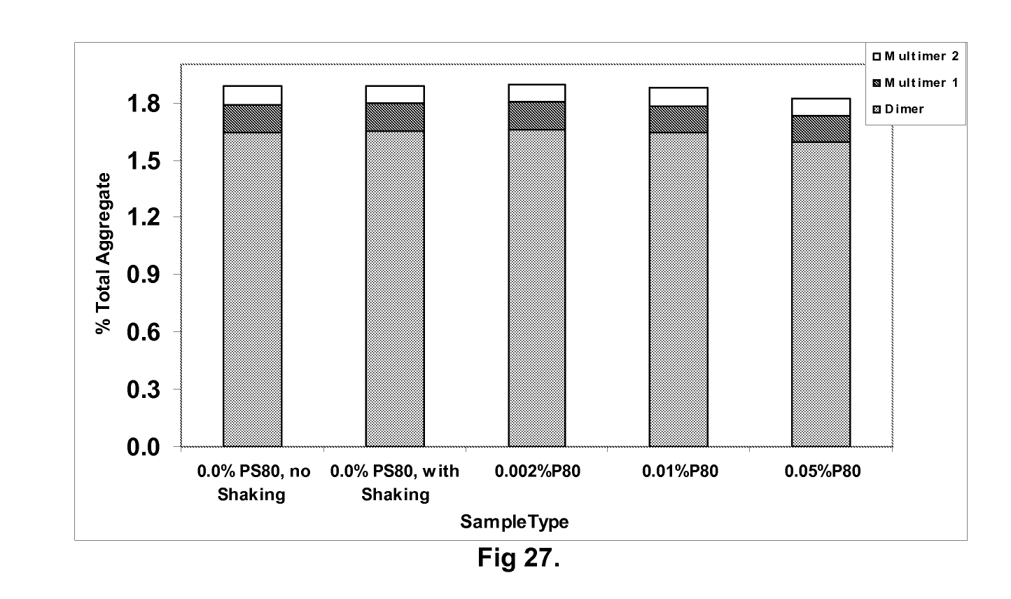

The formulation comprising 10 mM Histidine, 75 mM NaCl, 4% Trehalose, pH 6.0 and 10 mg/ml 551 antibody was subjected to mechanical stress by shaking. The study also included formulations comprising 0.002%, 0.01%, and 0.05% polysorbate 80. 3 ml vials with 4432/50 Serum Stoppers containing 1 ml formulation were used for the mechanical stress study. Samples were subjected to mechanical stress by shaking them for 4 hrs on a vortex (VWR Mini Vortexer #945300) at speed setting 1. Samples were analysed by visual inspection, SEC and absorbance measurements at 280 nm and 340 nm. Starting material contained some multimers; multimer concentration was monitored during the study. Results are shown in FIGS. 26 and 27. No change in visual appearance, dimer level, multimer level or recovery was seen during the course of the study. 551 is not susceptible to shaking induced aggregation in vials irrespective of the presence or absence of polysorbate 80.

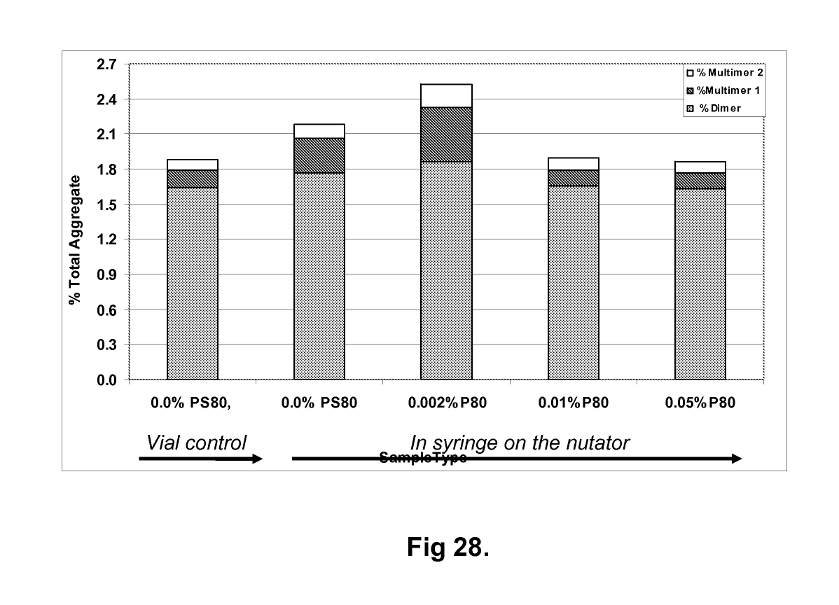

The formulation comprising 10 mM Histidine, 75 mM NaCl, 4% Trehalose, pH 6.0 and 10 mg/ml 551 antibody was subjected to mechanical stress by shaking in a 10 ml terumo syringe. The study also included formulations comprising 0.002%, 0.01%, and 0.05% polysorbate 80. 10 ml terumo syringes were filled with 0.7 ml formulation at a total plunger extension of 7 ml leaving a 7 ml air gap. Samples were gently shaken for 24 hrs on a nutator. Samples were visually inspected for any changes in opalescence or particulation at 15 mins and 6 hrs. After 24 hrs of shaking samples were analysed by visual inspection, SEC and absorbance measurements at 280 nm. Formulations were subjected to visual inspection both in the syringe and after transfer into a vial. Results are shown in FIG. 28. Formulation without polysorbate 80 didn't change visually at 6 hrs, but became opalescent at the end of 24 hrs. An increase in % multimer was seen in samples comprising less than 0.01% polysorbate 80. No change in % multimer was seen in samples comprising 0.01% or more polysorbate 80.

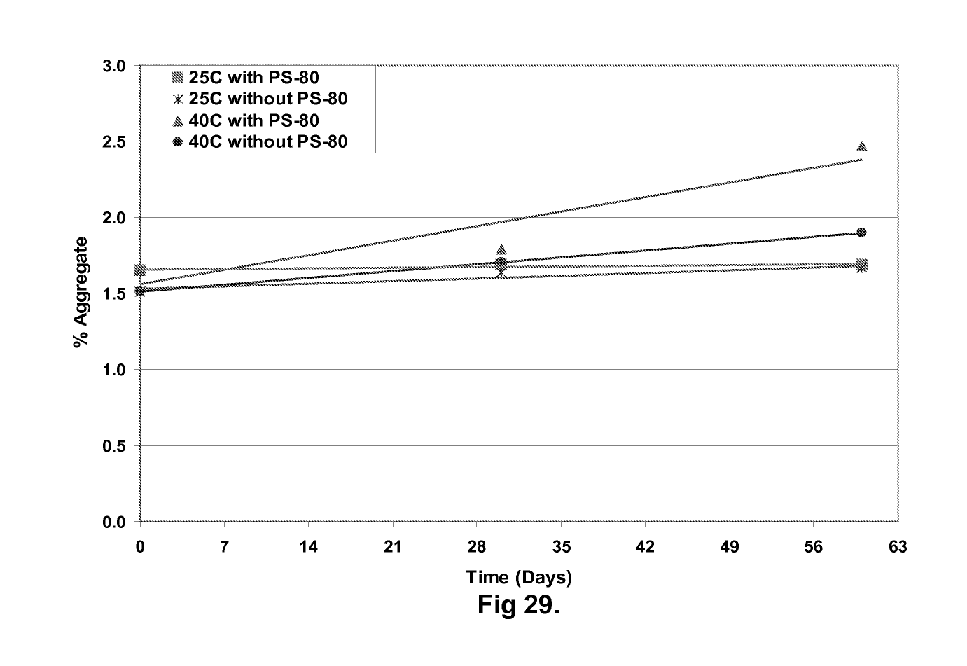

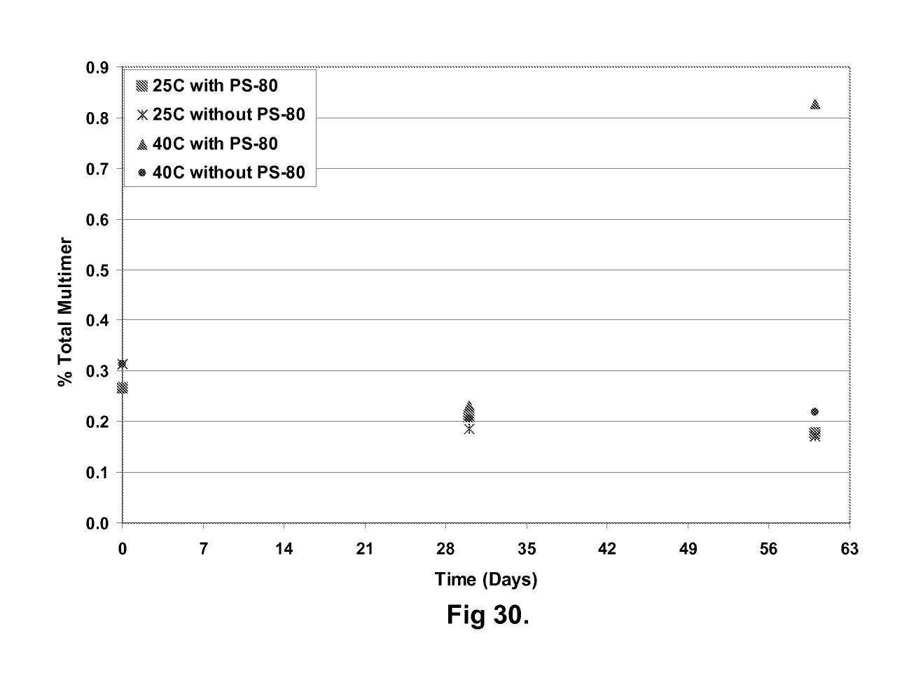

The formulation comprising 10 mg/ml 551 antibody, 10 mM Histidine, 75 mM NaCl, 4% Trehalose, pH 6.0 and 0% or 0.02% polysorbate 80 was put on stability studies at 25° C. and 40° C. Aggregation and multimer formation was followed using SEC. Results are displayed in FIGS. 29 and 30.

### 6.1.4 Formulation Stabilization Developments

Formulations were prepared to have varied concentrations of anti-CD19 antibody, 551 (10, 25, 50 and 100 mg/mL) in the formulation buffer (FB):10 mM histidine, 75 mM sodium chloride, 4% (w/v) trehalose dihydrate at pH 6.0, and either with or without 0.02% (w/v) polysorbate 80 (PS-80). The formulations were prepared according to the methods described above, stored at recommended storage temperatures, and analyzed by visual inspection. As shown in Table 4, particles were not observed in any of the formulations incorporating PS-80, including those with higher concentrations of the anti-CD19 antibody. However, particles were observed with the formulations that did not include PS-80.

TABLE 4
Particle Observations

| 551 | | | |
|---|---|---|---|
| Concentration | Contains | Storage Time | Particles |
| (mg/ml) | 0.02% PS-80 | (months) | Observed |
| 10 | No | 13 | Yes |
| 10 | Yes | 13 | No |
| 10 | No | 3 | Yes |
| 25 | No | 3 | Yes |
| 25 | Yes | 3 | No |
| 50 | No | 3 | Yes |
| 50 | Yes | 3 | No |
| 100 | No | 3 | Yes |
| 100 | Yes | 3 | No |
| 10 | No | 9 | Yes |
| 10 | Yes | 9 | No |

The number and size of particles were further analyzed to determine the nature of the particles using a FlowCAM instrumentation and HIAC liquid particle counter. The results of the Flow CAM data and HIAC data on the particles are shown in Tables 5 and 6, respectively.

TABLE 5
Flow CAM Data

| | Particle Size (Particle/mL) | | | | |
|---|---|---|---|---|---|
| Sample | >2 | >5 | >10 | >25 | >100 |
| 551 in FB buffer w/o | 20562 | 12241 | 6066 | 1397 | 16 |
| PS-80, TP = 13 mos | | | | | |
| 551 in FB buffer with | 1141 | 512 | 137 | 39 | 4 |
| 0.02% PS-80, TP = 13 mos | | | | | |

TABLE 6
HIAC Data on Particles

| | Particle Size (Particle/mL) | | | | |
|---|---|---|---|---|---|
| Sample | >2 | >5 | >10 | >25 | >100 |
| 551 in FB buffer w/o | 20562 | 12241 | 6066 | 1397 | 16 |
| PS-80, TP = 13 mos | | | | | |
| 551 in FB buffer with | 1141 | 512 | 137 | 39 | 4 |
| 0.02% PS-80, TP = 13 mos | | | | | |
| 551, DSP 55130, W/O | 18519 | 3509 | 481 | 0 | 0 |
| PS-80, TP = 9 mos | | | | | |

The formulation of 10 mg/ml 551 antibody, 10 mM Histidine, 75 mM NaCl, 4% Trehalose, pH 6.0 and 0% or 0.02% polysorbate 80 was further subjected to analysis. It was found that samples subjected to freeze-thaw cycles did not show a change in purity after 5×F/T. Moreover, no particulation was observed upon VI. Samples were further subjected to shaking induced aggregation in vials, which revealed that 551 is not susceptible to shaking induced aggregation irrespective of the presence or absence of PS-80. Shaking in Terrumo Syringes revealed no change in AUC, whereas the control sample containing no PS-80 became opalescent at the end of 24 hours. An increase in the percentage of multimer was seen in samples without PS-80 and at PS-80<0.01%, whereas no changes were seen in samples with PS-80≧0.01%.

### 6.1.5 Filtration Studies

Filtration studies were conducted to assess whether filtration would improve the sub-visible particle count without affecting other quality attributes. Sample vials from the lot exhibiting particles in the formulation of 10 mg/mL anti-CD19 antibody, 551, in the 10 mM histidine, 75 mM sodium chloride, and 4% (w/v) trehalose dihydrate at pH 6.0 were used for this study. PS-80 (2% stock) was spiked into these vials to give a final concentration of 0.02%. Samples were then filtered either before or after PS-80 addition using either a 0.22 μm Syringe PVDF or PEF filter. Assays were performed (pre and post filtration) including an initial A280 protein concentration, high performance size-exclusion chromatography (HPSEC), VI and HIAC testing.

Over a limited particle size range, particulate suspensions often exhibit a power-law size distribution (Kavanaugh et al., 1980), where the number (N) of larger particles falls off as a power (β) function of particle size (L).

dN/dL=A×(L)^{β}

Double logarithmic plots of particle count vs. size for such systems showed a linear relationship with a negative slope β that equals the exponent of the function. (typically, 1.6-4.6 for liquid suspensions, 3.2-4.0 for aerosols).

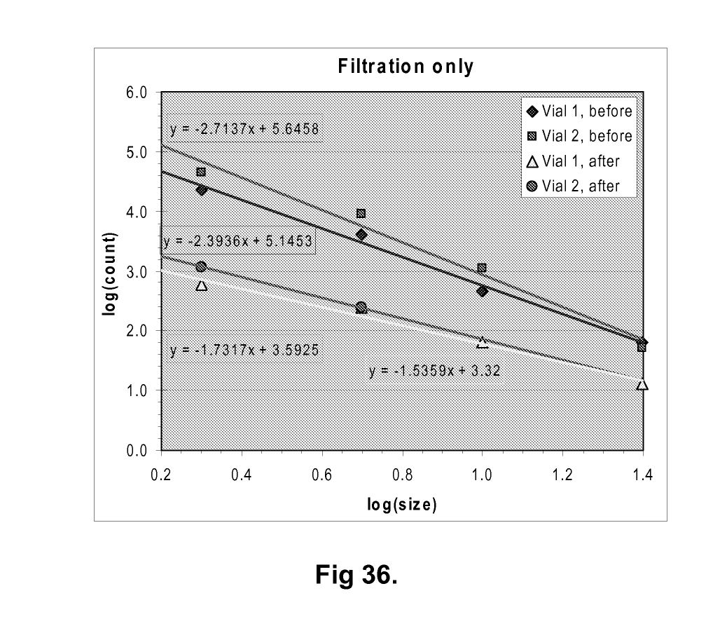

Vials of the formulations showed a particle size distribution that was well described by a power-law function with an exponent of ˜2.5 prior to filtration (Table 7 and FIG. 36). Filtration through a PVDF membrane decreased the particle count by nearly two orders of magnitude for 1 μm particles. Preferential clearance of smaller particles decreased the exponent of the power law distribution to ˜1.6.

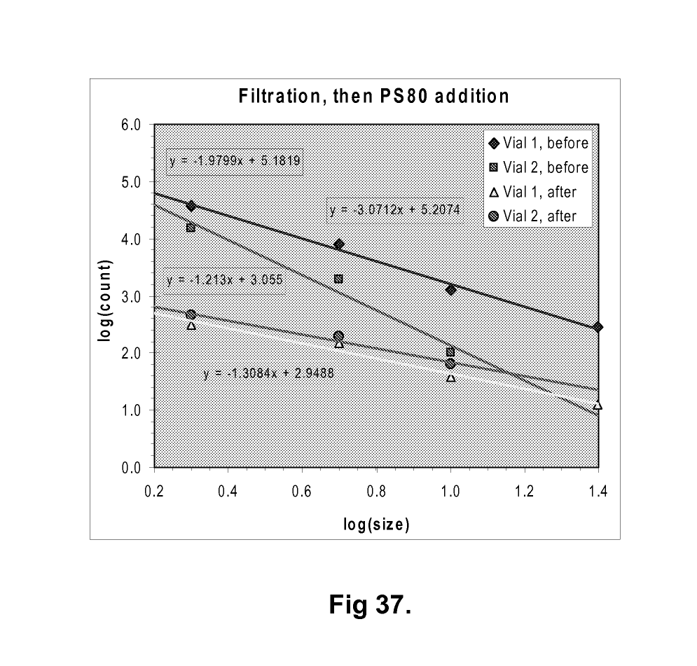

A two-step treatment of filtration followed by PS-80 addition resulted in further particle clearance by a factor of 2-3 over that achieved by filtration alone (FIG. 37 and Table 7). The additional reduction of the slope in the double-logarithmic plot from ˜2.5 to ˜1.2 at the end of the treatment (compared to ˜1.6 by filtration alone) suggested that addition of surfactant, PS80, may disaggregate particles into aggregates of smaller size.

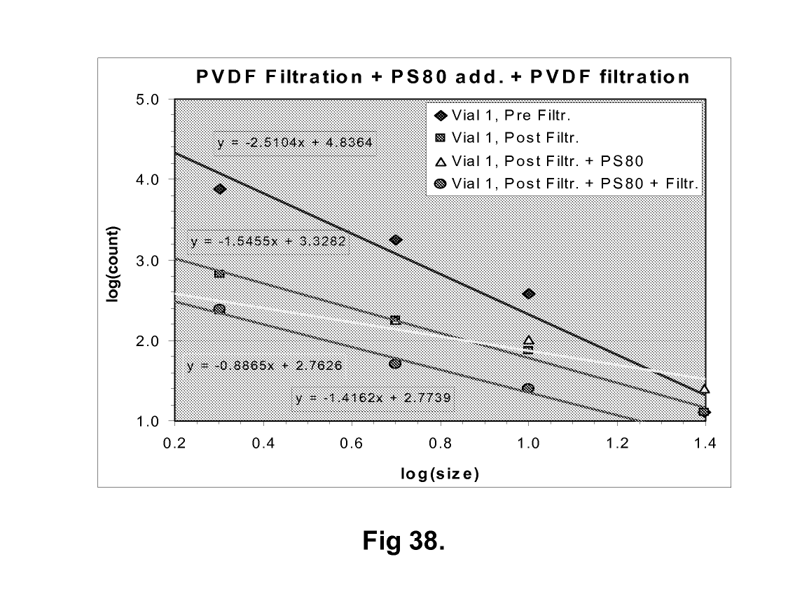

To better resolve the effect of the various treatment steps, the particle size distribution of the formulation sample subject to subsequent filtration and surfactant addition step was characterized (FIG. 38 and Table 7). The first filtration step resulted in a ˜30-fold drop in particle count (at 1 μm) and a decrease in slope from ˜2.5 to ˜1.5, as shown previously (FIG. 36). Further treatment with PS-80 showed a 4-fold reduction in particle count at 1 μm, with additional flatterning of the power law distribution, similar to what was described in FIG. 37. A final filtration step did not significantly affect the numbers of the smaller particles, but decreased somewhat the count for larger sub-visible particles; the effect on total particle count would be marginal, given the low number of particles in the sample in this size range.

TABLE 7
Filtration Data - 0.22 μm PVDF Filter

| 551 + PS-80 followed by Filtration through | | | | | | | |
|---|---|---|---|---|---|---|---|
| 0.22 μm PVDF Filter | | | | | | | |
| | | | HIAC (Cumulative | | | | |
| | | | Count/mL) | | | | |
| | | | >2 | >5 | >10 | >25 | Purity % |
| Sample | | VI | μm | μm | μm | μm | (HPSE) |
| 551, Pre-filtration | Vial | Fail | 7625 | 1773 | 380 | 13 | 99.6 |
| | 1 | | | | | | |
| 551, Post-filtration | Vial | Pass | 671 | 177 | 76 | 13 | 99.6 |
| | 1 | | | | | | |
| 551, Post-filtration + | Vial | Pass | 241 | 177 | 101 | 25 | 99.6 |
| PS-80 Addition | 1 | | | | | | |
| 551, Post-filtration + | Vial | Pass | 241 | 51 | 25 | 0 | 99.6 |
| PS-80 Addition + | 1 | | | | | | |
| Filiation | | | | | | | |

It was found that the initial A280 and purity results were within the expected range. Moreover, vial to vial variability for the sub visible particle count by HIAC was observed, the results of which are reflected in Table 7. PVDF filters were found to perform better than PES filters with one round of filtration (data not shown), while two rounds of filtration to reduce the sub-visible particle count was comparable for both filters. The particles in the sample vials followed an exponential relationship (linear double-logarithmic plots) as shown in FIGS. 36 (PVDF filtration only), 37 (PVDF filtration, then PS80 addition), and 38 (PVDF filtration+PS80 addition+PVDF filtration). Filtration through a 0.22 μm PVDF membrane decreased the particle count by >˜100-fold at 1 μm. Moreover, smaller particles were removed more readily, especially with the addition of 0.02% PS-80. The first PVDF filtration step was shown to exhibit a ˜30-fold drop in particles count (at 1 μm), whereas PS-80 addition clearly increased particle removal by an extra ˜4-fold. Furthermore, the final PVDF filtration step was shown only to decrease the count of the larger sub-visible and visible particles. It was concluded that filtration using the 0.22 μm PVDF filter reduced the number of sub-visible particles.

7.2. The Use of Anti-CD19 Antibodies in Multiple Myeloma Treatment

Multiple myeloma is a malignancy of plasma cells. It is generally understood that multiple myeloma cells CD19−CD138+. It has been noticed, however, that a small fraction of multiple myeloma cells have a CD19+CD138− phenotype. It has been proposed that the CD19+CD138− cells function as cancer stem cells whose progeny matures into CD19−CD138+ cells.

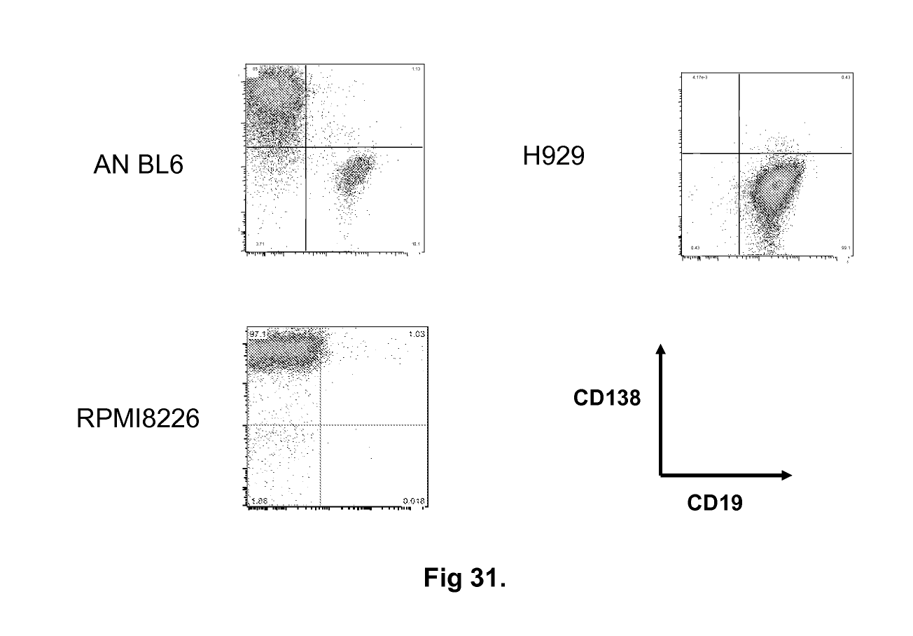

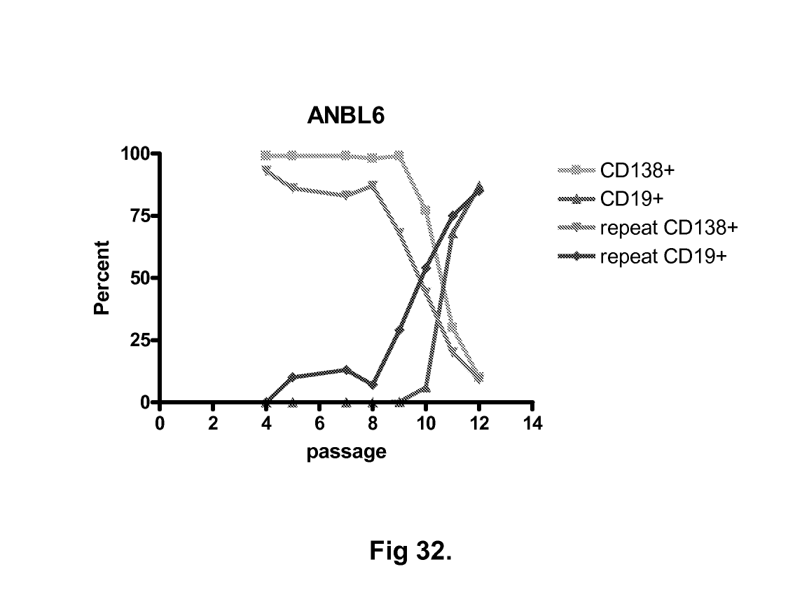

The CD19 and CD138 expression profile of various multiple myeloma cells was analyzed by flow cytometry using standard protocols. A representative sample of the results are shown in FIG. 31. The H929 and RPMI8226 cell lines comprise a mostly homogeneous population of CD19+CD138− and CD19−CD138+, respectively cells. The ANBL6 cell line on the other hand comprises two distinct cell populations, one of which is CD19+CD138− while the other is CD19−CD138+. The ratio of the two population two populations changes over time. As shown in FIG. 32, the ratio of CD19+CD138− increased over time at the expense of CD19−CD138+ cells.

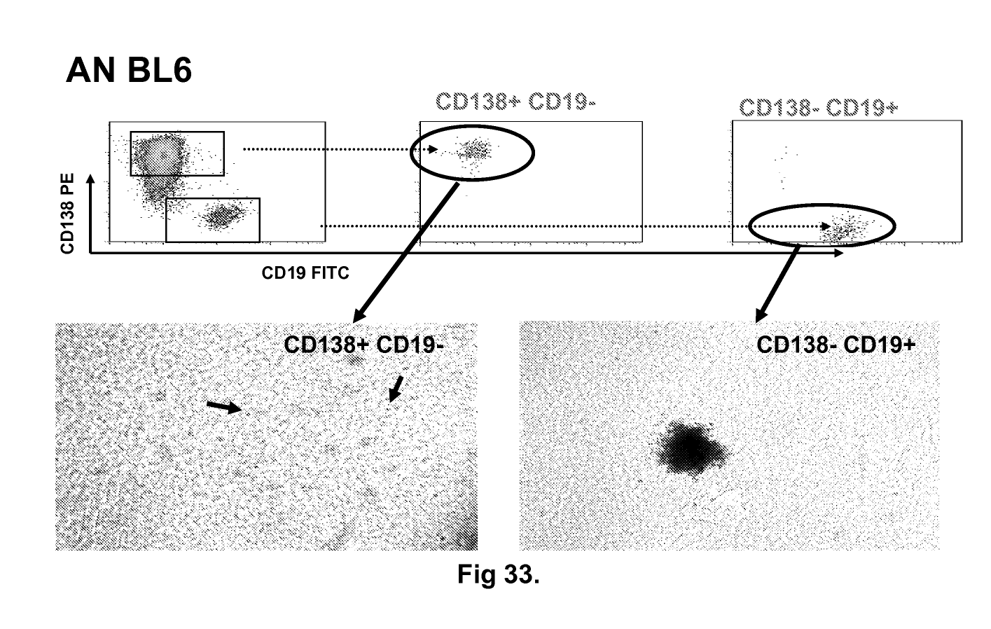

CD19+CD138− and CD19−CD138+ cells were purified from an ANBL6 culture. Flow cytometry data demonstrating the phenotype and purity of the isolated cells is shown in FIG. 33. The purified cells were plated in 0.4% agar containing medium and the cultures were incubated under appropriate conditions for 4-5 weeks. As shown in FIG. 33, only the CD19+CD138− cells were capable of colony formation in this assay.

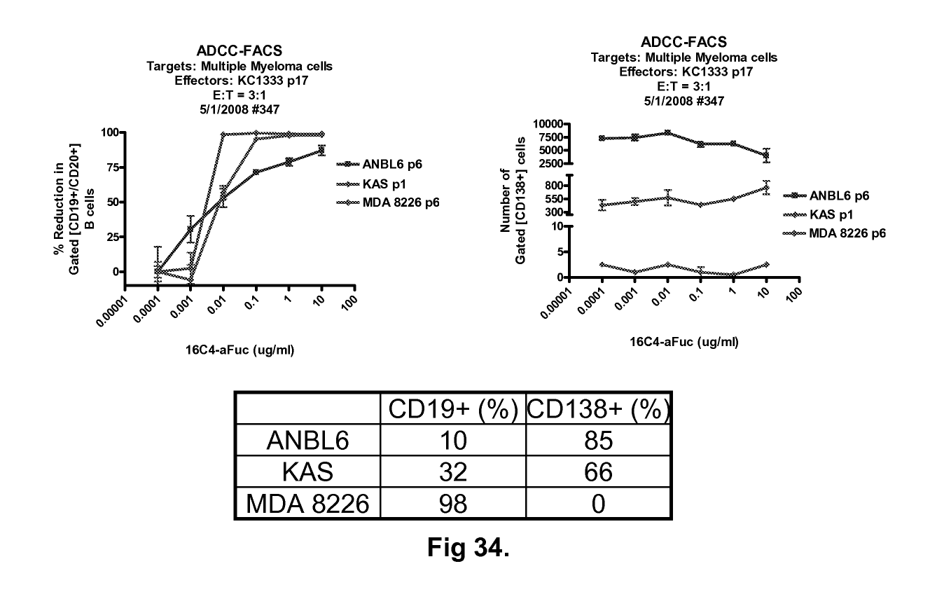

Anti-CD19-aFuc antibody specifically depletes CD19+ cells from unsorted multiple myeloma cell cultures. Unsorted populations of ANBL6, KAS and MDA8226 cells were used as targets in an in vitro anti-CD19 antibody mediated ADCC assay. ADCC assays were performed substantially as described in U.S. patent application Ser. No. 11/852,106, filed on Sep. 7, 2007. The ratio of the CD19+CD138− and CD19−CD138+ cells in the unsorted population is shown in a table at the bottom of FIG. 34. The 16C4-aFuc anti-CD19 antibody mediated ADCC specifically eliminated the CD19+ cells from the population. The % reduction in CD19+ cells as a function of antibody concentration is shown in FIG. 34. The total number of CD138+ cells did not decrease in the course of the assay.

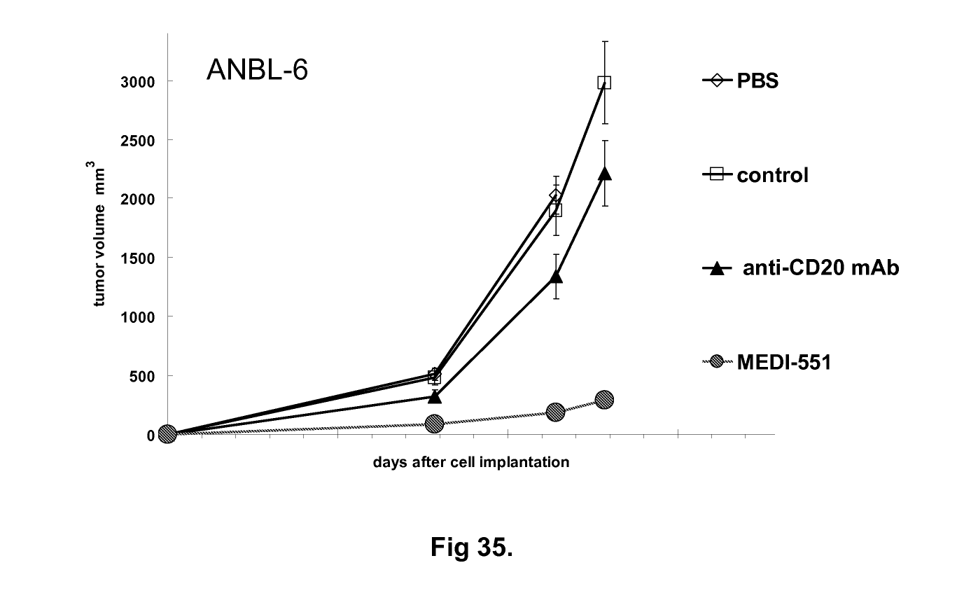

The 551 antibody's ability to control multiple myeloma cell proliferation was ascertained in an in vivo subcutaneous xenograft model in scid mice. Tumor bearing mice (cohorts of 10) were treated with 5 doses of 551, anti-CD20 or control antibody at 3 mg/kg. Antibody treatment was administered twice weekly. Tumor growth was followed by standard measurements. Result obtained using the ANBL-6 cell line is presented in FIG. 35. Tumor growth in 551 treated animals was significantly lower than in any of the control animals. 551 treatment resulted in an 80% reduction in tumor formation by ANBL-6 cells. Similar results were obtained using RPMI8226 and H929 cells. Tumor formation by RPMI8226 and H929 cells was reduced by 75% and 67%, respectively in 551 treated animals. Tumor formation was also reduced in anti-CD20 antibody treated animals; a 44%, 20% and 83% reduction in tumor size was seen in RPMI8226, ANBL-6 and H929, respectively, cell bearing mice.

Those skilled in the art will recognize, or be able to ascertain using no more than routine experimentation, many equivalents to the specific embodiments of the invention described herein. Such equivalents are intended to be encompassed by the following claims.

Various publications are cited herein, the disclosures of which are incorporated by reference in their entireties for all purposes. In addition, U.S. patent application Ser. No. 11/852,106 filed on Sep. 7, 2007 is hereby incorporated by reference in its entirety for all purposes.

SEQUENCES

SEQ ID NO: 2
EVQLQESGPELVKPGASVKMSCKASGYTFTSYVMHWVKQKPGQGLEWIGY
FNPYNDGTDYYEKFKGKATLTSDKSSSTAYMALSSLTSEDSAVYYCARGT
YYYGSSYPFDYWGQGTTLTVSS
SEQ ID NO: 4
DVGMTQTPLTLSVTIGQPASFSCKSSQSLLYSNGKTYLNWLLQRPGQSPK
RLIHLVSKLDSVPDRFTGSGSGTDFTLKIGRVEAEDLGVYYCVQGTHFPY
TFGGGTKLEIK
SEQ ID NO: 6
SYVMH
SEQ ID NO: 8
YFNPYNDGTDYYEKFKG
SEQ ID NO: 10
GTYYYGSSYPFDY
SEQ ID NO: 12
KSSQSLLYSNGKTYLN
SEQ ID NO: 14
LVSKLDS
SEQ ID NO: 16
VQGTHFPYT
SEQ ID NO: 18
QVQLQQSGPELVKPGASVKISCKASGYAFSSSWMNWVIQRPGQGLEWIGRIYPGDGDTNYNGKFKGKATL
TADKSSSTAYMQLSSLTSVDSAVYFCARSGFITTVLDFDYWGHGTTLTVSS
SEQ ID NO: 20
DIVLTQSPTSLAVSLGQRATISCRASESVDTFGISFMNWFQQKPGQPPKLLIHAASNQGSGVPARFSGSG
SGTDFSLNIHPMEEDDSAMYFCQQSKEVPFTFGSGTKLEIK
SEQ ID NO: 22
SSWMN
SEQ ID NO: 24
RIYPGDGDTNYNGKFKG
SEQ ID NO: 26
SGFITTVLDFDY
SEQ ID NO: 28
RASESVDTFGISFMN
SEQ ID NO: 30
AASNQGS
SEQ ID NO: 32
QQSKEVPFT
SEQ ID NO: 34
EVQLVESGGGLVQPGGSLRLSCAASGFTFSSSWMNWVRQAPGKGLEWVGRIYPGDGDTNYNGKFKGRFTI
SRDDSKNSLYLQMNSLKTEDTAVYYCARSGFITTVLDFDYWGQGTLVTVSS
SEQ ID NO: 36
EVQLVESGGGLVQPGGSLRLSCAASGFTFS
SEQ ID NO: 38
WVRQAPGKGLEWVG
SEQ ID NO: 40
RFTISRDDSKNSLYLQMNSLKTEDTAVYYCAR
SEQ ID NO: 42
WGQGTLVTVSS
SEQ ID NO: 44
EVQLVESGGGLVQPGGSLRISCAASGYAFSSSWMNWVIQAPGKGLEWIGRIYPGDGDTNYNGKFKGRATI
SADDSKNSLYMQMNSLKTEDTAVYICARSGFITTVLDFDYWGQGTLVTVSS
SEQ ID NO: 52
EIVLTQSPDFQSVTPKEKVTITCRASESVDTFGISFMNWYQQKPDQSPKLLIKAASNQGSGVPSRFSGSG
SGTDFTLTINSLEAEDAATYYCQQSKEVPFTFGGGTKVEIK
SEQ ID NO: 54
EIVLTQSPDFQSVTPKEKVTITC
SEQ ID NO: 56
WYQQKPDQSPKLLIK
SEQ ID NO: 58
GVPSRFSGSGSGTDFTLTINSLEAEDAATYYC
SEQ ID NO: 60
FGGGTKVEIK
SEQ ID NO: 62
EIVLTQSPDFQSVTPKEKVTITCRASESVDTFGISFMNWFQQKPDQSPKLLIHAASNQGSGVPSRFSGSG
SGTDFTLTINSLEAEDAATYFCQQSKEVPFTFGGGTKVEIK
SEQ ID NO: 64
WFQQKPDQSPKLLIH
SEQ ID NO: 66
GVPSRFSGSGSGTDFTLTINSLEAEDAATYFC
SEQ ID NO: 68
EIVLTQSPDFQSVTPKEKVTITCRASESVDTFGISFMNWFQQKPDQSPKLLIHAASNQGSGVPSRFSGSG
SGTDFTLTINSLEAEDAATYYCQQSKEVPFTFGGGTKVEIK
SEQ ID NO: 70
EIVLTQSPDFQSVTPKEKVTITCRASESVDTFGISFMNWFQQKPDQSPKLLIKAASNQGSGVPSRFSGSG
SGTDFTLTINSLEAEDAATYYCQQSKEVPFTFGGGTKVEIK
SEQ ID NO: 72
WFQQKPDQSPKLLIK
SEQ ID NO: 82
WYQQKPDQSPKLLIH
SEQ ID NO: 83
MGDNDIHFAFLSTGVHS
SEQ ID NO: 84
MDMRVPAQLLGLLLLWLPGAKC
SEQ ID NO: 102
EVQLVESGGGLVQPGGSLRLSCAASGFTFSSTWMNWVRQAPGKGLEWVGRIYPGDGDTNYNGKFKGRFTI
SRDDSKNSLYLQMNSLKTEDTAVYYCARSGFITTVYDFDYWGQGTLVTVSS
SEQ ID NO: 103
EVQLVESGGGLVQPGGSLRLSCAASGFTFSSSWMNWVRQAPGKGLEWVGRIYPGDGDTNYNGKFKGRFTI
SRDDSKNSLYLQMNSLKTEDTAVYYCARSGFITTVRDFDYWGQGTLVTVSS
SEQ ID NO: 104- amino acid sequence of 15D1 VH region
EVQLVESGGGLVQPGGSLRLSCAASGFTFSSSWMNWVRQAPGKGLEWVGRIYPGDGDTNYNAKFKGRFTI
SRDDSKNSLYLQMNSLKTEDTAVYYCARSGFITTVRDFDYWGQGTLVTVSS
SEQ ID NO: 105
EVQLVESGGGLVQPGGSLRLSCAASGFTFSSSWMNWVRQAPGKGLEWVGRIYPGDGDTNYNGKFKGRFTI
SRDDSKNSLYLQMNSLKTEDTAVYYCARSGFITTVHDFDYWGQGTLVTVSS
SEQ ID NO: 106
EVQLVESGGGLVQPGGSLRLSCAASGFTFSSSWMNWVRQAPGKGLEWVGRIYPGDGDTNYNVKFKGRFTI
SRDDSKNSLYLQMNSLKTEDTAVYYCARSGFITTVRDFDYWGQGTLVTVSS
SEQ ID NO: 107
EVQLVESGGGLVQPGGSLRLSCAASGFTFSSSWMNWVRQAPGKGLEWVGRIYPGDGDTNYYGKFKGRFTI
SRDDSKNSLYLQMNSLKTEDTAVYYCARSGFITTVRDFDYWGQGTLVTVSS
SEQ ID NO: 108
EVQLVESGGGLVQPGGSLRLSCAASGFTFSSSWMNWVRQAPGKGLEWVGRIYPGDGDTNYDGKFKGRFTI
SRDDSKNSLYLQMNSLKTEDTAVYYCARSGFITTVRDFDYWGQGTLVTVSS
SEQ ID NO: 109
EVQLVESGGGLVQPGGSLRLSCAASGFTFSSSWMNWVRQAPGKGLEWVGRIYPGDGDTNYLGKFKGRFTI
SRDDSKNSLYLQMNSLKTEDTAVYYCARSGFITTVRDFDYWGQGTLVTVSS
SEQ ID NO: 110
EIVLTQSPDFQSVTPKEKVTITCRASESVDTFGISFINWFQQKPDQSPKLLIHEASNQGSGVPSRFSGSG
SGTDFTLTINSLEAEDAATYYCQQTKEVPFTFGGGTKVEIK
SEQ ID NO: 111
EIVLTQSPDFQSVTPKEKVTITCRASESVDTFGISFMNWFQQKPDQSPKLLIHEASNQGSGVPSRFSGSG
SGTDFTLTINSLEAEDAATYYCQQSKEVPFTFGGGTKVEIK
SEQ ID NO: 112
EIVLTQSPDFQSVTPKEKVTITCRASESVDHFGISFMNWFQQKPDQSPKLLIHEASNQGSGVPSRFSGSG
SGTDFTLTINSLEAEDAATYYCQQSKEVPITFGGGTKVEIK
SEQ ID NO: 113
EIVLTQSPDFQSVTPKEKVTITCRASESVDTFGISFMNWFQQKPDQSPKLLIHAASNQGSGVPSRFSGSG
SGTDFTLTINSLEAEDAATYYCQQSKEVPITFGGGTKVEIK
SEQ ID NO: 114
STWMN
SEQ ID NO: 115
RIYPGDGDTNYNAKFKG
SEQ ID NO: 116
RIYPGDGDTNYNVKFKG
SEQ ID NO: 117
RIYPGDGDTNYYGKFKG
SEQ ID NO: 118
RIYPGDGDTNYDGKFKG
SEQ ID NO: 119
RIYPGDGDTNYLGKFKG
SEQ ID NO: 120
SGFITTVYDFDY
SEQ ID NO: 121
SGFITTVRDFDY
SEQ ID NO: 122
SGFITTVHDFDY
SEQ ID NO: 123
RASESVDTFGISFIN
SEQ ID NO: 124
RASESVDHFGISFMN
SEQ ID NO: 125
EASNQGS
SEQ ID NO: 126
QQTKEVPFT
SEQ ID NO: 127
QQSKEVPIT
SEQ ID NO: 191
EVQLVESGGGLVQPGGSLRLSCAASGFTFSSVWMNWVRQAPGKGLEWVGRIYPGDGDTNYNVKFKGRFTI
SRDDSKNSLYLQMNSLKTEDTAVYYCARSGFITTVRDFDYWGQGTLVTVSS
SEQ ID NO: 192
EVQLVESGGGLVQPGGSLRLSCAASGFTFSSVWMNWVRQAPGKGLEWVGRIYLGDGDTNYNVKFKGRFTI
SRDDSKNSLYLQMNSLKTEDTAVYYCARSGFITTVRDFDYWGQGTLVTVSS
SEQ ID NO: 193
EIVLTQSPDFQSVTPKEKVTITCRASESVDHFGISFINWFQQKPDQSPKLLIHEASNPYSGVPSRFSGSG
SGTDFTLTINSLEAEDAATYYCAQSKEVPITFGGGTKVEIK
SEQ ID NO: 194
EIVLTQSPDFQSVTPKEKVTITCRASESVDTFGISFMNWFQQKPDQSPKLLIHEASNQGSGVPSRFSGSG
SGTDFTLTINSLEAEDAATYYCAQSKEVPFTFGGGTKVEIK
SEQ ID NO: 195
EIVLTQSPDFQSVTPKEKVTITCRASESVDTFGISFMNWFQQKPDQSPKLLIHEASNQGSGVPSRFSGSG
SGTDFTLTINSLEAEDAATYYCAQSKRVPFTFGGGTKVEIK
SEQ ID NO: 196
EIVLTQSPDFQSVTPKEKVTITCRASESVITFGISFMNWFQQKPDQSPKLLIHEASNQGSGVPSRFSGSG
SGTDFTLTINSLEAEDAATYYCQQSKEVPFTFGGGTKVEIK
SEQ ID NO: 197
EIVLTQSPDFQSVTPKEKVTITCRASESVDTFGISFMNWFQQKPDQSPKLLIHEASNQGSGVPSRFSGSG
SGTDFTLTINSLEAEDAATYYCAQTKRVPFTFGGGTKVEIK
SEQ ID NO: 198
EIVLTQSPDFQSVTPKEKVTITCRASESVDTFGISFMNWFQQKPDQSPKLLIHEASNQGSGVPSRFSGSG
SGTDFTLTINSLEAEDAATYYCQQSKEVPITFGGGTKVEIK
SEQ ID NO: 199
EIVLTQSPDFQSVTPKEKVTITCRASESVDTFGISFINWFQQKPDQSPKLLIHEASNPYSGVPSRFSGSG
SGTDFTLTINSLEAEDAATYYCQQSKEVPFTFGGGTKVEIK
SEQ ID NO: 200
EIVLTQSPDFQSVTPKEKVTITCRASESVDTFGISFMNWFQQKPDQSPKLLIHEASNQGSGVPSRFSGSG
SGTDFTLTINSLEAEDAATYYCAQTKEVPFTFGGGTKVEIK
SEQ ID NO: 201
EIVLTQSPDFQSVTPKEKVTITCRASESVDTFGISFMNWFQQKPDQSPKLLIHEASNQGSGVPSRFSGSG
SGTDFTLTINSLEAEDAATYYCAQTKEVPNTFGGGTKVEIK
SEQ ID NO: 202
EIVLTQSPDFQSVTPKEKVTITCRASESVITFGISFMNWFQQKPDQSPKLLIHEASNTYSGVPSRFSGSG
SGTDFTLTINSLEAEDAATYYCAQSKRVPFTFGGGTKVEIK
SEQ ID NO: 203
EIVLTQSPDFQSVTPKEKVTITCRASESVDTFGISFRNWFQQKPDQSPKLLIHEASNQGSGVPSRFSGSG
SGTDFTLTINSLEAEDAATYYCQQSKEVPFTFGGGTKVEIK
SEQ ID NO: 204
EIVLTQSPDFQSVTPKEKVTITCRASESVDTFGISFMNWFQQKPDQSPKLLIHEASNPGSGVPSRFSGSG
SGTDFTLTINSLEAEDAATYYCQQTKRVPFTFGGGTKVEIK
SEQ ID NO: 205
EIVLTQSPDFQSVTPKEKVTITCRASESVIHFGISFMNWFQQKPDQSPKLLIHEASNRGSGVPSRFSGSG
SGTDFTLTINSLEAEDAATYYCAQSKEVPITFGGGTKVEIK
SEQ ID NO: 206
EIVLTQSPDFQSVTPKEKVTITCRASESVDTFGLSFMNWFQQKPDQSPKLLIHEASNPYSGVPSRFSGSG
SGTDFTLTINSLEAEDAATYYCQQSKEVPFTFGGGTKVEIK
SEQ ID NO: 207
EIVLTQSPDFQSVTPKEKVTITCRASESVITFGISFINWFQQKPDQSPKLLIHEASNPYSGVPSRFSGSG
SGTDFTLTINSLEAEDAATYYCAQTKRVPFTFGGGTKVEIK
SEQ ID NO: 208
SVWMN
SEQ ID NO: 209
STWMN
SEQ ID NO: 210
RIYLGDGDTNYNVKFKG
SEQ ID NO: 211
RASESVDHFGISFIN
SEQ ID NO: 212
RASESVITFGISFMN
SEQ ID NO: 213
RASESVDTFGISFIN
SEQ ID NO: 214
RASESVDTFGISFRN
SEQ ID NO: 215
RASESVIHFGISFMN
SEQ ID NO: 216
RASESVDTFGLSFMN
SEQ ID NO: 217
RASESVITFGISFIN
SEQ ID NO: 218
EASNPYS
SEQ ID NO: 219
EASNTYS
SEQ ID NO: 220
EASNPGS
SEQ ID NO: 221
EASNRGS
SEQ ID NO: 222
AQSKEVPIT
SEQ ID NO: 223
AQSKEVPIT
SEQ ID NO: 224
AQSKRVPFT
SEQ ID NO: 225
AQTKRVPFT
SEQ ID NO: 226
QQSKEVPIT
SEQ ID NO: 227
AQTKEVPFT
SEQ ID NO: 228
AQTKEVPNT
SEQ ID NO: 229
QQTKRVPFT
SEQ ID NO: 230
S(S/T/V)WMN
SEQ ID NO: 231
RIY(P/L)GDGDTNY(N/Y/D/L)(G/A/V)KFKG
SEQ ID NO: 232
SGFITTV(L/R/Y/H)DFDY
SEQ ID NO: 233
RASESV(D/I)(T/H)FG(I/L)SF(M/I/R)N
SEQ ID NO: 234
(A/E)ASN(Q/P/T)(G/Y)S
SEQ ID NO: 235
(Q/A)Q(S/T)K(E/R)VP(F/I/N)T
SEQ ID NO: 236
EVQLVESGGGLVQPGGSLRLSCAASGFTFSSTWMNWVRQAPGKGLEWVGRIYPGDGDTNYNVKFKGRFTI
SRDDSKNSLYLQMNSLKTEDTAVYYCARSGFITTVRDFDYWGQGTLVTVSS
SEQ ID NO: 237
EVQLVESGGGLVQPGGSLRLSCAASGFTFSSSWMNWVRQAPGKGLEWVGRIYPGDGDTNYNGKFKGRFTI
SRDDSKNSLYLQMNSLKTEDTAVYYCARSGFITTVLDFDYWGQGTLVTVSS
SEQ ID NO: 238
EIVLTQSPDFQSVTPKEKVTITCRASESVDTFGISFMNWFQQKPDQSPKLLIHAASNQGSGVPSRFSGSG
SGTDFTLTINSLEAEDAATYYCQQSKEVPFTFGGGTKVEIK

1. A sterile, stable aqueous formulation comprising a chimeric, humanized or human anti-CD19 antibody.

2. (canceled)

3. The formulation of claim 1, wherein said antibody is from an immunoglobulin type selected from the group consisting of IgA, IgE, IgM, IgD, IgY and IgG.

4. The formulation of claim 3, wherein said antibody is of IgG1, IgG2, IgG3, or IgG4 human isotype.

5. The formulation of claim 1, wherein the antibody comprises an Fc region having complex N-glycoside-linked sugar chains having a reducing end in which fucose is not bound to N-acetylglucosamine in the reducing end in the sugar chain.

6. The formulation of claim 1, wherein said antibody comprises:
(i) a heavy chain variable region comprising a sequence of SEQ ID NO: 104; and/or
(ii) a light chain variable region comprising a sequence of SEQ ID NO: 111.

7-8. (canceled)

9. The formulation of claim 6, wherein said antibody comprises an Fc region having complex N-glycoside-linked sugar chains with a reducing end in which fucose is not bound to N-acetylglucosamine in the reducing end in the sugar chain.

10-17. (canceled)

18. The formulation of claim 1, wherein said formulation further comprises at least about one buffering component selected from histidine, citrate, phosphate, glycine, and acetate and at least about one excipient selected from a saccharide, a salt, and a surfactant.

19-84. (canceled)

85. The sterile, stable aqueous formulation of claim 1 comprising a chimeric, humanized or human anti-CD19 antibody, and further comprising histidine, sodium chloride, and trehalose, wherein the antibody comprises a heavy chain variable region comprising a sequence of SEQ ID NO: 104, a light chain variable region comprising a sequence of SEQ ID NO: 111 and an Fc region having complex N-glycoside-linked sugar chains having a reducing end in which fucose is not bound to N-acetylglucosamine in the reducing end in the sugar chain.

86-87. (canceled)

88. The formulation of claim 1, wherein said formulation comprises about 10 mg/ml of the anti-CD19 antibody, about 10 mM histidine, and about 4% trehalose, and wherein the formulation has a pH of about 6.

89-122. (canceled)

123. A method of treating a B cell disease or disorder in a human, comprising administering to a human in need thereof a therapeutically-effective amount of the formulation of claim 1.

124. The method of claim 123, wherein said B cell disease or disorder is selected from a group consisting of: B cell malignancy, autoimmune disease, autoimmune disorder, humoral rejection in a human transplant patient, graft-versus-host disease (GVHD) and post-transplantation lymphoproliferative disorder in a human transplant recipient.

125-158. (canceled)

159. A pharmaceutical unit dosage form suitable for parenteral administration to a human which comprises an antibody formulation of claim 88 in a suitable container.

160. The pharmaceutical unit dosage form of claim 159, wherein the antibody formulation is administered intravenously, subcutaneously, or intramuscularly.

161-172. (canceled)

173. The sterile, stable aqueous formulation of claim 1 comprising a chimeric, humanized or human anti-CD19 antibody, wherein said antibody comprises:
(i) a heavy chain variable region comprising a VH CDR1 (SEQ ID NO: 22), a VH CDR2 (SEQ ID NO: 115), and a VH CDR3 (SEQ ID NO: 121);
(ii) a light chain variable region comprising a VK CDR1 (SEQ ID NO: 28), a VK CDR2 (SEQ ID NO: 125), and a VK CDR3 (SEQ ID NO: 32);
(iii) an Fc region having complex N-glycoside-linked sugar chains having a reducing end in which fucose is not bound to N-acetylglucosamine in the reducing end in the sugar chain; and
(iv) a buffering agent, a salt, a carbohydrate excipient and a surfactant.

174-232. (canceled)

233. A method of enhancing storage stability of an aqueous formulation wherein the formulation comprises a chimeric, humanized or human anti-CD19 antibody comprising,
a. a heavy chain variable region comprising a sequence of SEQ ID NO: 104,
b. a light chain variable region comprising a sequence of SEQ ID NO: 111; and
c. an Fc region having complex N-glycoside-linked sugar chains having a reducing end in which fucose is not bound to N-acetylglucosamine in the reducing end in the sugar chain; and
wherein said method comprises adding a surfactant to said formulation in an amount effective to enhance the storage stability of the formulation.

234. The method of claim 233 wherein the surfactant is polysorbate 80.

235-246. (canceled)


SEQUENCE LISTING (239 sequences)

SEQ ID NO: 1 - skipped

SEQ ID NO: 2 - protein, 122 aa, Artificial Sequence
  Description of Artificial Sequence Synthetic polypeptide
EVQLQESGPELVKPGASVKMSCKASGYTFTSYVMHWVKQKPGQGLEWIGYFNPYNDGTDYYEKFKGKATLTSDKSSSTAYMALSSLTSEDSAVYYCARGTYYYGSSYPFDYWGQGTTLTVSS

SEQ ID NO: 3 - skipped

SEQ ID NO: 4 - protein, 111 aa, Artificial Sequence
  Description of Artificial Sequence Synthetic polypeptide
DVGMTQTPLTLSVTIGQPASFSCKSSQSLLYSNGKTYLNWLLQRPGQSPKRLIHLVSKLDSVPDRFTGSGSGTDFTLKIGRVEAEDLGVYYCVQGTHFPYTFGGGTKLEIK

SEQ ID NO: 5 - skipped

SEQ ID NO: 6 - protein, 5 aa, Artificial Sequence
  Description of Artificial Sequence Synthetic peptide
SYVMH

SEQ ID NO: 7 - skipped

SEQ ID NO: 8 - protein, 17 aa, Artificial Sequence
  Description of Artificial Sequence Synthetic peptide
YFNPYNDGTDYYEKFKG

SEQ ID NO: 9 - skipped

SEQ ID NO: 10 - protein, 13 aa, Artificial Sequence
  Description of Artificial Sequence Synthetic peptide
GTYYYGSSYPFDY

SEQ ID NO: 11 - skipped

SEQ ID NO: 12 - protein, 16 aa, Artificial Sequence
  Description of Artificial Sequence Synthetic peptide
KSSQSLLYSNGKTYLN

SEQ ID NO: 13 - skipped

SEQ ID NO: 14 - protein, 7 aa, Artificial Sequence
  Description of Artificial Sequence Synthetic peptide
LVSKLDS

SEQ ID NO: 15 - skipped

SEQ ID NO: 16 - protein, 9 aa, Artificial Sequence
  Description of Artificial Sequence Synthetic peptide
VQGTHFPYT

SEQ ID NO: 17 - skipped

SEQ ID NO: 18 - protein, 121 aa, Artificial Sequence
  Description of Artificial Sequence Synthetic polypeptide
QVQLQQSGPELVKPGASVKISCKASGYAFSSSWMNWVIQRPGQGLEWIGRIYPGDGDTNYNGKFKGKATLTADKSSSTAYMQLSSLTSVDSAVYFCARSGFITTVLDFDYWGHGTTLTVSS

SEQ ID NO: 19 - skipped

SEQ ID NO: 20 - protein, 111 aa, Artificial Sequence
  Description of Artificial Sequence Synthetic polypeptide
DIVLTQSPTSLAVSLGQRATISCRASESVDTFGISFMNWFQQKPGQPPKLLIHAASNQGSGVPARFSGSGSGTDFSLNIHPMEEDDSAMYFCQQSKEVPFTFGSGTKLEIK

SEQ ID NO: 21 - skipped

SEQ ID NO: 22 - protein, 5 aa, Artificial Sequence
  Description of Artificial Sequence Synthetic peptide
SSWMN

SEQ ID NO: 23 - skipped

SEQ ID NO: 24 - protein, 17 aa, Artificial Sequence
  Description of Artificial Sequence Synthetic peptide
RIYPGDGDTNYNGKFKG

SEQ ID NO: 25 - skipped

SEQ ID NO: 26 - protein, 12 aa, Artificial Sequence
  Description of Artificial Sequence Synthetic peptide
SGFITTVLDFDY

SEQ ID NO: 27 - skipped

SEQ ID NO: 28 - protein, 15 aa, Artificial Sequence
  Description of Artificial Sequence Synthetic peptide
RASESVDTFGISFMN

SEQ ID NO: 29 - skipped

SEQ ID NO: 30 - protein, 7 aa, Artificial Sequence
  Description of Artificial Sequence Synthetic peptide
AASNQGS

SEQ ID NO: 31 - skipped

SEQ ID NO: 32 - protein, 9 aa, Artificial Sequence
  Description of Artificial Sequence Synthetic peptide
QQSKEVPFT

SEQ ID NO: 33 - skipped

SEQ ID NO: 34 - protein, 121 aa, Artificial Sequence
  Description of Artificial Sequence Synthetic polypeptide
EVQLVESGGGLVQPGGSLRLSCAASGFTFSSSWMNWVRQAPGKGLEWVGRIYPGDGDTNYNGKFKGRFTISRDDSKNSLYLQMNSLKTEDTAVYYCARSGFITTVLDFDYWGQGTLVTVSS

SEQ ID NO: 35 - skipped

SEQ ID NO: 36 - protein, 30 aa, Artificial Sequence
  Description of Artificial Sequence Synthetic polypeptide
EVQLVESGGGLVQPGGSLRLSCAASGFTFS

SEQ ID NO: 37 - skipped

SEQ ID NO: 38 - protein, 14 aa, Artificial Sequence
  Description of Artificial Sequence Synthetic peptide
WVRQAPGKGLEWVG

SEQ ID NO: 39 - skipped

SEQ ID NO: 40 - protein, 32 aa, Artificial Sequence
  Description of Artificial Sequence Synthetic polypeptide
RFTISRDDSKNSLYLQMNSLKTEDTAVYYCAR

SEQ ID NO: 41 - skipped

SEQ ID NO: 42 - protein, 11 aa, Artificial Sequence
  Description of Artificial Sequence Synthetic peptide
WGQGTLVTVSS

SEQ ID NO: 43 - skipped

SEQ ID NO: 44 - protein, 121 aa, Artificial Sequence
  Description of Artificial Sequence Synthetic polypeptide
EVQLVESGGGLVQPGGSLRISCAASGYAFSSSWMNWVIQAPGKGLEWIGRIYPGDGDTNYNGKFKGRATISADDSKNSLYMQMNSLKTEDTAVYICARSGFITTVLDFDYWGQGTLVTVSS

SEQ ID NO: 45-51 - skipped (7 entries)

SEQ ID NO: 52 - protein, 111 aa, Artificial Sequence
  Description of Artificial Sequence Synthetic polypeptide
EIVLTQSPDFQSVTPKEKVTITCRASESVDTFGISFMNWYQQKPDQSPKLLIKAASNQGSGVPSRFSGSGSGTDFTLTINSLEAEDAATYYCQQSKEVPFTFGGGTKVEIK

SEQ ID NO: 53 - skipped

SEQ ID NO: 54 - protein, 23 aa, Artificial Sequence
  Description of Artificial Sequence Synthetic peptide
EIVLTQSPDFQSVTPKEKVTITC

SEQ ID NO: 55 - skipped

SEQ ID NO: 56 - protein, 15 aa, Artificial Sequence
  Description of Artificial Sequence Synthetic peptide
WYQQKPDQSPKLLIK

SEQ ID NO: 57 - skipped

SEQ ID NO: 58 - protein, 32 aa, Artificial Sequence
  Description of Artificial Sequence Synthetic polypeptide
GVPSRFSGSGSGTDFTLTINSLEAEDAATYYC

SEQ ID NO: 59 - skipped

SEQ ID NO: 60 - protein, 10 aa, Artificial Sequence
  Description of Artificial Sequence Synthetic peptide
FGGGTKVEIK

SEQ ID NO: 61 - skipped

SEQ ID NO: 62 - protein, 111 aa, Artificial Sequence
  Description of Artificial Sequence Synthetic polypeptide
EIVLTQSPDFQSVTPKEKVTITCRASESVDTFGISFMNWFQQKPDQSPKLLIHAASNQGSGVPSRFSGSGSGTDFTLTINSLEAEDAATYFCQQSKEVPFTFGGGTKVEIK

SEQ ID NO: 63 - skipped

SEQ ID NO: 64 - protein, 15 aa, Artificial Sequence
  Description of Artificial Sequence Synthetic peptide
WFQQKPDQSPKLLIH

SEQ ID NO: 65 - skipped

SEQ ID NO: 66 - protein, 32 aa, Artificial Sequence
  Description of Artificial Sequence Synthetic polypeptide
GVPSRFSGSGSGTDFTLTINSLEAEDAATYFC

SEQ ID NO: 67 - skipped

SEQ ID NO: 68 - protein, 111 aa, Artificial Sequence
  Description of Artificial Sequence Synthetic polypeptide
EIVLTQSPDFQSVTPKEKVTITCRASESVDTFGISFMNWFQQKPDQSPKLLIHAASNQGSGVPSRFSGSGSGTDFTLTINSLEAEDAATYYCQQSKEVPFTFGGGTKVEIK

SEQ ID NO: 69 - skipped

SEQ ID NO: 70 - protein, 111 aa, Artificial Sequence
  Description of Artificial Sequence Synthetic polypeptide
EIVLTQSPDFQSVTPKEKVTITCRASESVDTFGISFMNWFQQKPDQSPKLLIKAASNQGSGVPSRFSGSGSGTDFTLTINSLEAEDAATYYCQQSKEVPFTFGGGTKVEIK

SEQ ID NO: 71 - skipped

SEQ ID NO: 72 - protein, 15 aa, Artificial Sequence
  Description of Artificial Sequence Synthetic peptide
WFQQKPDQSPKLLIK

SEQ ID NO: 73-81 - skipped (9 entries)

SEQ ID NO: 82 - protein, 15 aa, Artificial Sequence
  Description of Artificial Sequence Synthetic peptide
WYQQKPDQSPKLLIH

SEQ ID NO: 83 - protein, 17 aa, Artificial Sequence
  Description of Artificial Sequence Synthetic peptide
MGDNDIHFAFLSTGVHS

SEQ ID NO: 84 - protein, 22 aa, Artificial Sequence
  Description of Artificial Sequence Synthetic peptide
MDMRVPAQLLGLLLLWLPGAKC

SEQ ID NO: 85-101 - skipped (17 entries)

SEQ ID NO: 102 - protein, 121 aa, Artificial Sequence
  Description of Artificial Sequence Synthetic polypeptide
EVQLVESGGGLVQPGGSLRLSCAASGFTFSSTWMNWVRQAPGKGLEWVGRIYPGDGDTNYNGKFKGRFTISRDDSKNSLYLQMNSLKTEDTAVYYCARSGFITTVYDFDYWGQGTLVTVSS

SEQ ID NO: 103 - protein, 121 aa, Artificial Sequence
  Description of Artificial Sequence Synthetic polypeptide
EVQLVESGGGLVQPGGSLRLSCAASGFTFSSSWMNWVRQAPGKGLEWVGRIYPGDGDTNYNGKFKGRFTISRDDSKNSLYLQMNSLKTEDTAVYYCARSGFITTVRDFDYWGQGTLVTVSS

SEQ ID NO: 104 - protein, 121 aa, Artificial Sequence
  Description of Artificial Sequence Synthetic polypeptide
EVQLVESGGGLVQPGGSLRLSCAASGFTFSSSWMNWVRQAPGKGLEWVGRIYPGDGDTNYNAKFKGRFTISRDDSKNSLYLQMNSLKTEDTAVYYCARSGFITTVRDFDYWGQGTLVTVSS

SEQ ID NO: 105 - protein, 121 aa, Artificial Sequence
  Description of Artificial Sequence Synthetic polypeptide
EVQLVESGGGLVQPGGSLRLSCAASGFTFSSSWMNWVRQAPGKGLEWVGRIYPGDGDTNYNGKFKGRFTISRDDSKNSLYLQMNSLKTEDTAVYYCARSGFITTVHDFDYWGQGTLVTVSS

SEQ ID NO: 106 - protein, 121 aa, Artificial Sequence
  Description of Artificial Sequence Synthetic polypeptide
EVQLVESGGGLVQPGGSLRLSCAASGFTFSSSWMNWVRQAPGKGLEWVGRIYPGDGDTNYNVKFKGRFTISRDDSKNSLYLQMNSLKTEDTAVYYCARSGFITTVRDFDYWGQGTLVTVSS

SEQ ID NO: 107 - protein, 121 aa, Artificial Sequence
  Description of Artificial Sequence Synthetic polypeptide
EVQLVESGGGLVQPGGSLRLSCAASGFTFSSSWMNWVRQAPGKGLEWVGRIYPGDGDTNYYGKFKGRFTISRDDSKNSLYLQMNSLKTEDTAVYYCARSGFITTVRDFDYWGQGTLVTVSS

SEQ ID NO: 108 - protein, 121 aa, Artificial Sequence
  Description of Artificial Sequence Synthetic polypeptide
EVQLVESGGGLVQPGGSLRLSCAASGFTFSSSWMNWVRQAPGKGLEWVGRIYPGDGDTNYDGKFKGRFTISRDDSKNSLYLQMNSLKTEDTAVYYCARSGFITTVRDFDYWGQGTLVTVSS

SEQ ID NO: 109 - protein, 121 aa, Artificial Sequence
  Description of Artificial Sequence Synthetic polypeptide
EVQLVESGGGLVQPGGSLRLSCAASGFTFSSSWMNWVRQAPGKGLEWVGRIYPGDGDTNYLGKFKGRFTISRDDSKNSLYLQMNSLKTEDTAVYYCARSGFITTVRDFDYWGQGTLVTVSS

SEQ ID NO: 110 - protein, 111 aa, Artificial Sequence
  Description of Artificial Sequence Synthetic polypeptide
EIVLTQSPDFQSVTPKEKVTITCRASESVDTFGISFINWFQQKPDQSPKLLIHEASNQGSGVPSRFSGSGSGTDFTLTINSLEAEDAATYYCQQTKEVPFTFGGGTKVEIK

SEQ ID NO: 111 - protein, 111 aa, Artificial Sequence
  Description of Artificial Sequence Synthetic polypeptide
EIVLTQSPDFQSVTPKEKVTITCRASESVDTFGISFMNWFQQKPDQSPKLLIHEASNQGSGVPSRFSGSGSGTDFTLTINSLEAEDAATYYCQQSKEVPFTFGGGTKVEIK

SEQ ID NO: 112 - protein, 111 aa, Artificial Sequence
  Description of Artificial Sequence Synthetic polypeptide
EIVLTQSPDFQSVTPKEKVTITCRASESVDHFGISFMNWFQQKPDQSPKLLIHEASNQGSGVPSRFSGSGSGTDFTLTINSLEAEDAATYYCQQSKEVPITFGGGTKVEIK

SEQ ID NO: 113 - protein, 111 aa, Artificial Sequence
  Description of Artificial Sequence Synthetic polypeptide
EIVLTQSPDFQSVTPKEKVTITCRASESVDTFGISFMNWFQQKPDQSPKLLIHAASNQGSGVPSRFSGSGSGTDFTLTINSLEAEDAATYYCQQSKEVPITFGGGTKVEIK

SEQ ID NO: 114 - protein, 5 aa, Artificial Sequence
  Description of Artificial Sequence Synthetic peptide
STWMN

SEQ ID NO: 115 - protein, 17 aa, Artificial Sequence
  Description of Artificial Sequence Synthetic peptide
RIYPGDGDTNYNAKFKG

SEQ ID NO: 116 - protein, 17 aa, Artificial Sequence
  Description of Artificial Sequence Synthetic peptide
RIYPGDGDTNYNVKFKG

SEQ ID NO: 117 - protein, 17 aa, Artificial Sequence
  Description of Artificial Sequence Synthetic peptide
RIYPGDGDTNYYGKFKG

SEQ ID NO: 118 - protein, 17 aa, Artificial Sequence
  Description of Artificial Sequence Synthetic peptide
RIYPGDGDTNYDGKFKG

SEQ ID NO: 119 - protein, 17 aa, Artificial Sequence
  Description of Artificial Sequence Synthetic peptide
RIYPGDGDTNYLGKFKG

SEQ ID NO: 120 - protein, 12 aa, Artificial Sequence
  Description of Artificial Sequence Synthetic peptide
SGFITTVYDFDY

SEQ ID NO: 121 - protein, 12 aa, Artificial Sequence
  Description of Artificial Sequence Synthetic peptide
SGFITTVRDFDY

SEQ ID NO: 122 - protein, 12 aa, Artificial Sequence
  Description of Artificial Sequence Synthetic peptide
SGFITTVHDFDY

SEQ ID NO: 123 - protein, 15 aa, Artificial Sequence
  Description of Artificial Sequence Synthetic peptide
RASESVDTFGISFIN

SEQ ID NO: 124 - protein, 15 aa, Artificial Sequence
  Description of Artificial Sequence Synthetic peptide
RASESVDHFGISFMN

SEQ ID NO: 125 - protein, 7 aa, Artificial Sequence
  Description of Artificial Sequence Synthetic peptide
EASNQGS

SEQ ID NO: 126 - protein, 9 aa, Artificial Sequence
  Description of Artificial Sequence Synthetic peptide
QQTKEVPFT

SEQ ID NO: 127 - protein, 9 aa, Artificial Sequence
  Description of Artificial Sequence Synthetic peptide
QQSKEVPIT

SEQ ID NO: 128-190 - skipped (63 entries)

SEQ ID NO: 191 - protein, 121 aa, Artificial Sequence
  Description of Artificial Sequence Synthetic polypeptide
EVQLVESGGGLVQPGGSLRLSCAASGFTFSSVWMNWVRQAPGKGLEWVGRIYPGDGDTNYNVKFKGRFTISRDDSKNSLYLQMNSLKTEDTAVYYCARSGFITTVRDFDYWGQGTLVTVSS

SEQ ID NO: 192 - protein, 121 aa, Artificial Sequence
  Description of Artificial Sequence Synthetic polypeptide
EVQLVESGGGLVQPGGSLRLSCAASGFTFSSVWMNWVRQAPGKGLEWVGRIYLGDGDTNYNVKFKGRFTISRDDSKNSLYLQMNSLKTEDTAVYYCARSGFITTVRDFDYWGQGTLVTVSS

SEQ ID NO: 193 - protein, 111 aa, Artificial Sequence
  Description of Artificial Sequence Synthetic polypeptide
EIVLTQSPDFQSVTPKEKVTITCRASESVDHFGISFINWFQQKPDQSPKLLIHEASNPYSGVPSRFSGSGSGTDFTLTINSLEAEDAATYYCAQSKEVPITFGGGTKVEIK

SEQ ID NO: 194 - protein, 111 aa, Artificial Sequence
  Description of Artificial Sequence Synthetic polypeptide
EIVLTQSPDFQSVTPKEKVTITCRASESVDTFGISFMNWFQQKPDQSPKLLIHEASNQGSGVPSRFSGSGSGTDFTLTINSLEAEDAATYYCAQSKEVPFTFGGGTKVEIK

SEQ ID NO: 195 - protein, 111 aa, Artificial Sequence
  Description of Artificial Sequence Synthetic polypeptide
EIVLTQSPDFQSVTPKEKVTITCRASESVDTFGISFMNWFQQKPDQSPKLLIHEASNQGSGVPSRFSGSGSGTDFTLTINSLEAEDAATYYCAQSKRVPFTFGGGTKVEIK

SEQ ID NO: 196 - protein, 111 aa, Artificial Sequence
  Description of Artificial Sequence Synthetic polypeptide
EIVLTQSPDFQSVTPKEKVTITCRASESVITFGISFMNWFQQKPDQSPKLLIHEASNQGSGVPSRFSGSGSGTDFTLTINSLEAEDAATYYCQQSKEVPFTFGGGTKVEIK

SEQ ID NO: 197 - protein, 111 aa, Artificial Sequence
  Description of Artificial Sequence Synthetic polypeptide
EIVLTQSPDFQSVTPKEKVTITCRASESVDTFGISFMNWFQQKPDQSPKLLIHEASNQGSGVPSRFSGSGSGTDFTLTINSLEAEDAATYYCAQTKRVPFTFGGGTKVEIK

SEQ ID NO: 198 - protein, 111 aa, Artificial Sequence
  Description of Artificial Sequence Synthetic polypeptide
EIVLTQSPDFQSVTPKEKVTITCRASESVDTFGISFMNWFQQKPDQSPKLLIHEASNQGSGVPSRFSGSGSGTDFTLTINSLEAEDAATYYCQQSKEVPITFGGGTKVEIK

SEQ ID NO: 199 - protein, 111 aa, Artificial Sequence
  Description of Artificial Sequence Synthetic polypeptide
EIVLTQSPDFQSVTPKEKVTITCRASESVDTFGISFINWFQQKPDQSPKLLIHEASNPYSGVPSRFSGSGSGTDFTLTINSLEAEDAATYYCQQSKEVPFTFGGGTKVEIK

SEQ ID NO: 200 - protein, 111 aa, Artificial Sequence
  Description of Artificial Sequence Synthetic polypeptide
EIVLTQSPDFQSVTPKEKVTITCRASESVDTFGISFMNWFQQKPDQSPKLLIHEASNQGSGVPSRFSGSGSGTDFTLTINSLEAEDAATYYCAQTKEVPFTFGGGTKVEIK

SEQ ID NO: 201 - protein, 111 aa, Artificial Sequence
  Description of Artificial Sequence Synthetic polypeptide
EIVLTQSPDFQSVTPKEKVTITCRASESVDTFGISFMNWFQQKPDQSPKLLIHEASNQGSGVPSRFSGSGSGTDFTLTINSLEAEDAATYYCAQTKEVPNTFGGGTKVEIK

SEQ ID NO: 202 - protein, 111 aa, Artificial Sequence
  Description of Artificial Sequence Synthetic polypeptide
EIVLTQSPDFQSVTPKEKVTITCRASESVITFGISFMNWFQQKPDQSPKLLIHEASNTYSGVPSRFSGSGSGTDFTLTINSLEAEDAATYYCAQSKRVPFTFGGGTKVEIK

SEQ ID NO: 203 - protein, 111 aa, Artificial Sequence
  Description of Artificial Sequence Synthetic polypeptide
EIVLTQSPDFQSVTPKEKVTITCRASESVDTFGISFRNWFQQKPDQSPKLLIHEASNQGSGVPSRFSGSGSGTDFTLTINSLEAEDAATYYCQQSKEVPFTFGGGTKVEIK

SEQ ID NO: 204 - protein, 111 aa, Artificial Sequence
  Description of Artificial Sequence Synthetic polypeptide
EIVLTQSPDFQSVTPKEKVTITCRASESVDTFGISFMNWFQQKPDQSPKLLIHEASNPGSGVPSRFSGSGSGTDFTLTINSLEAEDAATYYCQQTKRVPFTFGGGTKVEIK

SEQ ID NO: 205 - protein, 111 aa, Artificial Sequence
  Description of Artificial Sequence Synthetic polypeptide
EIVLTQSPDFQSVTPKEKVTITCRASESVIHFGISFMNWFQQKPDQSPKLLIHEASNRGSGVPSRFSGSGSGTDFTLTINSLEAEDAATYYCAQSKEVPITFGGGTKVEIK

SEQ ID NO: 206 - protein, 111 aa, Artificial Sequence
  Description of Artificial Sequence Synthetic polypeptide
EIVLTQSPDFQSVTPKEKVTITCRASESVDTFGLSFMNWFQQKPDQSPKLLIHEASNPYSGVPSRFSGSGSGTDFTLTINSLEAEDAATYYCQQSKEVPFTFGGGTKVEIK

SEQ ID NO: 207 - protein, 111 aa, Artificial Sequence
  Description of Artificial Sequence Synthetic polypeptide
EIVLTQSPDFQSVTPKEKVTITCRASESVITFGISFINWFQQKPDQSPKLLIHEASNPYSGVPSRFSGSGSGTDFTLTINSLEAEDAATYYCAQTKRVPFTFGGGTKVEIK

SEQ ID NO: 208 - protein, 5 aa, Artificial Sequence
  Description of Artificial Sequence Synthetic peptide
SVWMN

SEQ ID NO: 209 - protein, 5 aa, Artificial Sequence
  Description of Artificial Sequence Synthetic peptide
STWMN

SEQ ID NO: 210 - protein, 17 aa, Artificial Sequence
  Description of Artificial Sequence Synthetic peptide
RIYLGDGDTNYNVKFKG

SEQ ID NO: 211 - protein, 15 aa, Artificial Sequence
  Description of Artificial Sequence Synthetic peptide
RASESVDHFGISFIN

SEQ ID NO: 212 - protein, 15 aa, Artificial Sequence
  Description of Artificial Sequence Synthetic peptide
RASESVITFGISFMN

SEQ ID NO: 213 - protein, 15 aa, Artificial Sequence
  Description of Artificial Sequence Synthetic peptide
RASESVDTFGISFIN

SEQ ID NO: 214 - protein, 15 aa, Artificial Sequence
  Description of Artificial Sequence Synthetic peptide
RASESVDTFGISFRN

SEQ ID NO: 215 - protein, 15 aa, Artificial Sequence
  Description of Artificial Sequence Synthetic peptide
RASESVIHFGISFMN

SEQ ID NO: 216 - protein, 15 aa, Artificial Sequence
  Description of Artificial Sequence Synthetic peptide
RASESVDTFGLSFMN

SEQ ID NO: 217 - protein, 15 aa, Artificial Sequence
  Description of Artificial Sequence Synthetic peptide
RASESVITFGISFIN

SEQ ID NO: 218 - protein, 7 aa, Artificial Sequence
  Description of Artificial Sequence Synthetic peptide
EASNPYS

SEQ ID NO: 219 - protein, 7 aa, Artificial Sequence
  Description of Artificial Sequence Synthetic peptide
EASNTYS

SEQ ID NO: 220 - protein, 7 aa, Artificial Sequence
  Description of Artificial Sequence Synthetic peptide
EASNPGS

SEQ ID NO: 221 - protein, 7 aa, Artificial Sequence
  Description of Artificial Sequence Synthetic peptide
EASNRGS

SEQ ID NO: 222 - protein, 9 aa, Artificial Sequence
  Description of Artificial Sequence Synthetic peptide
AQSKEVPIT

SEQ ID NO: 223 - protein, 9 aa, Artificial Sequence
  Description of Artificial Sequence Synthetic peptide
AQSKEVPFT

SEQ ID NO: 224 - protein, 9 aa, Artificial Sequence
  Description of Artificial Sequence Synthetic peptide
AQSKRVPFT

SEQ ID NO: 225 - protein, 9 aa, Artificial Sequence
  Description of Artificial Sequence Synthetic peptide
AQTKRVPFT

SEQ ID NO: 226 - protein, 9 aa, Artificial Sequence
  Description of Artificial Sequence Synthetic peptide
QQSKEVPIT

SEQ ID NO: 227 - protein, 9 aa, Artificial Sequence
  Description of Artificial Sequence Synthetic peptide
AQTKEVPFT

SEQ ID NO: 228 - protein, 9 aa, Artificial Sequence
  Description of Artificial Sequence Synthetic peptide
AQTKEVPNT

SEQ ID NO: 229 - protein, 9 aa, Artificial Sequence
  Description of Artificial Sequence Synthetic peptide
QQTKRVPFT

SEQ ID NO: 230 - protein, 5 aa, Artificial Sequence
  Description of Artificial Sequence Synthetic peptide
SXWMN

SEQ ID NO: 231 - protein, 17 aa, Artificial Sequence
  Description of Artificial Sequence Synthetic peptide
RIYXGDGDTNYXXKFKG

SEQ ID NO: 232 - protein, 12 aa, Artificial Sequence
  Description of Artificial Sequence Synthetic peptide
SGFITTVXDFDY

SEQ ID NO: 233 - protein, 15 aa, Artificial Sequence
  Description of Artificial Sequence Synthetic peptide
RASESVXXFGXSFXN

SEQ ID NO: 234 - protein, 7 aa, Artificial Sequence
  Description of Artificial Sequence Synthetic peptide
XASNXXS

SEQ ID NO: 235 - protein, 9 aa, Artificial Sequence
  Description of Artificial Sequence Synthetic peptide
XQXKXVPXT

SEQ ID NO: 236 - protein, 121 aa, Artificial Sequence
  Description of Artificial Sequence Synthetic polypeptide
EVQLVESGGGLVQPGGSLRLSCAASGFTFSSTWMNWVRQAPGKGLEWVGRIYPGDGDTNYNVKFKGRFTISRDDSKNSLYLQMNSLKTEDTAVYYCARSGFITTVRDFDYWGQGTLVTVSS

SEQ ID NO: 237 - protein, 121 aa, Artificial Sequence; identical to SEQ ID NO: 34
  Description of Artificial Sequence Synthetic polypeptide

SEQ ID NO: 238 - protein, 111 aa, Artificial Sequence; identical to SEQ ID NO: 68
  Description of Artificial Sequence Synthetic polypeptide

SEQ ID NO: 239 - protein, 4 aa, Artificial Sequence
  Description of Artificial Sequence Synthetic peptide
MRFA